(19) United States
(12) Patent Application Publication
(10) Pub. No.: US 2007/0259888 A1
(43) Pub. Date: Nov. 8, 2007

(54) Novel Compounds

(75) Inventors: Yun-Xing Cheng, Montreal (CA); Xuehong Luo, Montreal (CA); Miroslaw Tomaszewski, Montreal (CA)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: ASTRAZENECA AB, Sodertalje (SE)

(21) Appl. No.: 11/741,888

(22) Filed: Apr. 30, 2007

Related U.S. Application Data
(60) Provisional application No. 60/746,187, filed on May 2, 2006.

(51) Int. Cl. A61K 31/519; A61K 31/497; A61K 31/4545; A61K 31/454; C07D 401/02; C07D 403/02

(52) U.S. Cl. 514/255.05; 514/326; 514/259.3; 514/320; 544/281; 544/405; 546/207; 546/209

(57) ABSTRACT

Compounds of Formulae I, or pharmaceutically acceptable salts thereof: 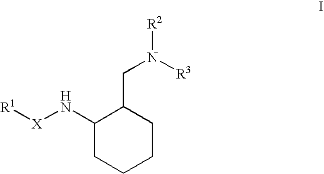
wherein X, R¹, R² and R³ are as defined in the specification as well as salts and pharmaceutical compositions including the compounds are prepared. They are useful in therapy, in particular in the management of pain.

This application claims benefit under 35 U.S.C. § 119(e) to Application No. 60/746,187, filed on May 2, 2006, which is hereby incorporated by reference in its entirety.

### BACKGROUND OF THE INVENTION

1. Field of the Invention

The present invention relates to agonists of muscarinic receptors. The present invention also provides compositions comprising such agonists, and methods therewith for treating muscarinic receptor mediated diseases. Particularly, the present invention is related to compounds that may be effective in treating pain, Alzheimer's disease, and/or schizophrenia.

2. Discussion of Relevant Technology

The neurotransmitter acetylcholine binds to two types of cholinergic receptors: the ionotropic family of nicotinic receptors and the metabotropic family of muscarinic receptors. Muscarinic receptors belong to the large superfamily of plasma membrane-bound G protein coupled receptors (GPCRs). and show a remarkably high degree of homology across species and receptor subtype. These M1-M5 muscarinic receptors are predominantly expressed within the parasympathetic nervous system which exerts excitatory and inhibitory control over the central and peripheral tissues and participate in a number of physiologic functions, including heart rate, arousal, cognition, sensory processing, and motor control.

Muscarinic agonists such as muscarine and pilocarpine, and antagonists, such as atropine have been known for over a century, but little progress has been made in the discovery of receptor subtype-selective compounds, thereby making it difficult to assign specific functions to the individual receptors. See, e.g., DeLapp, N. et al., “Therapeutic Opportunities for Muscarinic Receptors in the Central Nervous System,” J. Med. Chem., 43(23), pp. 4333-4353 (2000); Hulme, E. C. et al., “Muscarinic Receptor Subtypes,” Ann. Rev. Pharmacol. Toxicol., 30, pp. 633-673 (1990); Caulfield, M. P. et al., “Muscarinic Receptors-Characterization, Coupling, and Function,” Pharmacol. Ther., 58, pp. 319-379 (1993); Caulfield, M. P. et al., International Union of Pharmacology. XVII. Classification of Muscarinic Acetylcholine Receptors,” Pharmacol. Rev., 50, pp. 279-290 (1998).

The Muscarinic family of receptors is the target of a large number of pharmacological agents used for various diseases, including leading drugs for COPD, asthma, urinary incontinence, glaucoma, schizophrenia, Alzheimer's (AchE inhibitors), and Pain.

For example, direct acting muscarinic receptor agonists have been shown to be antinociceptive in a variety of animal models of acute pain (Bartolini A., Ghelardini C., Fantetti L., Malcangio M., Malmberg-Aiello P., Giotti A. Role of muscarinic receptor subtypes in central antinociception. Br. J. Pharmacol. 105:77-82, 1992; Capone F., Aloisi A. M., Carli G., Sacerdote P., Pavone F. Oxotremorine-induced modifications of the behavioral and neuroendocrine responses to formalin pain in male rats. Brain Res. 830:292-300, 1999).

A few studies have examined the role of muscarinic receptor activation in chronic or neuropathic pain states. In these studies, the direct and indirect elevation of cholinergic tone was shown to ameliorate tactile allodynia after intrathecal administration in a spinal ligation model of neuropathic pain in rats and these effects again were reversed by muscarinic antagonists (Hwang J.-H., Hwang K.-S., Leem J.-K., Park P.-H., Han S.-M., Lee D.-M. The antiallodynic effects of intrathecal cholinesterase inhibitors in a rat model of neuropathic pain. Anesthesiology 90:492-494, 1999; Lee E. J., Sim J. Y. Park J. Y., Hwang J. H., Park P. H., Han S. M. Intrathecal carbachol and clonidine produce a synergistic antiallodynic effect in rats with a nerve ligation injury. Can J Anaesth 49:178-84, 2002). Thus, direct or indirect activation of muscarinic receptors has been shown to elicit both acute analgesic activity and to ameliorate neuropathic pain. Muscarinic agonists and ACHE-Is are not widely used clinically owing to their propensity to induced a plethora of adverse events when administered to humans. The undesirable side-effects include excessive salivation and sweating, enhanced gastrointestinal motility, and bradycardia among other adverse events. These side-effects are associated with the ubiquitous expression of the muscarinic family of receptors throughout the body.

### DESCRIPTION OF THE EMBODIMENTS

To date, five subtypes of muscarinic receptors (M1-M5) have been cloned and sequenced from a variety of species, with differential distributions in the body.

Therefore, it was desirable to provide molecules would permit selective modulation, for example, of muscarinic receptors controlling central nervous function without also activating muscarinic receptors controlling cardiac, gastrointestinal or glandular functions.

There is also a need for methods for treating muscarinic receptor-mediated diseases.

There is also a need for modulators of muscarinic receptors that are selective as to subtypes M1-M5.

The term “Cₘ₋ₙ” or “Cₘ₋ₙ group” refers to any group having m to n carbon atoms.

The term “alkyl” refers to a saturated monovalent straight or branched chain hydrocarbon radical comprising 1 to about 12 carbon atoms. Illustrative examples of alkyls include, but are not limited to, C₁₋₆alkyl groups, such as methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, and hexyl, and longer alkyl groups, such as heptyl, and octyl. An alkyl can be unsubstituted or substituted with one or two suitable substituents.

The term “alkenyl” refers to a monovalent straight or branched chain hydrocarbon radical having at least one carbon-carbon double bond and comprising at least 2 up to about 12 carbon atoms. The double bond of an alkenyl can be unconjugated or conjugated to another unsaturated group. Suitable alkenyl groups include, but are not limited to C₂₋₆alkenyl groups, such as vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl. An alkenyl can be unsubstituted or substituted with one or two suitable substituents.

The term “cycloalkyl” refers to a saturated monovalent ring-containing hydrocarbon radical comprising at least 3 up to about 12 carbon atoms. Examples of cycloalkyls include, but are not limited to, C₃₋₇cycloalkyl groups, such as cyclopropyl, cyclobutyl, cyclopentyl, cyclohexyl, and cycloheptyl, and saturated cyclic and bicyclic terpenes. A cycloalkyl can be unsubstituted or substituted by one or two suitable substituents. Preferably, the cycloalkyl is a monocyclic ring or bicyclic ring.

The term “cycloalkenyl” refers to a monovalent ring-containing hydrocarbon radical having at least one carbon-carbon double bond and comprising at least 3 up to about 12 carbon atoms.

The term “aryl” refers to a monovalent hydrocarbon radical having one or more polyunsaturated carbon rings having aromatic character, (e.g., 4n+2 delocalized electrons) and comprising 5 up to about 14 carbon atoms.

The term “heterocycle” refers to a ring-containing structure or molecule having one or more multivalent heteroatoms, independently selected from N, O, P and S, as a part of the ring structure and including at least 3 and up to about 20 atoms in the ring(s). Heterocycle may be saturated or unsaturated, containing one or more double bonds, and heterocycle may contain more than one ring. When a heterocycle contains more than one ring, the rings may be fused or unfused. Fused rings generally refer to at least two rings share two atoms therebetween. Heterocycle may have aromatic character or may not have aromatic character.

The term “heteroaromatic” refers to a ring-containing structure or molecule having one or more multivalent heteroatoms, independently selected from N, O, P and S, as a part of the ring structure and including at least 3 and up to about 20 atoms in the ring(s), wherein the ring-containing structure or molecule has an aromatic character (e.g., 4n+2 delocalized electrons).

The term “heterocyclic group,” “heterocyclic moiety,” “heterocyclic,” or “heterocyclo” refers to a radical derived from a heterocycle by removing one or more hydrogens therefrom.

The term “heterocyclyl” refers a monovalent radical derived from a heterocycle by removing one hydrogen therefrom.

The term “heterocyclylene” refers to a divalent radical derived from a heterocycle by removing two hydrogens therefrom, which serves to links two structures together.

The term “heteroaryl” refers to a heterocyclyl having aromatic character.

The term “heterocycloalkyl” refers to a monocyclic or polycyclic ring comprising carbon and hydrogen atoms and at least one heteroatom, preferably, 1 to 3 heteroatoms selected from nitrogen, oxygen, and sulfur, and having no unsaturation. Examples of heterocycloalkyl groups include pyrrolidinyl, pyrrolidino, piperidinyl, piperidino, piperazinyl, piperazino, morpholinyl, morpholino, thiomorpholinyl, thiomorpholino, and pyranyl. A heterocycloalkyl group can be unsubstituted or substituted with one or two suitable substituents. Preferably, the heterocycloalkyl group is a monocyclic or bicyclic ring, more preferably, a monocyclic ring, wherein the ring comprises from 3 to 6 carbon atoms and form 1 to 3 heteroatoms, referred to herein as C₃₋₆heterocycloalkyl.

The term “heteroarylene” refers to a heterocyclylene having aromatic character.

The term “heterocycloalkylene” refers to a heterocyclylene that does not have aromatic character.

The term “six-membered” refers to a group having a ring that contains six ring atoms.

The term “five-membered” refers to a group having a ring that contains five ring atoms.

A five-membered ring heteroaryl is a heteroaryl with a ring having five ring atoms wherein 1, 2 or 3 ring atoms are independently selected from N, O and S.

Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl.

A six-membered ring heteroaryl is a heteroaryl with a ring having six ring atoms wherein 1, 2 or 3 ring atoms are independently selected from N, O and S.

Exemplary six-membered ring heteroaryls are pyridyl, pyrazinyl, pyrimidinyl, triazinyl and pyridazinyl.

Heterocycle includes, for example, monocyclic heterocycles such as: aziridine, oxirane, thiirane, azetidine, oxetane, thietane, pyrrolidine, pyrroline, imidazolidine, pyrazolidine, pyrazoline, dioxolane, sulfolane 2,3-dihydrofuran, 2,5-dihydrofuran tetrahydrofuran, thiophane, piperidine, 1,2,3,6-tetrahydro-pyridine, piperazine, morpholine, thiomorpholine, pyran, thiopyran, 2,3-dihydropyran, tetrahydropyran, 1,4-dihydropyridine, 1,4-dioxane, 1,3-dioxane, dioxane, homopiperidine, 2,3,4,7-tetrahydro-1H-azepine homopiperazine, 1,3-dioxepane, 4,7-dihydro-1,3-dioxepin, and hexamethylene oxide.

In addition, heterocycle includes aromatic heterocycles, for example, pyridine, pyrazine, pyrimidine, pyridazine, thiophene, furan, furazan, pyrrole, imidazole, thiazole, oxazole, pyrazole, isothiazole, isoxazole, 1,2,3-triazole, tetrazole, 1,2,3-thiadiazole, 1,2,3-oxadiazole, 1,2,4-triazole, 1,2,4-thiadiazole, 1,2,4-oxadiazole, 1,3,4-triazole, 1,3,4-thiadiazole, and 1,3,4-oxadiazole.

Additionally, heterocycle encompass polycyclic heterocycles, for example, indole, indoline, isoindoline, quinoline, tetrahydroquinoline, isoquinoline, tetrahydroisoquinoline, 1,4-benzodioxan, coumarin, dihydrocoumarin, benzofuran, 2,3-dihydrobenzofuran, isobenzofuran, chromene, chroman, isochroman, xanthene, phenoxathiin, thianthrene, indolizine, isoindole, indazole, purine, phthalazine, naphthyridine, quinoxaline, quinazoline, cinnoline, pteridine, phenanthridine, perimidine, phenanthroline, phenazine, phenothiazine, phenoxazine, 1,2-benzisoxazole, benzothiophene, benzoxazole, benzthiazole, benzimidazole, benztriazole, thioxanthine, carbazole, carboline, acridine, pyrolizidine, and quinolizidine.

In addition to the polycyclic heterocycles described above, heterocycle includes polycyclic heterocycles wherein the ring fusion between two or more rings includes more than one bond common to both rings and more than two atoms common to both rings. Examples of such bridged heterocycles include quinuclidine, diazabicyclo[2.2.1]heptane and 7-oxabicyclo[2.2.1]heptane.

Heterocyclyl includes, for example, monocyclic heterocyclyls, such as: aziridinyl, oxiranyl, thiiranyl, azetidinyl, oxetanyl, thietanyl, pyrrolidinyl, pyrrolinyl, imidazolidinyl, pyrazolidinyl, pyrazolinyl, dioxolanyl, sulfolanyl, 2,3-dihydrofuranyl, 2,5-dihydrofuranyl, tetrahydrofuranyl, thiophanyl, piperidinyl, 1,2,3,6-tetrahydro-pyridinyl, piperazinyl, morpholinyl, thiomorpholinyl, pyranyl, thiopyranyl, 2,3-dihydropyranyl, tetrahydropyranyl, 1,4-dihydropyridinyl, 1,4-dioxanyl, 1,3-dioxanyl, dioxanyl, homopiperidinyl, 2,3,4,7-tetrahydro-1H-azepinyl, homopiperazinyl, 1,3-dioxepanyl, 4,7-dihydro-1,3-dioxepinyl, and hexamethylene oxidyl.

In addition, heterocyclyl includes aromatic heterocyclyls or heteroaryl, for example, pyridinyl, pyrazinyl, pyrimidinyl, pyridazinyl, thienyl, furyl, furazanyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4 oxadiazolyl.

Additionally, heterocyclyl encompasses polycyclic heterocyclyls (including both aromatic or non-aromatic), for example, indolyl, indolinyl, isoindolinyl, quinolinyl, tetrahydroquinolinyl, isoquinolinyl, tetrahydroisoquinolinyl, 1,4-benzodioxanyl, coumarinyl, dihydrocoumarinyl, benzofuranyl, 2,3-dihydrobenzofuranyl, isobenzofuranyl, chromenyl, chromanyl, isochromanyl, xanthenyl, phenoxathiinyl, thianthrenyl, indolizinyl, isoindolyl, indazolyl, purinyl, phthalazinyl, naphthyridinyl, quinoxalinyl, quinazolinyl, cinnolinyl, pteridinyl, phenanthridinyl, perimidinyl, phenanthrolinyl, phenazinyl, phenothiazinyl, phenoxazinyl, 1,2-benzisoxazolyl, benzothiophenyl, benzoxazolyl, benzthiazolyl, benzimidazolyl, benztriazolyl, thioxanthinyl, carbazolyl, carbolinyl, acridinyl, pyrolizidinyl, and quinolizidinyl.

In addition to the polycyclic heterocyclyls described above, heterocyclyl includes polycyclic heterocyclyls wherein the ring fusion between two or more rings includes more than one bond common to both rings and more than two atoms common to both rings. Examples of such bridged heterocycles include quinuclidinyl, diazabicyclo[2.2.1]heptyl; and 7-oxabicyclo[2.2.1]heptyl.

The term “alkoxy” refers to radicals of the general formula —O—R, wherein R is selected from a hydrocarbon radical. Exemplary alkoxy includes methoxy, ethoxy, propoxy, isopropoxy, butoxy, t-butoxy, isobutoxy, cyclopropylmethoxy, allyloxy, and propargyloxy.

Halogen includes fluorine, chlorine, bromine and iodine.

“RT” or “rt” means room temperature.

In one aspect, an embodiment of the invention provides a compound of Formula I, a pharmaceutically acceptable salt thereof, diastereomers, enantiomers, or mixtures thereof: 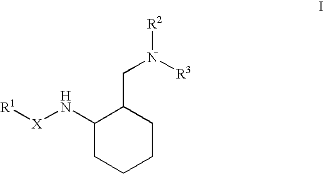
wherein

R¹ is selected from C₆₋₁₀aryl, C₂₋₉heteroaryl, C₃₋₅heterocycloalkyl, C₆₋₁₀aryl-C₁₋₃alkyl, C₂₋₉heteroaryl-C₁₋₃alkyl, C₃₋₅heterocycloalkyl-C₁₋₃alkyl, C₃₋₆cycloalkyl, C₃₋₆cycloalkyl-C₁₋₃alkyl, and C₁₋₆alkyl, wherein said C₆₋₁₀aryl, C₂₋₉heteroaryl, C₆₋₁₀aryl-C₁₋₃alkyl, C₆₋₁₀aryl-O—C₁₋₃alkyl, C₂₋₉heteroaryl-C₁₋₃alkyl, C₃₋₆cycloalkyl, C₃₋₆cycloalkyl-C₁₋₃alkyl, and C₁₋₆alkyl are optionally substituted with one or more group selected from C₆₋₁₀aryl, C₁₋₉heteroaryl, C₃₋₅heterocycloalkyl, C₆₋₁₀aryl-C₁₋₃alkyl, C₆₋₁₀aryl-O—C₁₋₃alkyl, C₂₋₉heteroaryl-C₁₋₃alkyl, C₃₋₅heterocycloalkyl-C₁₋₃alkyl, —CN, —SR, —OR, —O(CH₂)ₘ—OR, R, —C(═O)—R, —CO₂R, —SO₂R, —SO₂NR₂, halogen, —NO₂, —NR₂, —(CH₂)ₘNR₂, —(CH₂)ₘNHC(═O)—NR₂, —(CH₂)ₘNHC(═O)—R, —(CH₂)ₘN[C(═O)—R]₂, —NHC(═O)—R, —N[C(═O)R]₂, —(CH₂)ₘNHS(═O)₂—R, and —C(═O)—NR₂;

R² and R³ are independently selected from C₁₋₆alkyl, C₂₋₆alkenyl, and C₁₋₆alkoxy wherein said C₁₋₆alkyl, C₂₋₆alkenyl, and C₁₋₆alkoxy are optionally substituted by one or more groups selected from amino, halogen, C₁₋₆alkoxy and —CN; or R² and R³ together with the nitrogen connected thereto form a heterocycloalkyl, wherein said heterocycloalkyl is optionally substituted with one or more group selected from C₆₋₁₀aryl, C₂₋₉heteroaryl, C₃₋₆cycloalkyl, C₃₋₅heterocycloalkyl, C₆₋₁₀aryl-C₁₋₃alkyl, C₂₋₉heteroaryl-C₁₋₃alkyl, C₃₋₅heterocycloalkyl-C₁₋₃alkyl, —CN, —SR, —OR, —(CH₂)ₘOR, R, —CO₂R; —SO₂R; —SO₂NR₂, halogen, —NO₂, —NR₂, —(CH₂)ₘNR₂, and —C(═O)—NR₂;

each R is independently hydrogen, C₁₋₆alkyl, C₂₋₆alkenyl or halogenated C₁₋₆alkyl; and

X is selected from —C(═O)—, —C(═O)—NH—, —C(═O)—O— and —S(═O)₂—,

with a proviso that

when X is —C(═O)— and R² and R³ together with the nitrogen connected thereto form said piperidinyl; R¹ is not 4-amino-5-chloro-2-alkoxylphenyl, 4-amino-5-chloro-2-cycloalkoxyphenyl, 4-amino-5-chloro-2-cycloalkyl-alkoxy-phenyl, 4-butoxyphenyl, 3-butoxyphenyl, 4-pentyloxyphenyl, 4-isobutoxyphenyl, 4-benzoyloxyphenyl and 7-(2,3-dihydro)benzofuranyl.

In a particular embodiment, the R² and R³ of formula I together with the nitrogen connected thereto form a heterocycloalkyl, wherein said heterocycloalkyl is optionally substituted with one or more group selected from C₆₋₁₀aryl, C₂₋₉heteroaryl, C₃₋₆cycloalkyl, C₃₋₅heterocycloalkyl, C₆₋₁₀aryl-C₁₋₃alkyl, C₂₋₉heteroaryl-C₁₋₃alkyl, C₃₋₅heterocycloalkyl-C₁₋₃alkyl, —CN, —SR, —OR, —(CH₂)ₘOR, R, —CO₂R; —SO₂R; —SO₂NR₂, halogen, —NO₂, —NR₂, —(CH₂)ₘNR₂, and —C(═O)—NR₂.

In another embodiment, R² and R³ of formula I together with the nitrogen connected thereto form a group selected from piperidinyl, 1,4-dixo-8-azaspiro[4,5]dec-8-yl, piperazinyl, methyl (2-phenylethyl)amino, methyl(pyridin-3-ylmethyl)amino, (4-ethylbenzyl)(methyl)amino, methyl(1-methylpyrrolidin-3-yl)amino, methyl(3-methylbutyl)amino, methyl(propyl)amino, methyl(butyl)amino, butyl(ethyl)amino, diethylamino, benzyl(methyl)amino, morpholin-4-yl, pyrrolidin-1-yl, and azepan-1-yl, wherein said piperidinyl, 1,4-dixo-8-azaspiro[4,5]dec-8-yl, piperazinyl, methyl(2-phenylethyl)amino, methyl(pyridin-3-ylmethyl)amino, (4-ethylbenzyl)(methyl)amino, methyl(1-methylpyrrolidin-3-yl)amino, methyl(3-methylbutyl)amino, methyl(propyl)amino, methyl(butyl)amino, butyl(ethyl)amino, diethylamino, benzyl(methyl)amino, morpholin-4-yl, pyrrolidin-1-yl, and azepan-1-yl are optionally substituted with one or more group selected from C₆₋₁₀aryl, C₂₋₉heteroaryl, C₃₋₆cycloalkyl, C₃₋₅heterocycloalkyl, C₆₋₁₀aryl-C₁₋₃alkyl, C₂₋₉heteroaryl-C₁₋₃alkyl, C₃₋₅heterocycloalkyl-C₁₋₃alkyl, —CN, —SR, —OR, —(CH₂)ₘOR, R, —CO₂R; —SO₂R; —SO₂NR₂, halogen, —NO₂, —NR₂, —(CH₂)ₘNR₂, and —C(═O)—NR₂.

In another particular embodiment, R¹ of formula I is selected from 2-cyclopentylethyl, cyclopropylmethyl, methyl, cyclohexyl, cyclopentylmethyl, chromanyl, ethyl, pentyl, 2-phenylethyl, phenyl, benzyl, pyridinyl, pyridinylethyl, 1-benzofuranyl, benzothienyl, furyl, imidazolyl, pyrazolo[1,5-a]pyrimidinyl, pyrazinyl, 1,3-benzothiazolyl, indolyl, indazolyl, thienyl, 1,3-benzodioxinyl, tetrahydro-2H-pyran-4-ylmethyl, 1-H-1,2,3-benzotriazol-1-yl, 2-(thien-2-yl)ethyl, (1-benzofuran-4-yl)methyl, 1,3-oxazolyl, 1H-pyrazol-1-yl, 2,3-dihydro-1-benzofuran-5-yl, 1,3-benzodioxol-5-yl, 2-oxo-2,3-dihydro-2H-benzimidazolyl, isoxazolyl, imidazo[1,2,a]pyridinyl, 2-3-dioxo-2,3-dihydro-1H-indol-1-yl, 3,4-dihydro-2H-1,4-benzoxazinyl; pyrazolyl, 1H-tetrazol-1-yl-methyl, and 3,4-dihydro-2H-1,5-benzodioxepinyl, optionally substituted by 1H-pyrazol-1-yl, fluoro, chloro, trifluoromethyl, methoxy, difluoromethoxy, trifluoromethoxy, 2-methoxyethoxy, 2-ethoxyethoxy, t-butyl, cyano, bromo, 1,3-oxazol-5-yl, 1H-imidazol-1-yl, (4-oxopiperidin-1-yl)carbonyl, pyridin-3-ylmethyl, [(butylamino)carbonyl]amino, 1,1-dioxidothiomorpholin-4-yl, aminosulfonyl, morpholin-4-yl, diethylaminomethyl, acetyl, (3-oxo-2,3-dihydro-4H-1,4-benzoxazin-4-yl)methyl, 1-oxo-indan-4-yl, dimethylaminomethyl, methyl, pyrrolidin-1-yl, ethylthio, acetylamino, dimethylamino, 1H-pyrrol-1-yl, ethyl, ethoxy, fluorophenoxy, propyl, phenyl, methoxycarbonyl, diacetylamino, (methylsulfonylamino)methyl, (cyclopropylsulfonylamino)methyl, 1H-tetrazol-1-yl, pyrazolyl, methylaminocarbonylamino, dimethylaminocarbonylamino, and (methylthio)pyrimidin-4-yl.

In another particular embodiment, R² and R³ formula I together with the nitrogen connected thereto form a group selected from piperidinyl, 1,4-dixo-8-azaspiro[4,5]dec-8-yl, piperazinyl, methyl(2-phenylethyl)amino, methyl(pyridin-3-ylmethyl)amino, (4-ethylbenzyl)(methyl)amino, methyl(1-methylpyrrolidin-3-yl)amino, methyl(3-methylbutyl)amino, methyl(propyl)amino, methyl(butyl)amino, butyl(ethyl)amino, diethylamino, benzyl(methyl)amino, morpholin-4-yl, pyrrolidin-1-yl, and azepan-1-yl, wherein said piperidinyl, 1,4-dixo-8-azaspiro[4,5]dec-8-yl, piperazinyl, methyl(2-phenylethyl)amino, methyl(pyridin-3-ylmethyl)amino, (4-ethylbenzyl)(methyl)amino, methyl(1-methylpyrrolidin-3-yl)amino, methyl(3-methylbutyl)amino, methyl(propyl)amino, methyl(butyl)amino, butyl(ethyl)amino, diethylamino, benzyl(methyl)amino, morpholin-4-yl, pyrrolidin-1-yl, and azepan-1-yl are optionally substituted with one or more group selected from phenyl, benzyl, methyl, fluoro, trifluoromethyl, methoxy, allyloxy, (2E)-but-2-en-1-yloxy, (allyloxy)methyl, methoxymethyl, ethoxymethyl, propyl, butyl, pentyl, hexyl, cyclopentyl, pyridin-4-ylmethyl, ethoxy, butoxy, 2-methoxyethoxy, cyclohexyl, and thienylmethyl.

In another particular embodiment, R² and R³ of formula I together with the nitrogen connected thereto form a group selected from piperidinyl, wherein said piperidinyl is optionally substituted with one or more group selected from phenyl, benzyl, methyl, fluoro, trifluoromethyl, methoxy, allyloxy, (2E)-but-2-en-1-yloxy, (allyloxy)methyl, methoxymethyl, ethoxymethyl, propyl, butyl, pentyl, hexyl, cyclopentyl, pyridin-4-ylmethyl, ethoxy, butoxy, 2-methoxyethoxy, cyclohexyl, and thienylmethyl.

In a further particular embodiment, the compounds are selected from
trans-(+/−)-4-fluoro-N-[2-(piperidin-1-ylmethyl)cyclohexyl]benzamide;
trans-(+/−)-N-[2-(piperidin-1-ylmethyl)cyclohexyl]-6-(1H-pyrazol-1-yl)nicotinamide;
trans-(+/−)-N-[2-(piperidin-1-ylmethyl)cyclohexyl]-6-(trifluoromethyl)nicotinamide;
trans-(+/−)-N-[2-(piperidin-1-ylmethyl)cyclohexyl]-4-(1H-pyrazol-1-yl)benzamide;
trans-(+/−)-5-chloro-N-[2-(piperidin-1-ylmethyl)cyclohexyl]-1-benzofuran-2-carboxamide;
trans-(+/−)-2-(4-methoxyphenyl)-N-[2-(piperidin-1-ylmethyl)cyclohexyl]acetamide;
trans-(+/−)-4-(difluoromethoxy)-N-[2-(piperidin-1-ylmethyl)cyclohexyl]benzamide;
trans-(+/−)-4-(2-methoxyethoxy)-N-[2-(piperidin-1-ylmethyl)cyclohexyl]benzamide;
trans-(+)-4-(2-methoxyethoxy)-N-[2-(piperidin-1-ylmethyl)cyclohexyl]benzamide;
trans-(−)4-(2-methoxyethoxy)-N-[2-(piperidin-1-ylmethyl)cyclohexyl]benzamide;
trans-(+/−)-3-cyclopentyl-N-[2-(piperidin-1-ylmethyl)cyclohexyl]propanamide;
trans-(+/−)-3-(4-chlorophenyl)-N-[2-(piperidin-1-ylmethyl)cyclohexyl]propanamide;
trans-(+/−)-3-(2-methoxyphenyl)-N-[2-(piperidin-1-ylmethyl)cyclohexyl]propanamide;
trans-(+/−)-4-tert-butyl-N-[2-(piperidin-1-ylmethyl)cyclohexyl]benzamide;
trans-(+/−)-4-methoxy-N-[2-(piperidin-1-ylmethyl)cyclohexyl]benzamide;
trans-(+/−)-4-cyano-N-[2-(piperidin-1-ylmethyl)cyclohexyl]benzamide;
trans-(+/−)-4-bromo-N-[2-(piperidin-1-ylmethyl)cyclohexyl]benzamide;
trans-(+/−)-4-chloro-N-[2-(piperidin-1-ylmethyl)cyclohexyl]benzamide;
trans-(+/−)-6-(1H-imidazol-1-yl)-N-[2-(piperidin-1 ylmethyl)cyclohexyl]nicotinamide;
trans-(+/−)-4-(1,3-oxazol-5-yl)-N-[-2-(piperidin-1-ylmethyl)cyclohexyl]benzamide;
trans-(+/−)-6-methoxy-N-[2-(piperidin-1-ylmethyl)cyclohexyl]nicotinamide;
trans-(+/−)-4-(1H-imidazol-1-yl)-N-[2-(piperidin-1-ylmethyl)cyclohexyl]benzamide;
trans-(+/−)-4-[(4-oxopiperidin-1-yl)carbonyl]-N-[2-(piperidin-1-ylmethyl)cyclohexyl]benzamide;
trans-(+/−)-N-[2-(piperidin-1-ylmethyl)cyclohexyl]-2-pyridin-3-ylacetamide;
trans-(+/−)-2-{[(butylamino)carbonyl]amino}-N-[2-(piperidin-1-ylmethyl)cyclohexyl]benzamide;
trans-(+/−)-4-(1,1-dioxidothiomorpholin-4-yl)-N-[2-(piperidin-1-ylmethyl)cyclohexyl]benzamide;
trans-(+/−)-4-(aminosulfonyl)-N-[2-(piperidin-1-ylmethyl)cyclohexyl]benzamide;
trans-(+/−)-2-morpholin-4-yl-N-[2-(piperidin-1-ylmethyl)cyclohexyl]isonicotinamide;
trans-(+/−)-4-[(diethylamino)methyl]-N-[2-(piperidin-1-ylmethyl)cyclohexyl]benzamide;
trans-(+/−)-N-[2-(piperidin-1-ylmethyl)cyclohexyl]-1-benzothiophene-3-carboxamide;
trans-(+/−)-4-acetyl-N-[2-(piperidin-1-ylmethyl)cyclohexyl]benzamide;
trans-(+/−)-4-[(3-oxo-2,3-dihydro-4H-1,4-benzoxazin-4-yl)methyl]-N-[2-(piperidin-1-ylmethyl)cyclohexyl]benzamide;
trans-(+/−)-1-oxo-N-[2-(piperidin-1-ylmethyl)cyclohexyl]indane-4-carboxamide;
trans-(+/−)-5-[(dimethylamino)methyl]-N-[2-(piperidin-1-ylmethyl)cyclohexyl]-2-furamide;
trans-(+/−)-1-methyl-N-[2-(piperidin-1-ylmethyl)cyclohexyl]-1H-imidazole-4-carboxamide;
trans-(+/−)-2-(4-chlorophenyl)-N-[2-(piperidin-1-ylmethyl)cyclohexyl]acetamide;
trans-(+/−)-N-[2-(piperidin-1-ylmethyl)cyclohexyl]-6-pyrrolidin-1-ylnicotinamide;
trans-(+/−)-5-methyl-N-[2-(piperidin-1-ylmethyl)cyclohexyl]-7-(trifluoromethyl)pyrazolo[1,5-a]pyrimidine-2-carboxamide;
trans-(+/−)-N-[2-(piperidin-1-ylmethyl)cyclohexyl]pyrazine-2-carboxamide;
trans-(+/−)-4-(ethylthio)-N-[2-(piperidin-1-ylmethyl)cyclohexyl]benzamide;
trans-(+/−)-N-[2-(piperidin-1-ylmethyl)cyclohexyl]-1,3-benzothiazole-6-carboxamide;
trans-(+/−)-4-(acetylamino)-N-[2-(piperidin-1-ylmethyl)cyclohexyl]benzamide;
trans-(+/−)-5-methoxy-N-[2-(piperidin-1-ylmethyl)cyclohexyl]-1H-indole-2-carboxamide;
trans-(+/−)-N-[2-(piperidin-1-ylmethyl)cyclohexyl]thiophene-3-carboxamide;
trans-(+/−)-2-phenyl-N-[2-(piperidin-1-ylmethyl)cyclohexyl]acetamide;
trans-(+/−)-N-[2-(piperidin-1-ylmethyl)cyclohexyl]-4-(trifluoromethoxy)benzamide;
trans-(+/−)-3-(2-chlorophenyl)-N-[2-(piperidin-1-ylmethyl)cyclohexyl]propanamide;
trans-(+/−)-N-[2-(piperidin-1-ylmethyl)cyclohexyl]pyrazolo[1,5-a]pyrimidine-3-carboxamide;
trans-(+/−)-N-[2-(piperidin-1-ylmethyl)cyclohexyl]-4-cyano benzamide;
trans-(+/−)-3-(3-chlorophenyl)-N-[2-(piperidin-1-ylmethyl)cyclohexyl]propanamide;
trans-(+/−)-6-fluoro-N-[2-(piperidin-1-ylmethyl)cyclohexyl]-4H-1,3-benzodioxine-8-carboxamide;
trans-(+/−)-N-[2-(piperidin-1-ylmethyl)cyclohexyl]-2-(tetrahydro-2H-pyran-4-yl)acetamide;
trans-(+/−)-4-chloro-2,5-difluoro-N-[2-(piperidin-1-ylmethyl)cyclohexyl]benzamide;
trans-(+/−)-N-[2-(piperidin-1-ylmethyl)cyclohexyl]-1H-indole-6-carboxamide;
trans-(+/−)-3-(1H-1,2,3-benzotriazol-1-yl)-N-[2-(piperidin-1-ylmethyl)cyclohexyl]propanamide;
trans-(+/−)-N-[2-(piperidin-1-ylmethyl)cyclohexyl]-3-(2-thienyl)propanamide;
trans-(+/−)-2-(1-benzofuran-4-yl)-N-[2-(piperidin-1-ylmethyl)cyclohexyl]acetamide;
trans-(+/−)-4-(dimethylamino)-N-[2-(piperidin-1-ylmethyl)cyclohexyl]benzamide;
trans-(+/−)-N-[2-(piperidin-1-ylmethyl)cyclohexyl]-3-pyridin-3-ylpropanamide;
trans-(+/−)-4,6-dimethyl-N-[2-(piperidin-1-ylmethyl)cyclohexyl]nicotinamide;
trans-(+/−)-3-(5-methyl-2-furyl)-N-[2-(piperidin-1-ylmethyl)cyclohexyl]-1H-pyrazole-5-carboxamide;
trans-(+/−)-2-cyclopropyl-N-[2-(piperidin-1-ylmethyl)cyclohexyl]acetamide;
trans-(+/−)-5-methoxy-N-[2-(piperidin-1-ylmethyl)cyclohexyl]-1-benzofuran-2-carboxamide;
trans-(+/−)-N-[2-(piperidin-1-ylmethyl)cyclohexyl]-1H-indazole-3-carboxamide;
trans-(+/−)-6-(ethylthio)-N-[2-(piperidin-1-ylmethyl)cyclohexyl]nicotinamide;
trans-(+/−)-N-[2-(piperidin-1-ylmethyl)cyclohexyl]-4-(1H-pyrrol-1-yl)benzamide;
trans-(+/−)-N-[2-(piperidin-1-ylmethyl)cyclohexyl]-1H-indole-4-carboxamide;
trans-(+/−)-2-chloro-N-[2-(piperidin-1-ylmethyl)cyclohexyl]benzamide;
trans-(+/−)-3-cyano-N-[2-(piperidin-1-ylmethyl)cyclohexyl]benzamide;
trans-(+/−)-2-methyl-N-[2-(piperidin-1-ylmethyl)cyclohexyl]-5-(trifluoromethyl)-1,3-oxazole-4-carboxamide;
trans-(+/−)-3-chloro-4-methyl-N-[2-(piperidin-1-ylmethyl)cyclohexyl]thiophene-2-carboxamide;
trans-(+/−)-3-(5-methyl-1H-pyrazol-1-yl)-N-[2-(piperidin-1-ylmethyl)cyclohexyl]propanamide;
trans-(+/−)-3-methoxy-N-[2-(piperidin-1-ylmethyl)cyclohexyl]benzamide;
trans-(+/−)-2-(2,3-dihydro-1-benzofuran-5-yl)-N-[2-(piperidin-1-ylmethyl)cyclohexyl]acetamide;
trans-(+/−)-N-[2-(piperidin-1-ylmethyl)cyclohexyl]-1,3-benzodioxole-5-carboxamide;
trans-(+/−)-5-methyl-N-[2-(piperidin-1-ylmethyl)cyclohexyl]thiophene-2-carboxamide;
trans-(+/−)-1-ethyl-5-methyl-N-[2-(piperidin-1-ylmethyl)cyclohexyl]-1H-pyrazole-4-carboxamide;
trans-(+/−)-5-ethoxy-N-[2-(piperidin-1-ylmethyl)cyclohexyl]-2-furamide;
trans-(+/−)-3-(4-fluorophenoxy)-N-[2-(piperidin-1-ylmethyl)cyclohexyl]propanamide;
trans-(+/−)-3-fluoro-4-methoxy-N-[2-(piperidin-1-ylmethyl)cyclohexyl]benzamide;
trans-(+/−)-N-[2-(piperidin-1-ylmethyl)cyclohexyl]-4-propylbenzamide;
trans-(+/−)-N-[2-(piperidin-1-ylmethyl)cyclohexyl]hexanamide;
trans-(+/−)-4-butoxy-N-[2-(piperidin-1-ylmethyl)cyclohexyl]benzamide;
trans-(+/−)-4-chloro-2-fluoro-N-[2-(piperidin-1-ylmethyl)cyclohexyl]benzamide;
trans-(+/−)-2-oxo-N-[2-(piperidin-1-ylmethyl)cyclohexyl]-2,3-dihydro-1H-benzimidazole-5-carboxamide;
trans-(+/−)-2-(4-ethoxyphenyl)-N-[2-(piperidin-1-ylmethyl)cyclohexyl]acetamide;
trans-(+/−)-3-phenyl-N-[2-(piperidin-1-ylmethyl)cyclohexyl]isoxazole-5-carboxamide;
trans-(+/−)-2-methoxy-5-methyl-N-[2-(piperidin-1-ylmethyl)cyclohexyl]benzamide;
trans-(+/−)-4-methoxy-N-{2-[(4-phenylpiperidin-1-yl)methyl]cyclohexyl}benzamide;
trans-(+/−)-N-[2-(1,4-dioxa-8-azaspiro[4,5]dec-8-ylmethyl)cyclohexyl]-4-methoxybenzamide;
trans-(+/−)-N-{2-[(3,5-dimethylpiperidin-1-yl)methyl]cyclohexyl}-4-methoxybenzamide;
trans-(+/−)-N-{2-[(4-fluoropiperidin-1-yl)methyl]cyclohexyl}-4-methoxybenzamide;
trans-(+/−)-4-methoxy-N-(2-{[4-(trifluoromethyl)piperidin-1-yl]methyl}cyclohexyl)benzamide;
trans-(+/−)-4-methoxy-N-{2-[(4-methoxypiperidin-1-yl)methyl]cyclohexyl}benzamide;
trans-(+/−)-4-methoxy-N-(2-{[3-(trifluoromethyl)piperidin-1-yl]methyl}cyclohexyl)benzamide;
trans-(+/−)-4-methoxy-N-{2-[(3-phenylpiperidin-1-yl)methyl]cyclohexyl}benzamide;
trans-(+/−)-N-[2-({3-[(allyloxy)methyl]piperidin-1-yl}methyl)cyclohexyl]-4-methoxybenzamide;
trans-(+/−)-N-[2-({3-[(allyloxy)methyl]piperidin-1-yl}methyl)cyclohexyl]-6-(1H-pyrazol-1-yl)nicotinamide;
trans-(+/−)-N-(2-{[3-(methoxymethyl)piperidin-1-yl]methyl}cyclohexyl)-6-(1H-pyrazol-1-yl)nicotinamide;
trans-(+/−)-N-(2-{[3-(ethoxymethyl)piperidin-1-yl]methyl}cyclohexyl)-6-(1H-pyrazol-1-yl)nicotinamide;
trans-(+/−)-N-{2-[(3-pentylpiperidin-1-yl)methyl]cyclohexyl}-6-(1H-pyrazol-1-yl)nicotinamide;
trans-(+/−)-N-{2-[(3-pentylpiperidin-1-yl)methyl]cyclohexyl}-4-(1H-pyrazol-1-yl)benzamide;
trans-(+/−)-6-(1H-imidazol-1-yl)-N-{2-[(3-pentylpiperidin-1-yl)methyl]cyclohexyl}nicotinamide;
trans-(+/−)-N-{2-[(3-pentylpiperidin-1-yl)methyl]cyclohexyl}-6-pyrrolidin-1-ylnicotinamide;
trans(±)6-(1H-imidazol-1-yl)-N-(-2-{[(3R)-3-pentylpiperidin-1-yl]methyl}cyclohexyl)nicotinamide;
trans(±)6-(1H-imidazol-1-yl)-N-(2-{[(3S)-3-pentylpiperidin-1-yl]methyl}cyclohexyl)nicotinamide;
trans-(+/−)-N-{(2-[(3-hexylpiperidin-1-yl)methyl]cyclohexyl}-6-(1H-pyrazol-1-yl)nicotinamide;
trans-(+/−)-N-{2-[(3-hexylpiperidin-1-yl)methyl]cyclohexyl}-6-(1H-imidazol-1-yl)nicotinamide;
trans-(+/−)-N-{2-[(3-hexylpiperidin-1-yl)methyl]cyclohexyl}-4-(1H-pyrazol-1-yl)benzamide;
trans-(+/−)-N-{2-[(3-hexylpiperidin-1-yl)methyl]cyclohexyl}-4-pyrrolidin-1-ylbenzamide;
trans-(+/−)-N-{(2-[(3-butylpiperidin-1-yl)methyl]cyclohexyl}-6-(1H-pyrazol-1-yl)nicotinamide;
trans-(+/−)-N-{2-[(3-butylpiperidin-1-yl)methyl]cyclohexyl}-4-pyrrolidin-1-ylbenzamide;
trans-(+/−)-N-{2-[(3-butylpiperidin-1-yl)methyl]cyclohexyl}-6-(1H-imidazol-1-yl)nicotinamide;
trans-(+/−)-N-{2-[(3-butylpiperidin-1-yl)methyl]cyclohexyl}-4-(1H-pyrazol-1-yl)benzamide;
cis-(+/−)-N-{2-[(3-butylpiperidin-1-yl)methyl]cyclohexyl}-6-(1H-imidazol-1-yl)nicotinamide;
trans-(+/−)-N-(2-{[4-(Allyloxy)piperidin-1-yl]methyl}cyclohexyl)-6-(1H-pyrazol-1-yl)nicotinamide;
trans-(+/−)-N-[2-({4-[(2E)-But-2-en-1-yloxy]piperidin-1-yl}methyl)cyclohexyl]-6-(1H-pyrazol-1-yl)nicotinamide;
trans-(+/−)-N-[2-({3-[(Allyloxy)methyl]piperidin-1-yl}methyl)cyclohexyl]-6-pyrrolidin-1-ylnicotinamide;
trans-(+/−)-N-[2-({3-[(Allyloxy)methyl]piperidin-1-yl}methyl)cyclohexyl]-4-(1H-pyrazol-1-yl)benzamide;
trans-(+/−)-N-[2-({3-[(Allyloxy)methyl]piperidin-1-yl}methyl)cyclohexyl]-6-(1H-imidazol-1-yl)nicotinamide;
trans-(±)-N-2-({3-[(Allyloxy)methyl]piperidin-1-yl}methyl)cyclohexyl]-4-bromobenzamide;
Trans-(±)-(N-2-({3-[(Allyloxy)methyl]piperidin-1-yl}methyl)cyclohexyl]-3-(4-chlorophenyl)propanamide
Trans-(±)-N-[2-({3-[(Allyloxy)methyl]piperidin-1-yl}methyl)cyclohexyl]-3-(2-methoxyphenyl)propanamide
Trans-(±)-N-[2-({3-[(Allyloxy)methyl]piperidin-1-yl}methyl)cyclohexyl]-4-cyanobenzamide
Trans-(±)-N-[(2-({3-[(Allyloxy)methyl]piperidin-1-yl}methyl)cyclohexyl]-4-fluorobenzamide
Trans-(±)-N-[(2-({3-[(Allyloxy)methyl]piperidin-1-yl}methyl)cyclohexyl]-4-chlorobenzamide
Trans-(±)-N-[2-({3-[(Allyloxy)methyl]piperidin-1-yl}methyl)cyclohexyl]-4-[(diethylamino)methyl]benzamide
Trans-(±)-N-[2-({3-[(Allyloxy)methyl]piperidin-1-yl}methyl)cyclohexyl]-4-[(4-methylpiperazin-1-yl)methyl]benzamide;
Trans(±)[-2-({(3R)-3-[(Allyloxy)methyl]piperidin-1-yl}methyl)cyclohexyl]-6-(1H-imidazol-1-yl)nicotinamide;
Trans-(±)-[2-({(3S)-3-[(Allyloxy)methyl]piperidin-1-yl}methyl)cyclohexyl]-6-(1H-imidazol-1-yl)nicotinamide;
trans-(+/−)-N-{2-[(4-benzylpiperidin-1-yl)methyl]cyclohexyl}-6-(1H-pyrazol-1-yl)nicotinamide;
trans-(+/−)-N-{2-[(4-cyclopentylpiperazin-1-yl)methyl]cyclohexyl}-6-(1H-pyrazol-1-yl)nicotinamide;
trans-(+/−)-N-(2-{[methyl(2-phenylethyl)amino]methyl}cyclohexyl)-6-(1H-pyrazol-1-yl)nicotinamide;
trans-(+/−)-6-(1H-pyrazol-1-yl)-N-(2-{[4-(pyridin-4-ylmethyl)piperazin-1-yl]methyl}cyclohexyl)nicotinamide;
trans-(+/−)-N-(2-H[methyl(pyridin-3-ylmethyl)amino]methylcyclohexyl)-6-(1H-pyrazol-1-yl)nicotinamide;
trans-(+/−)-N-(2-{[(4-ethylbenzyl)(methyl)amino]methyl}cyclohexyl)-6-(1H-pyrazol-1-yl)nicotinamide;
trans-(+/−)-N-(2-{[methyl(1-methylpyrrolidin-3-yl)amino]methyl}cyclohexyl)-6-(1H-pyrazol-1-yl)nicotinamide;
trans-(+/−)-N-(2-{[methyl(3-methylbutyl)amino]methyl}cyclohexyl)-6-(1H-pyrazol-1-yl)nicotinamide;
trans-(+/−)-N-(2-{[methyl(propyl)amino]methyl}cyclohexyl)-6-(1H-pyrazol-1-yl)nicotinamide;
trans-(+/−)-N-(2-{[benzyl(methyl)amino]methyl}cyclohexyl)-6-(1H-pyrazol-1-yl)nicotinamide;
trans-(+/−)-N-{2-[(4-propylpiperidin-1-yl)methyl]cyclohexyl}-6-(1H-pyrazol-1-yl)nicotinamide;
trans-(+/−)-N-(2-{[2-(methoxymethyl)piperidin-1-yl]methyl}cyclohexyl)-6-(1H-pyrazol-1-yl)nicotinamide;
trans-(+/−)-N-(2-{[butyl(methyl)amino]methyl}cyclohexyl)-6-(1H-pyrazol-1-yl)nicotinamide;
trans-(+/−)-N-(2-{[butyl(ethyl)amino]methyl}cyclohexyl)-6-(1H-pyrazol-1-yl)nicotinamide;
trans-(+/−)-6-(1H-pyrazol-1-yl)-N-(2-{[2-(3-thienylmethyl)piperidin-1-yl]methyl}cyclohexyl)nicotinamide;
trans-(+/−)-N-{2-[(4,4-difluoropiperidin-1-yl)methyl]cyclohexyl}-4-methoxybenzamide;
trans-(+/−)-4-methoxy-N-{2-[(4-methylpiperidin-1-yl)methyl]cyclohexyl}benzamide;
trans-(+/−)-4-(2-methoxyethoxy)-N-{2-[(4-methyl piperidin-1-yl)methyl]cyclohexyl}benzamide;
trans-(+/−)-4-methoxy-N-[2-(morpholin-4-ylmethyl)cyclohexyl]benzamide;
cis-(+/−)-4-(2-ethoxyethoxy)-N-[2-(piperidin-1-ylmethyl)cyclohexyl]benzamide;
cis-(+/−)-4-(2-ethoxyethoxy)-N-[2-(pyrrolidin-1-ylmethyl)cyclohexyl]benzamide;
cis-(+/−)-N-{2-[(diethylamino)methyl]cyclohexyl}-4-(2-ethoxyethoxy)benzamide;
trans-(+/−)-4-(2-ethoxyethoxy)-N-[2-(piperidin-1-ylmethyl)cyclohexyl]benzamide;
trans-(+/−)-N-[2-(azepan-1-ylmethyl)cyclohexyl]-4-(2-ethoxyethoxy)benzamide;
trans-(+/−)-N-[2-[(diethylamino)methyl]cyclohexyl]-4-(2-ethoxyethoxy)benzamide;
trans-(+/−)-N-(4-chlorophenyl)-N′-[2-(piperidin-1-ylmethyl)cyclohexyl]urea;
trans-(+/−)-N-(4-cyanophenyl)-N′-[2-(piperidin-1-ylmethyl)cyclohexyl]urea;
trans-(+/−)-N-(4-methoxyphenyl)-N′-[2-(piperidin-1-ylmethyl)cyclohexyl]urea;
trans-(+/−)-2-methoxy-4-methyl-N-[2-(piperidin-1-ylmethyl)cyclohexyl]benzenesulfonamide;
trans-(+/−)-methyl 3-({[2-(piperidin-1-ylmethyl)cyclohexyl]amino}sulfonyl)thiophene-2-carboxylate;
trans-(+/−)-5-[2-(methylthio)pyrimidin-4-yl]-N-[2-(piperidin-1-ylmethyl)cyclohexyl]thiophene-2-sulfonamide;
trans-(+/−)-1-(4-chlorophenyl)-N-[2-(piperidin-1-ylmethyl)cyclohexyl]methanesulfonamide;
trans-(+/−)-N-{2-[(3-butylpiperidin-1-yl)methyl]cyclohexyl}-4-(1,3-oxazol-5-yl)benzamide;
trans-(+/−)-N-{2-[(3-butylpiperidin-1-yl)methyl]cyclohexyl}-6-(trifluoromethyl)nicotinamide;
trans-(+/−)-N-{2-[(3-butylpiperidin-1-yl)methyl]cyclohexyl}-4-(2-methoxyethoxy)benzamide;
trans-(+/−)-N-{2-[(3-butylpiperidin-1-yl)methyl]cyclohexyl}-3-(4-chlorophenyl)propanamide;
trans-(+/−)-N-{2-[(3-butylpiperidin-1-yl)methyl]cyclohexyl}-4-(1H-imidazol-1-yl)benzamide;
trans-(+/−)-N-(2-{[3-(ethoxymethyl)piperidin-1-yl]methyl}cyclohexyl)-6-(1H-imidazol-1-yl)nicotinamide;
trans-(+/−)-N-(2-{[3-(ethoxymethyl)piperidin-1-yl]methyl}cyclohexyl)-4-(1,3-oxazol-5-yl)benzamide;
trans-(+/−)-N-(2-{[3-(ethoxymethyl)piperidin-1-yl]methyl}cyclohexyl)-4-(1H-imidazol-1-yl)benzamide;
trans-(+/−)-N-2-{[3-(ethoxymethyl)piperidin-1-yl]methyl}cyclohexyl)-4-{[(methylsulfonyl)amino]methyl}benzamide;
trans-(+/−)-N-(2-{[3-propylpiperidin-1-yl]methyl}cyclohexyl)-6-(1H-imidazol-1-yl)nicotinamide;
trans-(+/−)-4-(1H-imidazol-1-yl)-N-{2-[(3-propylpiperidin-1-yl)methyl]cyclohexyl}benzamide;
trans-(+/−)-N-(2-{[3-isobutylpiperidin-1-yl]methyl}cyclohexyl)-6-(1H-imidazol-1-yl)nicotinamide;
trans-(+/−)-4-(1H-imidazol-1-yl)-N-{2-[(3-isobutylpiperidin-1-yl)methyl]cyclohexyl}benzamide;
trans-(+/−)-4-Bromo-N-{2-[(3-propylpiperidin-1-yl)methyl]cyclohexyl}benzamide;
trans-(+/−)-3-(4-Chlorophenyl)-N-{2-[(3-propylpiperidin-1-yl)methyl]cyclohexyl}propanamide;
trans-(+/−)-4-Bromo-N-{2-[(3-butylpiperidin-1-yl)methyl]cyclohexyl}benzamide;
trans-(+/−)-N-{2-[(3-Butylpiperidin-1-yl)methyl]cyclohexyl}-4-[(diethylamino)methyl]benzamide;
trans-(+/−)-3-(4-Chlorophenyl)-N-(2-{[3-(ethoxymethyl)piperidin-1-yl]methyl}cyclohexyl)propanamide;
N-[(1S,2R)-2-({4-[(2E)-But-2-en-1-yloxy]piperidin-1-yl}methyl)cyclohexyl]-6-(1H-pyrazol-1-yl)nicotinamide;
N-{(1S,2R)-2-[(4-Butoxypiperidin-1-yl)methyl]cyclohexyl}-6-(1H-pyrazol-1-yl)nicotinamide;
N-(1S,2R)-2-{[(3R)-3-(2-Methoxyethoxy)piperidin-1-yl]methyl}cyclohexyl)-4-(1H-pyrazol-1-yl)benzamide;
N-(1R,2S)-2-{[(3R)-3-(2-Methoxyethoxy)piperidin-1-yl]methyl}cyclohexyl)-4-(1H-pyrazol-1-yl)benzamide;
N-[(1S,2R)-2-({(3R)-3-[(Allyloxy)methyl]piperidin-1-yl}methyl)cyclohexyl]-6-(1H-pyrazol-1-yl)nicotinamide;
N-[(1R,2S)-2-({(3R)-3-[(Allyloxy)methyl]piperidin-1-yl}methyl)cyclohexyl]-6-(1H-pyrazol-1-yl)nicotinamide;
N-[(1R,2S)-2-({(3R)-3-[(Allyloxy)methyl]piperidin-1-yl}methyl)cyclohexyl]-6-(1H-pyrazol-1-yl)nicotinamide
N-[(1S,2R)-2-({(3R)-3-[(Allyloxy)methyl]piperidin-1-yl}methyl)cyclohexyl]-6-(1H-imidazol-1-yl)nicotinamide;
(N-((1S,2R)-2-{[(3R)-3-ethoxypiperidin-1-yl]methyl}cyclohexyl)pyrazine-2-carboxamide;
N-((1S,2R)-2-{[(3R)-3-ethoxypiperidin-1-yl]methyl}cyclohexyl)-6-(ethylthio)nicotinamide;
N-((1S,2R)-2-{[(3R)-3-ethoxypiperidin-1-yl]methyl}cyclohexyl)-6-pyrrolidin-1-ylnicotinamide;
N-[(1S,2R)-2-(azepan-1-ylmethyl)cyclohexyl]-4-(1H-pyrazol-1-yl)benzamide;
N-[(1S,2R)-2-(azepan-1-ylmethyl)cyclohexyl]-6-(1H-pyrazol-1-yl)nicotinamide;
N-((1S,2R)-2-{[(3R)-3-(allyloxy)piperidin-1-yl]methyl}cyclohexyl)-4-(1H-pyrazol-1-yl)benzamide;
N-((1R,2S)-2-{[(3R)-3-(ethoxymethyl)piperidin-1-yl]methyl}cyclohexyl)-4-(1H-pyrrol-1-yl)benzamide;
N-((1S,2R)-2-{[(3R)-3-(ethoxymethyl)piperidin-1-yl]methyl}cyclohexyl)-4-(1H-pyrrol-1-yl)benzamide;
N-((1R,2S)-2-{[(3R)-3-(ethoxymethyl)piperidin-1-yl]methyl}cyclohexyl)-6-pyrrolidin-1-ylnicotinamide;
N-((1S,2R)-2-{[(3R)-3-(ethoxymethyl)piperidin-1-yl]methyl}cyclohexyl)-6-pyrrolidin-1-ylnicotinamide;
N-[(1S,2R)-2-(piperidin-1-ylmethyl)cyclohexyl]-4-(1H-pyrazol-1-yl)benzamide;
N-[(1S,2R)-2-(piperidin-1-ylmethyl)cyclohexyl]-6-(1H-pyrazol-1-yl)nicotinamide;
N-((1S,2R)-2-{[(3R)-3-(allyloxy)piperidin-1-yl]methyl}cyclohexyl)-4-(1H-pyrrol-1-yl)benzamide;
N-((1S,2R)-2-{[(3R)-3-(allyloxy)piperidin-1-yl]-methyl}cyclohexyl)-3-cyclopentylpropanamide;
N-((1S,2R)-2-{[(3R)-3-(allyloxy)piperidin-1-yl]methyl}cyclohexyl)-6-(1H-pyrazol-1-yl)nicotinamide;
N-((1S,2R)-2-{[(3S)-3-(allyloxy)piperidin-1-yl]methyl}cyclohexyl)-6-(1H-pyrazol-1-yl)nicotinamide;
N-((1S,2R)-2-{[(3S)-3-(ethoxymethyl)piperidin-1-yl]methyl}cyclo-hexyl)-4-(2-methoxyethoxy)benzamide;
3-(4-chlorophenyl)-N-((1S,2R)-2-{[(3S)-3-(ethoxymethyl)piperidin-1-yl]methyl}cyclohexyl)propanamide;
N-((1S,2R)-2-{[(3S)-3-(ethoxymethyl)piperidin-1-yl]methyl}cyclo-hexyl)-4-{[(methylsulfonyl)amino]methyl}benzamide;
4-[(diethylamino)methyl]-N-((1S,2R)-2-{[(3S)-3-(ethoxymethyl)-piperidin-1-yl]methyl}cyclohexyl)benzamide;
N-[(1S,2R)-2-({(3R)-3-[(allyloxy)methyl]piperidin-1-yl}methyl)cyclohexyl]-6-(1H-imidazol-1-yl)nicotinamide;
4-chloro-N-((1S,2R)-2-{[(3R)-3-(ethoxymethyl)piperidin-1-yl]methyl}cyclohexyl)benzamide;
N-((1S,2R)-2-{[(3R)-3-(ethoxymethyl)piperidin-1-yl]methyl}cyclohexyl)benzamide;
N-((1S,2R)-2-{[(3R)-3-(ethoxymethyl)piperidin-1-yl]methyl}cyclohexyl)cyclohexanecarboxamide;
N-((1S,2R)-2-{[(3R)-3-(ethoxymethyl)piperidin-1-yl]methyl}cyclohexyl)-2-phenylacetamide;
N-((1S,2R)-2-{[(3R)-3-(ethoxymethyl)piperidin-1-yl]methyl}cyclohexyl)-3-phenylpropanamide;
N-((1S,2R)-2-{[(3R)-3-(ethoxymethyl)piperidin-1-yl]methyl}cyclohexyl)-2,3-dihydro-1-benzofuran-5-carboxamide;
2-cyclopentyl-N-((1S,2R)-2-{[(3R)-3-(ethoxymethyl)piperidin-1-yl]methyl}cyclohexyl)acetamide;
2-chloro-N-((1S,2R)-2-{[(3R)-3-(ethoxymethyl)piperidin-1-yl]methyl}cyclohexyl)-3-fluoroisonicotinamide hydrochloride salt;
(2S)—N-((1S,2R)-2-{[(3R)-3-(ethoxymethyl)piperidin-1-yl]methyl}cyclohexyl)chromane-2-carboxamide hydrochloride salt;
(2R)—N-((1S,2R)-2-{[(3R)-3-(ethoxymethyl)piperidin-1-yl]methyl}cyclohexyl)chromane-2-carboxamide hydrochloride salt;
N-((1S,2R)-2-{[(3R)-3-(ethoxymethyl)piperidin-1-yl]methyl}cyclohexyl)-4,6-dimethylnicotinamide hydrochloride salt;
N-((1S,2R)-2-{[(3R)-3-(ethoxymethyl)piperidin-1-yl]methyl}cyclohexyl)-2,7-dimethylimidazo[1,2-a]pyridine-3-carboxamide hydrochloride salt;
N-((1S,2R)-2-{[(3R)-3-(ethoxymethyl)piperidin-1-yl]methyl}cyclohexyl)-2-(3-methoxyphenyl)acetamide hydrochloride salt;
2-(2,3-dioxo-2,3-dihydro-1H-indol-1-yl)-N-((1S,2R)-2-{[(3R)-3-(ethoxymethyl)piperidin-1-yl]methyl}cyclohexyl)acetamide hydrochloride salt;
N2-acetyl-N1-((1S,2R)-2-{[(3R)-3-(ethoxymethyl)piperidin-1-yl]methyl}cyclohexyl)glycinamide hydrochloride salt;
N-((1S,2R)-2-{[(3R)-3-(ethoxymethyl)piperidin-1-yl]methyl}cyclohexyl)-2-(1H-tetrazol-1-yl)acetamide hydrochloride salt;
N-((1S,2R)-2-{[(3R)-3-(ethoxymethyl)piperidin-1-yl]methyl}cyclohexyl)-5,7-dimethylpyrazolo[1,5-a]pyrimidine-2-carboxamide hydrochloride salt;
N-((1S,2R)-2-{[(3R)-3-(ethoxymethyl)piperidin-1-yl]methyl}cyclohexyl)-3,4-dihydro-2H-1,5-benzodioxepine-6-carboxamide hydrochloride salt;
N-((1S,2R)-2-{[(3R)-3-(ethoxymethyl)piperidin-1-yl]methyl}cyclohexyl)-4-methyl-3,4-dihydro-2H-1,4-benzoxazine-7-carboxamide hydrochloride salt;
N-((1S,2R)-2-{[(3R)-3-(ethoxymethyl)piperidin-1-yl]methyl}cyclohexyl)-5-phenyl-1H-pyrazole-4-carboxamide hydrochloride salt;
N-((1S,2R)-2-{[(3R)-3-(ethoxymethyl)piperidin-1-yl]methyl}cyclohexyl)-4-(1H-tetrazol-1-yl)benzamide hydrochloride salt;
4-[(diethylamino)methyl]-N-((1S,2R)-2-{[(3R)-3-(ethoxymethyl)piperidin-1-yl]methyl}cyclohexyl)benzamide;
N-((1S,2R)-2-{[(3R)-3-(ethoxymethyl)piperidin-1-yl]methyl}cyclohexyl)-4-(2-methoxyethoxy)benzamide;
N-((1S,2R)-2-{[(3R)-3-(ethoxymethyl)piperidin-1-yl]methyl}cyclohexyl)-4-{[(methylsulfonyl)amino]methyl}benzamide;
4-[(acetylamino)methyl]-N-((1S,2R)-2-{[(3R)-3-(ethoxymethyl)piperidin-1-yl]methyl}cyclohexyl)benzamide;
4-[(diacetylamino)methyl]-N-((1S,2R)-2-{[(3R)-3-(ethoxymethyl)piperidin-1-yl]methyl}cyclohexyl)benzamide;
N-((1S,2R)-2-{[(3R)-3-(ethoxymethyl)piperidin-1-yl]methyl}cyclohexyl)-4-{[(ethylsulfonyl)amino]methyl}benzamide;
4-{[(cyclopropylsulfonyl)amino]methyl}-N-((1S,2R)-2-{[(3R)-3-(ethoxymethyl)piperidin-1-yl]methyl}cyclohexyl)benzamide;
N-((1S,2R)-2-{[(3R)-3-(ethoxymethyl)piperidin-1-yl]methyl}cyclohexyl)-4-({[(methylamino)carbonyl]amino}methyl)benzamide;
4-({[(dimethylamino)carbonyl]amino}methyl)-N-((1S,2R)-2-{[(3R)-3-(ethoxymethyl)piperidin-1-yl]methyl}cyclohexyl)benzamide;
N-((1S,2R)-2-{[(3R)-3-(ethoxymethyl)piperidin-1-yl]methyl}cyclohexyl)-4-[(isobutyrylamino)methyl]benzamide;
N-((1S,2R)-2-{[3-cyclohexylpiperidin-1-yl]methyl}cyclohexyl)-6-(1H-pyrazol-1-yl)nicotinamide;
N-((1S,2R)-2-{[3-phenylpiperidin-1-yl]methyl}cyclohexyl)-6-(1H-pyrazol-1-yl)nicotinamide;
   and pharmaceutically acceptable salts thereof.

In another embodiment, the invention provides a compound of formula V, a pharmaceutically acceptable salt thereof, diastereomer, enantiomer, or mixture thereof: 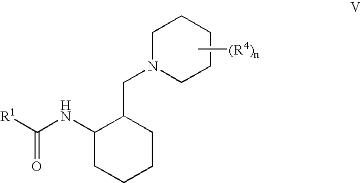
wherein

R¹ is selected from C₆₋₁₀aryl, C₂₋₉heteroaryl, C₃₋₅heterocycloalkyl, C₆₋₁₀aryl-C₁₋₃alkyl, C₂₋₉heteroaryl-C₁₋₃alkyl, C₃₋₅heterocycloalkyl-C₁₋₃alkyl, C₃₋₆cycloalkyl, C₃₋₆cycloalkyl-C₁₋₃alkyl, and C₁₋₆alkyl, wherein said C₆₋₁₀aryl, C₂₋₉heteroaryl, C₆₋₁₀aryl-C₁₋₃alkyl, C₆₋₁₀aryl-O—C₁₋₃alkyl, C₂₋₉heteroaryl-C₁₋₃alkyl, C₃₋₆cycloalkyl, C₃₋₆cycloalkyl-C₁₋₃alkyl, and C₁₋₆alkyl are optionally substituted with one or more group selected from C₆₋₁₀aryl, C₁₋₉heteroaryl, C₃₋₅heterocycloalkyl, C₆₋₁₀aryl-C₁₋₃alkyl, C₆₋₁₀aryl-O—C₁₋₃alkyl, C₂₋₉heteroaryl-C₁₋₃alkyl, C₃₋₅heterocycloalkyl-C₁₋₃alkyl, —CN, —SR, —OR, —O(CH₂)ₘ—OR, R, —C(═O)—R, —CO₂R, —SO₂R, —SO₂NR₂, halogen, —NO₂, —NR₂, —(CH₂)ₘNR₂, —(CH₂)ₘNHC(═O)—NR₂, —NHC(═O)—R, —N[C(═O)R]₂, —(CH₂)ₘNHC(═O)—R, —(CH₂)ₘN[C(═O)—R]₂—(CH₂)ₘNHS(═O)₂—R, and —C(═O)—NR₂; and

R⁴ is selected from C₆₋₁₀aryl, C₂₋₉heteroaryl, C₃₋₆cycloalkyl, C₃₋₅heterocycloalkyl, C₆₋₁₀aryl-C₁₋₃alkyl, C₂₋₉heteroaryl-C₁₋₃alkyl, C₃₋₅heterocycloalkyl-C₁₋₃alkyl, —CN, —SR, —OR, —(CH₂)ₘOR, —O(CH₂)ₘOR, —O(CH₂)ₘNR₂, —(CH₂)ₘ—O—(CH₂)ₙOR, —(CH₂)ₘ—O—(CH₂)ₙNR₂, R, —CO₂R; —SO₂R; —SO₂NR₂, halogen, —NO₂, —NR₂, —(CH₂)ₘNR₂, and —C(═O)—NR₂;

each R is independently hydrogen, C₁₋₆alkyl, C₂₋₆alkenyl or halogenated C₁₋₆alkyl;

with a proviso that

R¹ is not 4-amino-5-chloro-2-alkoxylphenyl, 4-amino-5-chloro-2-cycloalkoxyphenyl, 4-amino-5-chloro-2-cycloalkyl-alkoxy-phenyl, 4-butoxyphenyl, 3-butoxyphenyl, 4-pentyloxyphenyl, 4-isobutoxyphenyl, 4-benzoyloxyphenyl and 7-(2,3-dihydro)benzofuranyl.

In a particular embodiment, R¹ of formula V is selected from C₆₋₁₀aryl, C₂₋₉heteroaryl, C₃₋₅heterocycloalkyl, C₆₋₁₀aryl-C₁₋₃alkyl, C₂₋₉heteroaryl-C₁₋₃alkyl, C₃₋₅heterocycloalkyl-C₁₋₃alkyl, C₃₋₆cycloalkyl, C₃₋₆cycloalkyl-C₁₋₃alkyl, and C₃₋₆alkyl, wherein said C₆₋₁₀aryl, C₂₋₉heteroaryl, C₆₋₁₀aryl-C₁₋₃alkyl, C₆₋₁₀aryl-O—C₁₋₃alkyl, C₂₋₉heteroaryl-C₁₋₃alkyl, C₃₋₆cycloalkyl, C₃₋₆cycloalkyl-C₁₋₃alkyl, and C₃₋₆alkyl are optionally substituted by one or more groups selected from 1H-pyrazol-1-yl, fluoro, chloro, trifluoromethyl, methoxy, difluoromethoxy, trifluoromethoxy, 2-methoxyethoxy, 2-ethoxyethoxy, t-butyl, cyano, bromo, 1,3-oxazol-5-yl, 1H-imidazol-1-yl, (4-oxopiperidin-1-yl)carbonyl, pyridin-3-ylmethyl, [(butylamino)carbonyl]amino, 1,1-dioxidothiomorpholin-4-yl, aminosulfonyl, morpholin-4-yl, diethylaminomethyl, acetyl, (3-oxo-2,3-dihydro-4H-1,4-benzoxazin-4-yl)methyl, 1-oxo-indan-4-yl, dimethylaminomethyl, methyl, pyrrolidin-1-yl, ethylthio, acetylamino, dimethylamino, 1H-pyrrol-1-yl, ethyl, ethoxy, fluorophenoxy, propyl, phenyl, methoxycarbonyl, diacetylamino, (methylsulfonylamino)methyl, (cyclopropylsulfonylamino)methyl, 1H-tetrazol-1-yl, pyrazolyl, methylaminocarbonylamino, dimethylaminocarbonylamino, and (methylthio)pyrimidin-4-yl.

In another particular embodiment, R¹ of formula V is selected from 2-cyclopentylethyl, cyclopropylmethyl, ethyl, methyl, cyclohexyl, cyclopentylmethyl, chromanyl, pentyl, 2-phenylethyl, phenyl, benzyl, pyridinyl, pyridinylethyl, 1-benzofuranyl, benzothienyl, furyl, imidazolyl, pyrazolo[1,5-a]pyrimidinyl, pyrazinyl, 1,3-benzothiazolyl, indolyl, indazolyl, thienyl, 1,3-benzodioxinyl, tetrahydro-2H-pyran-4-ylmethyl, 1-H-1,2,3-benzotriazol-1-yl, 2-(thien-2-yl)ethyl, (1-benzofuran-4-yl)methyl, 1,3-oxazolyl, 1H-pyrazol-1-yl, 2,3-dihydro-1-benzofuran-5-yl, 1,3-benzodioxol-5-yl, 2-oxo-2,3-dihydro-2H-benzimidazolyl, isoxazolyl, imidazo[1,2,a]pyridinyl, 2-3-dioxo-2,3-dihydro-1H-indol-1-yl, 3,4-dihydro-2H-1,4-benzoxazinyl; pyrazolyl, 1H-tetrazol-1-yl-methyl, and 3,4-dihydro-2H-1,5-benzodioxepinyl, which are optionally substituted by one or more groups selected from C₆₋₁₀aryl, C₂₋₉heteroaryl, C₃₋₅heterocycloalkyl, C₆₋₁₀aryl-C₁₋₃alkyl, C₆₋₁₀aryl-O—C₁₋₃alkyl, C₂₋₉heteroaryl-C₁₋₃alkyl, C₃₋₅heterocycloalkyl-C₁₋₃alkyl, —CN, —SR, —OR, —O(CH₂)ₘ—OR, R, —C(═O)—R, —CO₂R, —SO₂R, —SO₂NR₂, halogen, —NO₂, —NR₂, —(CH₂)ₘNR₂, —(CH₂)ₘNHC(═O)—NR₂, —NHC(═O)—R, —(CH₂)ₘNHC(═O)—R, —(CH₂)ₘN[C(═O)—R]₂, —N[C(═O)R]₂, —(CH₂)ₘNS(═O)₂—R, and —C(═O)—NR₂.

In another particular embodiment, R¹ of formula V is selected from 2-cyclopentylethyl, cyclopropylmethyl, ethyl, methyl, cyclohexyl, cyclopentylmethyl, chromanyl, pentyl, 2-phenylethyl, phenyl, benzyl, pyridinyl, pyridinylethyl, 1-benzofuranyl, benzothienyl, furyl, imidazolyl, pyrazolo[1,5-a]pyrimidinyl, pyrazinyl, 1,3-benzothiazolyl, indolyl, indazolyl, thienyl, 1,3-benzodioxinyl, tetrahydro-2H-pyran-4-ylmethyl, 1-H-1,2,3-benzotriazol-1-yl, 2-(thien-2-yl)ethyl, (1-benzofuran-4-yl)methyl, 1,3-oxazolyl, 1H-pyrazol-1-yl, 2,3-dihydro-1-benzofuran-5-yl, 1,3-benzodioxol-5-yl, 2-oxo-2,3-dihydro-2H-benzimidazolyl, isoxazolyl, imidazo[1,2,a]pyridinyl, 2-3-dioxo-2,3-dihydro-1H-indol-1-yl, 3,4-dihydro-2H-1,4-benzoxazinyl; pyrazolyl, 1H-tetrazol-1-yl-methyl, and 3,4-dihydro-2H-1,5-benzodioxepinyl, which are optionally substituted by are optionally substituted by one or more groups selected from 1H-pyrazol-1-yl, fluoro, chloro, trifluoromethyl, methoxy, difluoromethoxy, trifluoromethoxy, 2-methoxyethoxy, 2-ethoxyethoxy, t-butyl, cyano, bromo, 1,3-oxazol-5-yl, 1H-imidazol-1-yl, (4-oxopiperidin-1-yl)carbonyl, pyridin-3-ylmethyl, [(butylamino)carbonyl]amino, 1,1-dioxidothiomorpholin-4-yl, aminosulfonyl, morpholin-4-yl, diethylaminomethyl, acetyl, (3-oxo-2,3-dihydro-4H-1,4-benzoxazin-4-yl)methyl, 1-oxo-indan-4-yl, dimethylaminomethyl, methyl, pyrrolidin-1-yl, ethylthio, acetylamino, dimethylamino, 1H-pyrrol-1-yl, ethyl, ethoxy, fluorophenoxy, propyl, phenyl, methoxycarbonyl, diacetylamino, (methylsulfonylamino)methyl, (cyclopropylsulfonylamino)methyl, 1H-tetrazol-1-yl, pyrazolyl, methylaminocarbonylamino, dimethylaminocarbonylamino, and (methylthio)pyrimidin-4-yl.

In another particular embodiment, R⁴ of formula V is selected from phenyl, benzyl, methyl, fluoro, trifluoromethyl, methoxy, allyloxy, (2E)-but-2-en-1-yloxy, (allyloxy)methyl, methoxymethyl, ethoxymethyl, propyl, butyl, pentyl, hexyl, cyclopentyl, pyridin-4-ylmethyl, ethoxy, butoxy, 2-methoxyethoxy, cyclohexyl, and thienylmethyl.

In a further embodiment, the two substitutents on the cyclohexyl ring of formula I or V are in trans positions.

It will be understood that when compounds of the present invention contain one or more chiral centers, the compounds of the invention may exist in, and be isolated as, enantiomeric or diastereomeric forms, or as a racemic mixture. The present invention includes any possible enantiomers, diastereomers, racemates or mixtures thereof, of a compound of Formula I or V. The optically active forms of the compound of the invention may be prepared, for example, by chiral chromatographic separation of a racemate, by synthesis from optically active starting materials or by asymmetric synthesis based on the procedures described thereafter.

It will also be appreciated that certain compounds of the present invention may exist as geometrical isomers, for example E and Z isomers of alkenes. The present invention includes any geometrical isomer of a compound of Formula I or V. It will further be understood that the present invention encompasses tautomers of the compounds of the Formula I or V.

It will also be understood that certain compounds of the present invention may exist in solvated, for example hydrated, as well as unsolvated forms. It will further be understood that the present invention encompasses all such solvated forms of the compounds of the Formula I or V.

Within the scope of the invention are also salts of the compounds of the Formula I or V. Generally, pharmaceutically acceptable salts of compounds of the present invention may be obtained using standard procedures well known in the art, for example by reacting a sufficiently basic compound, for example an alkyl amine with a suitable acid, for example, HCl or acetic acid, to afford a physiologically acceptable anion. It may also be possible to make a corresponding alkali metal (such as sodium, potassium, or lithium) or an alkaline earth metal (such as a calcium) salt by treating a compound of the present invention having a suitably acidic proton, such as a carboxylic acid or a phenol with one equivalent of an alkali metal or alkaline earth metal hydroxide or alkoxide (such as the ethoxide or methoxide), or a suitably basic organic amine (such as choline or meglumine) in an aqueous medium, followed by conventional purification techniques.

In one embodiment, the compound of Formula I or V above may be converted to a pharmaceutically acceptable salt or solvate thereof, particularly, an acid addition salt such as a hydrochloride, hydrobromide, phosphate, acetate, fumarate, maleate, tartrate, citrate, methanesulphonate or p-toluenesulphonate.

We have now found that the compounds of the invention have activity as pharmaceuticals, in particular as agonists of M1 receptors. More particularly, the compounds of the invention exhibit selective activity as agonist of the M1 receptors and are useful in therapy, especially for relief of various pain conditions such as chronic pain, neuropathic pain, acute pain, cancer pain, pain caused by rheumatoid arthritis, migraine, visceral pain etc. This list should however not be interpreted as exhaustive. Additionally, compounds of the present invention are useful in other disease states in which dysfunction of M1 receptors is present or implicated. Furthermore, the compounds of the invention may be used to treat cancer, multiple sclerosis, Parkinson's disease, Huntington's chorea, schizophrenia, Alzheimer's disease, anxiety disorders, depression, obesity, gastrointestinal disorders and cardiovascular disorders.

In a particular embodiment, the compounds may be used to treat schizophrenia or Alzheimer's disease.

In another embodiment, the compounds may be used to treat pain.

In another particular embodiment, the compounds may be used to treat neuropathic pain.

Compounds of the invention are useful as immunomodulators, especially for autoimmune diseases, such as arthritis, for skin grafts, organ transplants and similar surgical needs, for collagen diseases, various allergies, for use as anti-tumour agents and anti viral agents.

Compounds of the invention are useful in disease states where degeneration or dysfunction of M1 receptors is present or implicated in that paradigm. This may involve the use of isotopically labeled versions of the compounds of the invention in diagnostic techniques and imaging applications such as positron emission tomography (PET).

Compounds of the invention are useful for the treatment of diarrhea, depression, anxiety and stress-related disorders such as post-traumatic stress disorders, panic disorder, generalized anxiety disorder, social phobia, and obsessive compulsive disorder, urinary incontinence, premature ejaculation, various mental illnesses, cough, lung oedema, various gastrointestinal disorders, e.g. constipation, functional gastrointestinal disorders such as Irritable Bowel Syndrome and Functional Dyspepsia, Parkinson's disease and other motor disorders, traumatic brain injury, stroke, cardioprotection following miocardial infarction, obesity, spinal injury and drug addiction, including the treatment of alcohol, nicotine, opioid and other drug abuse and for disorders of the sympathetic nervous system for example hypertension.

Compounds of the invention are useful as an analgesic agent for use during general anaesthesia and monitored anaesthesia care. Combinations of agents with different properties are often used to achieve a balance of effects needed to maintain the anaesthetic state (e.g. amnesia, analgesia, muscle relaxation and sedation). Included in this combination are inhaled anaesthetics, hypnotics, anxiolytics, neuromuscular blockers and opioids.

Also within the scope of the invention is the use of any of the compounds according to the Formula I or V above, for the manufacture of a medicament for the treatment of any of the conditions discussed above.

A further aspect of the invention is a method for the treatment of a subject suffering from any of the conditions discussed above, whereby an effective amount of a compound according to the Formula I or V above, is administered to a patient in need of such treatment.

Thus, the invention provides a compound of Formula I or V or pharmaceutically acceptable salt or solvate thereof, as hereinbefore defined for use in therapy.

In a further aspect, the present invention provides the use of a compound of Formula I or V or a pharmaceutically acceptable salt or solvate thereof, as hereinbefore defined in the manufacture of a medicament for use in therapy.

In the context of the present specification, the term “therapy” also includes “prophylaxis” unless there are specific indications to the contrary. The term “therapeutic” and “therapeutically” should be construed accordingly. The term “therapy” within the context of the present invention further encompasses to administer an effective amount of a compound of the present invention, to mitigate either a pre-existing disease state, acute or chronic, or a recurring condition. This definition also encompasses prophylactic therapies for prevention of recurring conditions and continued therapy for chronic disorders.

The compounds of the present invention are useful in therapy, especially for the therapy of various pain conditions including, but not limited to: acute pain, chronic pain, neuropathic pain, back pain, cancer pain, and visceral pain. In a particular embodiment, the compounds are useful in therapy for neuropathic pain. In an even more particular embodiment, the compounds are useful in therapy for chronic neuropathic pain.

In use for therapy in a warm-blooded animal such as a human, the compound of the invention may be administered in the form of a conventional pharmaceutical composition by any route including orally, intramuscularly, subcutaneously, topically, intranasally, intraperitoneally, intrathoracially, intravenously, epidurally, intrathecally, transdermally, intracerebroventricularly and by injection into the joints.

In one embodiment of the invention, the route of administration may be oral, intravenous or intramuscular.

The dosage will depend on the route of administration, the severity of the disease, age and weight of the patient and other factors normally considered by the attending physician, when determining the individual regimen and dosage level at the most appropriate for a particular patient.

For preparing pharmaceutical compositions from the compounds of this invention, inert, pharmaceutically acceptable carriers can be either solid and liquid. Solid form preparations include powders, tablets, dispersible granules, capsules, cachets, and suppositories.

A solid carrier can be one or more substances, which may also act as diluents, flavoring agents, solubilizers, lubricants, suspending agents, binders, or table disintegrating agents; it can also be an encapsulating material.

In powders, the carrier is a finely divided solid, which is in a mixture with the finely divided compound of the invention, or the active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture in then poured into convenient sized moulds and allowed to cool and solidify.

Suitable carriers are magnesium carbonate, magnesium stearate, talc, lactose, sugar, pectin, dextrin, starch, tragacanth, methyl cellulose, sodium carboxymethyl cellulose, a low-melting wax, cocoa butter, and the like.

The term composition is also intended to include the formulation of the active component with encapsulating material as a carrier providing a capsule in which the active component (with or without other carriers) is surrounded by a carrier which is thus in association with it. Similarly, cachets are included.

Tablets, powders, cachets, and capsules can be used as solid dosage forms suitable for oral administration.

Liquid form compositions include solutions, suspensions, and emulsions. For example, sterile water or water propylene glycol solutions of the active compounds may be liquid preparations suitable for parenteral administration. Liquid compositions can also be formulated in solution in aqueous polyethylene glycol solution.

Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

Depending on the mode of administration, the pharmaceutical composition will preferably include from 0.05% to 99% w (percent by weight), more preferably from 0.10 to 50% w, of the compound of the invention, all percentages by weight being based on total composition.

A therapeutically effective amount for the practice of the present invention may be determined, by the use of known criteria including the age, weight and response of the individual patient, and interpreted within the context of the disease which is being treated or which is being prevented, by one of ordinary skills in the art.

Within the scope of the invention is the use of any compound of Formula I or V as defined above for the manufacture of a medicament.

Also within the scope of the invention is the use of any compound of Formula I or V for the manufacture of a medicament for the therapy of pain.

Additionally provided is the use of any compound according to Formula I or V for the manufacture of a medicament for the therapy of various pain conditions including, but not limited to: acute pain, chronic pain, neuropathic pain, back pain, cancer pain, and visceral pain.

A further aspect of the invention is a method for therapy of a subject suffering from any of the conditions discussed above, whereby an effective amount of a compound according to the Formula I or V above, is administered to a patient in need of such therapy.

Additionally, there is provided a pharmaceutical composition comprising a compound of Formula I or V or a pharmaceutically acceptable salt thereof, in association with a pharmaceutically acceptable carrier.

Particularly, there is provided a pharmaceutical composition comprising a compound of Formula I or V or a pharmaceutically acceptable salt thereof, in association with a pharmaceutically acceptable carrier for therapy, more particularly for therapy of pain.

Further, there is provided a pharmaceutical composition comprising a compound of Formula I or V or a pharmaceutically acceptable salt thereof, in association with a pharmaceutically acceptable carrier use in any of the conditions discussed above.

In a further aspect, the present invention provides a method of preparing the compounds of the present invention.

In one embodiment, the invention provides a process for preparing a compound of Formula II, comprising: 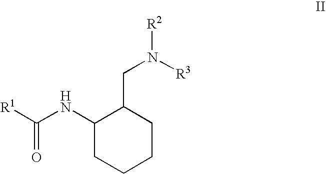
reacting a compound of Formula III with a compound of R¹—COCl or R¹—COOH,

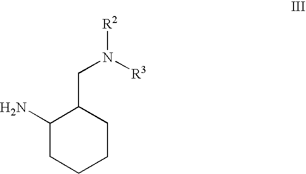

wherein R¹ R², and R³ are defined as those of formula I or V.

Optionally, the step of reacting a compound of formula III with a compound of R¹—COCl or R¹—COOH is carried out in the presence of a base, such as diisopropylethylamine, or triethylamine, optionally in the presence of catalyst such as HATU.

In another embodiment, the invention provides a process for preparing a compound of Formula IV, comprising: 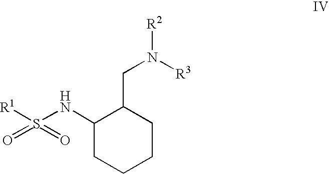
reacting a compound of Formula III with a compound of R¹SO₂Cl, 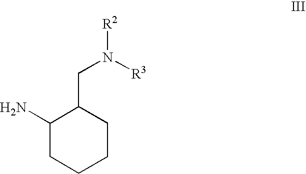
wherein R¹ R², and R³ are defined as those of formula I or V.

Optionally, the step of reacting a compound of formula III with a compound of R¹SO₂Cl is carried out in the presence of a base, such as diisopropylethylamine, or triethylamine.

In another embodiment, the invention provides a process for preparing a compound of Formula VI, comprising 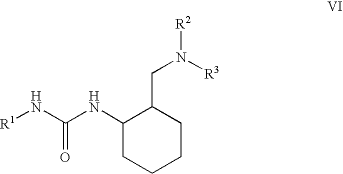
reacting a compound of Formula III with R¹NCO, 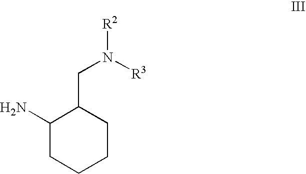
wherein R¹ R², and R³ are defined as those of formula I or V.

Compounds of the present invention may also be prepared according to the synthetic routes as depicted in Schemes 1-17. 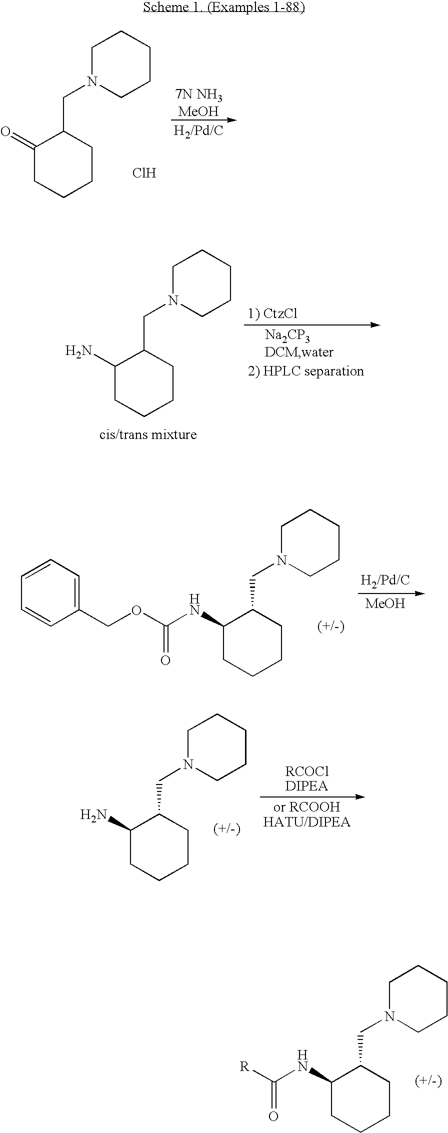 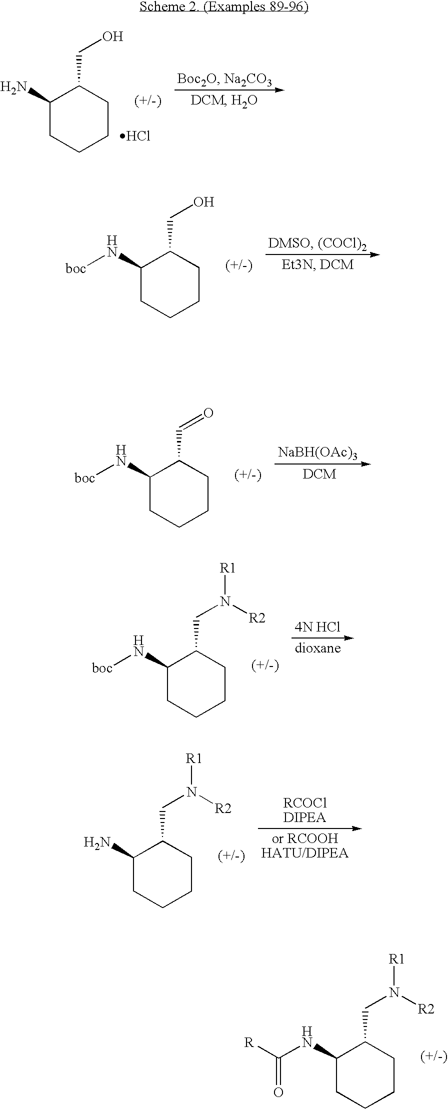 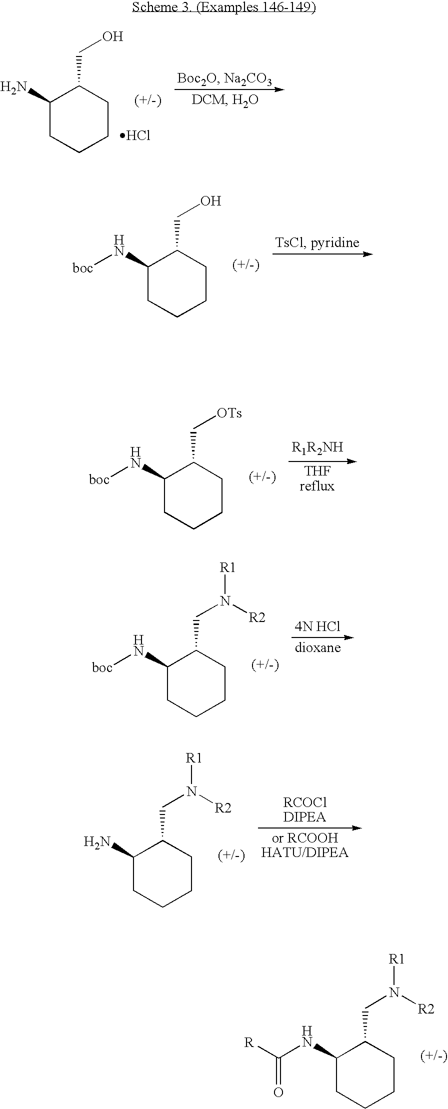 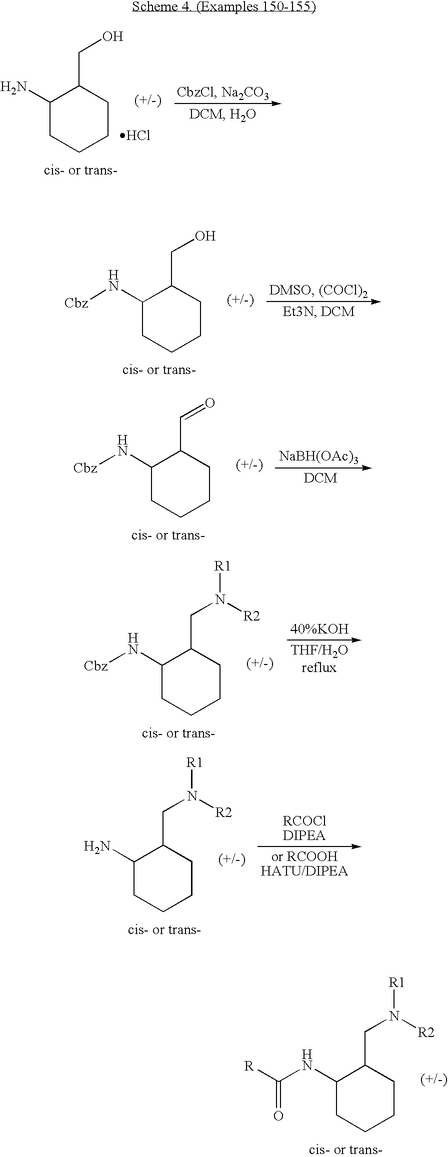 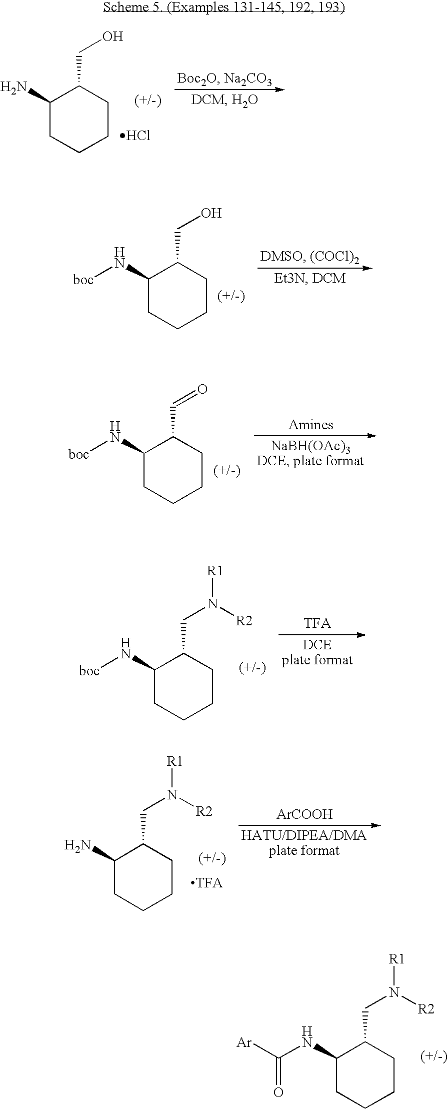 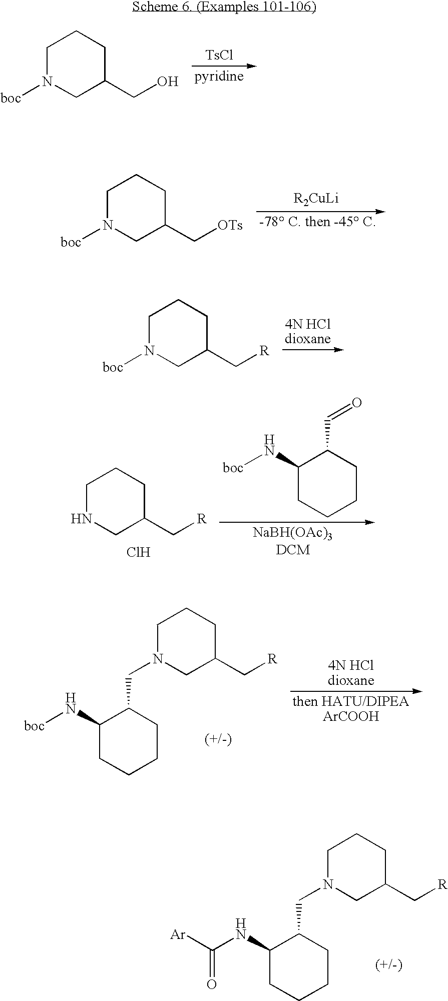 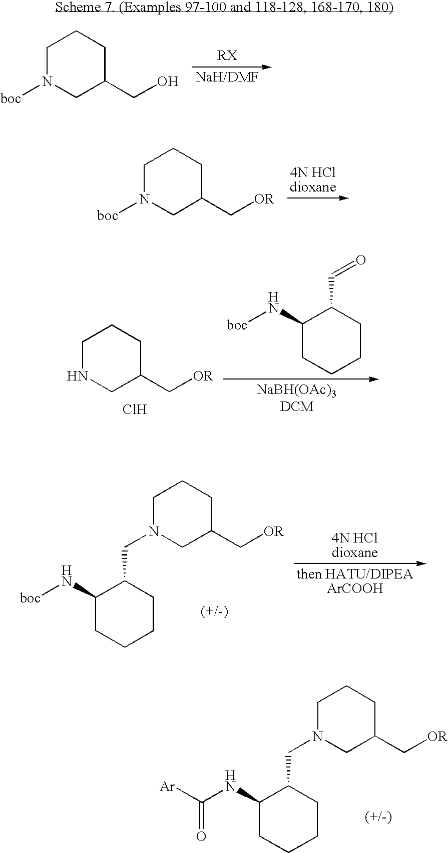 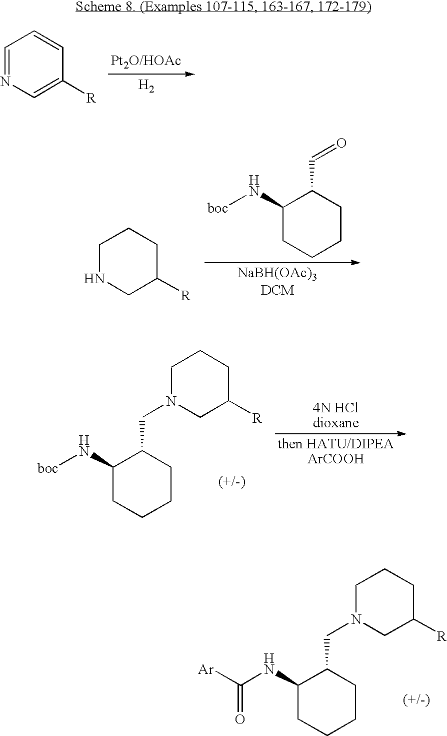 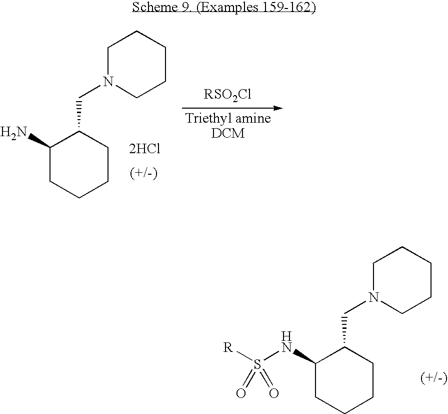 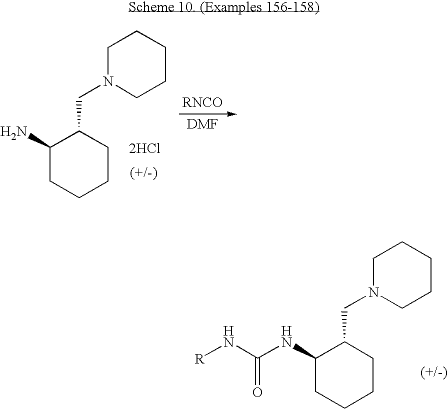 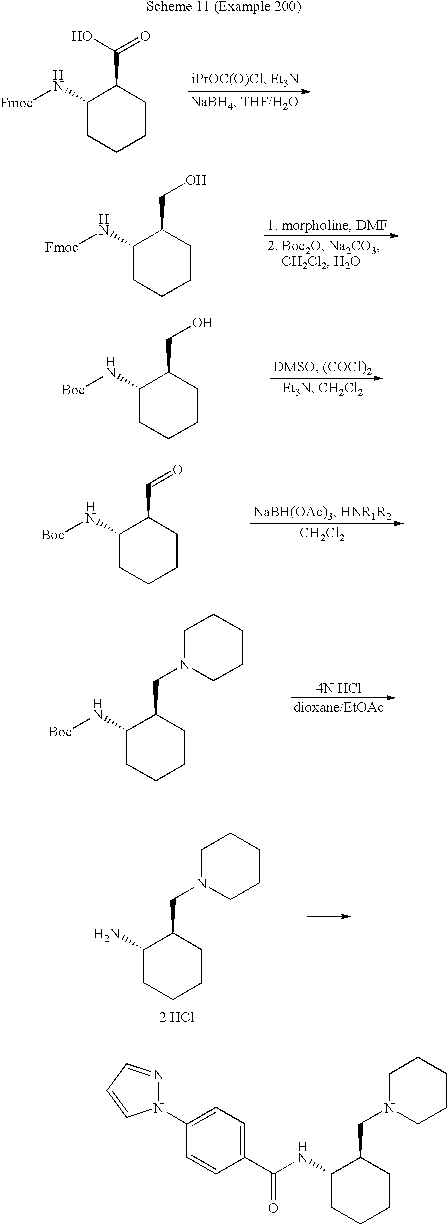 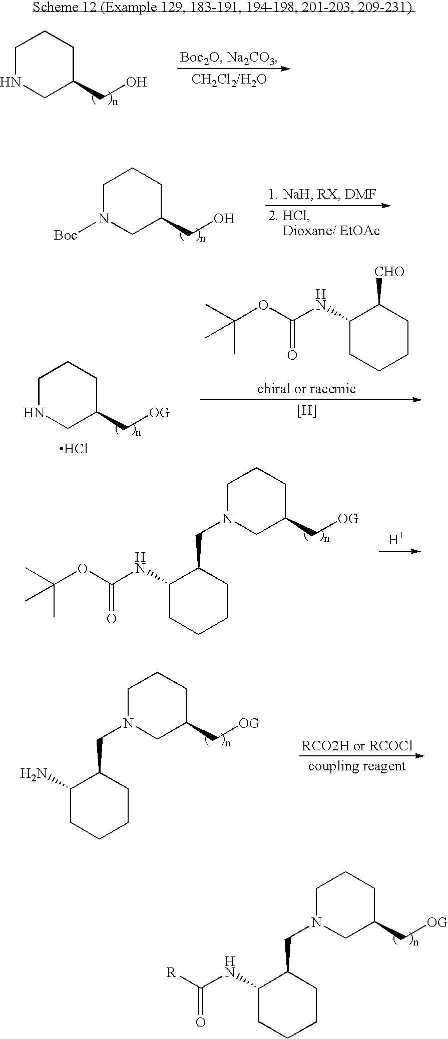 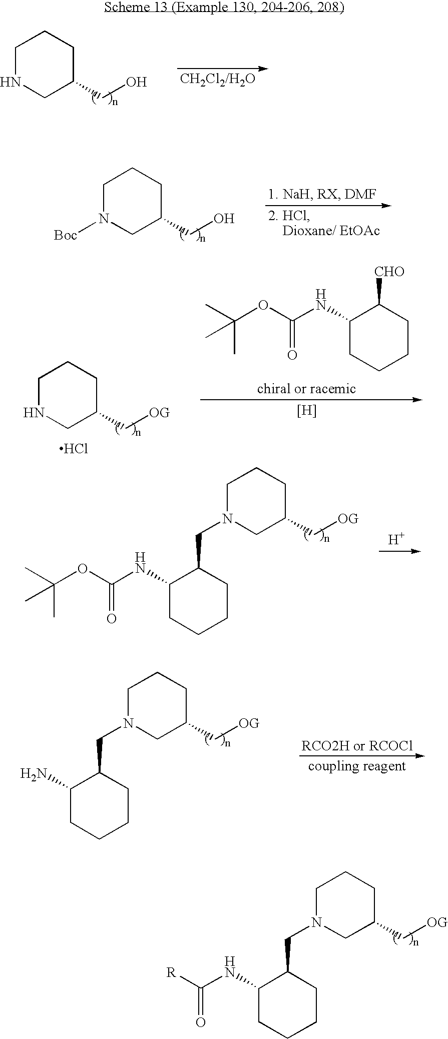 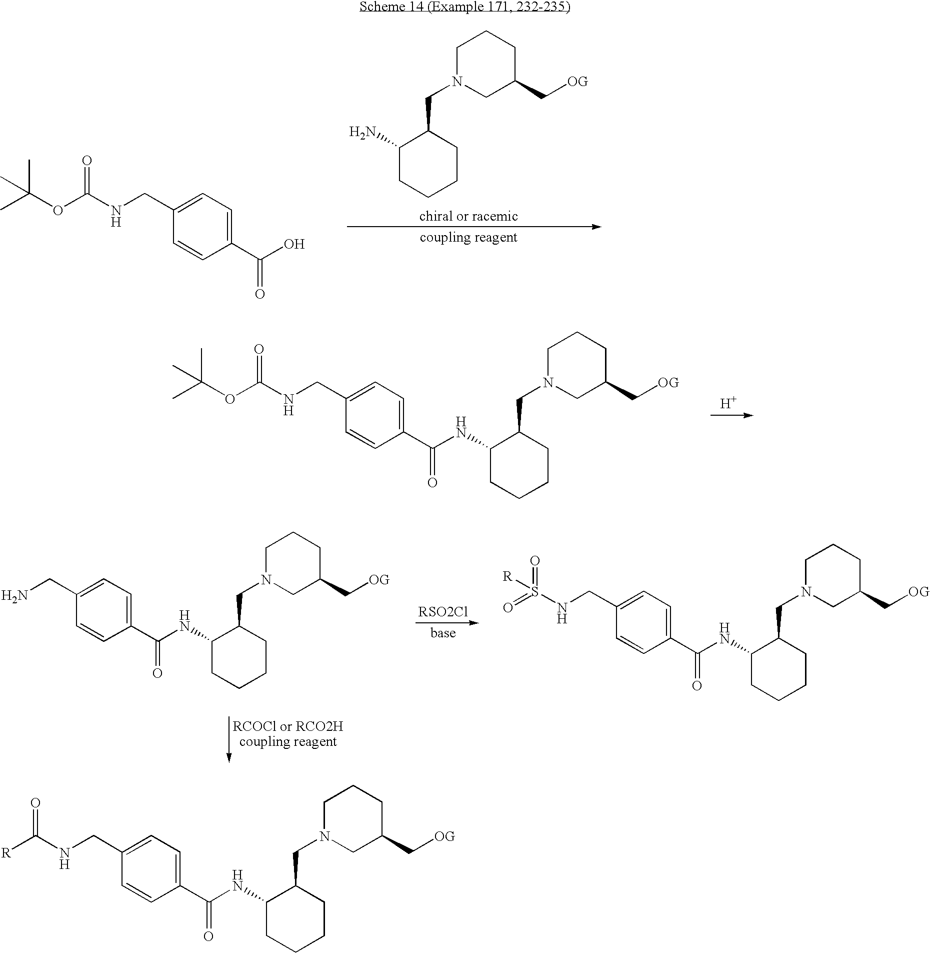 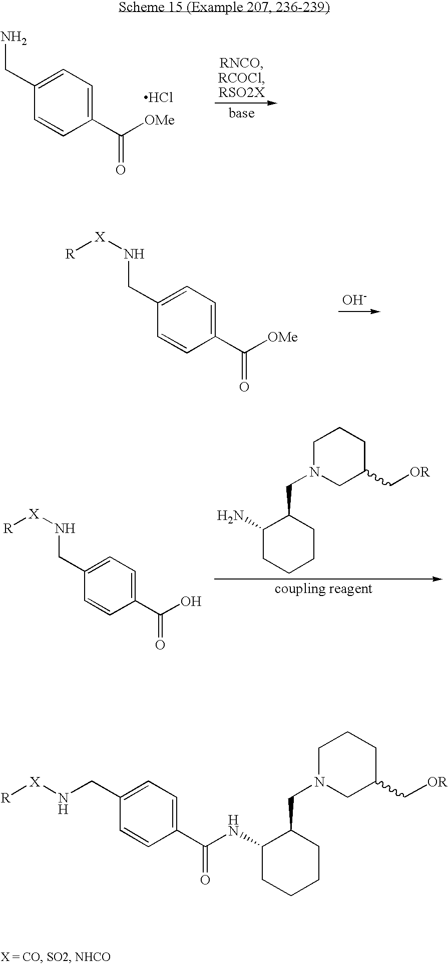 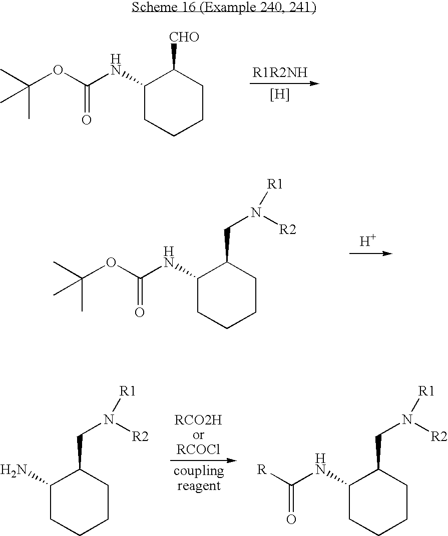 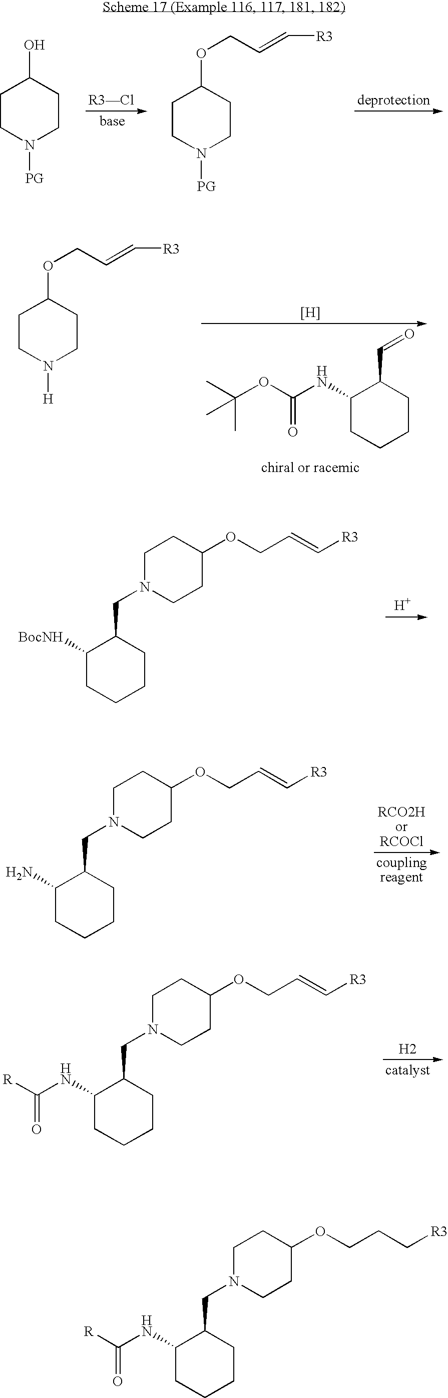
Biological Evaluation
Human M1, Rat M1, Human M3 and Human M5 Calcium Mobilization FLIPR™ Assay

The compound activity in the present invention (EC50 or IC₅₀) was measured using a 384 plate-based imaging assay that monitors drug induced intracellular Ca² release in whole cells. Activation of hM1 (human Muscarinic receptor subtype 1, gene bank access NM_{—}000738), rM1 (rat Muscarinic receptor subtype 1, gene bank access NM_{—}080773), hM3 (human Muscarinic receptor subtype 3, gene bank access NM_{—}000740NM_{—}000740) and hM5 (human Muscarinic receptor subtype 5, gene bank access NM_{—}0121258) receptors expressed in CHO cells (chinese hamster ovary cells, ATCC) was quantified in a Molecular Devices FLIPR II™ instrument as an increase in fluorescent signal. Inhibition of hM3 and hM5 by compounds was determined by the decrease in fluorescent signal in response to 2 nM acetylcholine activation.

CHO cells were plated in 384-black polylysine coated plate (Costar) at 8000 cells/well/50 μl for 24 hours or 4000 cells/well for 48 hours in a humidified incubator (5% CO₂ and 37° C.) in DMEM/F12 medium without selection agent. Prior to the experiment the cell culture medium was removed from the plates by inversion. A loading solution of 30 μl of Hank's balanced salt solution, 10 mM Hepes and 2.5 mM Probenicid at Ph 7.4 (Cat no. 311-520-VL, Wisent) with 2 μM calcium indicator dye (FLUO-3AM, Molecular Probes F14202) was added to each well. Plates were incubated at 37° C. for 60 minutes prior to start the experiment. The incubation was terminated by washing the cells four times in assay buffer, leaving a residual 25 μl buffer per well. Cell plates were then transferred to the FLIPR, ready for compound additions.

The day of experiment, acetylcholine and compounds were diluted in three-fold concentration range (10 points serial dilution) for addition by FLIPR instrument. For all calcium assays, a baseline reading was taken for 30 seconds followed by the addition of 12.5 μl (25 μl for hM1 and rM1) of compounds, resulting in a total well volume of 37.5 μl (50 μl for hM1 and rM1). Data were collected every 1.6 seconds for 300 seconds. For hM3 and hM5 an additional 12.5 μl of acetylcholine (2 nM final) was added at 300 seconds. After this addition of acetylcholine (producing a final volume of 50 μl), the FLIPR continued to collect data every 2 seconds for 240 seconds. The fluorescence emission was read using filter 1 (emission 520-545 nm) by the FLIPR on board CCD camera.

Calcium mobilization output data were calculated as the maximal relative fluorescence unit (RFU) minus the minimal value for both compound and agonist reading frame (except for hM1 and rM1 using only the maximal RFU). Data were analyzed using sigmoidal fits of a non-linear curve-fitting program (XLfit version 5.0.6 from ID Business Solutions Limited, Guildford, UK). All EC50 and IC50 values are reported as geometric means of ‘n’ independent experiments. Using the above-mentioned assays, the IC50 and EC50 towards human hM1, ratM1, hM3 and hM5 receptors for most compounds is measured to be in the range 1->30000 nM. The Eₘₐₓ (maximal effect, agonism or antagonist inhibition) towards human hM1, ratM1, hM3 and hM5 receptors for most compounds is measured to be in the range of 0-110%.

hM2 Receptor GTPyS Binding

Membranes produced from Chinese hamster ovary cells (CHO) expressing the cloned human M2 receptor (human Muscarinic receptor subtype 2, gene bank access NM_{—}000739), were obtained from Perkin-Elmer (RBHM2M). The membranes were thawed at 37° C., passed 3 times through a 23-gauge blunt-end needle, diluted in the GTPγS binding buffer (50 mM Hepes, 20 mM NaOH, 100 mM NaCl, 1 mM EDTA, 5 mM MgCl₂, pH 7.4, 100 μM DTT). The EC₅₀, IC₅₀ and Eₘₐₓ of the compounds of the invention were evaluated from 10-point dose-response curves (three fold concentration range) done in 60 μl in 384-well non-specific binding surface plate (Corning). Ten microliters from the dose-response curves plate (5× concentration) were transferred to another 384 well plate containing the following: 10 μg of hM2 membranes, 500 μg of Flashblue beads (Perkin-Elmer) and GDP in a 25 μl volume. An additional 15 μl containing 3.3× (55000 dpm) of GTPγ³⁵S (0.4 nM final) were added to the wells resulting in a total well volume of 50 μl. Basal and maximal stimulated GTPγ³⁵S binding was determined in absence and presence of 30 μM of acetylcholine agonist. The membranes/beads mix were pre-incubated for 15 minutes at room temperature with 25 μM GDP prior to distribution in plates (12.5 μM final). The reversal of acetylcholine-induced stimulation (2 μM final) of GTPγ³⁵S binding was used to assay the antagonist properties (IC₅₀) of the compounds. The plates were incubated for 60 minutes at room temperature with shaking, then centrifuged at 2000 rpm for 5 minutes. The radioactivity (cpm) was counted in a Trilux (Perkin-Elmer).

Values of EC₅₀, IC₅₀ and Eₘₐₓ were obtained using sigmoidal fits of a non-linear curve-fitting program (XLfit version 5.0.6 from ID Business Solutions Limited, Guildford, UK) of percent-stimulated GTPγ³⁵S binding vs. log (molar ligand).

All EC50 and IC50 values are reported as geometric means of ‘n’ independent experiments. Based on the above assays, the EC₅₀ towards human M2 receptors for most compounds of the invention is measured to be in the range of about between 200 and >30000 nM. The Eₘₐₓ (maximal effect, agonism or antagonist inhibition) towards human M2 receptors for most compounds of the invention were measured to be in the range of about 0-120%. The IC₅₀ was the concentration of the compound of the invention at which 50% inhibition of acetylcholine GTPγ³⁵S binding stimulation has been observed. The IC₅₀ towards human M2 receptors for most compounds of the invention was measured to be in the range of between 40 and >90000 nM.

hM4 Receptor GTPyS Binding

Membranes produced from Chinese hamster ovary cells (CHO) expressing the cloned human M4 receptor (human Muscarinic receptor subtype 4, gene bank access NM_{—}000741), were obtained from Perkin-Elmer (RBHM4M). The membranes were thawed at 37° C., passed 3 times through a 23-gauge blunt-end needle, diluted in the GTPγS binding buffer (50 mM Hepes, 20 mM NaOH, 100 mM NaCl, 1 mM EDTA, 5 mM MgCl₂, pH 7.4, 100 μM DTT). The EC₅₀, IC₅₀ and Eₘₐₓ of the compounds of the invention were evaluated from 10-point dose-response curves (three fold concentration range) done in 60 μl in 384-well non-specific binding surface plate (Corning). Ten microliters from the dose-response curves plate (5× concentration) were transferred to another 384 well plate containing the following: 10 μg of hM4 membranes, 500 μg of Flashblue beads (Perkin-Elmer) and GDP in a 25 μl volume. An additional 15 μl containing 3.3× (55000 dpm) of GTPγ³⁵S (0.4 nM final) were added to the wells resulting in a total well volume of 50 μl. Basal and maximal stimulated GTPγ³⁵S binding was determined in absence and presence of 30 μM of acetylcholine agonist. The membranes/beads mix were pre-incubated for 15 minutes at room temperature with 40 μM GDP prior to distribution in plates (20 μM final). The reversal of acetylcholine-induced stimulation (10 μM final) of GTPγ³⁵S binding was used to assay the antagonist properties (IC₅₀) of the compounds. The plates were incubated for 60 minutes at room temperature with shaking, then centrifuged at 2000 rpm for 5 minutes. The radioactivity (cpm) was counted in a Trilux (Perkin-Elmer).

Values of EC₅₀, IC₅₀ and Eₘₐₓ were obtained using sigmoidal fits of a non-linear curve-fitting program (XLfit version 5.0.6 from ID Business Solutions Limited, Guildford, UK) of percent-stimulated GTPγ³⁵S binding vs. log (molar ligand).

All EC50 and IC50 values are reported as geometric means of ‘n’ independent experiments. Based on the above assays, the EC50 towards human M4 receptors for most compounds of the invention is measured to be in the range of between 300 and >30000 nM. The Eₘₐₓ (maximal effect, agonism or antagonist inhibition) towards human M4 receptors for most compounds of the invention were measured to be in the range of about 0-120%. The IC₅₀ was the concentration of the compound of the invention at which 50% inhibition of acetylcholine GTPγ³⁵S binding stimulation has been observed. The IC₅₀ towards human M4 receptors for most compounds of the invention was measured to be in the range of between 3000 and >30000 nM.

Certain compounds of the invention were tested using one or more above assays. Some of the results are summarized in Table 1 below.

TABLE 1
Certain biological properties for certain compounds of the invention

| | hM1EC50 | hM1Emax | hM2_EC50 | hM2 Emax |
|---|---|---|---|---|
| Compound | (nM) | (%) | (nM) | (%) |
| Trans-(+/−)-N-[2-[(3-butyl-1- | 174 | 86 | 1233 | 38 |
| piperidyl)methyl]cyclohexyl]-3- | | | | |
| (4-chlorophenyl)propanamide | | | | |
| Trans-(+/−)-N-[2-[[3-(ethoxymethyl)- | 17 | 97 | 5481 | 25 |
| 1-piperidyl]methyl]cyclohexyl]-4- | | | | |
| 1,3-oxazol-5-yl-benzamide | | | | |
| trans-(+/−)-N-{-2-[(3-Butylpiperidin- | 103 | 94 | 165 | 2 |
| 1-yl)methyl]cyclohexyl}-4- | | | | |
| [(diethylamino)methyl]benzamide | | | | |
| Trans-(±)-N-[2-({3-[(Allyloxy)methyl]piperidin- | 49 | 80 | 390 | 19 |
| 1-yl}methyl)cyclohexyl]-4-[(4-methylpiperazin- | | | | |
| 1-yl)methyl]benzamide | | | | |
| N-[(1S,2R)-2-({(3R)-3-[(allyloxy)methyl]piperidin-1- | 5 | 95 | 216 | 22 |
| yl}methyl)cyclohexyl]-6-(1H-imidazol-1-yl)nicotinamide | | | | |
| N-[(1S,2R)-2-(piperidin-1-ylmethyl)cyclohexyl]-6-(1H- | 26 | 82 | >30000 | 0 |
| pyrazol-1-yl)nicotinamide | | | | |
| (N-((1S,2R)-2-{[(3R)-3-ethoxypiperidin-1- | 1504 | 76 | Not | Not |
| yl]methyl}cyclohexyl)pyrazine-2-carboxamide | | | tested | tested |
| N-((1S,2R)-2-{[(3R)-3-(ethoxymethyl)piperidin-1- | 41 | 91 | 107 | 60 |
| yl]methyl}cyclohexyl)-6-pyrrolidin-1-ylnicotinamide | | | | |
| N-[(1S,2R)-2-(azepan-1-ylmethyl)cyclohexyl]-4-(1H- | 130 | 70 | >30000 | >12 |
| pyrazol-1-yl)benzamide | | | | |
| N-((1S,2R)-2-{[(3R)-3-(allyloxy)piperidin-1- | 33 | 97 | 4446 | 50 |
| yl]methyl}cyclohexyl)-6-(1H-pyrazol-1-yl)nicotinamide | | | | |
| N-((1S,2R)-2-{[(3R)-3-(ethoxymethyl)piperidin-1- | 20 | 95 | 1344 | 34 |
| yl]methyl}cyclohexyl)-4- | | | | |
| {[(methylsulfonyl)amino]methyl}benzamide | | | | |
| 4-[(diacetylamino)methyl]-N-((1S,2R)-2-{[(3R)-3- | 103 | 91 | 3305 | 18 |
| (ethoxymethyl)piperidin-1- | | | | |
| yl]methyl}cyclohexyl)benzamide | | | | |
| N-((1S,2R)-2-{[(3R)-3-(ethoxymethyl)piperidin-1- | 119 | 74 | 1905 | 34 |
| yl]methyl}cyclohexyl)benzamide | | | | |
| N-((1S,2R)-2-{[(3R)-3-(ethoxymethyl)piperidin-1- | 157 | 75 | 1075 | 30 |
| yl]methyl}cyclohexyl)cyclohexanecarboxamide | | | | |
| N-((1S,2R)-2-{[(3R)-3-(ethoxymethyl)piperidin-1- | 55 | 98 | 570 | 54 |
| yl]methyl}cyclohexyl)chromane-2-carboxamide | | | | |
| N-((1S,2R)-2-{[(3R)-3-(ethoxymethyl)piperidin-1- | 504 | 54 | Not | Not |
| yl]methyl}cyclohexyl)-4,6-dimethylnicotinamide | | | tested | tested |
| N²-acetyl-N¹-((1S,2R)-2-{[(3R)-3- | 322 | 91 | Not | Not |
| (ethoxymethyl)piperidin-1- | | | tested | tested |
| yl]methyl}cyclohexyl)glycinamide | | | | |
| N-((1S,2R)-2-{[(3R)-3-(ethoxymethyl)piperidin-1- | 22 | 82 | >30000 | 0 |
| yl]methyl}cyclohexyl)-5,7-dimethylpyrazolo[1,5- | | | | |
| a]pyrimidine-2-carboxamid | | | | |
| N-((1S,2R)-2-{[(3R)-3-(ethoxymethyl)piperidin-1- | 152 | 85 | >30000 | 0 |
| yl]methyl}cyclohexyl)-4-methyl-3,4-dihydro-2H-1,4- | | | | |
| benzoxazine-7-carboxamide | | | | |
| N-((1S,2R)-2-{[(3R)-3-(ethoxymethyl)piperidin-1- | 9 | 95 | 1211 | 27 |
| yl]methyl}cyclohexyl)-4-(1H-tetrazol-1-yl)benzamide | | | | |
| N-[(1S,2R)-2-[(3-phenyl-1- | 393.3 | 105.1 | Not | Not |
| piperidyl)methyl]cyclohexyl]-6- | | | tested | tested |
| pyrazol-1-yl-pyridine-3-carboxamide | | | | |
| 4-[(cyclopropylsulfonylamino)methyl]-N-[(1S,2R)-2- | 11 | 102 | 1431 | 40 |
| [[(3R)-3-(ethoxymethyl)-1- | | | | |
| piperidyl]methyl]cyclohexyl]benzamide | | | | |

### EXAMPLES

The invention will further be described in more detail by the following Examples which describe methods whereby compounds of the present invention may be prepared, purified, analyzed and biologically tested, and which are not to be construed as limiting the invention.

### Example 1

### trans-(+/−)-4-fluoro-N-[2-(piperidin-1-ylmethyl)cyclohexyl]benzamide

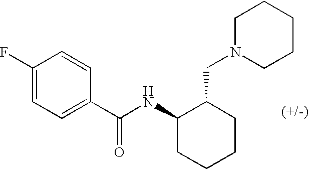

### Step A

### The Preparation of [2-(piperidin-1-ylmethyl)cyclohexyl]amine

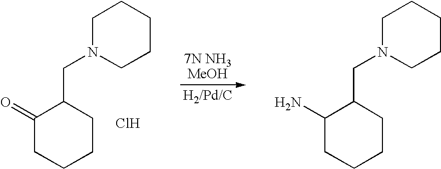

To a solution of 2-(piperidin-1-ylmethyl)cyclohexanone hydrochloride (5.0 g, 21.6 mmol) in 7N NH₃ in MeOH (50 mL) was added 10% Pd/C (0.5 g) and the mixture was hydrogenated at 40 psi overnight. Filtration of catalyst and concentration of MeOH afforded a cis/trans mixture of [2-(piperidin-1-ylmethyl)cyclohexyl]amine (3.94 g, 93%), which was used without further purification.

### Step B

### The Preparation of trans-(+/−)-benzyl[2-(piperidin-1-ylmethyl)cyclohexyl]carbamate

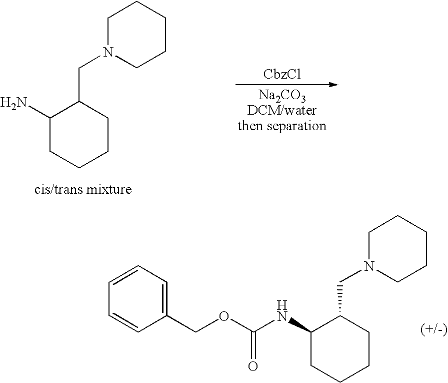

To a solution of [2-(piperidin-1-ylmethyl)cyclohexyl]amine (crude from Step A, 3.94 g, 20.1 mmol) in dichloromethane (80 mL) was added a solution of Na₂CO₃ (4.0 g) in water (100 mL), then benzyl chloroformate (3.44 g, 20.1 mmol) was added slowly in 5 min. The reaction mixture was stirred at room temperature for 1 h. The organic phase was separated, washed with water (50 mL) and brine (50 mL), dried over Na₂SO₄, yielded crude product as cis/trans mixture (˜1:3 ratio, 6.3 g), which was separated by using reverse phase HPLC to yield trans-(+/−)-isomer 4.8 g (54%) as its TFA salt. MS (M+1): 331.1.

### Step C

### The Preparation of trans-(+/−)-[2-(piperidin-1-ylmethyl)cyclohexyl]amine

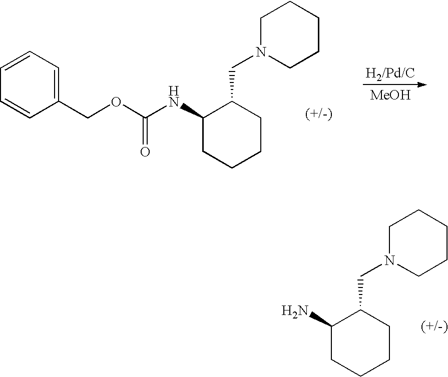

To a solution of trans-(+/−)-benzyl[2-(piperidin-1-ylmethyl)cyclohexyl]carbamate TFA salt (8.85 g, 20.0 mmol) in MeOH (50 mL) was added 10% Pd/C (1.0 g) and the mixture was hydrogenated at 40 psi for 6 h. Filtration of catalyst and concentration of MeOH afforded trans-(+/−)-[2-(piperidin-1-ylmethyl)cyclohexyl]amine as its TFA salt (6.18 g, 99%), which was used without further purification.

### Step D

### The Preparation of trans-(+/−)-4-fluoro-N-[2-(piperidin-1-ylmethyl)cyclohexyl]benzamide

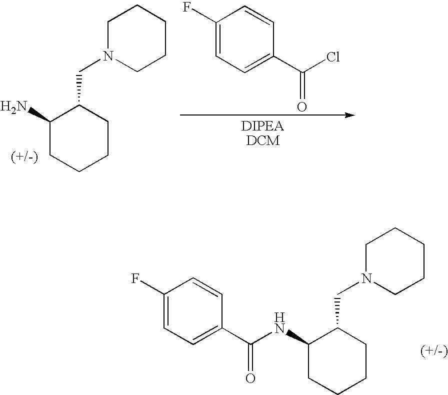

To the solution of trans-(+/−)-[2-(piperidin-1-ylmethyl)cyclohexyl]amine (0.4 mmol) in dry DCM (5 mL) was added 4-fluorobenzoyl chloride (0.5 mmol) followed by diisopropylethylamine (1.0 mmol), the mixture was stirred at room temperature for 1 h, and the reaction was quenched with water (5 mL). DCM (10 mL) was added and washed with saturated NaHCO₃ (5 mL) and brine (10 mL), dried over Na₂SO₄. The crude product was purified with reverse phase HPLC to yield trans-(+/−)-4-fluoro-N-[2-(piperidin-1-ylmethyl)cyclohexyl]benzamide (84 mg, 49%) as its TFA salt. MS (M+1): 319.3. 1H NMR (400 MHz, METHANOL-D4): [symbol] pp [symbol] 1.17-1.30 (m, 1H), 1.31-1.41 (m, 2H), 1.41-1.57 (m, 2H), 1.71-1.88 (m, 6H), 1.91-2.00 (m, 2H), 2.02-2.11 (m, 1H), 2.72-2.85 (m, 1H), 2.91-3.05 (m, 2H), 3.11-3.23 (m, 1H), 3.37-3.47 (m, 1H), 3.53-3.61 (m, 1H), 3.65-3.79 (m, 2H), 7.18 (t, J=8.79 Hz, 2H), 7.84-7.95 (m, 2H).

### Example 2

### trans-(+/−)-N-[2-(piperidin-1-ylmethyl)cyclohexyl]-6-(1H-pyrazol-1-yl)nicotinamide

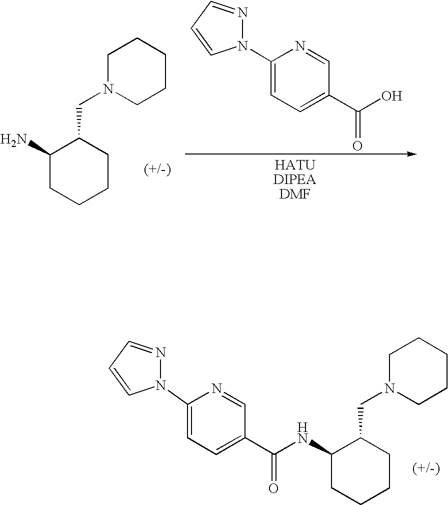

To the solution of trans-(+/−)-[2-(piperidin-1-ylmethyl)cyclohexyl]amine hydrochloride (116 mg, 0.5 mmol) in dry DMF (5 mL) was added 6-(1H-pyrazol-1-yl)nicotinic acid (113 mg, 0.6 mmol) followed by HATU (228 mg, 0.6 mmol) and diisopropylethylamine (0.18 mL, 1.0 mmol). The mixture was stirred at room temperature for 1 h, and the reaction was quenched with water (5 mL). The solvent was removed in vacuo. DCM (15 mL) was added and washed with saturated NaHCO₃ (10 mL) and brine (10 mL), dried over Na₂SO₄. The crude product was purified with reverse phase HPLC to yield trans-(+/−)-N-[2-(piperidin-1-ylmethyl)cyclohexyl]-6-(1H-pyrazol-1-yl)nicotinamide (156 mg, 71%) as its HCl salt. MS (M+1): 368.3. 1H NMR (400 MHz, METHANOL-D4): [symbol] pp [symbol] 1.20-1.62 (m, 5H), 1.72-1.93 (m, 7H), 1.95-2.15 (m, 3H), 2.76-2.90 (m, 1H), 2.94-3.06 (m, 2H), 3.16-3.24 (m, 1H), 3.39-3.50 (m, 1H), 3.59 (d, J=11.33 Hz, 1H), 3.74-3.85 (m, 1H), 6.55 (d, J=1.76 Hz, 1H), 7.79 (s, 1H), 8.01 (d, J=8.59 Hz, 1H), 8.38 (dd, J=8.59, 2.34 Hz, 1H), 8.64 (d, J=2.54 Hz, 1H), 8.91 (d, J=1.95 Hz, 1H).

### Example 3

### trans-(+/−)-N-[2-(piperidin-1-ylmethyl)cyclohexyl]-6-(trifluoromethyl)nicotinamide

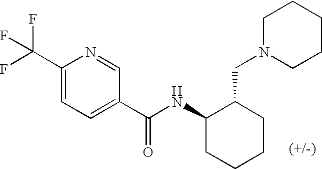

Following the same procedure as Example 2, yielded trans-(+/−)-N-[2-(piperidin-1-ylmethyl)cyclohexyl]-6-(trifluoromethyl)nicotinamide (143 mg, 65%) as its HCl salt. MS (M+1): 370.3. 1H NMR (400 MHz, METHANOL-D4): [symbol] ppm 1.17-1.62 (m, 5H), 1.71-1.93 (m, 8H), 1.95-2.11 (m, 2H), 2.81 (s, 1H), 2.94-3.08 (m, 2H), 3.15-3.24 (m, 1H), 3.39-3.49 (m, 1H), 3.54-3.63 (m, 1H), 3.75-3.86 (m, 1H), 7.93 (d, J=8.20 Hz, 1H), 8.44 (dd, J=8.20, 1.47 Hz, 1H), 9.12 (s, 1H).

### Example 4

### trans-(+/−)-N-[2-(piperidin-1-ylmethyl)cyclohexyl]-4-(1H-pyrazol-1-yl)benzamide

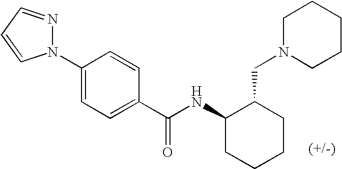

Following the same procedure as Example 2, yielded trans-(+/−)-N-[2-(piperidin-1-ylmethyl)cyclohexyl]-4-(1H-pyrazol-1-yl)benzamide (121 mg, 66%) as its free base. MS (M+1): 367.3. 1H NMR (400 MHz, METHANOL-D4): [symbol] pp [symbol] 0.99-1.18 (m, 1H), 1.26-1.46 (m, 4H), 1.47-1.62 (m, 4H), 1.65-1.83 (m, 3H), 1.94 (d, J=12.69 Hz, 1H), 2.06-2.23 (m, 2H), 2.31-2.53 (m, 6H), 3.54-3.64 (m, 1H), 6.54 (s, 1H), 7.74 (s, 1H), 7.83-7.89 (m, 2H), 7.91-7.98 (m, 2H), 8.31 (d, J=2.34 Hz, 1H).

### Example 5

### trans-(+/−)-5-chloro-N-[2-(piperidin-1-ylmethyl)cyclohexyl]-1-benzofuran-2-carboxamide

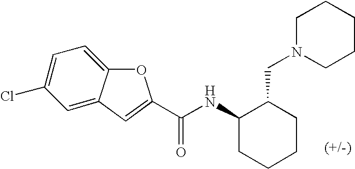

Following the same procedure as Example 2, yielded trans-(+/−)-5-chloro-N-[2-(piperidin-1-ylmethyl)cyclohexyl]-1-benzofuran-2-carboxamide (93 mg, 62%) as its free base. MS (M+1): 375.3. 1H NMR (400 MHz, METHANOL-D4): δ pp [symbol] 1.01-1.15 (m, 1H) 1.25-1.38 (m, 3H), 1.39-1.49 (m, 2H), 1.50-1.63 (m, 4H), 1.66-1.80 (m, 3H), 1.86 (d, J=13.28 Hz, 1H), 2.12 (dd, J=12.79, 5.18 Hz, 1H), 2.21 (d, J=11.33 Hz, 1H), 2.27-2.52 (m, 5H), 3.47-3.59 (m, 1H), 7.37-7.46 (m, 2H), 7.48-7.55 (m, 1H), 7.73 (d, J=1.95 Hz, 1H).

### Example 6

### trans-(+/−)-2-(4-methoxyphenyl)-N-[2-(piperidin-1-ylmethyl)cyclohexyl]acetamide

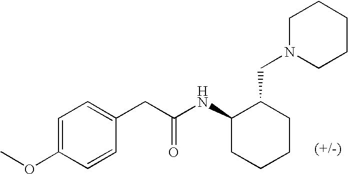

Following the same procedure as Example 2, yielded trans-(+/−)-2-(4-methoxyphenyl)-N-[2-(piperidin-1-ylmethyl)cyclohexyl]acetamide (94 mg, 68%) as its free base. MS (M+1): 345.3. 1H NMR (400 MHz, METHANOL-D4): δ pp [symbol] 0.90-1.05 (m, 1H), 1.13-1.31 (m, 3H), 1.33-1.46 (m, 3H), 1.46-1.58 (m, 4H), 1.61-1.76 (m, 2H), 1.82-1.91 (m, 1H), 1.92-2.04 (m, 2H), 2.07-2.19 (m, 3H), 2.21-2.36 (m, 2H), 3.31-3.36 (m, 1H), 3.37 (s, 2H), 3.74 (s, 3H), 6.84 (d, J=8.59 Hz, 2H), 7.21 (d, J=8.59 Hz, 2H).

### Example 7

### trans-(+/−)-4-(difluoromethoxy)-N-[2-(piperidin-1-ylmethyl)cyclohexyl]benzamide

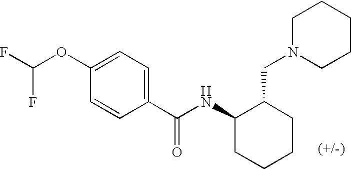

Following the same procedure as Example 2, yielded trans-(+/−)-4-(difluoromethoxy)-N-[2-(piperidin-1-ylmethyl)cyclohexyl]benzamide (163 mg, 67%) as its HCl salt. MS (M+1): 367.3. 1H NMR (400 MHz, METHANOL-D4): [symbol] pp [symbol] 1.00-1.15 (m, 1H), 1.24-1.45 (m, 5H), 1.44-1.59 (m, 4H), 1.58-1.69 (m, 1H), 1.69-1.82 (m, 2H), 1.93 (d, J=13.09 Hz, 1H), 2.05-2.18 (m, 2H), 2.28-2.46 (m, 5H), 3.49-3.61 (m, 1H), 6.92 (t, J=73.63 Hz, 1H), 7.20 (d, J=8.79 Hz, 2H), 7.85 (d, J=8.79 Hz, 2H).

### Example 8

### trans-(+/−)-4-(2-methoxyethoxy)-N-[2-(piperidin-1-ylmethyl)cyclohexyl]benzamide

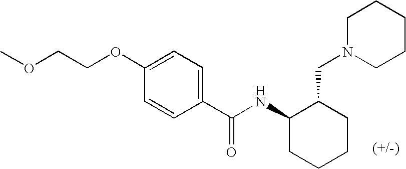

Following the same procedure as Example 2, yielded trans-(+/−)-4-(2-methoxyethoxy)-N-[2-(piperidin-1-ylmethyl)cyclohexyl]benzamide (194 mg, 47%) as its HCl salt. MS (M+1): 375.3. 1H NMR (400 MHz, METHANOL-D4): δ pp [symbol] 1.17-1.60 (m, 5H), 1.70-1.96 (m, 8H), 2.00-2.17 (m, 2H), 2.81 (t, J=11.13 Hz, 1H), 2.88-3.03 (m, 2H), 3.13 (d, J=12.50 Hz, 1H), 3.39 (s, 3H), 3.41 (d, J=11.71 Hz, 1H), 3.56 (d, J=11.71 Hz, 1H), 3.68-3.79 (m, 3H), 4.10-4.20 (m, 2H), 6.99 (d, J=8.59 Hz, 2H), 7.87 (d, J=8.59 Hz, 2H).

### Example 9

### trans-(+)-4-(2-methoxyethoxy)-N-[2-(piperidin-1-ylmethyl)cyclohexyl]benzamide (Isomer 1)

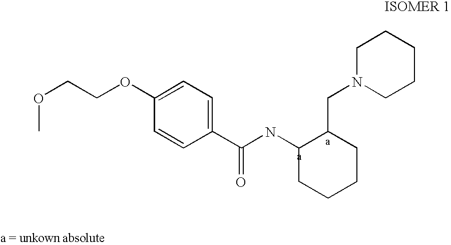

The racemic product from Example 8 (98 mg, HCl salt) was separated by chiral AD column (15% IPA in Hexanes) to yield trans-(+)-4-(2-methoxyethoxy)-N-[2-(piperidin-1-ylmethyl)cyclohexyl]benzamide (27 mg, 31%) as its free base. [α]²⁰_{D}+35.3 (c2.0, MeOH). MS (M+1): 375.3. 1H NMR (400 MHz, METHANOL-D4): [symbol] pp [symbol] 1.17-1.60 (m, 5H), 1.70-1.96 (m, 8H), 2.00-2.17 (m, 2H), 2.81 (t, J=11.13 Hz, 1H), 2.88-3.03 (m, 2H), 3.13 (d, J=12.50 Hz, 1H), 3.39 (s, 3H), 3.41 (d, J=11.71 Hz, 1H), 3.56 (d, J=11.71 Hz, 1H), 3.68-3.79 (m, 3H), 4.10-4.20 (m, 2H), 6.99 (d, J=8.59 Hz, 2H), 7.87 (d, J=8.59 Hz, 2H).

### Example 10

### trans-(−)4-(2-methoxyethoxy)-N-[2-(piperidin-1-ylmethyl)cyclohexyl]benzamide (Isomer 1)

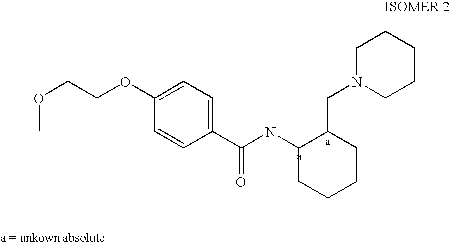

The racemic product from Example 8 (98 mg, HCl salt) was separated by chiral AD column (15% IPA in Hexanes) to yield trans-(−)-4-(2-methoxyethoxy)-N-[2-(piperidin-1-ylmethyl)cyclohexyl]benzamide (29 mg, 33%) as its free base. [α]²⁰_{D}−31.5 (c2.0, MeOH). MS (M+1): 375.3. 1H NMR (400 MHz, METHANOL-D4): [symbol] pp [symbol] 1.17-1.60 (m, 5H), 1.70-1.96 (m, 8H), 2.00-2.17 (m, 2H), 2.81 (t, J=11.13 Hz, 1H), 2.88-3.03 (m, 2H), 3.13 (d, J=12.50 Hz, 1H), 3.39 (s, 3H), 3.41 (d, J=11.71 Hz, 1H), 3.56 (d, J=11.71 Hz, 1H), 3.68-3.79 (m, 3H), 4.10-4.20 (m, 2H), 6.99 (d, J=8.59 Hz, 2H), 7.87 (d, J=8.59 Hz, 2H).

### Example 11

### trans-(+/−)-3-cyclopentyl-N-[2-(piperidin-1-ylmethyl)cyclohexyl]propanamide

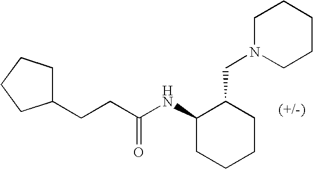

Following the same procedure as Example 2, yielded trans-(+/−)-3-cyclopentyl-N-[2-(piperidin-1-ylmethyl)cyclohexyl]propanamide (117 mg, 82%) as its HCl salt. MS (M+1): 321.3; 1H NMR (400 MHz, METHANOL-D4): δ ppm 1.09-1.41 (m, 5H), 1.46-1.66 (m, 7H), 1.71-2.02 (m, 14H), 2.19-2.26 (m, 2H), 2.76-2.85 (td, J=12.35, 3.03 Hz, 1H), 2.92 (dd, J=13.48, 9.57 Hz, 1H), 2.97 (td, J=11.91, 3.91 Hz, 1H), 3.06 (dd, J=13.28, 2.93 Hz, 1H), 3.39-3.45 (m, J=12.50 Hz, 1H), 3.47-3.59 (m, 2H).

### Example 12

### trans-(+/−)-3-(4-chlorophenyl)-N-[2-(piperidin-1-ylmethyl)cyclohexyl]propanamide

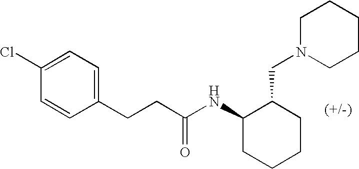

Following the same procedure as Example 2, yielded trans-(+/−)-3-(4-chlorophenyl)-N-[2-(piperidin-1-ylmethyl)cyclohexyl]propanamide (76 mg, 46%) as its HCl salt. MS (M+1): 363.1; 1H NMR (400 MHz, METHANOL-D4): δ ppm 1.09-1.36 (m, 4H), 1.45-1.56 (m, 1H), 1.62-1.94 (m, 10H), 2.48 (td, J=12.69, 2.93 Hz, 1H), 2.52 (t, J=7.23 Hz, 2H), 2.66-2.75 (m, 2H), 2.79 (dd, J=13.28, 9.57 Hz, 1H), 2.84-2.98 (m, 2H), 3.30-3.35 (m, J=13.09 Hz, 1H), 3.40-3.48 (m, 2H), 7.22 (d, J=8.59 Hz, 2H), 7.29 (d, J=8.59 Hz, 2H).

### Example 13

### trans-(+/−)-3-(2-methoxyphenyl)-N-[2-(piperidin-1-ylmethyl)cyclohexyl]propanamide

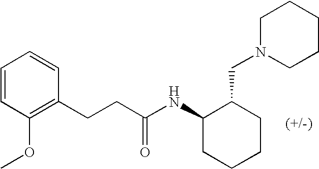

Following the same procedure as Example 2, yielded trans-(+/−)-3-(4-chlorophenyl)-N-[2-(piperidin-1-ylmethyl)cyclohexyl]propanamide (109 mg, 69%) as its HCl salt. MS (M+1): 359.3; 1H NMR (400 MHz, METHANOL-D4): δ ppm 1.10-1.35 (m, 4H), 1.43-1.52 (m, 1H), 1.64-1.89 (m, 9H), 1.94-2.01 (m, 1H), 2.43-2.58 (m, 3H), 2.77-2.83 (m, 3H), 2.84-2.97 (m, 2H), 3.30-3.35 (m, 1H), 3.40-3.49 (m, 2H), 3.81 (s, 3H), 6.84 (td, J=7.37, 1.07 Hz, 1H), 6.92 (d, J=8.20 Hz, 1H), 7.13 (dd, J=7.42, 1.56 Hz, 1H), 7.19 (td, J=7.81, 1.76 Hz, 1H).

### Example 14

### trans-(+/−)-4-tert-butyl-N-[2-(piperidin-1-ylmethyl)cyclohexyl]benzamide

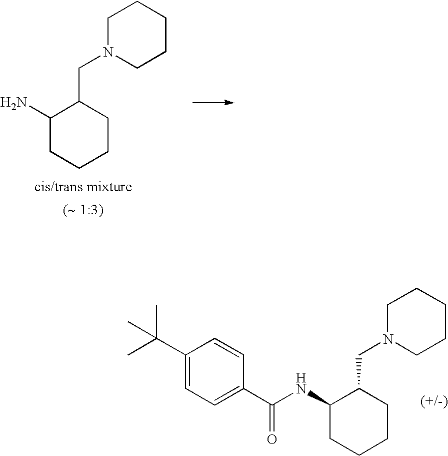

Following the same procedure as Example 2, but used cis/trans mixture of [2-(piperidin-1-ylmethyl)cyclohexyl]amine (˜1:3 ratio, 0.35 mmol). After the same work-up, the crude product was purified with reverse phase HPLC to yielded trans-(+/−)-4-tert-butyl-N-[2-(piperidin-1-ylmethyl)cyclohexyl]benzamide (34 mg, 21%) as its TFA salt. MS (M+1): 357.0. 1H NMR (400 MHz, METHANOL-D4): [symbol] pp [symbol] 1.32 (s, 9H), 1.30-1.59 (m, 6H), 1.67-1.89 (m, 6H), 1.90-2.01 (m, 2H), 2.03-2.08 (m, 1H), 2.72-2.84 (m, 1H), 2.90-3.04 (m, 2H), 3.06-3.19 (m, 1H), 3.40 (d, J=12.01 Hz, 1H), 3.57 (d, J=12.01 Hz, 1H), 3.70-3.81 (m, 1H), 7.50 (d, J=8.40 Hz, 2H), 7.77 (d, J=8.40 Hz, 2H).

### Example 15

### trans-(+/−)-4-methoxy-N-[2-(piperidin-1-ylmethyl)cyclohexyl]benzamide

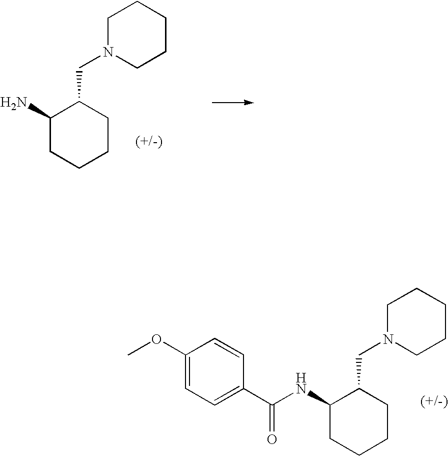

Following the same procedure as Example 1 (step D), yielded trans-(+/−)-4-methoxy-N-[2-(piperidin-1-ylmethyl)cyclohexyl]benzamide (134 mg, 82%) as its HCl salt. MS (M+1): 331.2. 1H NMR (400 MHz, METHANOL-D4): [symbol] pp [symbol] 1.17-1.59 (m, 5H), 1.68-1.89 (m, 7H), 1.90-1.99 (m, 2H), 2.05 (d, J=12.30 Hz, 1H), 2.73-2.84 (m, 1H), 2.93-3.04 (m, 2H), 3.13 (dd, J=13.28, 2.73 Hz, 1H), 3.40 (d, J=12.30 Hz, 1H), 3.58 (d, J=12.30 Hz, 1H), 3.71-3.80 (m, 1H), 3.84 (s, 3H), 6.98 (d, J=8.89 Hz, 2H), 7.81 (d, J=8.89 Hz, 2H).

### Example 16

### trans-(+/−)-4-cyano-N-[2-(piperidin-1-ylmethyl)cyclohexyl]benzamide

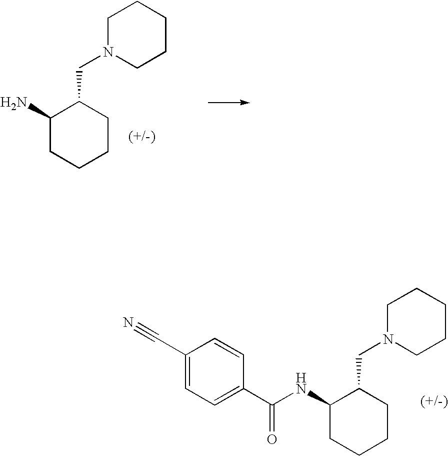

Following the same procedure as Example 1 (step D), yielded trans-(+/−)-4-cyano-N-[2-(piperidin-1-ylmethyl)cyclohexyl]benzamide (198 mg, 74%) as its HCl salt. MS (M+1): 326.0. 1H NMR (400 MHz, METHANOL-D4): [symbol] pp [symbol] 1.18-1.59 (m, 5H), 1.71-2.00 (m, 8H), 2.01-2.18 (m, 2H), 2.76-2.90 (m, 1H), 2.92-3.07 (m, 2H), 3.17 (d, J=11.91 Hz, 1H), 3.44 (d, J=12.11 Hz, 1H), 3.58 (d, J=12.11 Hz, 1H), 3.71-3.84 (m, 1H), 7.84 (d, J=8.20 Hz, 2H), 8.04 (d, J=8.20 Hz, 2H).

### Example 17

### trans-(+/−)-4-bromo-N-[2-(piperidin-1-ylmethyl)cyclohexyl]benzamide

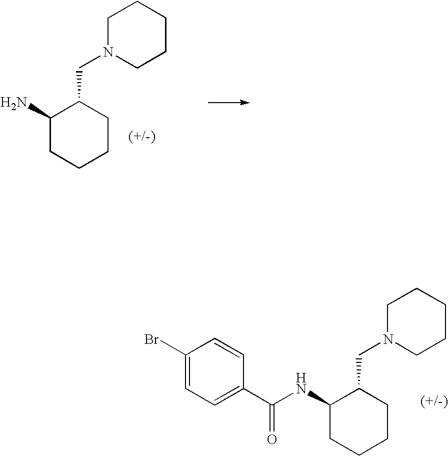

Following the same procedure as Example 1 (step D), yielded trans-(+/−)-4-bromo-N-[2-(piperidin-1-ylmethyl)cyclohexyl]benzamide (123 mg, 74%) as its HCl salt. MS (M+1): 379.0. 1H NMR (400 MHz, METHANOL-D4): [symbol] pp [symbol] 1.15-1.61 (m, 6H), 1.73-1.92 (m, 6H), 1.93-2.18 (m, 3H), 2.70-2.88 (m, 1H), 2.95-3.06 (m, 2H), 3.16 (dd, J=13.28, 2.73 Hz, 1H), 3.55-3.70 (m, 2H), 3.72-3.84 (m, 1H), 7.66 (d, J=8.59 Hz, 2H), 7.78 (d, J=8.59 Hz, 2H).

### Example 18

### trans-(+/−)-4-chloro-N-[2-(piperidin-1-ylmethyl)cyclohexyl]benzamide

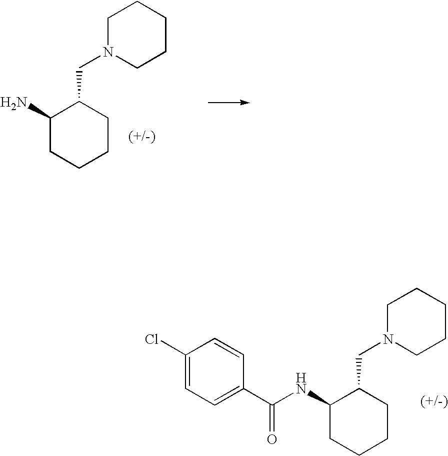

Following the same procedure as Example 1 (step D), yielded trans-(+/−)-4-chloro-N-[2-(piperidin-1-ylmethyl)cyclohexyl]benzamide (93 mg, 42%) as its HCl salt. MS (M+1): 335.3. 1H NMR (400 MHz, METHANOL-D4): [symbol] pp [symbol] 1.23-1.60 (m, 6H), 1.73-1.92 (m, 7H), 1.93-2.12 (m, 2H), 2.74-2.89 (m, 1H), 2.94-3.08 (m, 2H), 3.16 (dd, J=13.28, 2.73 Hz, 1H), 3.38-3.50 (m, 1H), 3.56-3.64 (m, 1H), 3.72-3.83 (m, 1H), 7.50 (d, J=8.59 Hz, 2H), 7.85 (d, J=8.59 Hz, 2H).

### Example 19

### trans-(+/−)-6-(1H-imidazol-1-yl)-N-[2-(piperidin-1ylmethyl)cyclohexyl]nicotinamide

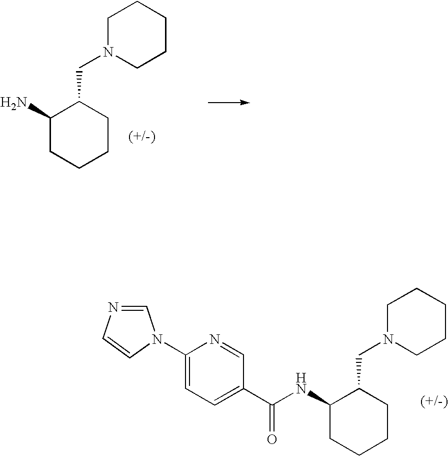

Following the same procedure as Example 2, yielded trans-(+/−)-6-(1H-imidazol-1-yl)-N-[2-(piperidin-1-ylmethyl)cyclohexyl]nicotinamide (94 mg, 51%) as white solids. MS (M+1): 368.3. 1H NMR (400 MHz, METHANOL-D4): δ ppm 0.99-1.18 (m, 1H), 1.26-1.45 (m, 4H), 1.45-1.62 (m, 4H), 1.61-1.70 (m, 1H), 1.70-1.82 (m, 2H), 1.90-1.99 (m, 1H), 2.07-2.17 (m, 2H), 2.23-2.49 (m, 6H), 3.54-3.66 (m, 1H), 7.16 (s, 1H), 7.80 (d, J=8.59 Hz, 1H), 7.95 (s, 1H), 8.34 (dd, J=8.50, 2.25 Hz, 1H), 8.60 (d, 1H), 8.91 (d, J=1.95 Hz, 1H).

### Example 20

### trans-(+/−)-4-(1,3-oxazol-5-yl)-N-[-2-(piperidin-1-ylmethyl)cyclohexyl]benzamide

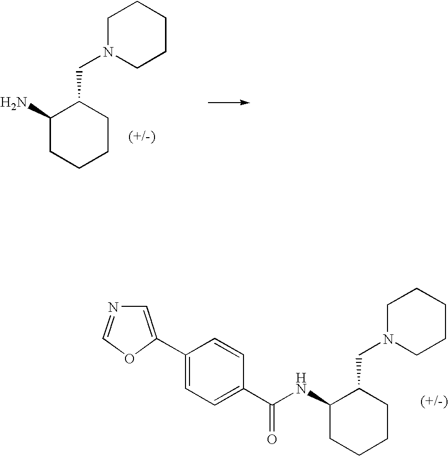

Following the same procedure as Example 2, yielded trans-(+/−)-4-(1,3-oxazol-5-yl)-N-[-2-(piperidin-1-ylmethyl)cyclohexyl]benzamide (123 mg, 67%) as white solids. MS (M+1): 368.3. 1H NMR (400 MHz, METHANOL-D4): δ ppm 1.02-1.19 (m, 1H), 1.24-1.44 (m, 4H), 1.44-1.60 (m, 4H), 1.61-1.71 (m, 1H), 1.71-1.82 (m, 2H), 1.88-1.99 (m, 1H), 2.07-2.18 (m, 2H), 2.24-2.48 (m, 6H), 3.51-3.63 (m, 1H), 7.64 (s, 1H), 7.77-7.85 (m, 2H), 7.88-7.94 (m, 2H), 8.29 (s, 1H).

### Example 21

### trans-(+/−)-6-methoxy-N-[2-(piperidin-1-ylmethyl)cyclohexyl]nicotinamide

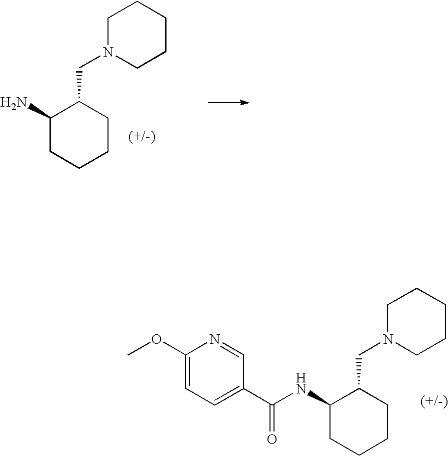

Following the same procedure as Example 2, yielded trans-(+/−)-6-methoxy-N-[2-(piperidin-1-ylmethyl)cyclohexyl]nicotinamide (56 mg, 42%) as white solids. MS (M+1): 332.3. 1H NMR (400 MHz, METHANOL-D4): δ ppm 0.96-1.19 (m, 1H), 1.27-1.41 (m, 3H), 1.43-1.51 (m, 2H), 1.54-1.68 (m, 4H), 1.70-1.85 (m, 3H), 1.90-2.09 (m, 2H), 2.30-2.46 (m, 1H), 2.50-2.81 (m, 5H), 3.56-3.67 (m, 1H), 3.94 (s, 3H), 6.84 (d, J=8.79 Hz, 1H), 8.07 (dd, J=8.69, 2.44 Hz, 1H), 8.62 (d, J=2.34 Hz, 1H).

### Example 22

### trans-(+/−)-4-(1H-imidazol-1-yl)-N-[2-(piperidin-1-ylmethyl)cyclohexyl]benzamide

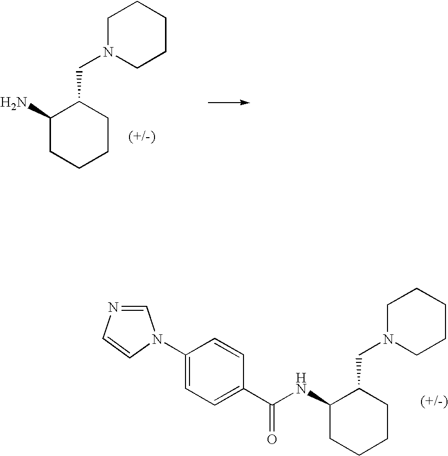

Following the same procedure as Example 2, yielded trans-(+/−)-4-(1H-imidazol-1-yl)-N-[2-(piperidin-1-ylmethyl)cyclohexyl]benzamide (56 mg, 42%) as white solids. MS (M+1): 367.3. 1H NMR (400 MHz, METHANOL-D4): δ ppm 1.00-1.23 (m, 1H), 1.24-1.50 (m, 6H), 1.50-1.66 (m, 5H), 1.66-1.86 (m, 3H), 1.89-2.22 (m, 2H), 2.28-2.77 (m, 4H), 3.51-3.70 (m, 1H), 7.16 (s, 1H), 7.60-7.73 (m, 3H), 7.98 (d, J=8.79 Hz, 2H), 8.25 (s, 1H).

### Examples 23-88

Compounds listed in the following table were prepared as described in Example 2:

| | | | | Retention time |
|---|---|---|---|---|
| Exp. No. | Structure | Name | MS (M + 1) | (min) |
| 23 | 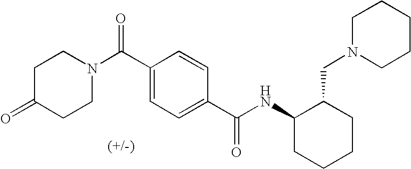 | trans-(+/−)-4-[(4- oxopiperidin-1-yl)carbonyl]- N-[2-(piperidin-1- ylmethyl)cyclohexyl]benza- mide | 426.42 | 1.37 |
| 24 | 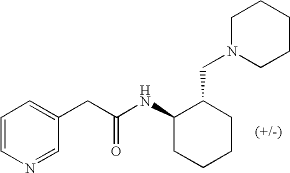 | trans-(+/−)-N-[2-(piperidin- 1-ylmethyl)cyclohexyl]-2- pyridin-3-ylacetamide | 315.97 | 1.17 |
| 25 | 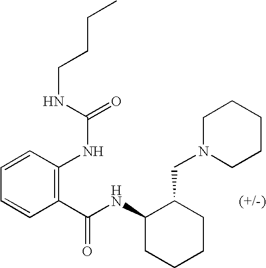 | trans-(+/−)-2- {[(butylamino)carbonyl]ami- no}-N-[2-(piperidin-1- ylmethyl)cyclohexyl]benza- mide | 414.96 | 1.98 |
| 26 | 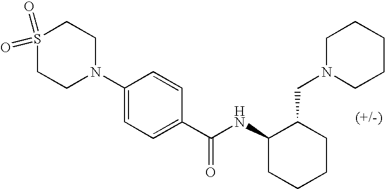 | trans-(+/−)-4-(1,1- dioxidothiomorpholin-4-yl)- N-[2-(piperidin-1- ylmethyl)cyclohexyl]benza- mide | 433.88 | 1.46 |
| 27 | 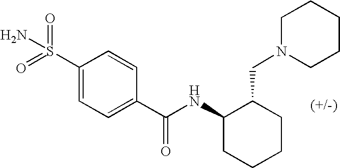 | trans-(+/−)-4- (aminosulfonyl)-N-[2- (piperidin-1- ylmethyl)cyclohexyl]benza- mide | 379.91 | 1.33 |
| 28 | 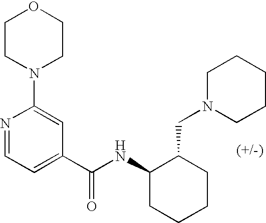 | trans-(+/−)-2-morpholin-4- yl-N-[2-(piperidin-1- ylmethyl)cyclohexyl]isonico- tinamide | 386.95 | 1.54 |
| 29 | 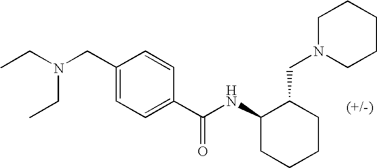 | trans-(+/−)-4- [(diethylamino)methyl]-N- [2-(piperidin-1- ylmethyl)cyclohexyl]benza- mide | 386 | 1.94 |
| 30 | 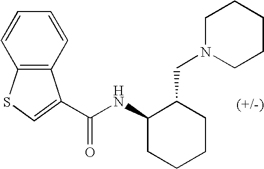 | trans-(+/−)-N-[2-(piperidin- 1-ylmethyl)cyclohexyl]-1- benzothiophene-3- carboxamide | 356.91 | 1.97 |
| 31 | 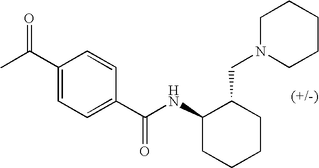 | trans-(+/−)-4-acetyl-N-[2- (piperidin-1- ylmethyl)cyclohexyl]benza- mide | 342.95 | 1.62 |
| 32 | 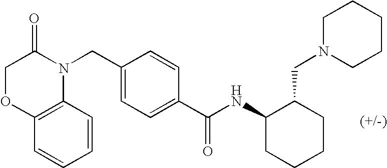 | trans-(+/−)-4-[(3-oxo-2,3- dihydro-4H-1,4- benzoxazin-4-yl)methyl]-N- [2-(piperidin-1- ylmethyl)cyclohexyl]benza- mide | 461.89 | 1.85 |
| 33 | 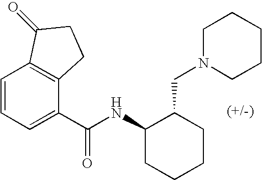 | trans-(+/−)-1-oxo-N-[2- (piperidin-1- ylmethyl)cyclohexyl]indane- 4-carboxamide | 354.94 | 1.55 |
| 34 | 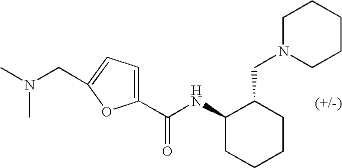 | trans-(+/−)-5- [(dimethylamino)methyl]-N- [2-(piperidin-1- ylmethyl)cyclohexyl]-2- furamide | 348.01 | 1.43 |
| 35 | 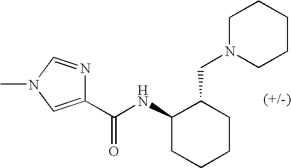 | trans-(+/−)-1-methyl-N-[2- (piperidin-1- ylmethyl)cyclohexyl]-1H- imidazole-4-carboxamide | 304.98 | 1.15 |
| 36 | 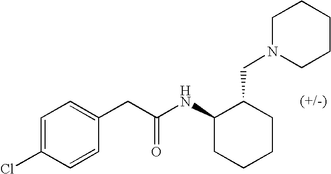 | trans-(+/−)-2-(4- chlorophenyl)-N-[2- (piperidin-1- ylmethyl)cyclohexyl]aceta- mide | 348.92 | 1.68 |
| 37 | 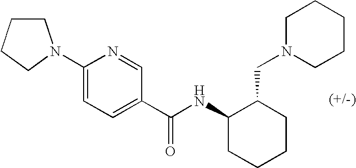 | trans-(+/−)-N-[2-(piperidin- 1-ylmethyl)cyclohexyl]-6- pyrrolidin-1-ylnicotinamide | 371 | 1.61 |
| 38 | 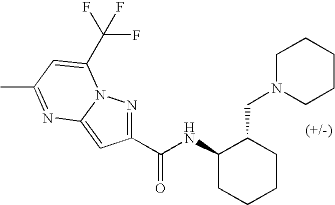 | trans-(+/−)-5-methyl-N-[2- (piperidin-1- ylmethyl)cyclohexyl]-7- (trifluoromethyl)pyrazolo[1, 5-a]pyrimidine-2- carboxamide | 424.44 | 1.79 |
| 39 | 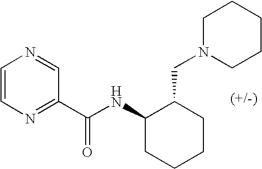 | trans-(+/−)-N-[2-(piperidin-1- ylmethyl)cyclohexyl]pyrazine-2- carboxamide | 303.02 | 1.46 |
| 40 | 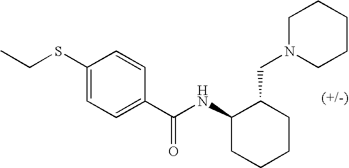 | trans-(+/−)-4-(ethylthio)-N- [2-(piperidin-1- ylmethyl)cyclohexyl]benza- mide | 360.94 | 1.99 |
| 41 | 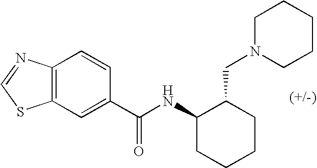 | trans-(+/−)-N-[2-(piperidin- 1-ylmethyl)cyclohexyl]-1,3- benzothiazole-6- carboxamide | 357.96 | 1.6 |
| 42 | 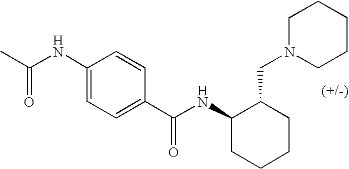 | trans-(+/−)-4-(acetylamino)- N-[2-(piperidin-1- ylmethyl)cyclohexyl]benza- mide | 357.96 | 1.36 |
| 43 | 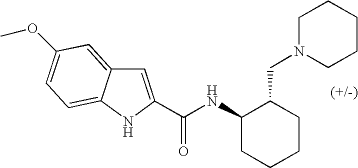 | trans-(+/−)-5-methoxy-N-[2- (piperidin-1- ylmethyl)cyclohexyl]-1H- indole-2-carboxamide | 370 | 1.75 |
| 44 | 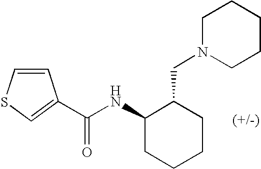 | trans-(+/−)-N-[2-(piperidin-1- ylmethyl)cyclohexyl]thiophene- 3-carboxamide | 306.97 | 1.58 |
| 45 | 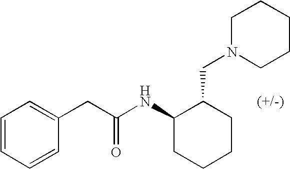 | trans-(+/−)-2-phenyl-N-[2- (piperidin-1- ylmethyl)cyclohexyl]aceta- mide | 314.98 | 1.53 |
| 46 | 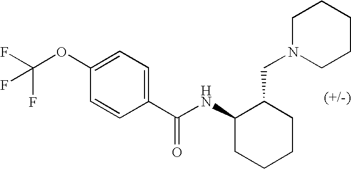 | trans-(+/−)-N-[2-(piperidin- 1-ylmethyl)cyclohexyl]-4- (trifluoromethoxy)benzamide | 384.87 | 2.04 |
| 47 | 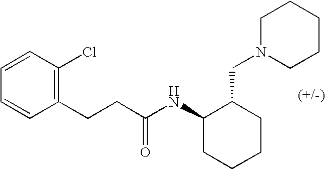 | trans-(+/−)-3-(2- chlorophenyl)-N-[2- (piperidin-1- ylmethyl)cyclohexyl]propan- amide | 362.9 | 1.82 |
| 48 | 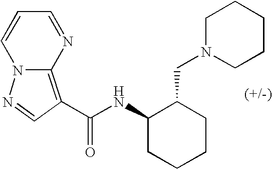 | trans-(+/−)-N-[2-(piperidin-1- ylmethyl)cyclohexyl]pyrazo- lo[1,5-a]pyrimidine-3- carboxamide | 341.93 | 1.32 |
| 49 | 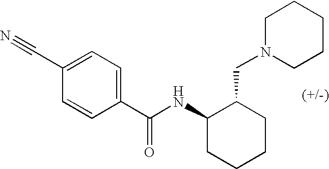 | trans-(+/−)-N-[2-(piperidin- 1-ylmethyl)cyclohexyl]-4- cyano benzamide | 324.93 | 1.86 |
| 50 | 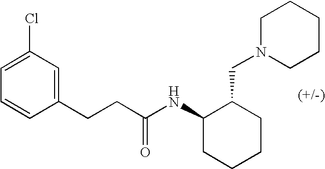 | trans-(+/−)-3-(3- chlorophenyl)-N-[2- (piperidin-1- ylmethyl)cyclohexyl]propan- amide | 362.89 | 1.81 |
| 51 | 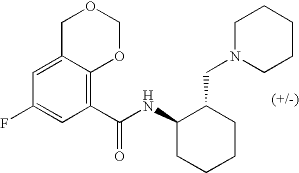 | trans-(+/−)-6-fluoro-N-[2-(pipe- ridin-1- ylmethyl)cyclohexyl]-4H- 1,3-benzodioxine-8- carboxamide | 376.88 | 1.67 |
| 52 | 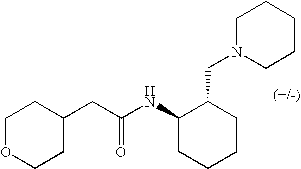 | trans-(+/−)-N-[2-(piperidin- 1-ylmethyl)cyclohexyl]-2- (tetrahydro-2H-pyran-4- yl)acetamide | 322.97 | 1.24 |
| 53 | 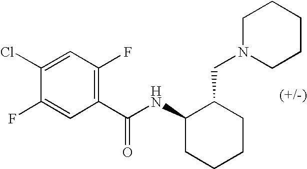 | trans-(+/−)-4-chloro-2,5- difluoro-N-[2-(piperidin-1- ylmethyl)cyclohexyl]benza- mide | 370.84 | 2.05 |
| 54 | 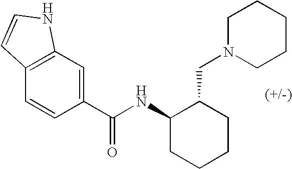 | trans-(+/−)-N-[2-(piperidin- 1-ylmethyl)cyclohexyl]-1H- indole-6-carboxamide | 339.92 | 1.68 |
| 55 | 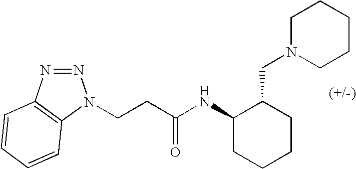 | trans-(+/−)-3-(1H-1,2,3- benzotriazol-1-yl)-N-[2- (piperidin-1- ylmethyl)cyclohexyl]propan- amide | 369.97 | 1.34 |
| 56 | 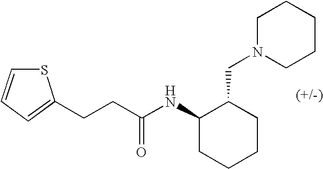 | trans-(+/−)-N-[2-(piperidin- 1-ylmethyl)cyclohexyl]-3- (2-thienyl)propanamide | 334.95 | 1.62 |
| 57 | 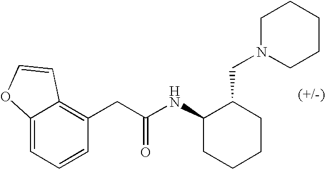 | trans-(+/−)-2-(1- benzofuran-4-yl)-N-[2- (piperidin-1- ylmethyl)cyclohexyl]aceta- mide | 354.92 | 1.62 |
| 58 | 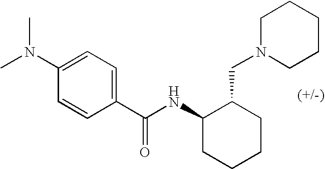 | trans-(+/−)-4- (dimethylamino)-N-[2- (piperidin-1- ylmethyl)cyclohexyl]benza- mide | 343.97 | 1.75 |
| 59 | 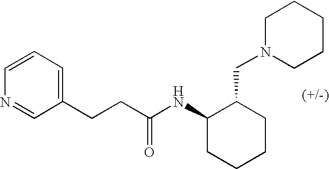 | trans-(+/−)-N-[2-(piperidin- 1-ylmethyl)cyclohexyl]-3- pyridin-3-ylpropanamide | 329.99 | 1.21 |
| 60 | 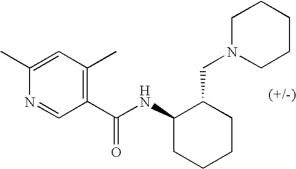 | trans-(+/−)-4,6-dimethyl-N- [2-(piperidin-1- ylmethyl)cyclohexyl]nicotin- amide | 329.94 | 1.39 |
| 61 | 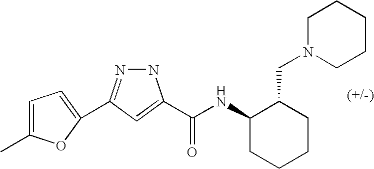 | trans-(+/−)-3-(5-methyl-2- furyl)-N-[2-(piperidin-1- ylmethyl)cyclohexyl]-1H- pyrazole-5-carboxamide | 370.89 | 1.66 |
| 62 | 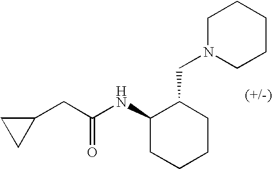 | trans-(+/−)-2-cyclopropyl-N- [2-(piperidin-1- ylmethyl)cyclohexyl]aceta- mide | 279.01 | 1.37 |
| 63 | 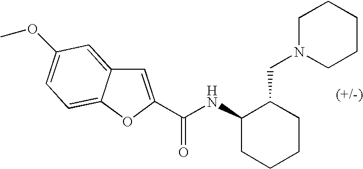 | trans-(+/−)-5-methoxy-N-[2- (piperidin-1- ylmethyl)cyclohexyl]-1- benzofuran-2-carboxamide | 370.89 | 2.02 |
| 64 | 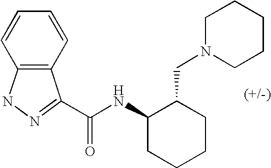 | trans-(+/−)-N-[2-(piperidin- 1-ylmethyl)cyclohexyl]-1H- indazole-3-carboxamide | 340.91 | 1.57 |
| 65 | 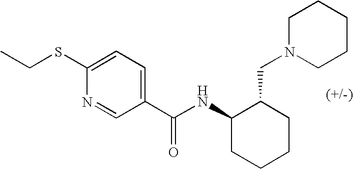 | trans-(+/−)-6-(ethylthio)-N- [2-(piperidin-1- ylmethyl)cyclohexyl]nicotin- amide | 361.92 | 1.85 |
| 66 | 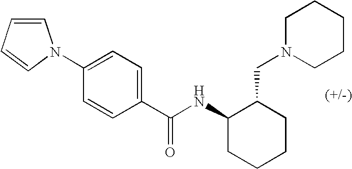 | trans-(+/−)-N-[2-(piperidin- 1-ylmethyl)cyclohexyl]-4- (1H-pyrrol-1-yl)benzamide | 365.93 | 1.94 |
| 67 | 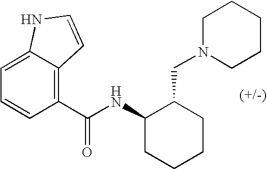 | trans-(+/−)-N-[2-(piperidin- 1-ylmethyl)cyclohexyl]-1H- indole-4-carboxamide | 339.91 | 1.61 |
| 68 | 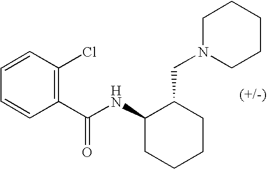 | trans-(+/−)-2-chloro-N-[2- (piperidin-1- ylmethyl)cyclohexyl]benza- mide | 334.87 | 1.74 |
| 69 | 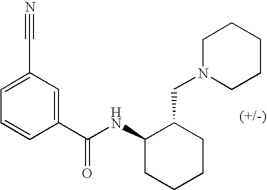 | trans-(+/−)-3-cyano-N-[2- (piperidin-1- ylmethyl)cyclohexyl]benza- mide | 325.92 | 1.73 |
| 70 | 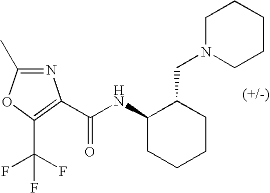 | trans-(+/−)-2-methyl-N-[2- (piperidin-1- ylmethyl)cyclohexyl]-5- (trifluoromethyl)-1,3- oxazole-4-carboxamide | 373.86 | 1.95 |
| 71 | 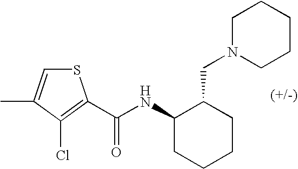 | trans-(+/−)-3-chloro-4- methyl-N-[2-(piperidin-1- ylmethyl)cyclohexyl]thiophene- 2-carboxamide | 354.82 | 2.04 |
| 72 | 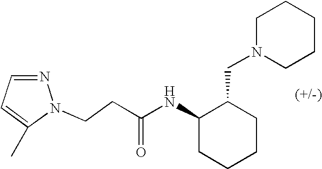 | trans-(+/−)-3-(5-methyl-1H- pyrazol-1-yl)-N-[2- (piperidin-1- ylmethyl)cyclohexyl]propan- amide | 332.95 | 1.27 |
| 73 | 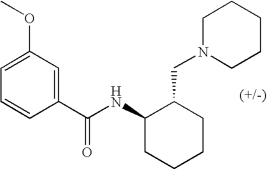 | trans-(+/−)-3-methoxy-N-[2- (piperidin-1- ylmethyl)cyclohexyl]benza- mide | 330.92 | 1.74 |
| 74 | 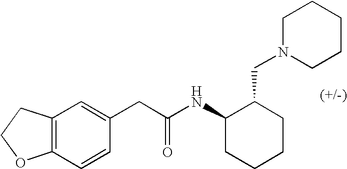 | trans-(+/−)-2-(2,3-dihydro- 1-benzofuran-5-yl)-N-[2- (piperidin-1- ylmethyl)cyclohexyl]aceta- mide | 356.96 | 1.48 |
| 75 | 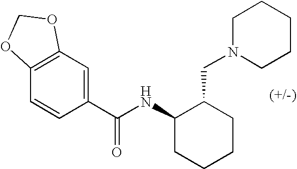 | trans-(+/−)-N-[2-(piperidin- 1-ylmethyl)cyclohexyl]-1,3- benzodioxole-5- carboxamide | 344.93 | 1.69 |
| 76 | 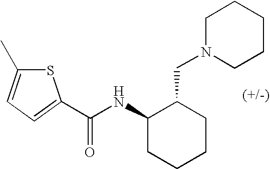 | trans-(+/−)-5-methyl-N-[2- (piperidin-1- ylmethyl)cyclohexyl]thio- phene-2-carboxamide | 320.89 | 1.76 |
| 77 | 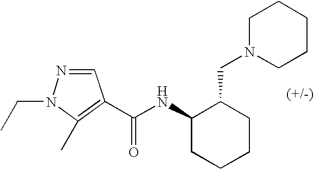 | trans-(+/−)-1-ethyl-5- methyl-N-[2-(piperidin-1- ylmethyl)cyclohexyl]-1H- pyrazole-4-carboxamide | 332.94 | 1.34 |
| 78 | 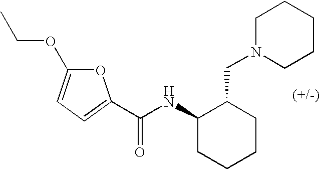 | trans-(+/−)-5-ethoxy-N-[2- (piperidin-1- ylmethyl)cyclohexyl]-2- furamide | 334.95 | 1.77 |
| 79 | 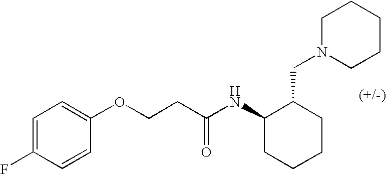 | trans-(+/−)-3-(4- fluorophenoxy)-N-[2- (piperidin-1- ylmethyl)cyclohexyl]propan- amide | 362.95 | 1.66 |
| 80 | 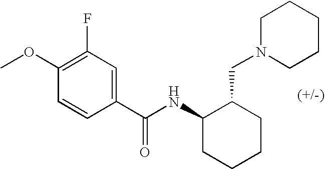 | trans-(+/−)-3-fluoro-4- methoxy-N-[2-(piperidin-1- ylmethyl)cyclohexyl]benza- mide | 348.93 | 1.76 |
| 81 | 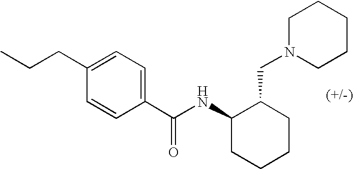 | trans-(+/−)-N-[2-(piperidin- 1-ylmethyl)cyclohexyl]-4- propylbenzamide | 342.96 | 2.12 |
| 82 | 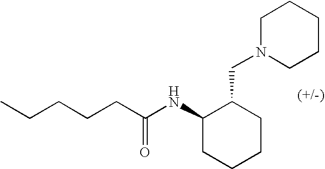 | trans-(+/−)-N-[2-(piperidin-1- ylmethyl)cyclohexyl]hexan- amide | 295.04 | 1.68 |
| 83 | 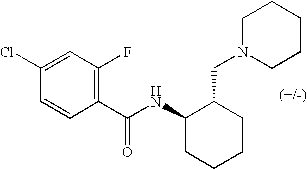 | trans-(+/−)-4-chloro-2- fluoro-N-[2-(piperidin-1- ylmethyl)cyclohexyl]benza- mide | 352.85 | 1.96 |
| 84 | 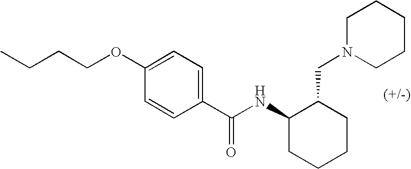 | trans-(+/−)-4-butoxy-N-[2- (piperidin-1- ylmethyl)cyclohexyl]benza- mide | 372.94 | 2.15 |
| 85 | 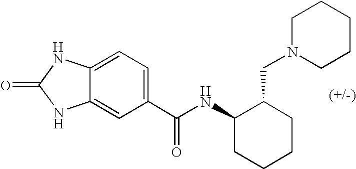 | trans-(+/−)-2-oxo-N-[2- (piperidin-1- ylmethyl)cyclohexyl]-2,3- dihydro-1H-benzimidazole- 5-carboxamide | 356.9 | 1.24 |
| 86 | 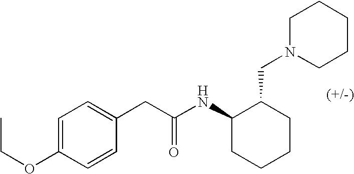 | trans-(+/−)-2-(4- ethoxyphenyl)-N-[2- (piperidin-1- ylmethyl)cyclohexyl]aceta- mide | 358.98 | 1.62 |
| 87 | 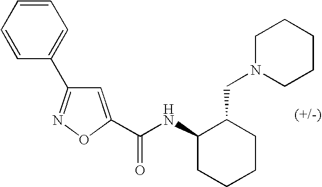 | trans-(+/−)-3-phenyl-N-[2- (piperidin-1- ylmethyl)cyclohexyl]isoxa- zole-5-carboxamide | 367.88 | 2.10 |
| 88 | 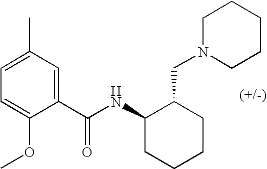 | trans-(+/−)-2-methoxy-5- methyl-N-[2-(piperidin-1- ylmethyl)cyclohexyl]benza- mide | 344.92 | 1.73 |

### Example 89

### trans-(+/−)-4-methoxy-N-{2-[(4-phenylpiperidin-1-yl)methyl]cyclohexyl}benzamide

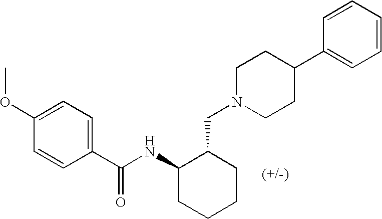

### Step A

### The Preparation of trans-(+/−)-tert-butyl[2-(hydroxymethyl)cyclohexyl]carbamate

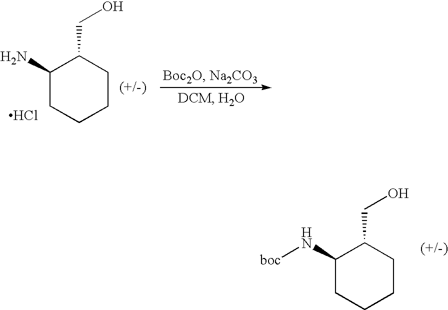

A solution of sodium carbonate (1.26 g, 12.2 mmol) in water (20 ml) was added to a suspension of trans-(+/−)-[2-aminocyclohexyl]methanol hydrochloride salt (1.00 g, 6.10 mmol) in dichloromethane (25 ml). The reaction was stirred at room temperature for 2 days. The solution was diluted with water (20 ml). The phases were separated and the aqueous was extracted with dichloromethane (2×75 ml). The combined organic phases were washed with brine, dried over Na₂SO₄, filtered, and concentrated in vacuo. A white solid was obtained (1.45 g). The product was used directly for the next step without further purification.

### Step B

### The Preparation of trans-(+/−)-tert-butyl[2-formylcyclohexyl]carbamate

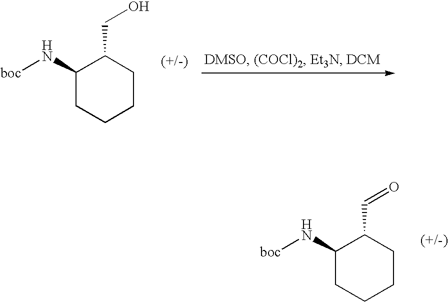

A 2M solution of oxalyl chloride in dichloromethane (4.57 ml, 9.14 mmol) was cooled to −78° C. under nitrogen and added to a solution of dimethylsulfoxide (1.30 ml, 18.3 mmol) in dichloromethane (6 ml) at −78° C. under nitrogen via cannula. After 10 minutes, a solution of the product from step A trans-(+/−)-(tert-butyl[2-(hydroxymethyl)cyclohexyl]carbamate, 6.10 mmol) in dichloromethane (6 ml) at −78° C. under nitrogen was added to the reaction mixture via cannula. The mixture was stirred at −78° C. under nitrogen for 10 minutes and then triethylamine (3.40 ml, 24.4 mmol) was added dropwise. The reaction was stirred at −78° C. under nitrogen for 20 minutes, then allowed to warm up to 0° C. over 1 hour. The reaction was quenched with water (25 ml) and diluted with dichloromethane (50 ml). The phases were separated and the aqueous was extracted with dichloromethane (2×75 ml). The combined organic phases were washed with saturated aqueous ammonium chloride, brine, dried over Na₂SO₄, filtered, and concentrated in vacuo. A yellow solid was obtained (1.34 g, 97%). 1H NMR (400 MHz, CHLOROFORM-D): δ ppm 1.12-1.27 (m, 2H), 1.29-1.52 (m, 2H), 1.40 (s, 9H), 1.70-1.82 (m, 3H), 1.96-2.10 (m, 2H), 3.68-3.80 (m, 1H), 4.42-4.49 (m, 1H), 9.56 (d, J=4.10 Hz, 1H).

### Step C

### The Preparation of trans-(+/−)-tert-butyl {2-[(4-phenylpiperidin-1-yl)methyl]cyclohexyl}carbamate

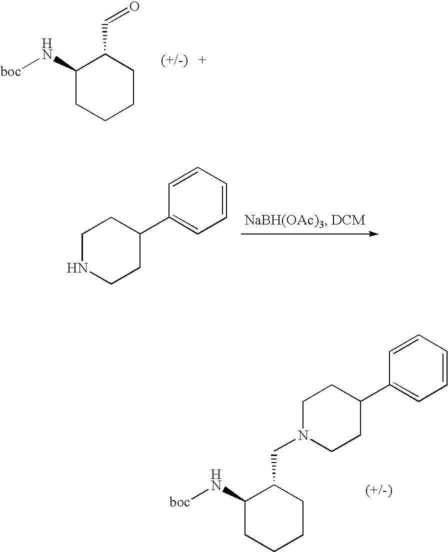

4-Phenylpiperidine (97 mg, 0.60 mmol) was added to a solution of trans-(+/−)-tert-butyl[2-formylcyclohexyl]carbamate (114 mg, 0.50 mmol) in dichloromethane (4 ml). The reaction was stirred at room temperature for 30 minutes, and then sodium triacetoxyborohydride (212 mg, 1.00 mmol) was added to the reaction mixture. The reaction was stirred at room temperature for 12 hours, and then cooled to 0° C. Water (1 ml) was added dropwise. A 1N sodium hydroxide solution (10 ml) and dichloromethane (30 ml) were added to the mixture. The phases were separated and the aqueous was extracted with dichloromethane (2×15 ml). The combined organic phases were washed with brine, dried over Na₂SO₄, filtered, and concentrated in vacuo. Yellow oil was obtained (200 mg). The product was used directly for the next step without further purification.

### Step D

### The Preparation of trans-(+/−)-{2-[(4-phenylpiperidin-1-yl)methyl]cyclohexyl}amine hydrochloride salt

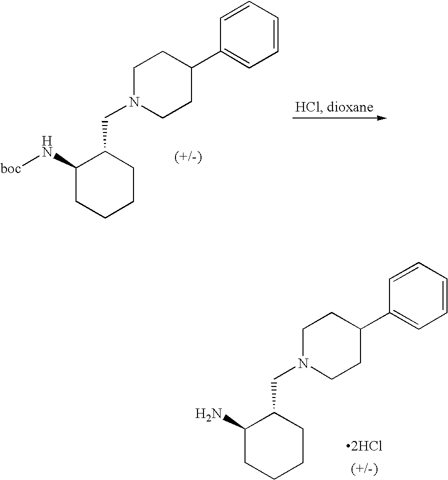

A 4N solution of hydrochloric acid in dioxane (2.0 ml, 8.0 mmol) was added to a solution of the crude product from step C trans-(+/−)-tert-butyl {-2-[(4-phenylpiperidin-1-yl)methyl]cyclohexyl}carbamate (0.50 mmol) in dioxane (5 ml). The reaction was stirred at room temperature for 3 days. The mixture was concentrated in vacuo. The product was used directly for the next step without further purification. MS (M+1): 273.2.

### Step E

### The Preparation of trans-(+/−)-4-methoxy-N-{2-[(4-phenylpiperidin-1-yl)methyl]cyclohexyl}benzamide

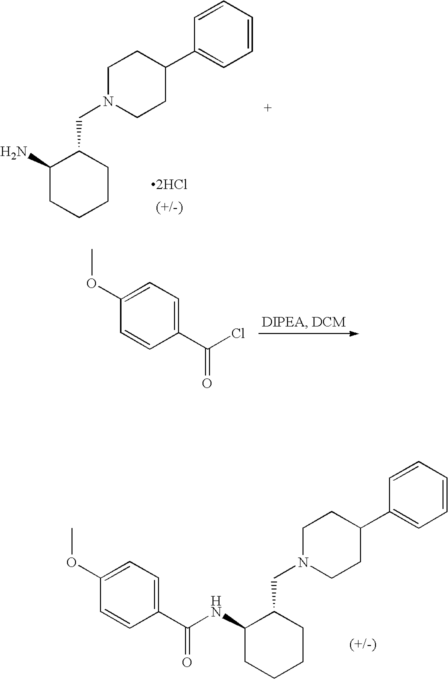

4-Methoxybenzoyl chloride (94 mg, 0.55 mmol) was added to a solution of the crude product from step D trans-(+/−)-{2-[(4-phenylpiperidin-1-yl)methyl]cyclohexyl}amine hydrochloride salt (0.50 mmol) and diisopropylethylamine (0.348 ml, 2.0 mmol) in dichloromethane (3 ml). The reaction was stirred at room temperature for 12 hours. The reaction mixture was diluted with dichloromethane. The solution washed with saturated aqueous sodium bicarbonate, brine, dried over Na₂SO₄, filtered, and concentrated in vacuo. The residue was purified by reverse phase HPLC. The combined pure fractions were concentrated in vacuo. The residue was dissolved in dioxane (2 ml) and a 4N solution of hydrochloric acid in dioxane (0.5 ml, 2.0 mmol) was added. The solution was concentrated in vacuo. The product was lyophilized. The HCl salt of the title compound was obtained as a white solid in a 68% yield over 3 steps (149 mg). MS (M+1): 407.3; 1H NMR (400 MHz, METHANOL-D4): δ ppm 1.23-1.61 (m, 4H), 1.83 (dd, 2H), 1.92-2.17 (m, 7H), 2.84 (tt, J=11.69, 4.44, 4.20 Hz, 1H), 2.99 (td, J=12.35, 4.20 Hz, 1H), 3.07 (dd, J=13.28, 9.37 Hz, 1H), 3.14-3.23 (m, 2H), 3.53-3.60 (m, 1H), 3.71-3.76 (m, 1H), 3.79 (td, J=10.94, 3.91 Hz, 1H), 3.83 (s, 3H), 6.99 (d, J=8.98 Hz, 2H), 7.12-7.25 (m, 3H), 7.26-7.33 (m, 2H), 7.86 (d, J=8.98 Hz, 2H).

### Example 90

### trans-(+/−)-N-[2-(1,4-dioxa-8-azaspiro[4,5]dec-8-ylmethyl)cyclohexyl]-4-methoxybenzamide

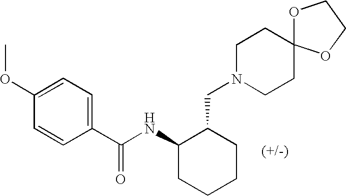

Following the procedure described in Example 89 (steps C to E), the HCl salt of the title compound was obtained as a white solid in a 24% yield over 3 steps (50 mg). MS (M+1): 389.3; 1H NMR (400 MHz, METHANOL-D4): δ ppm 1.21-1.58 (m, 4H), 1.75-2.17 (m, 9H), 2.99-3.08 (m, 1H), 3.15-3.25 (m, 1H), 3.45-3.53 (m, 1H), 3.58-3.80 (m, 4H), 3.83 (s, 3H), 3.92-3.98 (m, 4H), 6.98 (d, J=8.79 Hz, 2H), 7.84 (d, J=8.98 Hz, 2H).

### Example 91

### trans-(+/−)-N-{2-[(3,5-dimethylpiperidin-1-yl)methyl]cyclohexyl}-4-methoxybenzamide

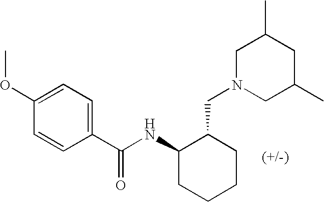

Following the procedure described in Example 89 (steps C to E), the HCl salt of the title compound was obtained as a white solid in a 43% yield over 3 steps (84 mg). MS (M+1): 359.3; 1H NMR (400 MHz, METHANOL-D4): δ ppm 0.82 (q, J=12.43 Hz, 1H), 0.89-0.97 (m, 6H), 1.15-1.66 (m, 5H), 1.75-2.13 (m, 7H), 2.33 (t, J=12.21 Hz, 1H), 2.55 (t, J=12.11 Hz, 1H), 3.01 (s, 1H), 3.09-3.15 (m, 1H), 3.30-3.39 (m, 1H), 3.48 (s, J=11.91 Hz, 1H), 3.75 (td, J=10.89, 4.00 Hz, 1H), 3.83 (s, 3H), 6.98 (d, J=8.79 Hz, 2H), 7.84 (s, 2H).

### Example 92

### trans-(+/−)-N-{2-[(4-fluoropiperidin-1-yl)methyl]cyclohexyl}-4-methoxybenzamide

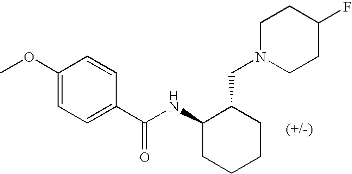

Following the procedure described in Example 89 (steps C to E), the title compound was obtained as a white solid in a 59% yield over 3 steps (51 mg). MS (M+1): 349.3. 1H NMR (400 MHz, METHANOL-D4): [symbol] pp [symbol] 0.99-1.17 (m, 1H), 1.23-1.45 (m, 3H), 1.58-1.87 (m, 7H), 1.91-2.00 (m, 1H), 2.03-2.11 (m, 1H), 2.17 (dd, J=12.79, 6.54 Hz, 1H), 2.26-2.40 (m, 2H), 2.44 (dd, J=12.69, 5.47 Hz, 1H), 2.49-2.62 (m, 2H), 3.54-3.64 (m, 1H), 3.84 (s, 3H), 4.49-4.68 (m, 1H), 6.98 (d, J=8.79 Hz, 2H), 7.78 (d, J=8.79 Hz, 2H).

### Example 93

### trans-(+/−)-4-methoxy-N-(2-{[4-(trifluoromethyl)piperidin-1-yl]methyl}cyclohexyl)benzamide

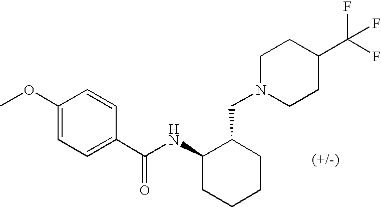

Following the procedure described in Example 89 (steps C to E), the title compound was obtained as a white solid in a 48% yield over 3 steps (48 mg). MS (M+1): 399.3. 1H NMR (400 MHz, METHANOL-D4): [symbol] pp [symbol] 1.20-1.64 (m, 4H) 1.74-1.91 (m, 3H) 1.92-2.05 (m, 3H), 2.06-2.20 (m, 3H), 2.49-2.69 (m, 1H), 2.94 (t, J=12.50 Hz, 1H), 3.05-3.20 (m, 3H), 3.60 (d, J=11.13 Hz, 1H), 3.73-3.82 (m, 2H), 3.83-3.87 (m, 3H), 7.00 (d, J=8.40 Hz, 2H), 7.88 (d, J=8.40 Hz, 2H).

### Example 94

### trans-(+/−)-4-methoxy-N-{2-[(4-methoxypiperidin-1-yl)methyl]cyclohexyl}benzamide

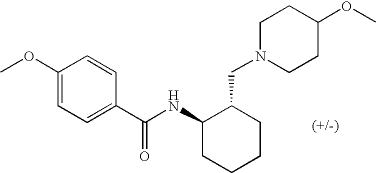

Following the procedure described in Example 89 (steps C to E), the title compound was obtained as a white solid in a 58% yield over 3 steps (52 mg). MS (M+1): 361.3. 1H NMR (400 MHz, METHANOL-D4): [symbol] pp [symbol] 0.98-1.17 (m, 4H), 1.22-1.48 (m, 4H), 1.51-1.68 (m, 2H), 1.70-1.99 (m, 5H), 2.04-2.19 (m, 4H), 2.42 (dd, J=12.69, 5.47 Hz, 1H), 2.62-2.82 (m, 2H), 3.15-3.26 (m, 1H), 3.30 (s 3H), 3.51-3.61 (m, 1H), 3.84 (s, 3H), 6.98 (d, J=8.79 Hz, 2H), 7.78 (d, J=8.79 Hz, 2H).

### Example 95

### trans-(+/−)-4-methoxy-N-(2-{[3-(trifluoromethyl)piperidin-1-yl]methyl}cyclohexyl)benzamide

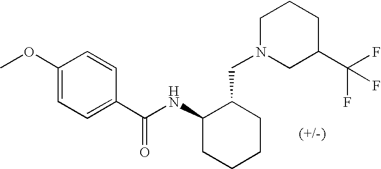

Following the procedure described in Example 89 (steps C to E), the title compound was obtained as a white solid in a 73% yield over 3 steps (58 mg). MS (M+1): 399.3. 1H NMR (400 MHz, METHANOL-D4): [symbol] pp [symbol] 1.21-1.65 (m, 6H), 1.76-1.91 (m, 3H), 1.90-2.15 (m, 5H), 2.76-2.88 (m, 1H), 2.95-3.18 (m, 2H), 3.19-3.26 (m, 1H), 3.45-3.67 (m, 1H), 3.66-3.81 (m, 2H), 3.83 (s, 3H), 6.98 (d, J=8.89 Hz, 2H), 7.80 (dd, J=8.89, 2.34 Hz, 2H).

### Example 96

### trans-(+/−)-4-methoxy-N-{2-[(3-phenylpiperidin-1-yl)methyl]cyclohexyl}benzamide

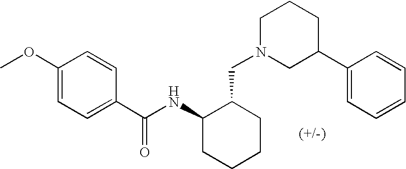

Following the procedure described in Example 89 (steps C to E), the title compound was obtained as a white solid in a 77% yield over 3 steps (63 mg). MS (M+1): 407.3. 1H NMR (400 MHz, METHANOL-D4): [symbol] pp [symbol] 1.21-1.58 (m, 5H) 1.66-1.86 (m, 3H) 1.86-2.03 (m, 5H) 2.03-2.15 (m, 1H) 2.79-2.98 (m, 1H) 3.00-3.12 (m, 2H) 3.13-3.24 (m, 2H) 3.66-3.80 (m, 2H) 3.83 (d, J=0.98 Hz, 3H) 6.91-7.00 (m, 2H) 7.19-7.35 (m, 5H) 7.73 (d, J=8.79 Hz, 1H) 7.80 (d, J=8.79 Hz, 1H).

### Example 97

### trans-(+/−)-N-[2-({3-[(allyloxy)methyl]piperidin-1-yl}methyl)cyclohexyl]-4-methoxybenzamide

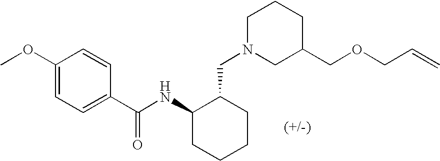

### Step A

### The Preparation of tert-butyl 3-[(allyloxy)methyl]piperidine-1-carboxylate

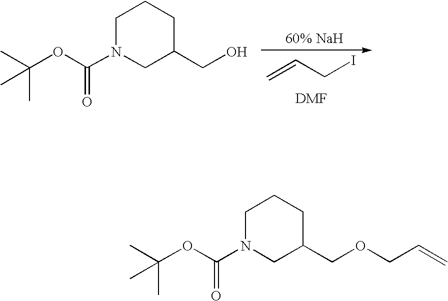

To a solution of tert-butyl 3-(hydroxymethyl)piperidine-1-carboxylate (0.86 g, 4.0 mmol) in dry DMF (15 mL) was added NaH (60%, 0.24 g, 6.0 mmol) at 0° C. under nitrogen and the suspension was stirred at room temperature for 30 min. Allyl iodide (1.51 g, 9.0 mmol) was added to the reaction mixture and stirred over night at room temperature. The solvent was removed in vacuo and the residue was dissolved in dichloromethane (50 mL), washed with water (30 mL), dried over Na₂SO₄. Removal of solvent gave the crude product, which was used for the next step without further purification.

### Step B

### The Preparation of 3-[(allyloxy)methyl]piperidine hydrochloride

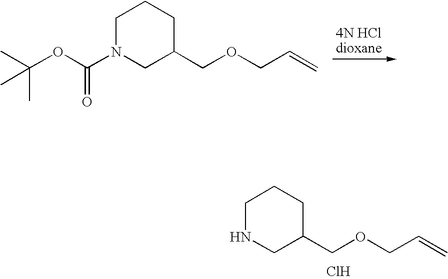

The crude tert-butyl 3-[(allyloxy)methyl]piperidine-1-carboxylate from step A was stirred in 4N HCl in dioxane (10 mL) at room temperature for 4 h. The solvent was removed in vacuo and the residue was added diethyl ether to form solid, filtered to give 3-[(allyloxy)methyl]piperidine hydrochloride as yellow powders (0.62 g, 81% for two steps).

### Step C

### The Preparation of trans-(+/−)-tert-butyl[2-({3-[(allyloxy)methyl]piperidin-1-yl}methyl)cyclohexyl]carbamate

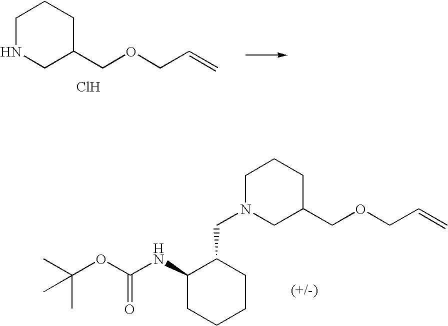

Following the procedure described in Example 89 (steps C), 3-[(allyloxy)methyl]piperidine hydrochloride (0.25 mmol) was added to a solution of trans-(+/−)-tert-butyl[2-formylcyclohexyl]carbamate (57 mg, 0.25 mmol) in dichloromethane (4 ml). The reaction was stirred at room temperature for 30 minutes, and then sodium triacetoxyborohydride (106 mg, 0.5 mmol) was added to the reaction mixture. The reaction was stirred at room temperature for 12 hours, and then cooled to 0° C. After the same work-up, the yellow oil was used directly for the next step without further purification.

### Step D

### The Preparation of trans-(+/−)-2-({3-[(allyloxy)methyl]piperidin-1-yl}methyl)cyclohexyl]amine hydrochloride

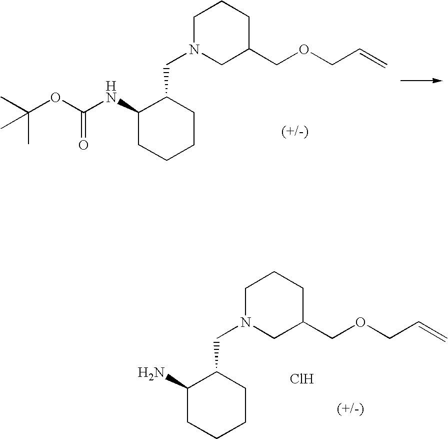

Following the procedure described in Example 89 (steps D), the HCl salt was obtained and used for the next step without further purification.

### Step E

### The Preparation of trans-(+/−)-N-[2-({3-[(allyloxy)methyl]piperidin-1-yl}methyl)cyclohexyl]-4-methoxybenzamide

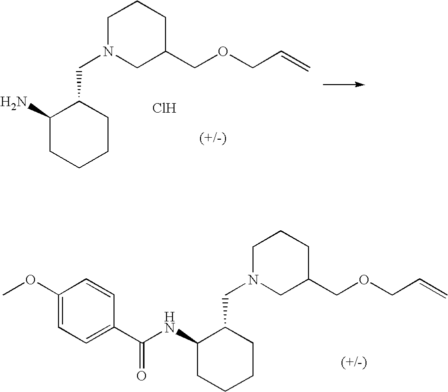

Following the procedure described in Example 1 (step D), the TFA salt of the title compound was obtained as a white solid in a 37% yield over 3 steps (48 mg). MS (M+1): 401.3. 1H NMR (400 MHz, METHANOL-D4): [symbol] pp [symbol] 1.22-1.60 (m, 5H), 1.74-1.87 (m, 4H), 1.90-2.01 (m, 3H), 2.02-2.23 (m, 2H), 2.55-2.97 (m, 2H), 3.00-3.08 (m, 1H), 3.11-3.18 (m, 1H), 3.22-3.27 (m, 1H), 3.36-3.52 (m, 2H), 3.59-3.68 (m, 1H), 3.71-3.80 (m, 1H), 3.83 (s, 3H), 3.88-3.98 (m, 2H), 5.07-5.29 (m, 2H), 5.78-5.94 (m, 1H), 6.98 (d, J=8.79 Hz, 2H), 7.81 (d, J=8.79 Hz, 2H).

### Example 98

### trans-(+/−)-N-[2-({3-[(allyloxy)methyl]piperidin-1-yl}methyl)cyclohexyl]-6-(1H-pyrazol-1-yl)nicotinamide

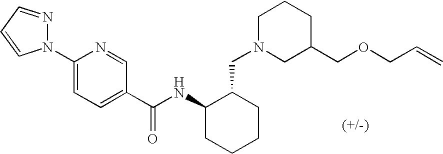

Following the procedure described in Example 2, the title compound was obtained as a white solid in a 29% yield over 3 steps (32 mg). MS (M+1): 438.0. 1H NMR (400 MHz, METHANOL-D4): δ ppm 0.83-1.00 (m, 1H), 1.01-1.17 (m, 1H), 1.25-1.43 (m, 4H), 1.53-1.79 (m, 6H), 1.77-1.99 (m, 3H), 2.04-2.19 (m, 2H), 2.34-2.47 (m, 1H), 2.68-3.04 (m, 2H), 3.07-3.26 (m, 2H), 3.55-3.66 (m, 1H), 3.78 (d, J=5.47 Hz, 1H), 3.88-3.94 (m, 1H), 4.98-5.28 (m, 2H), 5.61-5.98 (m, 1H), 6.54 (s, 1H), 7.78 (s, 1H), 8.00 (d, J=8.59 Hz, 1H), 8.26-8.34 (m, 1H), 8.63 (d, J=2.15 Hz, 1H), 8.85 (d, J=1.76 Hz, 1H).

### Example 99

### trans-(+/−)-N-(2-{[3-(methoxymethyl)piperidin-1-yl]methyl}cyclohexyl)-6-(1H-pyrazol-1-yl)nicotinamide

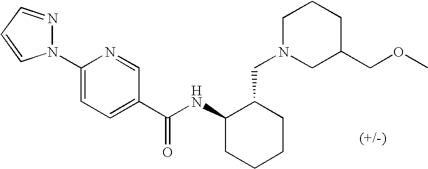

### Step A

### The Preparation of tert-butyl 3-[(methoxy)methyl]piperidine-1-carboxylate

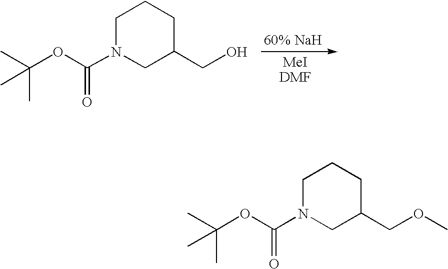

Following the same procedure as Example 97 (step A): To a solution of tert-butyl 3-(hydroxymethyl)piperidine-1-carboxylate (1.72 g, 8.0 mmol) in dry DMF (30 mL) was added NaH (60%, 0.48 g, 12.0 mmol) at 0° C. under nitrogen and the suspension was stirred at room temperature for 30 min. Methyl iodide (12.0 mmol) was added to the reaction mixture and stirred over night at room temperature. The solvent was removed in vacuo and the residue was dissolved in dichloromethane (80 mL), washed with water (50 mL), dried over Na₂SO₄. Removal of solvent gave the crude product (1.75 g, 95%), which was used for the next step without further purification.

### Step B

### The Preparation of 3-[(methoxy)methyl]piperidine hydrochloride

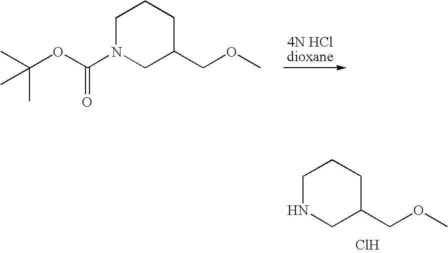

Following the same procedure as Example 97 (step B), the crude tert-butyl 3-[(methoxy)methyl]piperidine-1-carboxylate from step A was treated with 4N HCl in dioxane to give 3-[(methoxy)methyl]piperidine hydrochloride as white powders (1.18 g, 94%).

### Step C

### The Preparation of trans-(+/−)-tert-butyl[2-({3-[(methoxy)methyl]piperidin-1-yl}methyl)cyclohexyl]carbamate

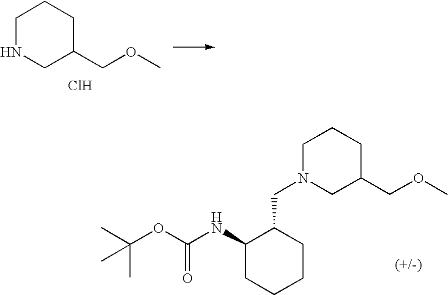

Following the procedure described in Example 89 (steps C)):3-[(methoxy)methyl]piperidine hydrochloride (0.2 mmol) was added to a solution of trans-(+/−)-tert-butyl[2-formylcyclohexyl]carbamate (0.2 mmol) in dichloromethane (4 ml). The reaction was stirred at room temperature for 30 minutes, and then sodium triacetoxyborohydride (85 mg, 0.4 mmol) was added to the reaction mixture. The reaction was stirred at room temperature for 12 hours, and then cooled to 0° C. After the same work-up, the yellow oil was used directly for the next step without further purification.

### Step D

### The Preparation of trans-(+/−)-2-({3-[(methoxy)methyl]piperidin-1-yl}methyl)cyclohexyl]amine hydrochloride

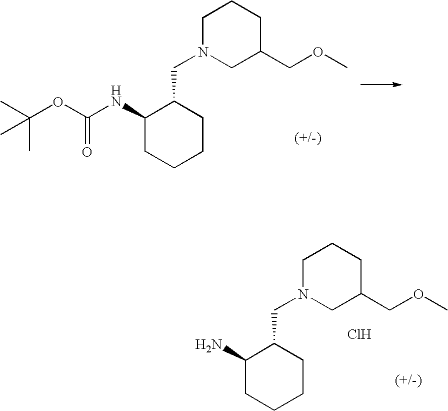

Following the procedure described in Example 89 (steps D), the HCl salt was obtained and used for the next step without further purification.

### Step E

### The Preparation of trans-(+/−)-N-(2-{[3-(methoxymethyl)piperidin-1-yl]methyl}cyclohexyl)-6-(1H-pyrazol-1-yl)nicotinamide

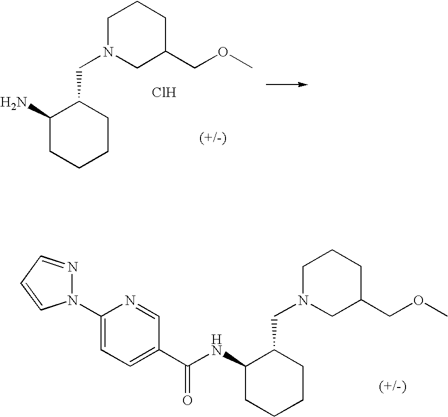

Following the procedure described in Example 2, the title compound was obtained as a white solid in a 51% yield over 3 steps (42 mg). MS (M+1): 412.3. 1H NMR (400 MHz, METHANOL-D4): δ ppm 0.82-0.98 (m, 1H), 1.01-1.14 (m, 1H), 1.30-1.43 (m, 3H), 1.53-1.81 (m, 7H), 1.83-1.90 (m, 1H), 1.91-2.01 (m, 1H), 2.06-2.18 (m, 2H), 2.36-2.48 (m, 1H), 2.71-3.00 (m, 2H), 3.04-3.11 (m, 1H), 3.11-3.15 (m, 1H), 3.16 (s, 3H), 3.21-3.26 (m, 1H), 3.50-3.70 (m, 1H), 6.54 (s, 1H), 7.78 (s, 1H), 8.00 (d, J=8.59 Hz, 1H), 8.30 (d, J=8.40 Hz, 1H), 8.63 (s, 1H) 8.84 (s, 1H).

### Example 100

### trans-(+/−)-N-(2-{[3-(ethoxymethyl)piperidin-1-yl]methyl}cyclohexyl)-6-(1H-pyrazol-1-yl)nicotinamide

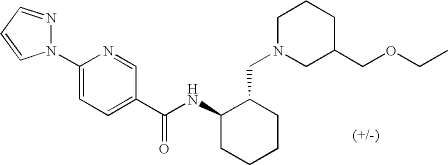

### Step A

### The Preparation of tert-butyl 3-[(ethoxy)methyl]piperidine-1-carboxylate

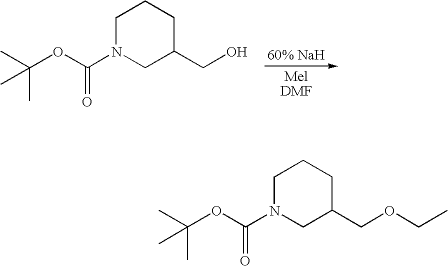

Following the same procedure as Example 97 (step A): To a solution of tert-butyl 3-(hydroxymethyl)piperidine-1-carboxylate (1.72 g, 8.0 mmol) in dry DMF (30 mL) was added NaH (60%, 0.48 g, 12.0 mmol) at 0° C. under nitrogen and the suspension was stirred at room temperature for 30 min. ethyl iodide (12.0 mmol) was added to the reaction mixture and stirred over night at room temperature. The solvent was removed in vacuo and the residue was dissolved in dichloromethane (80 mL), washed with water (50 mL), dried over Na₂SO₄. Removal of solvent gave the crude product (1.86 g, 95%), which was used for the next step without further purification.

### Step B

### The Preparation of 3-[(ethoxy)methyl]piperidine hydrochloride

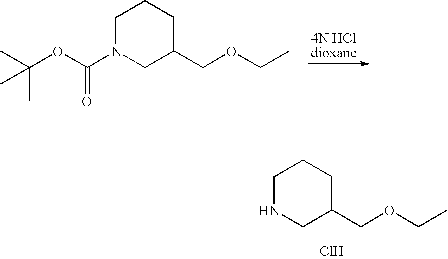

Following the same procedure as Example 97 (step B), the crude tert-butyl 3-[(ethoxy)methyl]piperidine-1-carboxylate from step A was treated with 4N HCl in dioxane to give 3-[(ethoxy)methyl]piperidine hydrochloride as white powders (1.31 g, 96%).

### Step C

### The Preparation of trans-(+/−)-tert-butyl[2-({3-[(ethoxy)methyl]piperidin-1-yl}methyl)cyclohexyl]carbamate

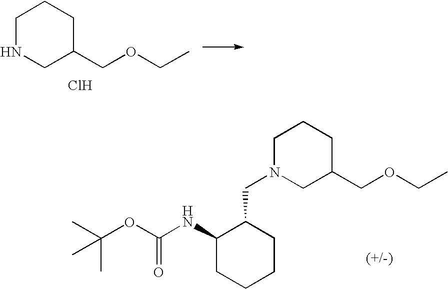

Following the procedure described in Example 89 (steps C), 3-[(ethoxy)methyl]piperidine hydrochloride (0.2 mmol) was added to a solution of trans-(+/−)-tert-butyl[2-formylcyclohexyl]carbamate (0.2 mmol) in dichloromethane (4 ml). The reaction was stirred at room temperature for 30 minutes, and then sodium triacetoxyborohydride (85 mg, 0.4 mmol) was added to the reaction mixture. The reaction was stirred at room temperature for 12 hours, and then cooled to 0° C. After the same work-up, the yellow oil was used directly for the next step without further purification.

### Step D

### The Preparation of trans-(+/−)-2-({3-[(ethoxy)methyl]piperidin-1-yl}methyl)cyclohexyl]amine hydrochloride

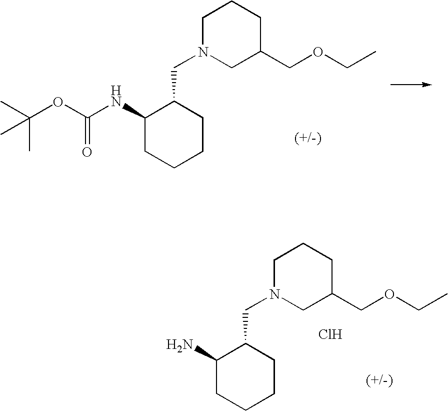

Following the procedure described in Example 89 (steps D), the HCl salt was obtained and used for the next step without further purification.

### Step E

### The Preparation of trans-(+/−)-N-(2-{[3-(ethoxymethyl)piperidin-1-yl]methyl}cyclohexyl)-6-(1H-pyrazol-1-yl)nicotinamide

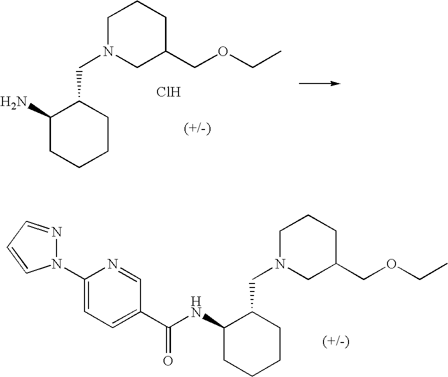

Following the procedure described in Example 2, the title compound was obtained as a white solid in a 45% yield over 3 steps (38 mg). MS (M+1): 426.2. 1H NMR (400 MHz, METHANOL-D4): δ ppm 0.85-0.96 (m, 1H), 1.03 (t, J=6.93 Hz, 2H), 1.06-1.11 (m, 1H), 1.14 (t, J=7.03 Hz, 2H), 1.25-1.45 (m, 4H), 1.54-1.82 (m, 6H), 1.83-1.99 (m, 3H), 2.05-2.20 (m, 2H), 2.38-2.49 (m, 1H), 2.71-3.03 (m, 2H), 3.07-3.24 (m, 2H), 3.36-3.49 (m, 1H), 3.54-3.67 (m, 1H), 6.55 (d, J=1.95 Hz, 1H), 7.78 (s, 1H), 8.00 (d, J=8.59 Hz, 1H), 8.30 (dd, J=8.59, 1.37 Hz, 1H), 8.63 (s, 1H), 8.85 (s, 1H).

### Example 101

### trans-(+/−)-N-{2-[(3-pentylpiperidin-1-yl)methyl]cyclohexyl}-6-(1H-pyrazol-1-yl)nicotinamide

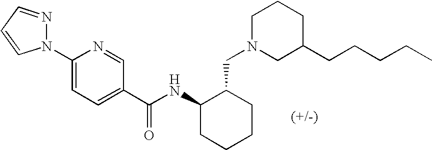

### Step A

### The Preparation of tert-butyl 3-({[(4-methylphenyl)sulfonyl]oxy}methyl)piperidine-1-carboxylate

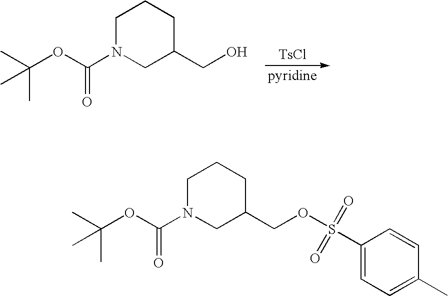

To a solution of tert-butyl 3-(hydroxymethyl)piperidine-1-carboxylate (2.15 g, 10.0 mmol) in dry pyridine (15 mL) was added Tosyl chloride (2.29 g, 12.0 mmol) at 0° C., the reaction mixture was stirred at 0° C. for 5 h and then at room temperature for 48 h. Ice water was added, extracted with DCM (50 mL), dried over Na₂SO₄. After removal of the solvent, the residue was purified with flash chromatography to give the title product as white solids (3.24 g, 88%).

### Step B

### The Preparation of tert-butyl 3-pentylpiperidine-1-carboxylate

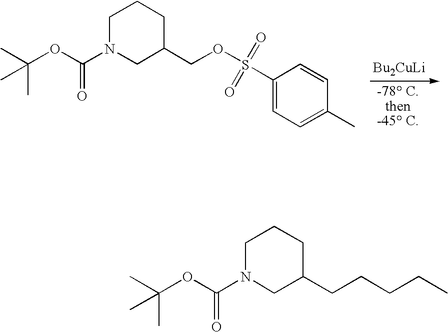

n-BuLi (1.6M in Hexanes, 18.8 mL, 30 mmol) was added dropwise to a stirred slurry of CuI (2.83 g, 15 mmol) in dry Et₂O (30 mL) at −78° C., then warmed up to −45° C. and stirred for 40 min to give a homogeneous solution. The temperature was lowered to −78° C. and to the mixture was slowly added a solution of tert-butyl 3-({[(4-methylphenyl)sulfonyl]oxy}methyl)piperidine-1-carboxylate (from step A, 1.11 g, 3.0 mmol) in Et₂O (3 mL), then warmed up to −45° C. and stirred for 20 min, poured into saturated aq. NH₄Cl (30 mL). NH₄OH (28%, 10 mL) was added, extracted with Et₂O (3×50 mL), the organic phase was separated, dried over Na₂SO₄, concentrated to give the crude product (570 mg, 74%), which was used without further purification.

### Step C

### The Preparation of 3-pentylpiperidine hydrochloride

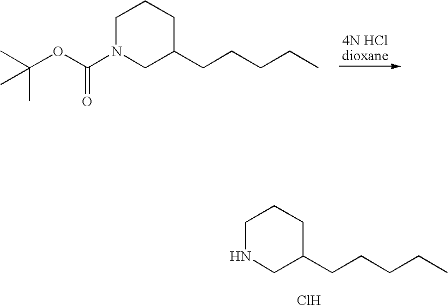

Following the same procedure as Example 97 (step B), the crude tert-butyl 3-pentylpiperidine-1-carboxylate from step B was treated with 4N HCl in dioxane to give 3-pentylpiperidine hydrochloride as white powders (423 mg, 99%).

### Step D

### The Preparation of trans-(+/−)-tert-butyl{2-[(3-pentylpiperidin-1-yl)methyl]cyclohexyl}carbamate

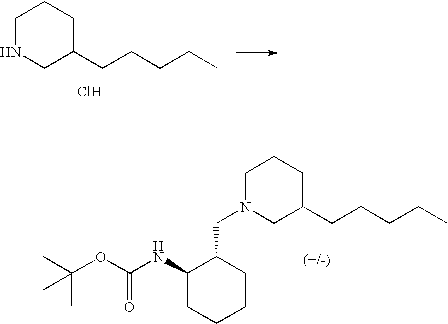

Following the procedure described in Example 89 (steps C), 3-pentylpiperidine hydrochloride (2.2 mmol) was added to a solution of trans-(+/−)-tert-butyl[2-formylcyclohexyl]carbamate (2.2 mmol) in dichloromethane (30 ml). The reaction was stirred at room temperature for 30 minutes, and then sodium triacetoxyborohydride (935 mg, 4.4 mmol) was added to the reaction mixture. The reaction was stirred at room temperature for 12 hours, and then cooled to 0° C. After the same work-up, the yellow oil (746 mg, 92%) was used directly for the next step without further purification.

### Step E

### The Preparation of trans-(+/−)-2-[(3-pentylpiperidin-1-yl)methyl]cyclohexylamine hydrochloride

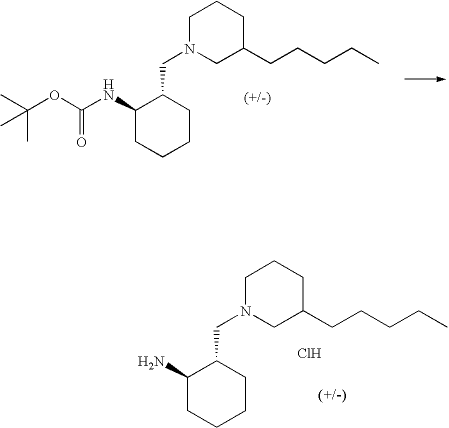

Following the procedure described in Example 89 (steps D), the crude trans-(+/−)-tert-butyl {2-[(3-pentylpiperidin-1-yl)methyl]cyclohexyl}carbamate from step D was treated with 4N HCl in dioxane, the HCl salt (2.0 mmol) was obtained and its stock solution in DMF (0.1M) was made to used for the next step.

### Step F

### The Preparation of trans-(+/−)-N-{2-[(3-pentylpiperidin-1-yl)methyl]cyclohexyl}-6-(1H-pyrazol-1-yl)nicotinamide

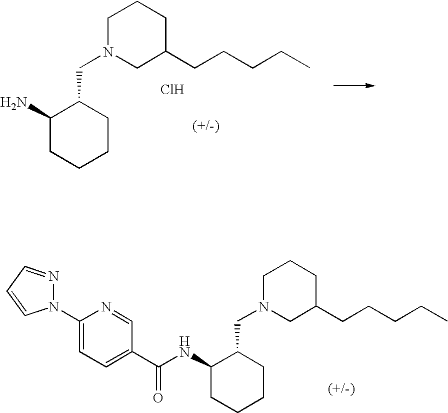

Following the procedure described in Example 2, the title compound was obtained as white solids (98 mg, 56%). MS (M+1): 438.3. 1H NMR (400 MHz, METHANOL-D4): δ ppm 0.72 (t, J=7.23 Hz, 2H), 0.84 (t, J=6.93 Hz, 2H), 0.92-1.15 (m, 6H), 1.16-1.39 (m, 7H), 1.49-1.57 (m, 2H), 1.59-1.78 (m, 6H), 1.82-2.00 (m, 1H), 2.02-2.19 (m, 2H), 2.31-2.45 (m, 1H), 2.60-3.02 (m, 2H), 3.45-3.70 (m, 1H), 6.49-6.56 (m, 1H), 7.76 (s, 1H), 7.99 (d, J=8.59 Hz, 1H), 8.29 (dd, J=8.59, 2.34 Hz, 1H), 8.61 (d, J=2.15 Hz, 1H), 8.84 (d, J=1.95 Hz, 1H).

### Example 102

### trans-(+/−)-N-{2-[(3-pentylpiperidin-1-yl)methyl]cyclohexyl}-4-(1H-pyrazol-1-yl)benzamide

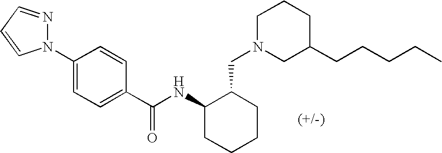

Following the procedure described in Example 2, the title compound was obtained as white solids (93 mg, 53%). MS (M+1): 437.3. 1H NMR (400 MHz, METHANOL-D4): δ ppm 0.68-0.90 (m, 4H), 0.94-1.17 (m, 6H), 1.19-1.40 (m, 7H), 1.48-1.56 (m, 2H), 1.61-1.80 (m, 6H), 1.89 (m, 1H), 2.02-2.21 (m, 2H), 2.32-2.44 (m, 1H), 2.61-3.02 (m, 2H), 3.45-3.64 (m, 1H), 6.53 (s, 1H), 7.73 (s, 1H), 7.80-7.88 (m, 2H), 7.90-7.95 (m, 2H), 8.31 (d, J=2.54 Hz, 1H).

### Example 103

### trans-(+/−)-6-(1H-imidazol-1-yl)-N-{2-[(3-pentylpiperidin-1-yl)methyl]cyclohexyl}nicotinamide

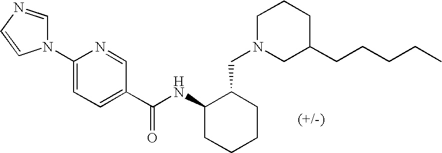

Following the procedure described in Example 2, the title compound was obtained as white solids (84 mg, 48%). MS (M+1): 438.3. 1H NMR (400 MHz, METHANOL-D4): δ ppm 0.71-0.90 (m, 4H), 0.96-1.17 (m, 6H), 1.20-1.41 (m, 7H), 1.46-1.60 (m, 2H), 1.60-1.81 (m, 6H), 1.82-2.00 (m, 1H), 2.03-2.19 (m, 2H), 2.29-2.45 (m, 1H), 2.62-3.02 (m, 2H), 3.51-3.68 (m, 1H), 7.16 (s, 1H), 7.80 (dd, J=8.50, 4.78 Hz, 1H), 7.95 (s, 1H), 8.30-8.37 (m, 1H), 8.60 (s, 1H), 8.90 (d, J=1.95 Hz, 1H).

### Example 104

### trans-(+/−)-N-{2-[(3-pentylpiperidin-1-yl)methyl]cyclohexyl}-6-pyrrolidin-1-ylnicotinamide

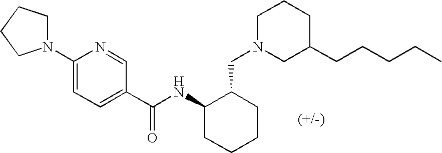

Following the procedure described in Example 2, the title compound was obtained as white solids (79 mg, 45%). MS (M+1): 441.3. 1H NMR (400 MHz, METHANOL-D4): δ ppm 0.76-0.90 (m, 4H), 0.95-1.15 (m, 6H), 1.16-1.35 (m, 8H), 1.36-1.48 (m, 1H), 1.51-1.65 (m, 3H), 1.66-1.79 (m, 4H), 1.81-1.96 (m, 1H), 1.98-2.06 (m, 4H), 2.06-2.16 (m, 1H), 2.28-2.41 (m, 1H), 2.64-3.01 (m, 2H), 3.40-3.52 (m, 4H), 3.49-3.60 (m, 1H), 6.47 (d, J=8.79 Hz, 1H), 7.89 (dd, J=8.89, 1.86 Hz, 1H), 8.51 (d, J=1.95 Hz, 1H).

### Example 105

### trans-(±)-6-(1H-imidazol-1-yl)-N-(-2-{[(3R)-3-pentylpiperidin-1-yl]methyl}cyclohexyl)nicotinamide

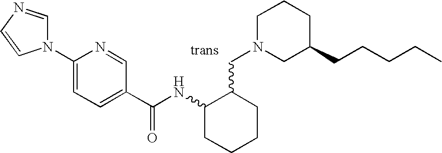

### Step A

### The Preparation of tert-butyl (3R)-3-({[(4-methylphenyl)sulfonyl]oxy}methyl)piperidine-1-carboxylate

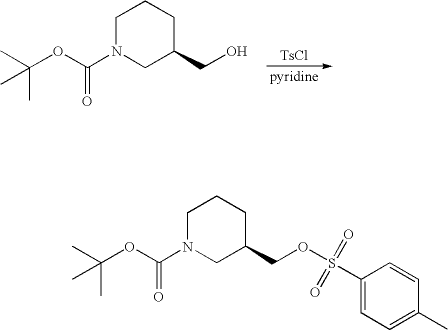

Following the same procedure as Example 101 (step A), the title product was obtained as white solids (820 mg, 96%).

### Step B

### The Preparation of tert-butyl (3R)-3-pentylpiperidine-1-carboxylate

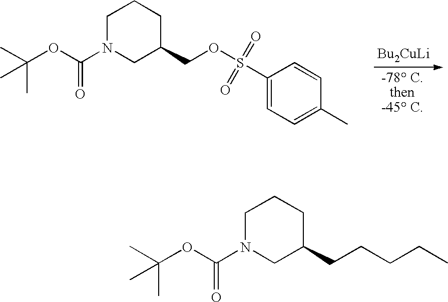

Following the same procedure as Example 101 (step B), the title product was obtained as a crude oil (460 mg, 81%).

### Step C

### The Preparation of (3R)-3-pentylpiperidine hydrochloride

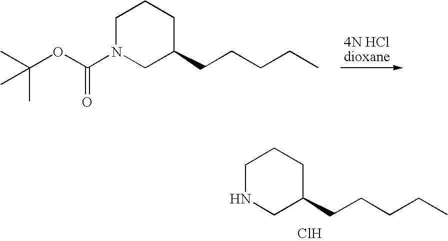

Following the same procedure as Example 97 (step B), the title product was obtained as a crude HCl salt (307 mg, 89%).

### Step D

### The Preparation of trans-(±)-tert-butyl (2-{[(3R)-3-pentylpiperidin-1-yl]methyl}cyclohexyl)carbamate

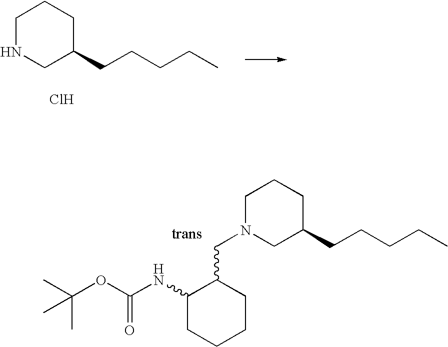

Following the procedure described in Example 89 (steps C), yielded title compound as a crude oil, which was used for the next step without further purification.

### Step E

### The Preparation of trans-(±)(2-{[(3R)-3-pentylpiperidin-1-yl]methyl}cyclohexyl)aminehydrochloride

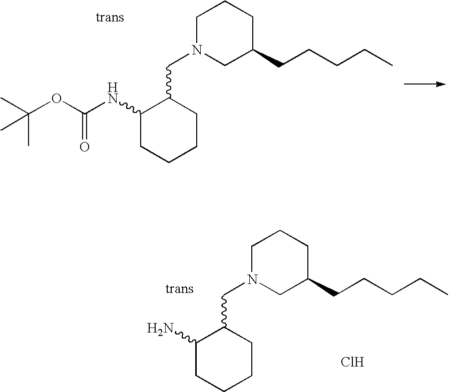

Following the procedure described in Example 89 (steps D), the crude trans (±)-tert-butyl (2-{[(3R)-3-pentylpiperidin-1-yl]methyl}cyclohexyl)carbamate from step D was treated with 4N HCl in dioxane, the HCl salt (˜1.6 mmol) was obtained and its stock solution in DMF (0.1M) was made to used for the next step.

### Step F

### The Preparation of trans-(±)-6-(1H-imidazol-1-yl)-N-(2-{[(3R)-3-pentylpiperidin-1-yl]methyl}cyclohexyl)nicotinamide

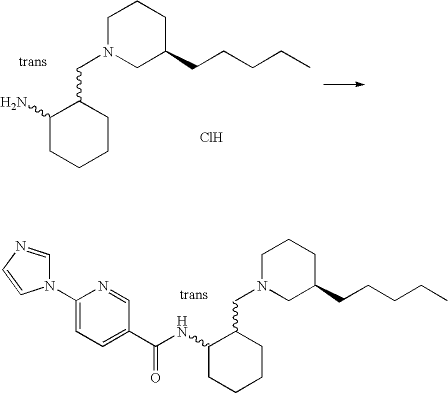

Following the procedure described in Example 2, the title compound was obtained as white solids (43 mg, 39% over 3 steps). MS (M+1): 438.3. 1H NMR (400 MHz, METHANOL-D4): δ ppm 0.69-0.90 (m, 4H), 0.94-1.19 (m, 6H), 1.22-1.40 (m, 6H), 1.47-1.60 (m, 2H), 1.60-1.83 (m, 6H), 1.83-2.00 (m, 1H), 2.04-2.20 (m, 2H), 2.32-2.48 (m, 1H), 2.63-2.87 (m, 1H), 2.88-3.06 (m, 2H), 3.51-3.69 (m, 1H), 7.16 (s, 1H), 7.81 (dd, J=8.50, 4.98 Hz, 1H), 7.95 (s, 1H), 8.29-8.38 (m, 1H), 8.60 (s, 1H), 8.88-8.94 (m, 1H).

### Example 106

### Trans (±)-6-(1H-imidazol-1-yl)-N-(2-{[(3S)-3-pentylpiperidin-1-yl]methyl}cyclohexyl)nicotinamide

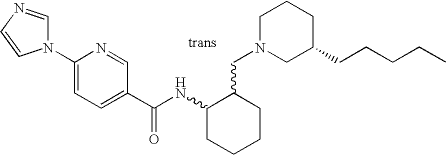

### Step A

### The Preparation of tert-butyl (3S)-3-({[(4-methylphenyl)sulfonyl]oxy}methyl)piperidine-1-carboxylate

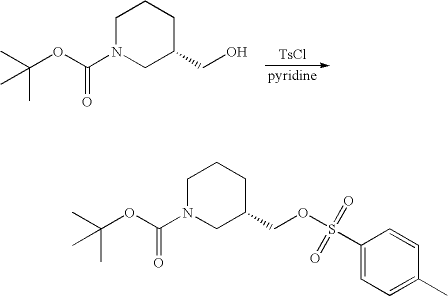

Following the same procedure as Example 101 (step A), the title product was obtained as white solids (818 mg, 96%).

### Step B

### The Preparation of tert-butyl (3S)-3-pentylpiperidine-1-carboxylate

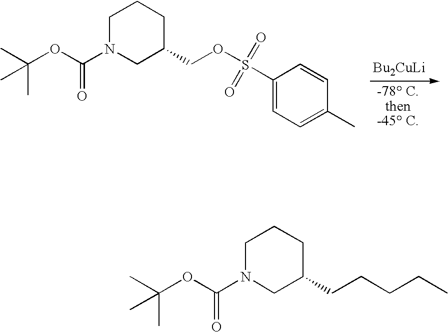

Following the same procedure as Example 101 (step B), the title product was obtained as a crude oil (510 mg, 90%).

### Step C

### The Preparation of (3S)-3-pentylpiperidine hydrochloride

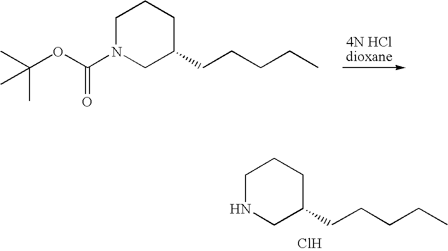

Following the same procedure as Example 97 (step B), the title product was obtained as a crude HCl salt (345 mg, 90%).

### Step D

### The Preparation of trans-(±)-tert-butyl (2-{[(3S)-3-pentylpiperidin-1-yl]methyl}cyclohexyl)carbamate

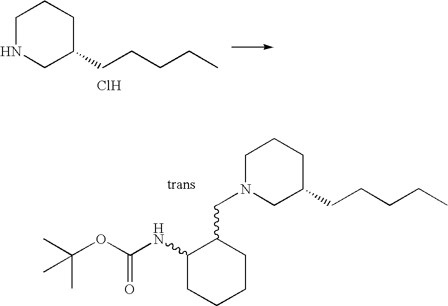

Following the procedure described in Example 89 (steps C), yielded title compound as a crude oil, which was used for the next step without further purification.

### Step E

### The Preparation of trans-(±)-(2-{[(3S)-3-pentylpiperidin-1-yl]methyl}cyclohexyl)aminehydrochloride

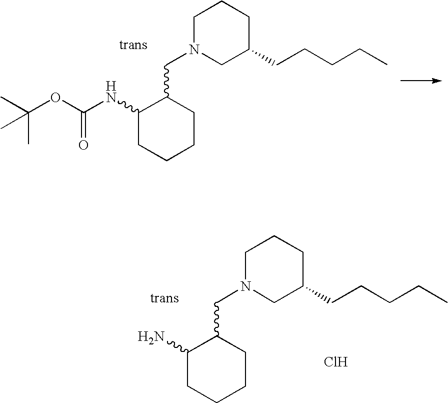

Following the procedure described in Example 89 (steps D), the crude trans-(±)-tert-butyl (2-{[(3R)-3-pentylpiperidin-1-yl]methyl}cyclohexyl)carbamate from step D was treated with 4N HCl in dioxane, the HCl salt (˜1.8 mmol) was obtained and its stock solution in DMF (0.1M) was made to used for the next step.

### Step F

### The Preparation of trans-(±)-6-(1H-imidazol-1-yl)-N-(2-{[(3S)-3-pentylpiperidin-1-yl]methyl}cyclohexyl)nicotinamide

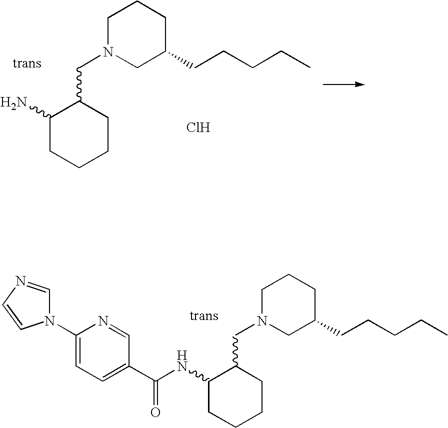

Following the procedure described in Example 2, the title compound was obtained as white solids (38 mg, 35% over 3 steps). MS (M+1): 438.3. 1H NMR (400 MHz, METHANOL-D4): δ ppm 0.68-0.89 (m, 4H), 0.96-1.17 (m, 6H), 1.20-1.43 (m, 6H), 1.41-1.60 (m, 2H), 1.61-1.82 (m, 6H), 1.82-2.02 (m, 1H), 2.04-2.21 (m, 2H), 2.32-2.49 (m, 1H), 2.59-2.87 (m, 1H), 2.90-3.10 (m, Hz, 2H), 3.52-3.69 (m, 1H), 7.16 (s, 1H), 7.81 (dd, J=8.50, 4.98 Hz, 1H), 7.95 (s, 1H), 8.29-8.37 (m, 1H), 8.60 (s, 1H), 8.83-8.95 (m, 1H).

### Example 107

### trans-(+/−)-N-{(2-[(3-hexylpiperidin-1-yl)methyl]cyclohexyl}-6-(1H-pyrazol-1-yl)nicotinamide

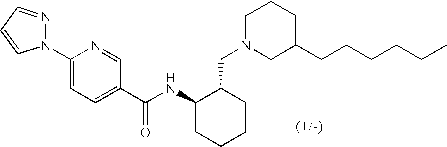

### Step A

### The Preparation of 3-hexylpiperidine hydrochloride

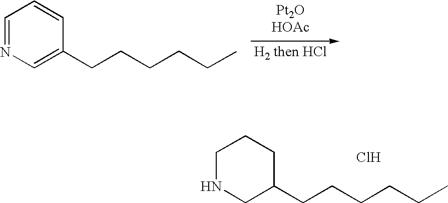

To a solution of 3-hexylpyridine (2.28 g, 14.0 mmol) in HOAc (40 mL) was added Pt₂O (0.15 g) and the mixture was hydrogenated at room temperature (40 psi) for 5 h. After being filtered and concentrated, 40% aq. NaOH (20 mL) was added, extracted with EtOAc (3×30 mL), dried over Na₂SO₄, then treated with 4N HCl in dioxane, evaporated to give the HCl salt as white powders (2.54 g, 88%).

### Step B

### The Preparation of trans-(+/−)-tert-butyl{2-[(3-hexylpiperidin-1-yl)methyl]cyclohexyl}carbamate

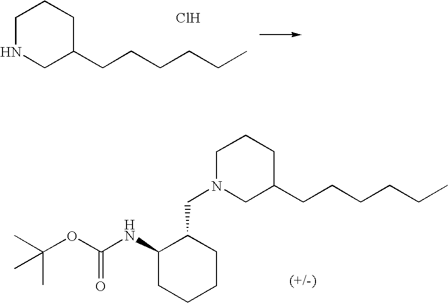

Following the procedure described in Example 89 (steps C), yielded title compound as a crude oil (635 mg, 93%), which was used for the next step without further purification.

### Step C

### The Preparation of trans-(+/−)-tert-butyl{2-[(3-hexylpiperidin-1-yl)methyl]cyclohexyl}carbamate

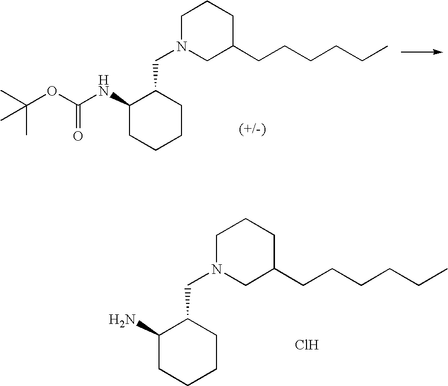

Following the procedure described in Example 89 (steps D), the crude trans-(+/−)-tert-butyl{2-[(3-hexylpiperidin-1-yl)methyl]cyclohexyl}carbamate from step B was treated with 4N HCl in dioxane, the HCl salt (505 mg, 100%) was obtained and its stock solution in DMF (0.1M) was made to used for the next step.

### Step D

### The Preparation of trans-(+/−)-N-{(2-[(3-hexylpiperidin-1-yl)methyl]cyclohexyl}-6-(1H-pyrazol-1-yl)nicotinamide

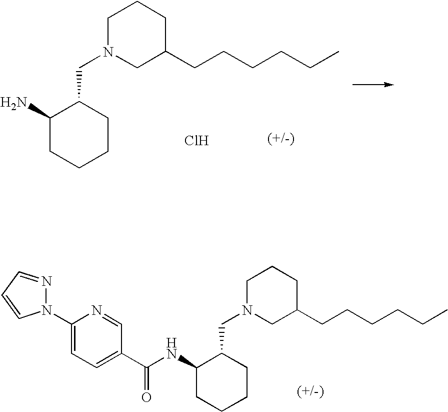

Following the procedure described in Example 2, the title compound was obtained as white solids (108 mg, 60%). MS (M+1): 452.3. 1H NMR (400 MHz, METHANOL-D4): δ ppm 0.74-0.90 (m, 4H), 0.97-1.19 (m, 8H), 1.21-1.44 (m, 8H), 1.50-1.81 (m, 6H), 1.82-2.01 (m, 2H), 2.05-2.22 (m, 2H), 2.33-2.49 (m, 1H), 2.63-3.01 (m, 2H), 3.46-3.69 (m, 1H), 6.53-6.56 (m, 1H), 7.78 (s, 1H), 8.01 (dd, J=8.59, 0.78 Hz, 1H), 8.26-8.33 (m, 1H), 8.63 (d, J=2.54 Hz, 1H), 8.82-8.87 (m, 1H).

### Example 108

### trans-(+/−)-N-{2-[(3-hexylpiperidin-1-yl)methyl]cyclohexyl}-6-(1H-imidazol-1-yl)nicotinamide

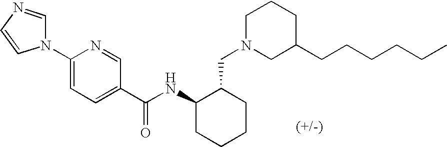

Following the procedure described in Example 2, the title compound was obtained as white solids (104 mg, 57%). MS (M+1): 452.3. 1H NMR (400 MHz, METHANOL-D4): δ ppm 0.73-0.88 (m, 4H), 0.95-1.17 (m, 7H), 1.19-1.29 (m, 5H), 1.31-1.41 (m, 3H), 1.47-1.58 (m, 2H), 1.61-1.80 (m, 6H), 1.81-2.00 (m, 1H), 2.03-2.21 (m, 2H), 2.32-2.45 (m, 1H), 2.61-3.03 (m, 2H), 3.51-3.68 (m, 1H), 7.16 (s, 1H), 7.80 (dd, J=8.50, 4.98 Hz, 1H), 7.95 (s, 1H), 8.33 (d, J=8.40 Hz, 1H), 8.60 (s, 1H), 8.90 (s, 1H).

### Example 109

### trans-(+/−)-N-{2-[(3-hexylpiperidin-1-yl)methyl]cyclohexyl}-4-(1H-pyrazol-1-yl)benzamide

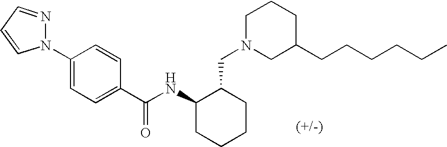

Following the procedure described in Example 2, the title compound was obtained as white solids (113 mg, 63%). MS (M+1): 451.2. 1H NMR (400 MHz, METHANOL-D4): δ ppm 0.72-0.91 (m, 4H), 0.95-1.18 (m, 7H), 1.19-1.41 (m, 8H), 1.49-1.56 (m, 2H), 1.61-1.80 (m, 6H), 1.81-1.98 (m, 1H), 2.02-2.24 (m, 2H), 2.31-2.43 (m, 1H), 2.60-3.01 (m, 2H), 3.47-3.63 (m, 1H), 6.50-6.56 (m, 1H), 7.73 (s, 1H), 7.82-7.89 (m, 2H), 7.90-7.94 (m, 2H), 8.31 (d, J=2.15 Hz, 1H).

### Example 110

### trans-(+/−)-N-{2-[(3-hexylpiperidin-1-yl)methyl]cyclohexyl}-4-pyrrolidin-1-ylbenzamide

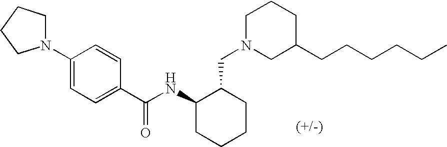

Following the procedure described in Example 2, the title compound was obtained as white solids (99 mg, 54%). MS (M+1): 455.3. 1H NMR (400 MHz, METHANOL-D4): δ ppm 0.74-0.92 (m, 4H), 0.99-1.16 (m, 6H), 1.19-1.34 (m, 8H), 1.34-1.49 (m, 2H), 1.51-1.67 (m, 4H), 1.68-1.80 (m, 4H), 1.81-1.97 (m, 1H), 1.99-2.06 (m, 4H), 2.08-2.17 (m, 1H), 2.26-2.44 (m, 1H), 2.63-3.00 (m, 2H), 3.42-3.62 (m, 5 H), 6.49 (d, J=8.98 Hz, 1H), 7.90 (dd, J=8.98, 2.34 Hz, 1H), 8.51 (d, J=2.34 Hz, 1H).

### Example 111

### trans-(+/−)-N-{(2-[(3-butylpiperidin-1-yl)methyl]cyclohexyl}-6-(1H-pyrazol-1-yl)nicotinamide

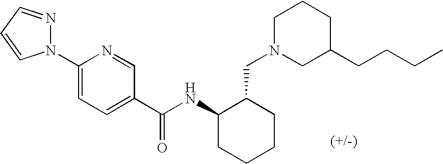

### Step A

### The Preparation of 3-butylpiperidine hydrochloride

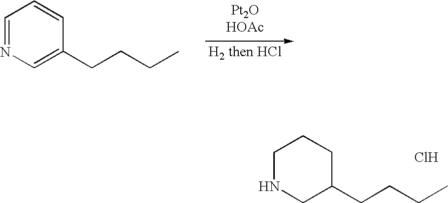

To a solution of 3-butylpyridine (1.35 g, 10.0 mmol) in HOAc (30 mL) was added Pt₂O (0.12 g) and the mixture was hydrogenated at room temperature (40 psi) for 5 h. After being filtered and concentrated, 40% aq. NaOH (20 mL) was added, extracted with EtOAc (3×30 mL), dried over Na₂SO₄, then treated with 4N HCl in dioxane, evaporated to give the HCl salt as white powders (1.68 g, 94%).

### Step B

### The Preparation of trans-(+/−)-tert-butyl{2-[(3-butylpiperidin-1-yl)methyl]cyclohexyl}carbamate

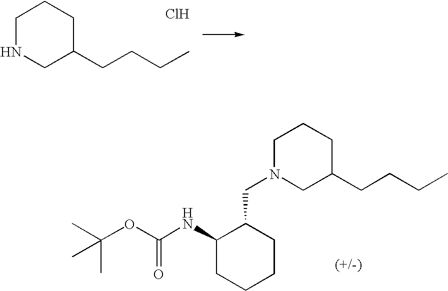

Following the procedure described in Example 89 (steps C), yielded title compound as a crude oil (597 mg, 94%), which was used for the next step without further purification.

### Step C

### The Preparation of trans-(+/−)-tert-butyl{2-[(3-butylpiperidin-1-yl)methyl]cyclohexyl}carbamate

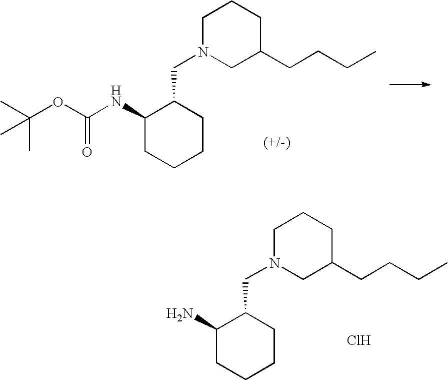

Following the procedure described in Example 89 (steps D), the crude trans-(+/−)-tert-butyl{2-[(3-butylpiperidin-1-yl)methyl]cyclohexyl}carbamate from step B was treated with 4N HCl in dioxane, the HCl salt (490 mg, 100%) was obtained and its stock solution in DMF (0.1M) was made to used for the next step.

### Step D

### The Preparation of trans-(+/−)-N-{(2-[(3-butylpiperidin-1-yl)methyl]cyclohexyl}-6-(1H-pyrazol-1-yl)nicotinamide

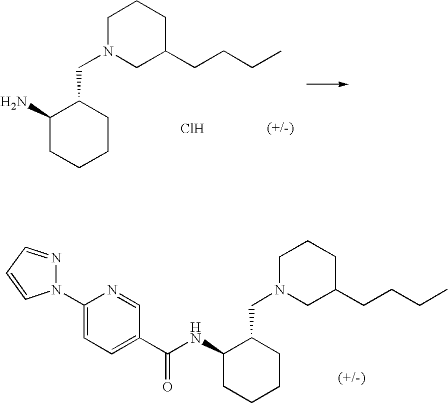

Following the procedure described in Example 2, the title compound was obtained as white solids (73 mg, 49%). MS (M+1): 424.3. 1H NMR (400 MHz, METHANOL-D4): δ ppm 0.64-0.89 (m, 4H), 0.97-1.16 (m, 5H), 1.24-1.40 (m, 6H), 1.47-1.60 (m, 2H), 1.62-1.80 (m, 6H), 1.82-2.00 (m, 1H), 2.03-2.23 (m, 2H), 2.31-2.45 (m, 1H), 2.64-3.05 (m, 2H), 3.49-3.69 (m, 1H), 6.51-6.59 (m, 1H), 7.78 (s, 1H), 8.00 (dd, J=8.59, 1.95 Hz, 1H), 8.30 (dd, J=8.59, 2.15 Hz, 1H), 8.63 (d, J=2.73 Hz, 1H), 8.85 (d, J=2.15 Hz, 1H).

### Example 112

### trans-(+/−)-N-{2-[(3-butylpiperidin-1-yl)methyl]cyclohexyl}-4-pyrrolidin-1-ylbenzamide

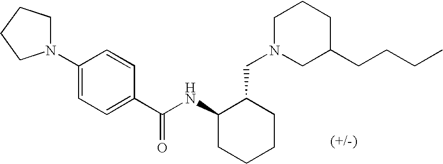

Following the procedure described in Example 2, the title compound was obtained as white solids (86 mg, 58%). MS (M+1): 427.2. 1H NMR (400 MHz, METHANOL-D4): δ ppm 0.72-0.89 (m, 4H), 0.96-1.20 (m, 6H), 1.22-1.36 (m, 6H), 1.48-1.66 (m, 3H), 1.67-1.80 (m, 4H), 1.82-1.98 (m, 1H), 2.00-2.07 (m, 5H), 2.08-2.17 (m, 1H), 2.30-2.44 (m, 1H), 2.59-3.00 (m, 2H), 3.38-3.62 (m, 5H), 6.49 (d, J=8.98 Hz, 1H), 7.89 (dd, J=8.98, 2.15 Hz, 1H), 8.51 (s, 1H).

### Example 113

### trans-(+/−)-N-{2-[(3-butylpiperidin-1-yl)methyl]cyclohexyl}-6-(1H-imidazol-1-yl)nicotinamide

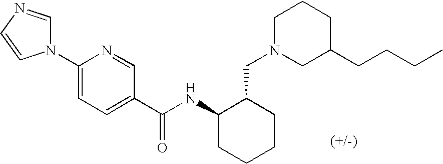

Following the procedure described in Example 2, the title compound was obtained as white solids (69 mg, 47%). MS (M+1): 424.3. 1H NMR (400 MHz, METHANOL-D4): δ ppm 0.65-0.92 (m, 4H), 0.96-1.20 (m, 6H), 1.21-1.40 (m, 6H), 1.41-1.60 (m, 3H), 1.61-1.81 (m, 6H), 1.83-2.00 (m, 1H), 2.04-2.21 (m, 2H), 2.33-2.43 (m, 1H), 2.58-3.04 (m, 2H), 3.51-3.69 (m, 1H), 7.16 (s, 1H), 7.81 (dd, J=8.50, 5.37 Hz, 1H), 7.95 (s, 1H), 8.30-8.38 (m, 1H), 8.60 (s, 1H), 8.87-8.93 (m, 1H).

### Example 114

### trans-(+/−)-N-{2-[(3-butylpiperidin-1-yl)methyl]cyclohexyl}-4-(1H-pyrazol-1-yl)benzamide

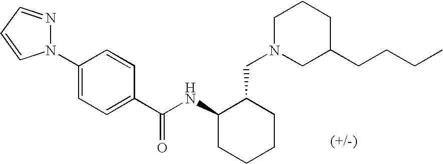

Following the procedure described in Example 2, the title compound was obtained as white solids (76 mg, 51%). MS (M+1): 423.3. 1H NMR (400 MHz, METHANOL-D4): δ ppm 0.66-0.91 (m, 4H), 0.97-1.10 (m, 4H), 1.19-1.38 (m, 6H), 1.41-1.56 (m, 3H), 1.61-1.81 (m, 6H), 1.80-1.98 (m, 1H), 2.03-2.22 (m, 2H), 2.32-2.43 (m, 1H), 2.58-3.05 (m, 2H), 3.46-3.70 (m, 1H), 6.53 (s, 1H), 7.73 (s, 1H), 7.82-7.89 (m, 2H), 7.89-7.95 (m, 2H), 8.31 (d, J=2.34 Hz, 1H).

### Example 115

### cis-(+/−)-N-{2-[(3-butylpiperidin-1-yl)methyl]cyclohexyl}-6-(1H-imidazol-1-yl)nicotinamide

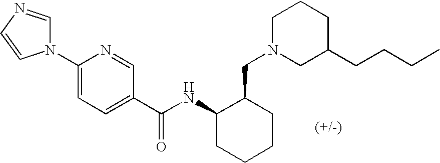

### Step A

### The Preparation of cis-(+/−)-tert-butyl[2-(hydroxymethyl)cyclohexyl]carbamate

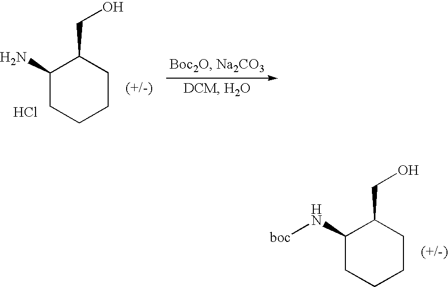

Following the same procedure as Example 89 (step A), the title compound was obtained as white solids (386 mg, 96%) and was used directly for the next step without further purification.

### Step B

### The Preparation of cis-(+/−)-tert-butyl[2-formylcyclohexyl]carbamate

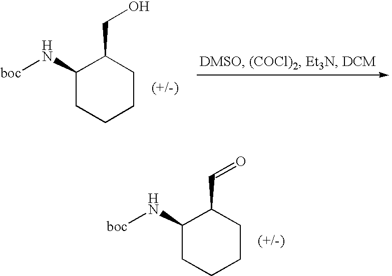

Following the same procedure as Example 89 (step B), the title compound was obtained as white solids (365 mg, 99%) and was used directly for the next step without further purification.

### Step C

### The Preparation of cis-(+/−)-tert-butyl{2-[(3-butylpiperidin-1-yl)methyl]cyclohexyl}carbamate

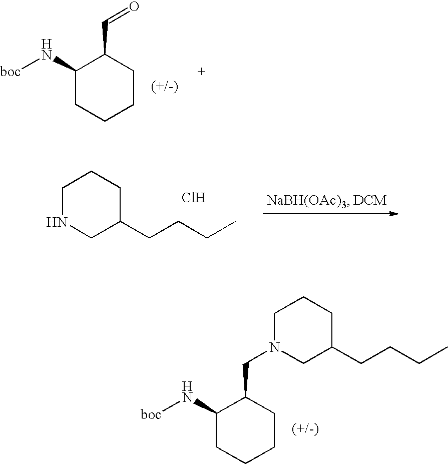

Following the same procedure as Example 89 (step C), the title compound was obtained as colorless oils (543 mg, 96%) and was used directly for the next step without further purification.

The product was used directly for the next step without further purification.

### Step D

### The Preparation of trans-(+/−)-{2-[(3-butylpiperidin-1-yl)methyl]cyclohexyl}amine hydrochloride

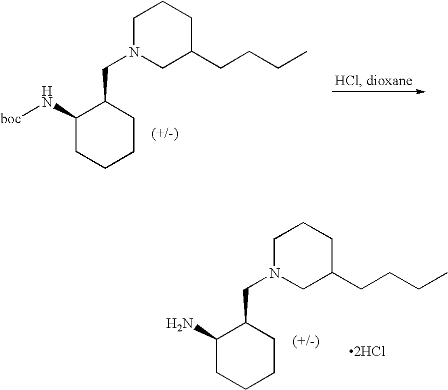

Following the same procedure as Example 89 (step D), the title compound was obtained as HCl salt (389 mg, 79%) and was used directly for the next step without further purification.

The product was used directly for the next step without further purification.

### Step E

### The Preparation of cis-(+/−)-N-{2-[(3-butylpiperidin-1-yl)methyl]cyclohexyl}-6-(1H-imidazol-1-yl)nicotinamide

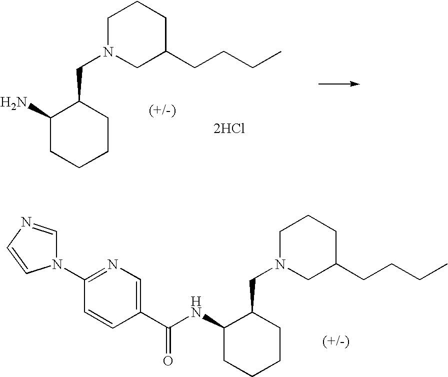

Following the same procedure as Example 2, yielded the title compound 92 mg (54%). MS (M+1): 424.3. 1H NMR (400 MHz, METHANOL-D4): δ ppm 0.71-0.94 (m, 4H), 0.99-1.35 (m, 8H), 1.41-1.65 (m, 6H), 1.69-1.94 (m, 8H), 2.21-2.38 (m, 1H), 2.79-3.12 (m, 2H), 4.04-4.31 (m, 1H), 7.17 (s, 1H), 7.81 (dd, J=8.59, 2.54 Hz, 1H), 7.95 (d, J=1.17 Hz, 1H), 8.32 (d, J=8.20 Hz, 1H), 8.60 (s, 1H) 8.88 (s, 1H).

### Example 116

### trans-(+/−)-N-(2-{[4-(Allyloxy)piperidin-1-yl]methyl}cyclohexyl)-6-(1H-pyrazol-1-yl)nicotinamide

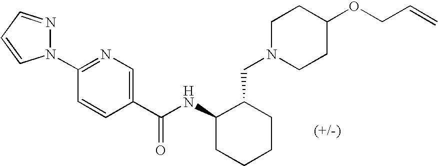

### Step A

### The Preparation of tert-butyl 4-(allyloxy)piperidin-1-carboxylate

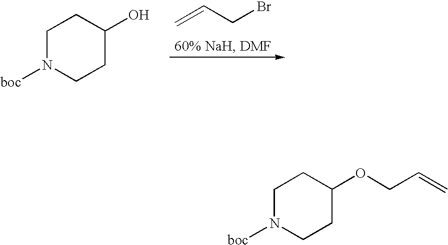

To a solution of tert-butyl 4-(hydroxy)piperidin-1-carboxylate (1.0 g, 5.0 mmol) in dry DMF (20 mL) was added NaH (60%, 0.38 g, 10 mmol) at 0° C. under nitrogen and the suspension was stirred at room temperature for 30 min. Allyl bromide (0.52 ml, 6.0 mmol) was added to the reaction mixture and stirred over night at room temperature. The solvent was removed in vacuo and the residue was dissolved in dichloromethane (50 mL), washed with water (30 mL), dried over Na₂SO₄. Removal of solvent gave the crude product, which was used for the next step without further purification.

### Step B

### The Preparation of 4-(allyloxy)piperidine hydrochloride

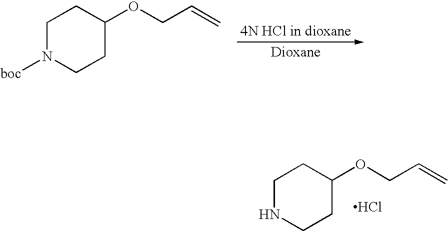

Following the same procedure as Example 97 Step B, the title compound was obtained as a white solid in a 61% yield over 2 steps (545 mg).

### Step C

### The Preparation of trans-(+/−)-tert-butyl (2-{[4-(allyloxy)piperidin-1-yl]methyl}cyclohexyl)carbamate

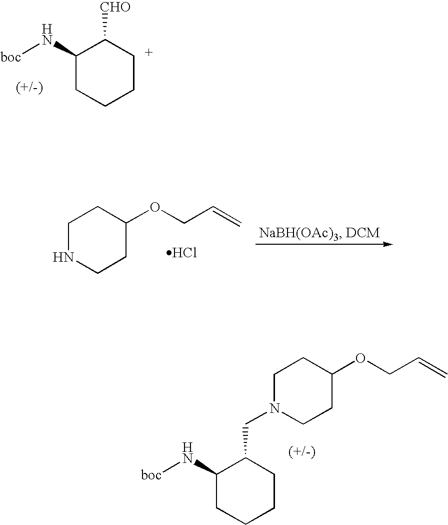

The title compound was prepared following the same procedure as Example 89 Step C. The product was used directly for the next step without further purification.

### Step D

### The Preparation of trans-(+/−)-(2-{[4-(allyloxy)piperidin-1-yl]methyl}cyclohexyl)amine hydrochloride

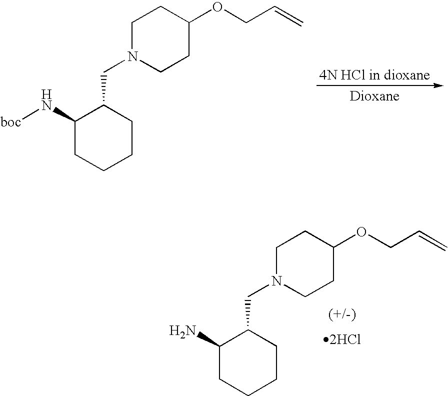

The title compound was prepared following the same procedure as Example 89 Step D. The product was used directly for the next step without further purification.

### Step E

### The Preparation of trans-(+/−)-N-(2-{[4-(Allyloxy)piperidin-1-yl]methyl}cyclohexyl)-6-(1H-pyrazol-1-yl)nicotinamide

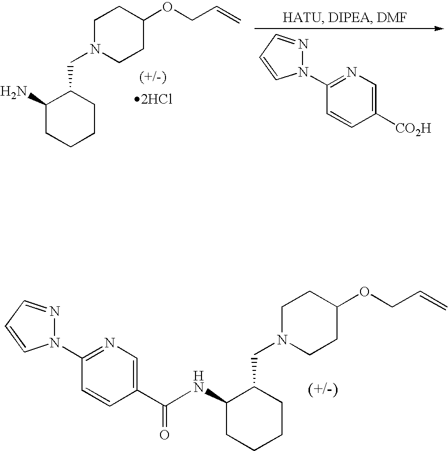

Following the procedure described in Example 2, the HCl salt of the title compound was obtained as a yellow solid in a 34% yield over 3 steps (75 mg). MS (M+1): 424.0. 1H NMR (400 MHz, METHANOL-D [symbol] ): [symbol] ppm 1.23-1.58 (m, 4H), 1.69-2.25 (m, 9H), 2.89-3.14 (m, 2H), 3.18-3.30 (m, 2H), 3.42-3.83 (m, 4H), 3.95-4.04 (m, 2H), 5.07-5.15 (m, 1H), 5.21-5.28 (m, 1H), 5.82-5.93 (m, 1H), 6.56 (s, 1H), 7.79 (s, 1H), 8.02 (d, J=8.59 Hz, 1H), 8.37 (dd, J=8.59, 2.15 Hz, 1H), 8.64 (d, J=2.15 Hz, 1H), 8.91 (s, 1H). Anal. Calcd for C₂₄H₃₃N₅O₂.2HCl.0.55C₄H₈O₂ C, 57.75; H, 7.29; N, 12.85. Found: C, 58.07; H, 7.63; N, 13.10.

### Example 117

### trans-(+/−)-N-[2-({4-[(2E)-But-2-en-1-yloxy]piperidin-1-yl}methyl)cyclohexyl]-6-(1H-pyrazol-1-yl)nicotinamide

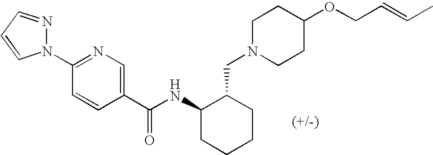

### Step A

### The Preparation of tert-butyl 4-[(2E)-but-2-en-1-yloxy]piperidin-1-carboxylate

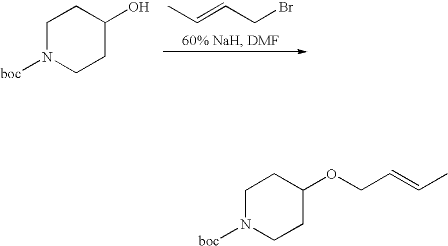

The title compound was prepared following the same procedure as Example 115 (Step A). The product was used directly for the next step without further purification.

### Step B

### The Preparation of 4-[(2E)-but-2-en-1-yloxy]piperidine hydrochloride

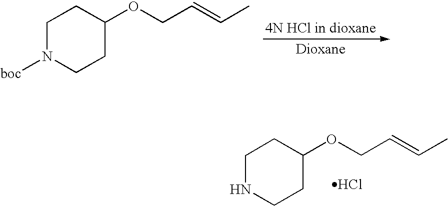

Following the same procedure as 97 Step B, the hydrochloride salt of the title compound was obtained as a white solid in a 76% yield over 2 steps (725 mg).

### Step C

### The Preparation of trans-(+/−)-tert-butyl (2-{[4-[(2E)-but-2-en-1-yloxy]piperidin-1-yl]methyl}cyclohexyl)carbamate

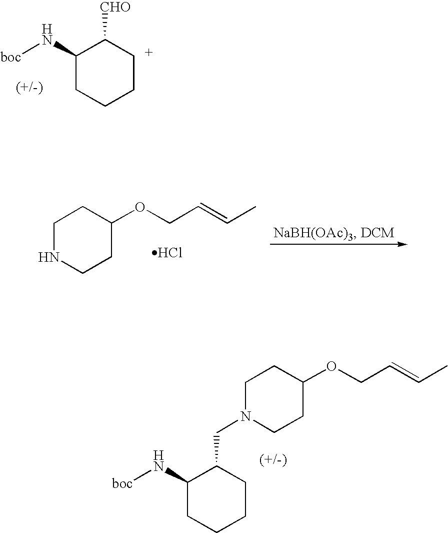

The title compound was prepared following the same procedure as Example 89 Step C. The product was used directly for the next step without further purification.

### Step D

### The Preparation of trans-(+/−)-(2-{[4-[(2E)-but-2-en-1-yloxy]piperidin-1-yl]methyl}cyclohexyl)amine hydrochloride

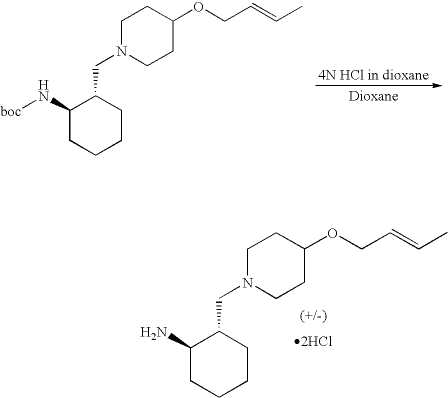

The title compound was prepared following the same procedure as Example 89 Step D. The product was used directly for the next step without further purification. MS (M+1): 267.0.

### Step E

### The Preparation of trans-(+/−)-N-(2-{[4-[(2E)-but-2-en-1-yloxy]piperidin-1-yl]methyl}cyclohexyl)-6-(1H-pyrazol-1-yl)nicotinamide

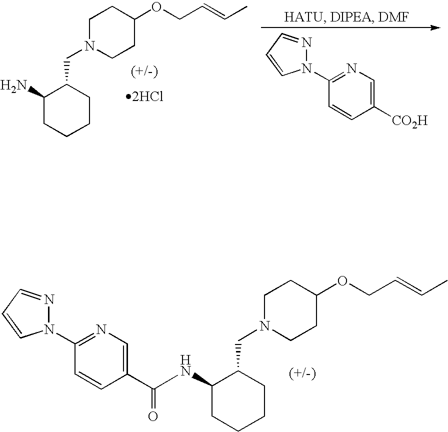

Following the procedure described in Example 2, the HCl salt of the title compound was obtained as a white solid in a 40% yield over 3 steps (75 mg). MS (M+1): 438.3. 1H NMR (400 MHz, METHANOL-D4): [symbol] ppm 1.22-2.19 (m, 16H), 2.97-3.12 (m, 2H), 3.17-3.26 (m, J=13.28, 13.28, 1.95 Hz, 1H), 3.42-3.83 (m, 5H), 3.88-3.96 (m, 2H), 5.47-5.58 (m, 1H), 5.64-5.74 (m, 1H), 6.56 (dd, J=2.54, 1.76 Hz, 1H), 7.79 (d, J=1.37 Hz, 1H), 8.02 (d, J=8.59 Hz, 1H), 8.34-8.39 (m, 1H), 8.64 (d, J=2.34 Hz, 1H), 8.90 (s, 1H). Anal. Calcd for C₂₅H₃₅N₅O₂.2.55HCl.0.7C₄H₈O₂ C, 56.38; H, 7.34; N, 11.83. Found: C, 56.18; H, 7.70; N, 12.18.

### Example 118

### trans-(+/−)-N-[2-({3-[(Allyloxy)methyl]piperidin-1-yl}methyl)cyclohexyl]-6-pyrrolidin-1-ylnicotinamide

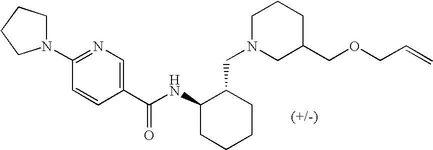

Following the procedure described in Example 2, the HCl salt of the title compound was obtained as a white solid in a 40% yield over 3 steps (101 mg). MS (M+1): 441.3. 1H NMR (400 MHz, METHANOL-D [symbol] ): [symbol] ppm 1.20-1.55 (m, 5H), 1.74-1.85 (m, 3H), 1.91-2.00 (m, 2H), 2.03-2.24 (m, 6H), 2.68-2.88 (m, 1H), 2.92-3.00 (m, 2H), 3.19-3.27 (m, 1H), 3.42 (dd, J=9.28, 4.59 Hz, 1H), 3.47-3.70 (m, 8H), 3.75 (td, J=10.40, 2.44 Hz, 1H), 3.90-3.95 (m, 2H), 5.09-5.15 (m, 1H), 5.20 (dq, J=5.49, 1.68 Hz, 1H), 5.24 (dq, J=5.42, 1.71 Hz, 1H), 5.79-5.91 (m, 1H), 7.14 (d, J=9.57 Hz, 1H), 8.40-8.45 (m, 1H), 8.52 (dd, J=6.45, 1.56 Hz, 1H).

### Example 119

### trans-(+/−)-N-[2-({3-[(Allyloxy)methyl]piperidin-1-yl}methyl)cyclohexyl]-4-(1H-pyrazol-1-yl)benzamide

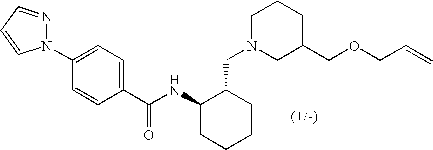

Following the procedure described in Example 2, the free base of the title compound was obtained as a white solid in a 61% yield over 3 steps (80 mg). MS (M+1): 437.3. 1H NMR (400 MHz, CHLOROFORM-D): [symbol] ppm 0.83-1.02 (m, 1H), 1.02-1.18 (m, 2H), 1.23-1.52 (m, 3H), 1.56-1.84 (m, 8H), 1.86-1.99 (m, 1H), 2.07 (dd, J=12.60, 6.15 Hz, 1H), 2.37-2.48 (m, 1H), 2.56-2.74 (m, 2H), 3.03-3.27 (m, 2H), 3.30-3.49 (m, 2H), 3.69 (dt, J=5.47, 1.37 Hz, 1H), 3.98 (dt, J=5.66, 1.37 Hz, 1H), 5.00-5.11 (m, 1H), 5.22 (dq, J=10.35, 1.51, 1.27 Hz, one diast 1H), 5.30 (dq, J=17.28, 1.59 Hz, one diast 1H) 5.71 (ddt, J=17.19, 10.35, 5.66 Hz, one diast 1H), 5.94 (ddt, J=17.38, 10.55, 5.66 Hz, one diast 1H), 6.46-6.55 (m, 1H), 7.73-7.78 (m, 3H), 7.95 (dd, J=8.69, 3.42 Hz, 2H), 7.99 (dd, J=5.96, 2.05 Hz, 1H), 8.93 (s, one diast 1H), 9.03 (s, one diast 1H).

### Example 120

### trans-(+/−)-N-[2-({3-[(Allyloxy)methyl]piperidin-1-yl}methyl)cyclohexyl]-6-(1H-imidazol-1-yl)nicotinamide

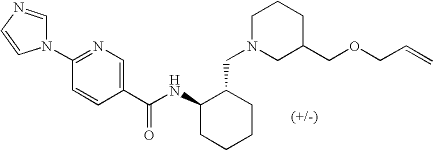

Following the procedure described in Example 2, the free base of the title compound was obtained as a white solid in a 45% yield over 3 steps (59 mg). MS (M+1): 438.3. 1H NMR (400 MHz, CHLOROFORM-D): δ ppm 0.86-1.01 (m, 1H), 1.03-1.19 (m, 2H), 1.23-1.84 (m, 11H), 1.90-1.99 (m, 1H), 2.09 (dd, J=12.79, 4.39 Hz, 1H), 2.43 (t, J=11.43 Hz, 1H), 2.55-2.75 (m, 2H), 3.03-3.28 (m, 2H), 3.31-3.48 (m, 2H), 3.71 (d, J=5.47 Hz, 1H), 4.00 (dt, J=5.81, 1.29 Hz, 1H), 5.02-5.10 (m, 1H), 5.23 (dq, J=10.35, 1.46, 1.17 Hz, one diast 1H), 5.30 (dq, J=17.19, 1.63 Hz, one diast 1H), 5.70 (ddt, J=17.19, 10.35, 5.47 Hz, one diast 1H), 5.95 (ddt, J=17.19, 10.35, 5.66 Hz, one diast 1H), 7.21-7.24 (m, 1H), 7.39 (ddd, J=8.50, 1.66, 0.78 Hz, 1H), 7.68 (dt, J=6.25, 1.46 Hz, 1H), 8.30 (ddd, J=8.40, 3.52, 2.34 Hz, 1H), 8.41 (dt, J=7.62, 0.98 Hz, 1H), 8.89 (s, 1H), 9.21 (s, one diast 1H), 9.29 (s, one diast 1H).

### Example 121-128

The same procedure described in Example 120 was followed to prepare Examples 121-128

| Ex- | | | |
|---|---|---|---|
| am- | | | |
| ple | | | |
| No. | Structure | Name | Data |
| 121 | 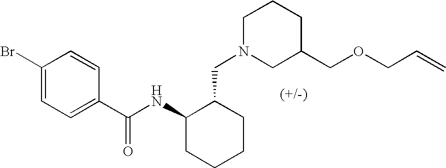 | trans-(±)- N-2-({3- [(Allyloxy)meth- yl]pipe- ridin-1- yl}methyl)cyclo- hexyl]-4- bromobenz- amide | 1H NMR(400 MHz, CHLOROFORM-D) δ ppm 0.85-2.15(m, 16H), 2.32- 2.71(m, 3H), 3.03-3.48(m, 4H), 3.69-3.77(m, |
| #J=5.52, 2.78, 1.51, 1.51Hz, 1H), 3.98(dt, J=5.81, 1.39Hz, 1H), 5.09-5.33((m, 2H), 5.73- 6.00(m, 1H), 7.52-7.56 (m, 2H), 7.71(dd, J=8.50, 1.66Hz, 2H), 8.92(s, 1H one isomer), 9.00(s, 1H one isomer). MS: 449.3(M + 1). | | | |
| 122 | 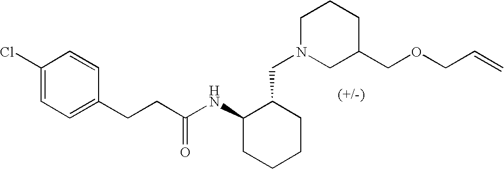 | Trans-(±)- (N-2-({3- [(Allyloxy)meth- yl]pipe- ridin-1- yl}methyl)cyclo- hexyl]-3-(4- chlorophe- nyl)propan- amide | 1H NMR(400 MHz, CHLOROFORM-D) δ ppm 0.85-1.07(m, 3H), 1.15- 1.95(m, 13H), |
| #1.95-2.06 (m, 1H), 2.19-2.49(m, 4H), 2.64-3.35(m, 6H), 3.87- 4.00(m, 2H), 5.13-5.31(m, 2H), 5.80-5.98(m, 1H), 7.11-7.18(m, 2H), 7.21- 7.25(m, 2H), 7.99(s, 1H), 8.02(s, 1H). MS: 433.3 (M + 1). | | | |
| 123 | 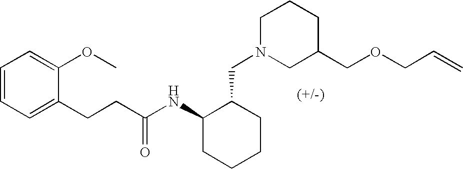 | Trans-(±)- N-[2-({3- [(Allyloxy)meth- yl]pipe- ridin-1- yl}methyl)cyclo- hexyl]-3-(2- methoxy- phenyl)prop- anamide | 1H NMR(400 MHz, CHLOROFORM-D) δ ppm 0.86-1.07(m, 3H), 1.16-2.05(m, 15H), 2.22-2.47 (m, |
| #4H), 2.56(d, J=11.52Hz, 1H one isomer), 2.70(dd, J=5.37, 2.83Hz, 1H one isomer), 2.80-2.86(m, 1H one isomer), 2.91-2.98(m, 2H), 3.08(d, J=10.94Hz, 1H one isomer), 3.19-3.33(m, 3H), 3.81(s, 3H one isomer), 3.82(s, 3H one isomer), 3.89 (dq, | | | |
| #J=5.54, 1.34Hz, 1H), 3.95(dt, J=5.66, 1.37Hz, 1H), 5.11-5.30(m, 2H), 5.79- 5.96(m, 1H), 6.81-6.90 (m, 2H), 7.15-7.20(m, 2H), 7.70(d, J=3.91Hz, 1H one isomer), 7.84(s, 1H one isomer). MS: 429.3(M + 1). | | | |
| 124 | 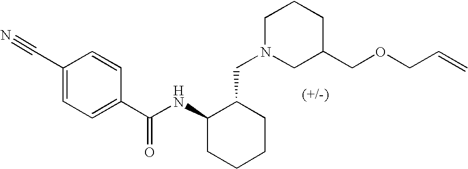 | Trans-(±)- N-[2-({3- [(Allyloxy)meth- yl]pipe- ridin-1- yl}methyl)cyclo- hexyl]-4- cyanobenz- amide | 1H NMR(400 MHz, CHLOROFORM-D) δ ppm 0.82-1.17(m, 3H), 1.23- 1.97(m, 12H), 2.08(dd, J=12.79, 3.22Hz, 1H), |
| #2.34- 2.74(m, 3H), 3.02-3.26(m, 2H), 3.29-3.48(m, 2H), 3.76(dt, J=5.47, 1.37Hz, 1H), 3.99(dt, J=5.66, 1.37Hz, 1H), 5.10-5.34(m, 2H), 5.72-6.01(m, 1H), 7.68- 7.73(m, 2H), 7.92(dd, J=7.91, 4.79Hz, 2H), 9.16 (s, 1H one isomer), 9.26(s, 1H one isomer). MS: 396.3 (M + 1). | | | |
| 125 | 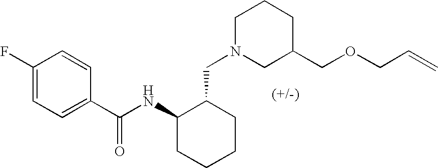 | Trans-(±)- N-[(2-({3- [(Allyloxy)meth- yl]pipe- ridin-1- yl}methyl)cyclo- hexyl]-4- fluorobenz- amide | 1H NMR(400 MHz, CHLOROFORM-D) δ ppm 1.08(d, J=10.94Hz, 3H), 1.23-1.84(m, 11H), 1.85- 1.97(m, 1H), |
| #1.98-2.13(m, 1H), 2.34-2.49(m, 1H), 2.50-2.70(m, 2H), 3.02- 3.50(m, 4H), 3.74(d, J=5.47Hz, 2H one isomer), 3.98(dt, J=5.66, 1.27Hz, 2H one isomer), 5.08-5.34(m, 2H), 5.72-6.00(m, 1H), 7.04- 7.12(m, 2H), 7.83(t, J=5.66Hz, 2H), 8.87(s, 1H one isomer), 8.95(s, 1H one isomer). MS: 389.3(M + 1). | | | |
| 126 | 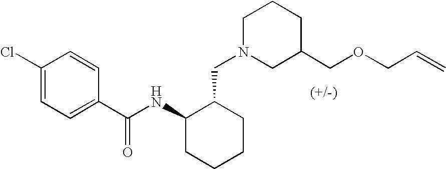 | Trans-(±)- N-[(2-({3- [(Allyloxy)meth- yl]pipe- ridin-1- yl}methyl)cyclo- hexyl]-4- chlorobenz- amide | 1H NMR(400 MHz, CHLOROFORM-D) δ ppm 0.82-1.18(m, 3H), 1.22- 1.44(m, 2H), 1.44-1.85(m, 9H), 1.91(t, J=10.84Hz, |
| #1H), 2.06(dd, J=12.79, 6.74Hz, 1H), 2.34-2.46(m, 1H), 2.50-2.73(m, 2H), 3.04- 3.47(m, 4H), 3.73(dq, J=5.44, 1.57Hz, 2H one isomer), 3.98(dt, J=5.66, 1.37Hz, 2H one isomer), 5.09-5.34(m, 2H), 5.73- 6.00(m, 1H), 7.35-7.40(m, 2H), 7.78(dd, J=8.59, 2.15Hz, 2H), 8.92(s, 1H | | | |
| #one isomer), 9.00(s, 1H one isomer). MS: 405.3(M + 1). | | | |
| 127 | 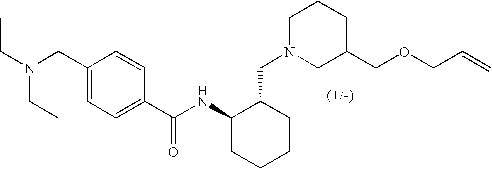 | Trans-(±)- N-[2-({3- [(Allyloxy)meth- yl]pipe- ridin-1- yl}methyl)cyclo- hexyl]-4- [(diethyla- mino)meth- yl]benzamide | 1H NMR(400 MHz, CHLOROFORM-D) δ ppm 0.82-1.00(m, 1H), 1.03(t, J=7.13Hz, 6H), 1.06-1.51 (m, |
| #4H), 1.52-1.95(m, 10H), 2.04(ddd, J=12.94, 8.35, 1.56Hz, 1H), 2.35-2.45(m, 1H), 2.50(q, J=7.23Hz, 4H), 2.54-2.67(m, 2H), 3.03 (d, J=11.72Hz, 1H one isomer), 3.12(d, J=6.25Hz, 1H), 3.22(dd, J=9.18, 7.81Hz, 1H one isomer), 3.26(d, J=10.35Hz, 1H one isomer), 3.34(dd, J=9.18, | | | |
| #5.08Hz, 1H one isomer), 3.38-3.48 (m, 1H), 3.59(s, 2H), 3.72 (dt, J=5.47, 1.37Hz, 2H one isomer), 3.98(dt, J=5.66, 1.37Hz, 2H one isomer), 5.06-5.35(m, 2H), 5.71- 6.00(m, 1H), 7.37(d, J=7.81Hz, 2H), 7.77(d, J=8.20Hz, 2H), 8.76(s, 1H one isomer), 8.86(s, 1H one isomer). MS: 456.3(M + 1). | | | |
| 128 | 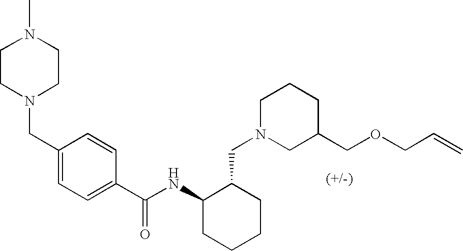 | Trans-(±)- N-[2-({3- [(Allyloxy)meth- yl]pipe- ridin-1- yl}methyl)cyclo- hexyl]-4-[(4- methylpipe- razin-1- yl)methyl]benz- amide | 1H NMR(400 MHz, CHLOROFORM-D) δ ppm 0.82-1.51(m, 6H), 1.52- 1.93(m, 9H), 2.00-2.09(m, 2H), |
| #2.28(s, 3H), 2.35-2.68 (m, 10H), 3.04(d, J=10.16Hz, 1H one isomer), 3.11(d, J=6.25Hz, 1H), 3.23(dd, J=9.18, 7.81Hz, 1H one isomer), 3.27(d, J=9.96Hz, 1H one isomer), 3.34(dd, J=9.28, 5.18Hz, 1H one isomer), 3.38-3.47(m, 1H), 3.52(s, 2H one isomer), 3.53 (s, 2H one isomer), 3.72(dt, J=5.47, | | | |
| #1.46Hz, 2H one isomer), 3.98(dt, J=5.66, 1.37Hz, 2H one isomer), 5.07-5.34(m, 2H), 5.71- 6.00(m, 1H), 7.36(d, J=8.40Hz, 2H), 7.77(d, J=8.20Hz, 2H), 8.76(d, J=2.54Hz, 1H one isomer), 8.85(d, J=2.73Hz, 1H one isomer). MS: 483.3(M + 1). | | | |

### Example 129

### Trans-(±)-[2-({(3R)-3-[(allyloxy)methyl]piperidin-1-yl}methyl)cyclohexyl]-6-(1H-imidazol-1-yl)nicotinamide

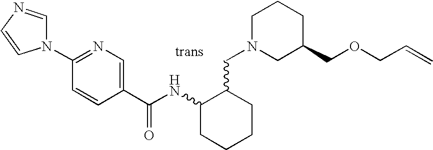

### Step A

### The Preparation of tert-butyl (3R)-3-[(allyloxy)methyl]piperidin-1-carboxylate

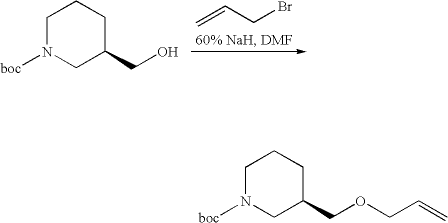

The title compound was prepared following the same procedure as Example 115 (Step A). The product was used directly for the next step without further purification.

### Step B

### The Preparation of (3R)-3-[(allyloxy)methyl]piperidine hydrochloride

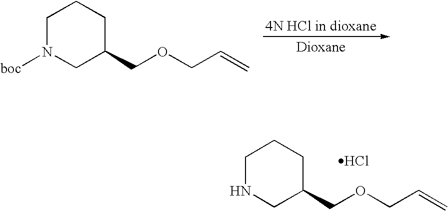

Following the same procedure as Example 97 Step B, the hydrochloride salt of the title compound was obtained as a white solid in a 80% yield over 2 steps (397 mg).

### Step C

### The Preparation of Trans-(±)-tert-butyl[2-({(3R)-3-[(allyloxy)methyl]piperidin-1-yl}methyl)cyclohexyl]carbamate trans

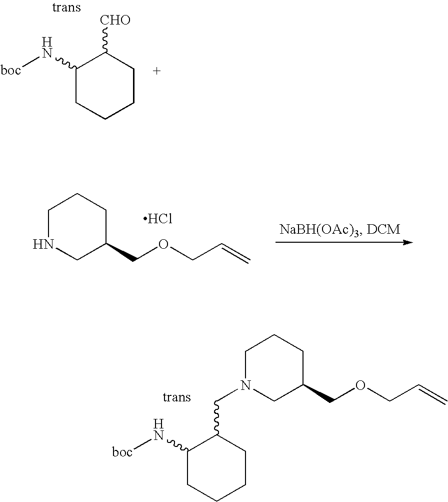

The title compound was prepared following the same procedure as Example 89 Step C. The product was used directly for the next step without further purification.

### Step D

### The Preparation of Trans-(±)-[2-({(3R)-3-[(allyloxy)methyl]piperidin-1-yl}methyl)cyclohexyl]amine hydrochloride

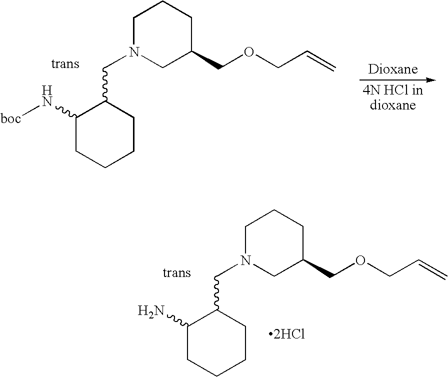

The title compound was prepared following the same procedure as Example 89 Step D. The product was used directly for the next step without further purification. MS (M+1): 267.2.

### Step E

### The Preparation of trans-(±) [2-({(3R)-3-[(allyloxy)methyl]piperidin-1-yl}methyl)cyclohexyl]-6-(1H-imidazol-1-yl)nicotinamide

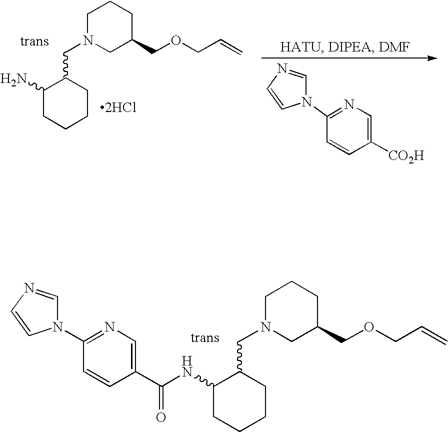

Following the procedure described in Example 2, the free base of the title compound was obtained as a white solid in a 36% yield over 3 steps (130 mg). MS (M+1): 438.3. 1H NMR (400 MHz, CHLOROFORM-D): δ ppm 0.86-1.02 (m, 1H), 1.08-1.80 (m, 13H), 1.94 (t, J=10.74 Hz, 1H), 2.10 (dd, J=13.28, 4.88 Hz, 1H), 2.42 (t, J=10.25 Hz, 1H), 2.56-2.75 (m, 2H), 3.06-3.18 (m, 1H and one diast 1H) 3.24 (dd, J=9.18, 8.01 Hz, one diast 1H), 3.32-3.48 (m, 1H), 3.38 (dd, J=9.28, 4.98 Hz, 1H), 3.71 (d, J=5.47 Hz, 1H), 3.99 (dt, J=5.81, 1.29 Hz, 1H), 5.01-5.11 (m, 1H), 5.22 (dq, J=10.35, 1.46, 1.17 Hz, one diast 1H), 5.30 (dq, J=17.26, 1.60 Hz, one diast 1H), 5.70 (ddt, J=17.19, 10.55, 5.47 Hz, one diast 1H), 5.94 (ddt, J=17.19, 10.35, 5.66 Hz, one diast 1H), 7.22 (s, 1H), 7.39 (ddd, J=8.59, 1.76, 0.78 Hz, 1H), 7.68 (dt, J=6.05, 1.17 Hz, 1H), 8.28-8.32 (m, 1H), 8.41 (d, J=7.42 Hz, 1H), 8.89 (s, 1H), 9.21 (s, one diast 1H), 9.29 (s, one diast 1H).

### Example 130

### Trans-(±)-[2-({(3S)-3-[(Allyloxy)methyl]piperidin-1-yl}methyl)cyclohexyl]-6-(1H-imidazol-1-yl)nicotinamide

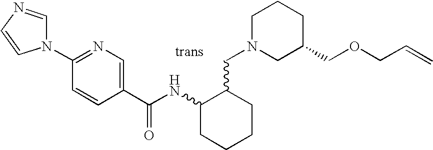

### Step A

### The Preparation of tert-butyl (3S)-3-[(allyloxy)methyl]piperidine-1-carboxylate

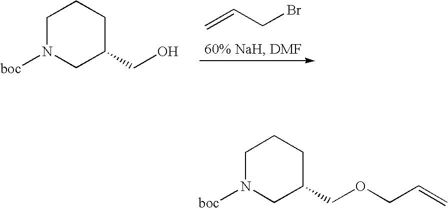

The title compound was prepared following the same procedure as Example 115 (Step A). The product was used directly for the next step without further purification.

### Step B

### The Preparation of (3S)-3-[(allyloxy)methyl]piperidine hydrochloride

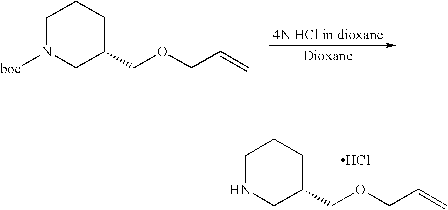

Following the same procedure as Example 97 Step B, the hydrochloride salt of the title compound was obtained as a white solid in a 75% yield over 2 steps (372 mg).

### Step C

### The Preparation of trans-(±)-tert-butyl[2-({(3S)-3-[(allyloxy)methyl]piperidin-1-yl}methyl)cyclohexyl]carbamate

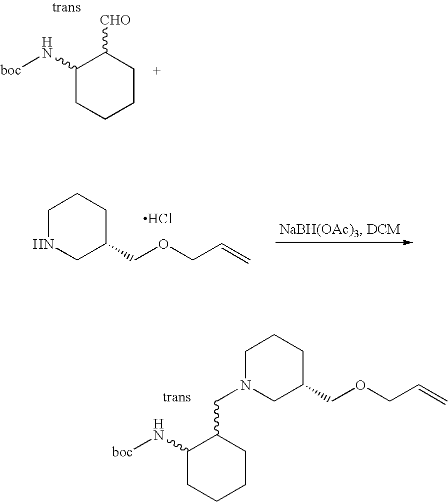

The title compound was prepared following the same procedure as Example 89 Step C. The product was used directly for the next step without further purification.

### Step D

### The Preparation of trans-(±)[2-({(3S)-3-[(allyloxy)methyl]piperidin-1-yl}methyl)cyclohexyl]amine hydrochloride

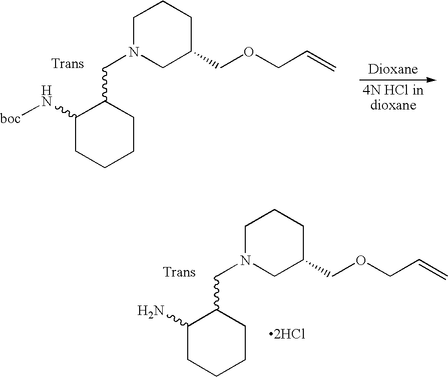

The title compound was prepared following the same procedure as Example 89 Step D. The product was used directly for the next step without further purification. MS (M+1): 267.2.

### Step E

### The Preparation of trans-(±)[2-({(3S)-3-[(allyloxy)methyl]piperidin-1-yl}methyl)cyclohexyl]-6-(1H-imidazol-1-yl)nicotinamide

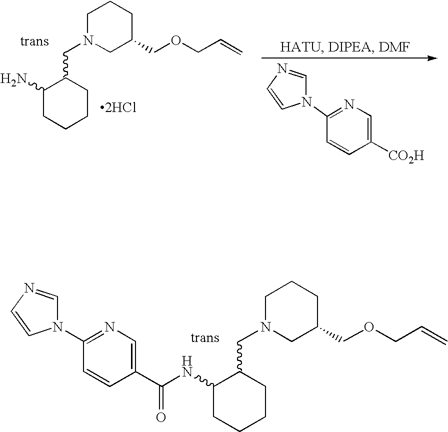

Following the procedure described in Example 2, the free base of the title compound was obtained as a white solid in a 41% yield over 3 steps (205 mg). MS (M+1): 438.3. MS (M+1): 438.3. 1H NMR (400 MHz, CHLOROFORM-D) [symbol] [symbol] ppm 0.86-1.02 (m, 1H), 1.08-1.80 (m, 13H), 1.94 (t, J=10.74 Hz, 1H), 2.10 (dd, J=13.28, 4.88 Hz, 1H), 2.42 (t, J=10.25 Hz, 1H), 2.56-2.75 (m, 2H), 3.06-3.18 (m, 1H and one diast 1H) 3.24 (dd, J=9.18, 8.01 Hz, one diast 1H), 3.32-3.48 (m, 1H), 3.38 (dd, J=9.28, 4.98 Hz, 1H), 3.71 (d, J=5.47 Hz, 1H), 3.99 (dt, J=5.81, 1.29 Hz, 1H), 5.01-5.11 (m, 1H), 5.22 (dq, J=10.35, 1.46, 1.17 Hz, one diast 1H), 5.30 (dq, J=17.26, 1.60 Hz, one diast 1H), 5.70 (ddt, J=17.19, 10.55, 5.47 Hz, one diast 1H), 5.94 (ddt, J=17.19, 10.35, 5.66 Hz, one diast 1H), 7.22 (s, 1H), 7.39 (ddd, J=8.59, 1.76, 0.78 Hz, 1H), 7.68 (dt, J=6.05, 1.17 Hz, 1H), 8.28-8.32 (m, 1H), 8.41 (d, J=7.42 Hz, 1H), 8.89 (s, 1H), 9.21 (s, one diast 1H), 9.29 (s, one diast 1H).

### Examples 131-145

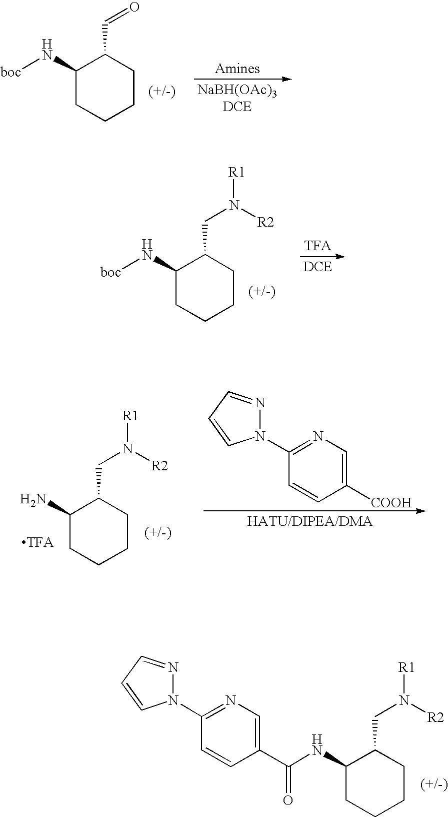 Procedure:

In a plate format, a 0.30M solution of amine in dichloroethane (0.80 ml, 0.22 mmol) was added to a 0.40M solution of trans-(+/−)-tert-butyl[2-formylcyclohexyl]carbamate in dichloroethane (0.50 ml, 0.20 mmol). Solid sodium triacetoxyborohydride (85 mg, 0.40 mmol) was added to the reaction mixtures. The mixtures were stirred at room temperature for 72 hours. A 1N sodium hydroxide solution (0.45 ml, 0.45 mmol) was added. The mixtures were filtered on Hydromatrix and washed with dichloromethane. The mixtures were concentrated.

The crude compounds were dissolved in dichloroethane (0.80 ml) and trifluoroacetic acid (0.15 ml) was added. The reactions were stirred at room temperature for 8 hours and concentrated.

A 0.2M solution of 6-(1H-pyrazol-1-yl)-nicotinic acid in dimethylacetamide (1.1 ml, 0.22 mmol) was added to the crude compounds, followed by diisopropylethylamine (0.14 ml, 0.8 mmol) and a 0.55M solution of HATU in dimethylacetamide (0.41 ml, 0.22 mmol). The reactions were stirred at room temperature for 16 hours and concentrated. The crude compounds were dissolved in 0.60 ml dichloromethane. A 1N sodium hydroxide solution (0.20 ml) was added. The mixtures were filtered on Hydromatrix and washed three times with dichloromethane. The mixtures were concentrated.

The compounds were purified by high pH reverse phase prep LC-MS.

| | | | | Retention |
|---|---|---|---|---|
| | | | | time |
| Exp. No. | Structure | Name | MS (M + 1) | (min) |
| 131 | 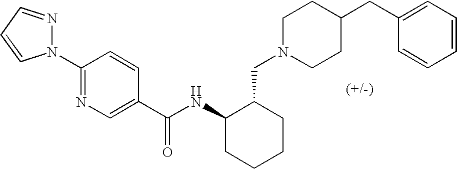 | trans-(+/−)-N-{2-[(4- benzylpiperidin-1- yl)methyl]cyclohexyl}-6- (1H-pyrazol-1- yl)nicotinamide | 457.87 | 2.19 |
| 132 | 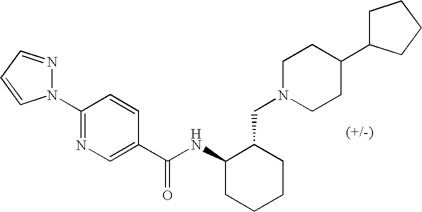 | trans-(+/−)-N-{2-[(4- cyclopentylpiperazin-1- yl)methyl]cyclohexyl}-6- (1H-pyrazol-1- yl)nicotinamide | 436.89 | 1.57 |
| 133 | 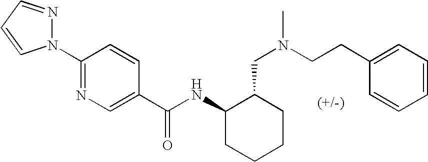 | trans-(+/−)-N-(2-{[methyl(2- phenylethyl)amino]methyl}cyclo- hexyl)-6-(1H-pyrazol- 1-yl)nicotinamide | 417.88 | 1.87 |
| 134 | 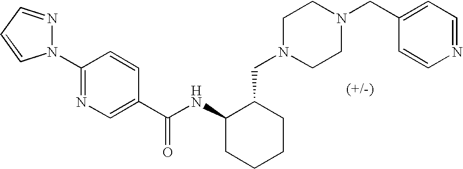 | trans-(+/−)-6-(1H-pyrazol-1- yl)-N-(2-{[4-(pyridin-4- ylmethyl)piperazin-1- yl]methyl}cyclohexyl)nicotin- amide | 459.92 | 1.34 |
| 135 | 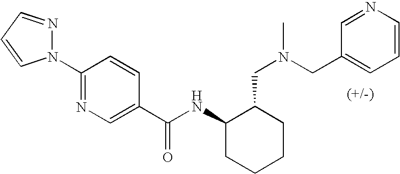 | trans-(+/−)-N-(2- {[methyl(pyridin-3- ylmethyl)amino]methyl}cyclo- hexyl)-6-(1H-pyrazol-1- yl)nicotinamide | 404.86 | 1.36 |
| 136 | 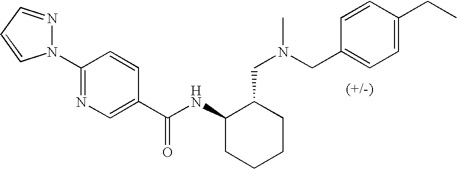 | trans-(+/−)-N-(2-{[(4- ethylbenzyl)(methyl)amino]meth- yl}cyclohexyl)-6-(1H- pyrazol-1-yl)nicotinamide | 431.88 | 2.07 |
| 137 | 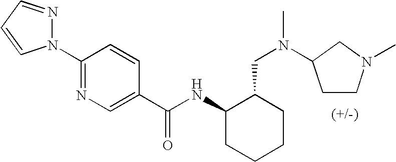 | trans-(+/−)-N-(2-{[methyl(1- methylpyrrolidin-3- yl)amino]methyl}cyclohexyl)- 6-(1H-pyrazol-1- yl)nicotinamide | 396.89 | 1.27 |
| 138 | 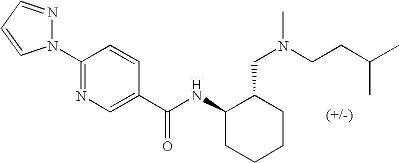 | trans-(+/−)-N-(2-{[methyl(3- methylbutyl)amino]methyl}cylcohexyl)-6-(1H-pyrazol- 1-yl)nicotinamide | 383.91 | 1.92 |
| 139 | 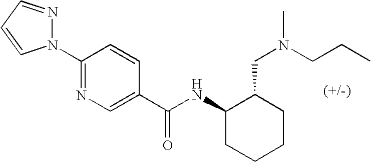 | trans-(+/−)-N-(2- {[methyl(propyl)amino]meth- yl}cyclohexyl)-6-(1H- pyrazol-1-yl)nicotinamide | 355.95 | 1.64 |
| 140 | 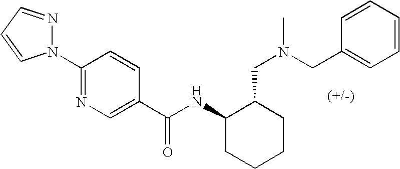 | trans-(+/−)-N-(2- {[benzyl(methyl)amino]meth- yl}cyclohexyl)-6-(1H- pyrazol-1-yl)nicotinamide | 403.86 | 1.83 |
| 141 | 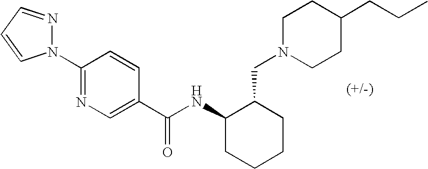 | trans-(+/−)-N-{2-[(4- propylpiperidin-1- yl)methyl]cyclohexyl}-6- (1H-pyrazol-1- yl)nicotinamide | 409.9 | 2.24 |
| 142 | 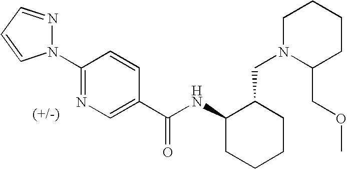 | trans-(+/−)-N-(2-{[2- (methoxymethyl)piperidin- 1-yl]methyl}cyclohexyl)-6- (1H-pyrazol-1- yl)nicotinamide | 411.88 | 1.78 |
| 143 | 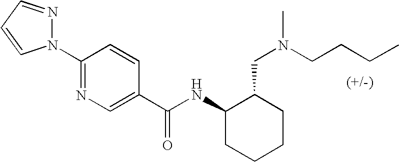 | trans-(+/−)-N-(2- {[butyl(methyl)amino]meth- yl}cyclohexyl)-6-(1H- pyrazol-1-yl)nicotinamide | 369.91 | 1.8 |
| 144 | 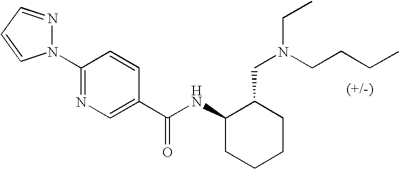 | trans-(+/−)-N-(2- {[butyl(ethyl)amino]methyl}cyclohexyl)-6-(1H-pyrazol- 1-yl)nicotinamide | 383.93 | 1.96 |
| 145 | 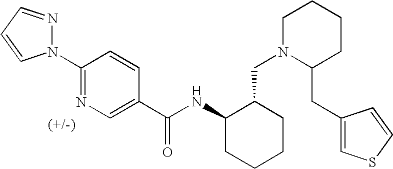 | trans-(+/−)-6-(1H-pyrazol-1- yl)-N-(2-{[2-(3- thienylmethyl)piperidin-1- yl]methyl}cyclohexyl)nicotin- amide | 463.81 | 2.09 |

### Example 146

### trans-(+/−)-N-{2-[(4,4-difluoropiperidin-1-yl)methyl]cyclohexyl}-4-methoxybenzamide

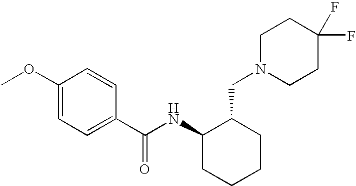

### Step A

### The Preparation of trans-(+/−)-{2-[(tert-butoxycarbonyl)amino]cyclohexyl}methyl 4-methylbenzenesulfonate

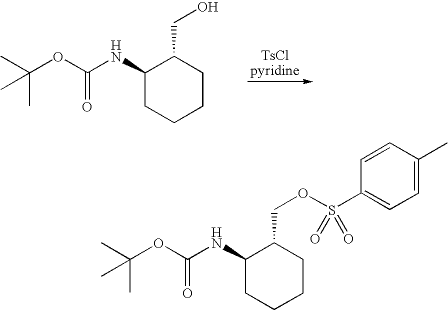

To a solution of 2.41 g (10.6 mmol) of trans-(+/−)-tert-butyl[2-(hydroxymethyl)cyclohexyl]carbamate (Example 89, step A) in dry pyridine (20 mL) was added Tosyl chloride (2.53 g, 13.25 mmol) at 0° C., the reaction mixture was stirred at 0° C. for 5 h and then at room temperature for 48 h. Ice water was added, extracted with DCM (50 mL), dried over Na₂SO₄. After removal of the solvent, the residue was purified with flash chromatography to give the title product as white solids (4.02 g, 87%).

### Step B

### The Preparation of trans-(+/−)-tert-butyl {2-[(4,4-difluoropiperidin-1-yl)methyl]cyclohexyl}carbamate

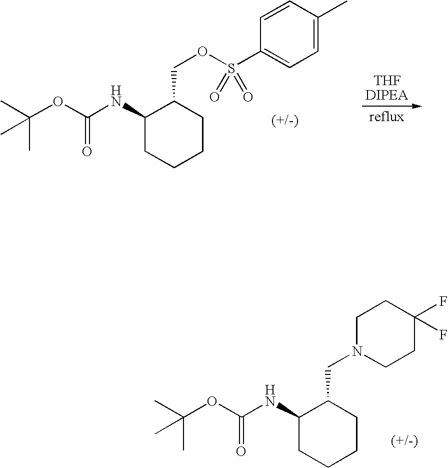

To a solution of trans-(+/−)-{2-[(tert-butoxycarbonyl)amino]cyclohexyl}methyl 4-methylbenzenesulfonate (192 mg, 0.5 mmol) in THF (5 mL) was added 4,4-difluoropiperidine hydrochloride (95 mg, 0.6 mmol) followed by DIPEA (1.5 mmol). The solution was refluxed for 5 h. After being cooled to room temperature, DCM (30 mL) was added, extracted with 1N NaOH (10 mL), dried over Na₂SO₄. After removal of the solvent, the crude product was used for the next step without further purification.

### Step C

### The Preparation of trans-(+/−)-N-{2-[(4,4-difluoropiperidin-1-yl)methyl]cyclohexyl}-4-methoxybenzamide

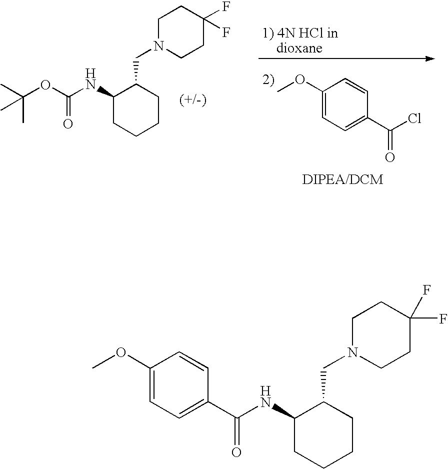

Following the procedure described in Example 89 (steps D to E), the title compound was obtained in as a white solid in a 27% yield over 2 steps (32 mg, TFA salt). MS (M+1): 367.3. 1H NMR (400 MHz, METHANOL-D4): [symbol] pp [symbol] 1.22-1.63 (m, 4H), 1.78-1.90 (m, 2H), 1.93-2.03 (m, 2H), 2.04-2.12 (m, 1H), 2.26-2.45 (m, 4H), 3.10-3.20 (m, 2H), 3.25-3.29 (m, 1H), 3.33-3.45 (m, 1H), 3.54-3.68 (m, 1H), 3.73-3.83 (m, 2H), 3.85 (s, 3H), 7.00 (d, J=8.79 Hz, 2H), 7.83 (d, J=8.79 Hz, 2H).

### Example 147

### trans-(+/−)-4-meth oxy-N-{2-[(4-methylpiperidin-1-yl)methyl]cyclohexyl}benzamide

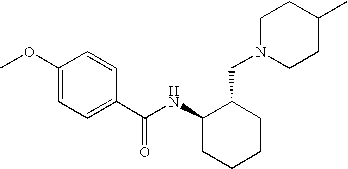

Following the same procedure as described in Example 146 (steps B to C), the title compound was obtained as its TFA salt (18 mg, 16% for 3 steps). MS (M+1): 345.3. 1H NMR (400 MHz, METHANOL-D4): [symbol] pp [symbol] 0.99 (d, J=6.45 Hz, 3H), 1.21-1.56 (m, 6H), 1.60-1.73 (m, 1H), 1.77-1.90 (m, 4H), 1.91-2.01 (m, 2H), 2.01-2.10 (m, 1H), 2.73-2.87 (m, 1H), 2.95-3.17 (m, 3H), 3.37-3.47 (m, 1H), 3.59-3.67 (m, 1H), 3.73-3.82 (m, 1H), 3.85 (s, 3H), 7.00 (d, J=8.79 Hz, 2H), 7.82 (d, J=8.79 Hz, 2H).

### Example 148

### trans-(+/−)-4-(2-methoxyethoxy)-N-{2-[(4-methylpiperidin-1-yl)methyl]cyclohexyl}benzamide

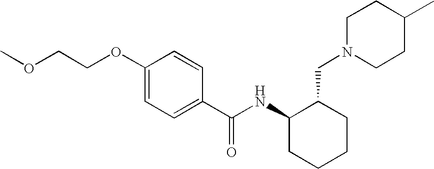

Following the same procedure as described in Example 146 (steps B to C), the title compound was obtained as its TFA salt (14 mg, 11% for 3 steps). MS (M+1): 389.3. 1H NMR (400 MHz, METHANOL-D4): [symbol] pp [symbol] 0.99 (d, J=6.44 Hz, 3H), 1.22-1.58 (m, 6H), 1.62-1.74 (m, 1H), 1.76-1.91 (m, 4H), 1.91-2.01 (m, 2H), 2.02-2.12 (m, 1H), 2.71-2.86 (m, 1H), 2.97-3.17 (m, 3H), 3.38-3.48 (m, 1H), 3.41-3.44 (m, 3H), 3.58-3.69 (m, 1H), 3.73-3.83 (m, 3H), 4.11-4.22 (m, 2H), 7.02 (d, J=8.79 Hz, 2H), 7.82 (d, J=8.79 Hz, 2H).

### Example 149

### trans-(+/−)-4-methoxy-N-[2-(morpholin-4-ylmethyl)cyclohexyl]benzamide

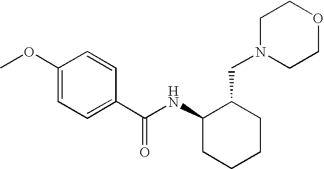

Following the same procedure as described in Example 146 (steps B to C), the title compound was obtained as its TFA salt (42 mg, 31% for 3 steps). MS (M+1): 333.3. 1H NMR (400 MHz, METHANOL-D4): [symbol] pp [symbol] 1.20-1.61 (m, 4H), 1.78-1.91 (m, 2H), 1.93-2.03 (m, 2H), 2.04-2.13 (m, 1H), 2.97-3.14 (m, 2H), 3.15-3.27 (m, 2H), 3.39 (d, J=12.20 Hz, 1H), 3.57 (d, J=12.20 Hz, 1H), 3.73-3.84 (m, 3H), 3.85 (s, 3H), 3.94-4.08 (m, 2H), 7.00 (d, J=8.89 Hz, 2H), 7.83 (d, J=8.89 Hz, 2H).

### Example 150

### cis-(+/−)-4-(2-ethoxyethoxy)-N-[2-(piperidin-1-ylmethyl)cyclohexyl]benzamide

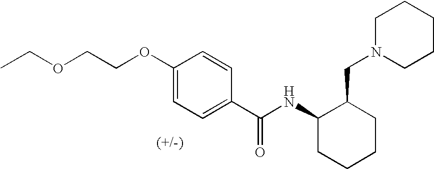

### Step A

### The Preparation of cis-(+/−)-benzyl[2-(hydroxymethyl)cyclohexyl]carbamate

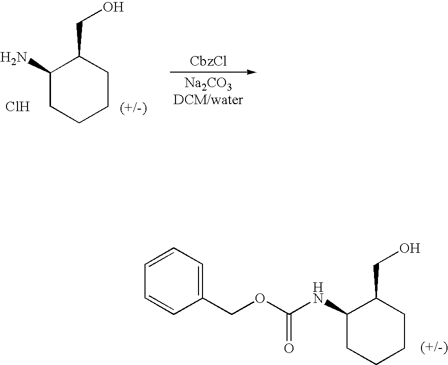

Following the same procedure as Example 1 (Step B), 612 mg of (+/−) cis-[2-aminocyclohexyl]methanol hydrochloride (3.69 mmol) was treated with Na₂CO₃ and benzyl chloroformate to yield crude cis-(+/−)-benzyl[2-(hydroxymethyl)cyclohexyl]carbamate 0.95 g (98%).

### Step B

### The Preparation of cis-(+/−)-benzyl[2-formylcyclohexyl]carbamate

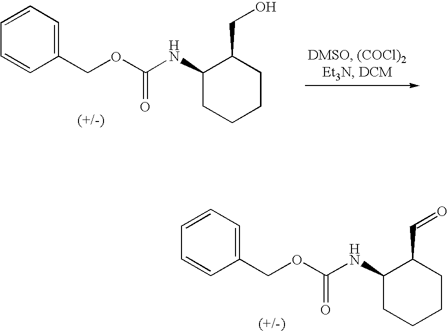

Following the same procedure as Example 89 (step B), yielded crude cis-(+/−)-benzyl[2-formylcyclohexyl]carbamate 923 mg (98%), which was used for the next step without further purification.

### Step C

### The Preparation of cis-(+/−)-benzyl[2-(piperidin-1-ylmethyl)cyclohexyl]carbamate

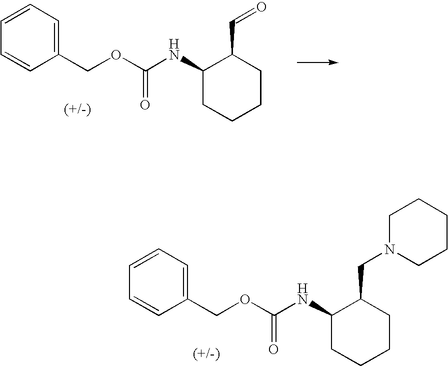

Following the same procedure as Example 89 (step C), cis-(+/−)-benzyl[2-formylcyclohexyl]carbamate from step B (1.8 mmol) was treated with NaBH(OAc)₃ to yielded cis-(+/−)-benzyl[2-(piperidin-1-ylmethyl)cyclohexyl]carbamate 520 mg (88%), which was used for the next step without further purification.

### Step D

### The Preparation of cis-(+/−)-[2-(piperidin-1-ylmethyl)cyclohexyl]amine

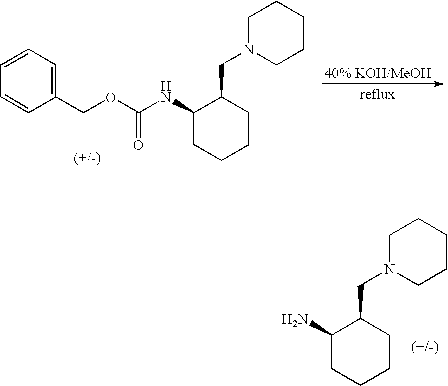

The solution of crude cis-(+/−)-benzyl[2-(piperidin-1-ylmethyl)cyclohexyl]carbamate (0.3 mmol) in 40% KOH/MeOH (8 mL, 1:1 v/v) was stirred at reflux for 5 h. The reaction mixture was cooled to room temperature, extracted with DCM (3×10 mL), dried over Na₂SO₄, concentrated to yield crude cis-(+/−)-[2-(piperidin-1-ylmethyl)cyclohexyl]amine (50 mg, 85%), which was used for the next step without further purification.

### Step E

### The Preparation of cis-(+/−)-4-(2-ethoxyethoxy)-N-[2-(piperidin-1-ylmethyl)cyclohexyl]benzamide

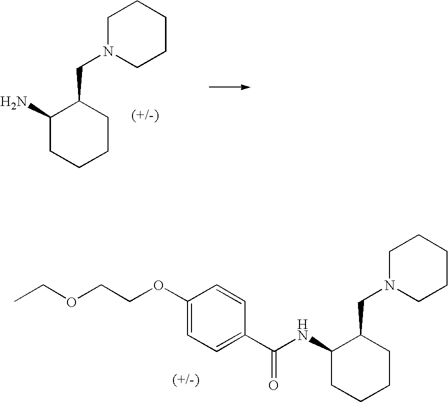

Following the same procedure as Example 2, the crude cis-(+/−)-[2-(piperidin-1-ylmethyl)cyclohexyl]amine from step D was converted to amide to yield cis-(+/−)-4-(2-ethoxyethoxy)-N-[2-(piperidin-1-ylmethyl)cyclohexyl]benzamide (49 mg, 38%). MS (M+1): 389.0. 1H NMR (400 MHz, CHLOROFORM-D): [symbol] pp [symbol] 1.25 (t, J=6.95 Hz, 3H), 1.31-1.61 (m, 4H), 1.63-1.78 (m, 4H), 1.82-2.02 (m, 6H), 2.30-2.44 (m, 1H), 2.73-2.90 (m, 2H), 2.91-3.02 (m, 2H), 3.36-3.49 (m, 1H), 3.54-3.60 (m, 1 H), 3.61 (q, J=6.95 Hz, 2H), 3.78-3.85 (m, 2H), 4.14-4.20 (m, 2H), 4.23-4.31 (m, 1H), 6.97 (d, J=8.40 Hz, 2H), 7.09 (d, J=7.03 Hz, 1H), 7.80 (d, J=8.40 Hz, 2H).

### Example 151

### cis-(+/−)-4-(2-ethoxyethoxy)-N-[2-(pyrrolidin-1-ylmethyl)cyclohexyl]benzamide

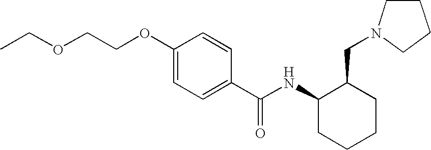

Following the same procedure as Example 150 (from step C to step E), yielded cis-(+/−)-4-(2-ethoxyethoxy)-N-[2-(pyrrolidin-1-ylmethyl)cyclohexyl]benzamide (38 mg, 27% for 3 steps) as its TFA salt. MS (M+1): 375.0. 1H NMR (400 MHz, CHLOROFORM-D): [symbol] pp [symbol] 1.25 (t, J=6.99 Hz, 3H), 1.29-1.58 (m, 3H), 1.64-1.82 (m, 4H), 1.83-1.94 (m, 1H), 2.02-2.19 (m, 4H), 2.18-2.29 (m, 1H), 2.95-3.14 (m, 4H), 3.61 (q, J=6.99 Hz, 2H), 3.66-3.78 (m, 2H), 3.79-3.85 (m, 2H), 4.14-4.21 (m, 2H), 4.27-4.38 (m, 1H), 6.86 (d, J=8.01 Hz, 1H), 6.97 (d, J=8.79 Hz, 2H), 7.76 (d, J=8.79 Hz, 2H).

### Example 152

### cis-(+/−)-N-{2-[(diethylamino)methyl]cyclohexyl}-4-(2-ethoxyethoxy)benzamide

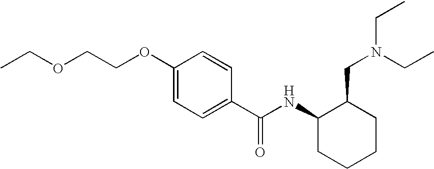

Following the same procedure as Example 150 (from step C to step E), yielded cis-(+/−)-N-{2-[(diethylamino)methyl]cyclohexyl}-4-(2-ethoxyethoxy)benzamide (24 mg, 16% for 3 steps) as its TFA salt. MS (M+1): 377.0. 1H NMR (400 MHz, METHANOL-D4): [symbol] pp [symbol] 1.20 (t, J=7.03 Hz, 3H) 1.24-1.37 (m, 7H) 1.43-1.56 (m, 2H) 1.61-1.71 (m, 1H) 1.74-1.91 (m, 4H) 2.20-2.31 (m, J=3.71 Hz, 1H) 2.73-2.88 (m, 1H) 2.92-3.01 (m, 1H) 3.05-3.16 (m, 1H) 3.18-3.26 (m, 2H) 3.36-3.47 (m, 1H) 3.58 (q, J=6.97 Hz, 2H) 3.75-3.81 (m, 2H) 4.11-4.21 (m, 2H) 4.24-4.32 (m, 1H) 7.02 (d, J=8.79 Hz, 2H) 7.84 (d, J=8.79 Hz, 2H)

### Example 153

### trans-(+/−)-4-(2-ethoxyethoxy)-N-[2-(piperidin-1-ylmethyl)cyclohexyl]benzamide

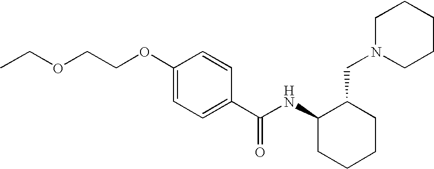

### Step A

### The Preparation of trans-(+/−)-benzyl[2-(hydroxymethyl)cyclohexyl]carbamate

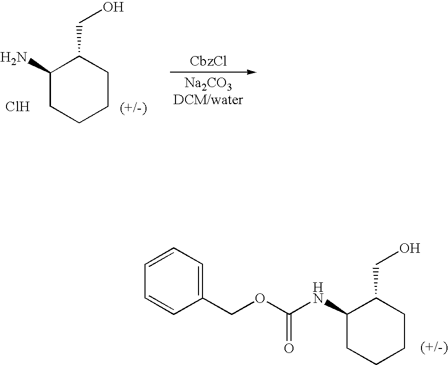

Following the same procedure as Example 150 (Step A), 612 mg of trans-(+/−)-[2-aminocyclohexyl]methanol hydrochloride (3.69 mmol) was treated with Na₂CO₃ and benzyl chloroformate to yield crude trans-(+/−)-benzyl[2-(hydroxymethyl)cyclohexyl]carbamate 0.92 g (95%).

### Step B

### The Preparation of trans-(+/−)-benzyl[2-formylcyclohexyl]carbamate

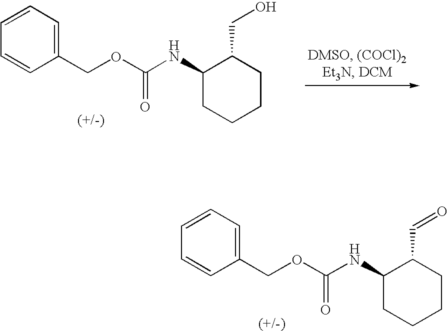

Following the same procedure as Example 89 (step B), yielded crude trans-(+/−)-benzyl[2-formylcyclohexyl]carbamate 890 mg (97%), which was used for the next step without further purification.

### Step C

### The Preparation of trans-(+/−)-benzyl[2-(piperidin-1-ylmethyl)cyclohexyl]carbamate

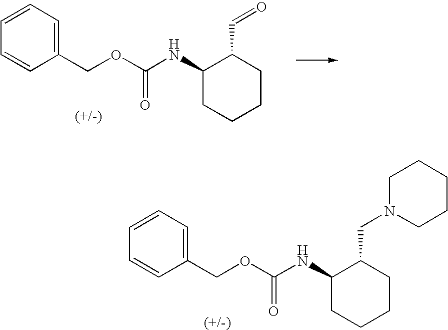

Following the same procedure as Example 89 (step C), the aldehyde from step B (1.8 mmol) was treated with NaBH(OAc)₃ to yielded crude trans-(+/−)-benzyl[2-(piperidin-1-ylmethyl)cyclohexyl]carbamate 543 mg (92%), which was used for the next step without further purification.

### Step D

### The Preparation of trans-(+/−)-[2-(piperidin-1-ylmethyl)cyclohexyl]amine

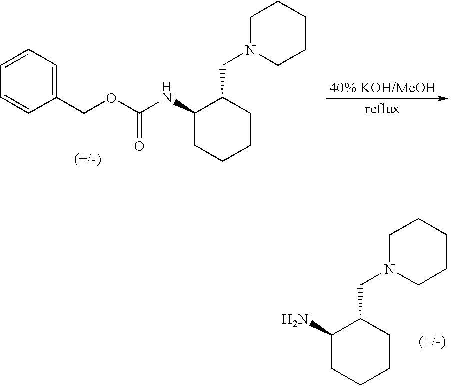

The solution of crude trans-(+/−)-benzyl[2-(piperidin-1-ylmethyl)cyclohexyl]carbamate (0.25 mmol) in 40% KOH/MeOH (6 mL, 1:1 v/v) was stirred at reflux for 5 h. The reaction mixture was cooled to room temperature, extracted with DCM (3×10 mL), dried over Na₂SO₄, concentrated to yield crude trans-(+/−)-[2-(piperidin-1-ylmethyl)cyclohexyl]amine, which was used for the next step without further purification.

### Step E

### The Preparation of trans-(+/−)-4-(2-ethoxyethoxy)-N-[2-(piperidin-1-ylmethyl)cyclohexyl]benzamide

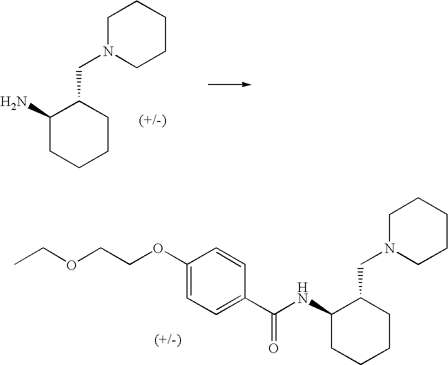

Following the same procedure as Example 2, the crude trans-(+/−)-[2-(piperidin-1-ylmethyl)cyclohexyl]amine from step D was converted to amide to yield trans-(+/−)-N-{2-[(diethylamino)methyl]cyclohexyl}-4-(2-ethoxyethoxy)benzamide (33 mg, 26% for 2 steps) as its TFA salt. MS (M+1): 389.0. 1H NMR (400 MHz, CHLOROFORM-D): δ pp [symbol] 1.25 (t, J=6.95 Hz, 3H), 1.29-1.45 (m, 4H), 1.69-1.90 (m, 6H), 1.93-2.05 (m, 2H), 2.06-2.16 (m, 2H), 2.53-2.66 (m, 2H), 3.18-3.35 (m, 4H), 3.61 (q, J=6.95 Hz, 2H), 3.64-3.70 (m, 1H), 3.77-3.83 (m, 2H), 3.84-3.92 (m, 1H), 4.14-4.19 (m, 2H), 6.94 (d, J=8.79 Hz, 2H), 7.93 (d, J=8.79 Hz, 2H), 7.96 (d, J=7.03 Hz, 1H).

### Example 154

### trans-(+/−)-N-[2-(azepan-1-ylmethyl)cyclohexyl]-4-(2-ethoxyethoxy)benzamide

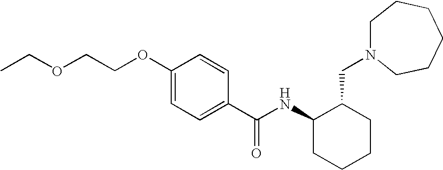

Following the same procedure as Example 153 (from step C to step E), yielded trans-(+/−)-N-[2-(azepan-1-ylmethyl)cyclohexyl]-4-(2-ethoxyethoxy)benzamide (32 mg, 21% for 3 steps) as its TFA salt. MS (M+1): 403.0. 1H NMR (400 MHz, METHANOL-D4): [symbol] ppm 1.19 (t, J=7.03 Hz, 3H), 1.26-1.54 (m, 4H), 1.60-1.72 (m, 4H), 1.75-1.91 (m, 7H), 1.91-1.99 (m, 1H), 2.05 (d, J=11.72 Hz, 1H), 2.92-3.01 (m, 1H), 3.10-3.20 (m, 2H), 3.22-3.27 (m, 1H), 3.39-3.49 (m, 2H), 3.58 (q, J=7.03 Hz, 2H), 3.70-3.76 (m, 1H), 3.76-3.81 (m, 2H), 4.12-4.20 (m, 2H), 7.01 (d, J=8.79 Hz, 2H), 7.80 (d, J=8.79 Hz, 2H).

### Example 155

### trans-(+/−)-N-{2-[(diethylamino)methyl]cyclohexyl}-4-(2-ethoxyethoxy)benzamide

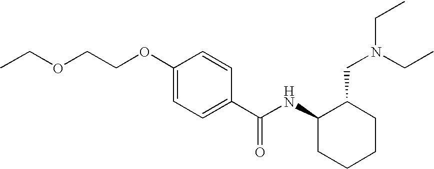

Following the same procedure as Example 153 (from step C to step E), yielded trans-(+/−)-N-{2-[(diethylamino)methyl]cyclohexyl}-4-(2-ethoxyethoxy)benzamide (28 mg, 19% for 3 steps) as its TFA salt. MS (M+1): 377.0. 1H NMR (400 MHz, METHANOL-D4): [symbol] pp [symbol] 1.16-1.24 (m, 6H), 1.28 (t, J=7.13 Hz, 3H), 1.30-1.61 (m, 4H), 1.76-1.89 (m, 3H), 1.90-1.98 (m, 1H), 2.05 (d, J=11.91 Hz, 1H), 2.95-3.05 (m, 1H), 3.10-3.26 (m, 5H), 3.58 (q, J=7.13 Hz, 2H), 3.71-3.81 (m, 3H), 4.11-4.20 (m, 2H), 7.00 (d, J=8.79 Hz, 2H), 7.80 (d, J=8.79 Hz, 2H).

### Example 156

### trans-(+/−)-N-(4-chlorophenyl)-N′-[2-(piperidin-1-ylmethyl)cyclohexyl]urea

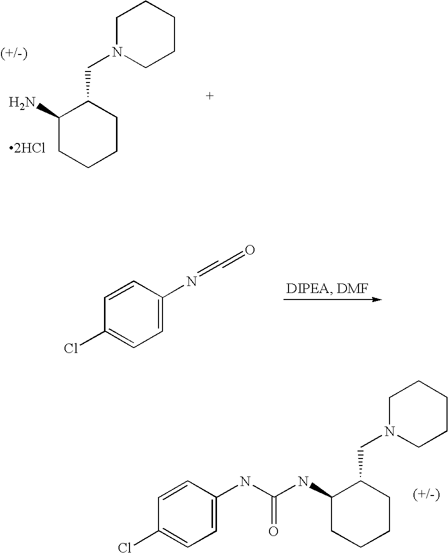

Diisopropylethylamine (0.127 ml, 0.732 mmol) was added to a suspension of trans-(+/−)-[2-(piperidin-1-ylmethyl)cyclohexyl]amine hydrochloride salt (98 mg, 0.37 mmol) in DMF (2 ml). The reaction mixture was added to 1-chloro-4-isocyanatobenzene (54 mg, 0.36 mmol). The reaction was stirred at room temperature under nitrogen for 12 hours. The solution was concentrated in vacuo. The product was purified by preparative LC/MS at high pH (water and acetonitrile buffered at pH10 with ammonium bicarbonate and ammonium hydroxide). The pure product crystallized out of the fractions obtained after preparative LC/MS. The free base of the title compound was obtained as white needles (30 mg, 24% yield). MS (M+1): 350.3; 1H NMR (400 MHz, CHLOROFORM-D): δ ppm 0.95-1.11 (m, 2H), 1.18-1.38 (m, 2H), 1.37-1.53 (m, 7H), 1.55-1.76 (m, 5H), 2.06 (dd, J=12.89, 2.15 Hz, 1H), 2.23 (s, 1H), 2.35 (dd, J=12.99, 9.67 Hz, 1H), 2.39-2.44 (m, 1H), 2.51 (s, 1H), 3.21 (td, J=10.79, 3.22 Hz, 1H), 6.03 (s, 1H), 7.21-7.32 (m, 4H), 7.85 (s, 1H).

### Example 157

### trans-(+/−)-N-(4-cyanophenyl)-N′-[2-(piperidin-1-ylmethyl)cyclohexyl]urea

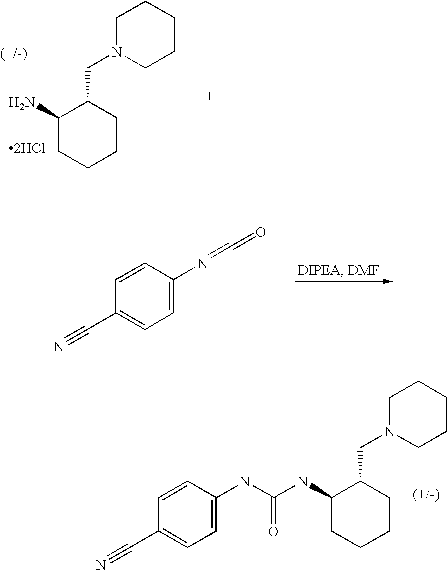

The procedure described in Example 156 was followed. The fractions from preparative LC/MS had to be evaporated as the product did not crystallize out. The free base of trans-(+/−)-N-(4-cyanophenyl)-N′-[2-(piperidin-1-ylmethyl)cyclohexyl]urea was obtained as a white solid (47 mg, 66% yield). MS (M+1): 341.3; 1H NMR (400 MHz, CHLOROFORM-D): δ ppm 1.02-1.12 (m, 2H), 1.21-1.38 (m, 2H), 1.43-1.77 (m, 12H), 2.14 (d, J=11.72 Hz, 1H), 2.27-2.42 (m, 2H), 2.43-2.51 (m, 1H), 3.24 (td, J=10.89, 3.61 Hz, 1H), 3.24 (td, J=10.89, 3.61 Hz, 1H), 6.54 (s, 1H), 7.44-7.51 (m, 2H), 7.51-7.57 (m, 2H), 8.05 (s, 1H).

### Example 158

### trans-(+/−)-N-(4-methoxyphenyl)-N′-[2-(piperidin-1-ylmethyl)cyclohexyl]urea

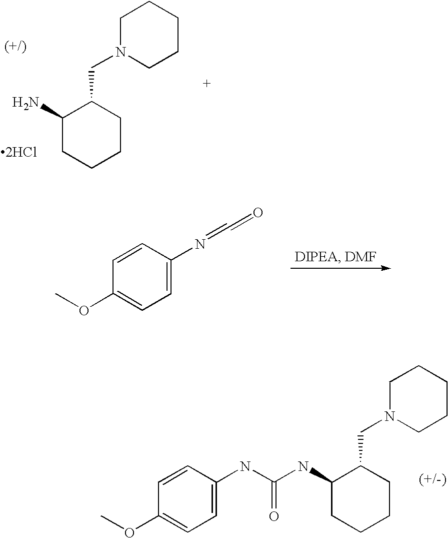

Following the same procedure as Example 156, yielded the free base of trans-(+/−)-N-(4-methoxyphenyl)-N′-[2-(piperidin-1-ylmethyl)cyclohexyl]urea (40 mg, 34%) as white needles. MS (M+1): 346.3; 1H NMR (400 MHz, CHLOROFORM-D): δ ppm 0.95-1.10 (m, 2H), 1.17-1.32 (m, 2H), 1.31-1.44 (m, 7H), 1.55-1.73 (m, 5H), 2.02 (dd, J=12.79, 2.64 Hz, 1H), 2.19 (s, 1H), 2.35 (dd, J=12.79, 9.08 Hz, 1H), 2.37-2.47 (m, 2H), 3.24 (s, 1H), 3.78 (s, 3H), 5.91 (s, 1H), 6.81-6.88 (d, J=8.98 Hz, 2H), 7.22 (d, J=8.98 Hz, 2H), 7.29 (s, 1H).

### Example 159

### trans-(+/−)-2-methoxy-4-methyl-N-[2-(piperidin-1-ylmethyl)cyclohexyl]benzenesulfonamide

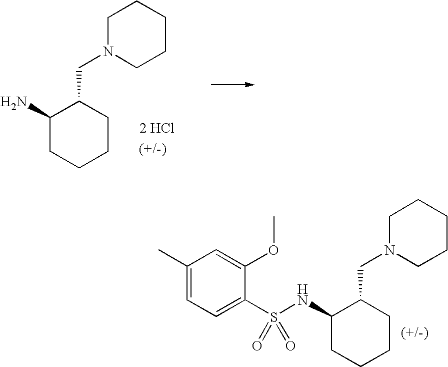

To a solution of trans-(+/−)-[2-(piperidin-1-ylmethyl)cyclohexyl]amine hydrochloride (81 mg, 0.3 mmol) in dichloromethane (4 mL) was added 2-methoxy-4-methylbenzenesulfonyl chloride (66 mg, 0.3 mmol) followed by triethylamine (37 mg, 0.36 mmol). The mixture was stirred at room temperature for 5 h, quenched with water (5 mL), extracted with saturated aq. NaHCO₃, dried over Na₂SO₄, concentrated to yield crude product which was purified with reverse phase HPLC. The title compound was obtained as white solids (84 mg, 74%). MS (M+1): 381.3. 1H NMR (400 MHz, METHANOL-D4): δ ppm 0.82-0.95 (m, 1H), 1.00-1.25 (m, 3H), 1.40-1.50 (m, 3H), 1.52-1.64 (m, 7H), 1.69-1.84 (m, 2H), 2.02 (dd, J=11.91, 6.25 Hz, 1H), 2.22-2.35 (m, 2H), 2.40 (s, 3H), 2.41-2.49 (m, 2H), 2.69-2.79 (m, 1H), 3.92 (s, 3H), 6.87 (d, J=7.81 Hz, 1H), 7.01 (s, 1H), 7.67 (d, J=7.81 Hz, 1H).

### Example 160-162

The same procedure described in Example 151 was followed to make Examples 160-162.

| Exp. No. | Structure | Name | MS (M + 1) | Retention time (min) |
|---|---|---|---|---|
| 160 | 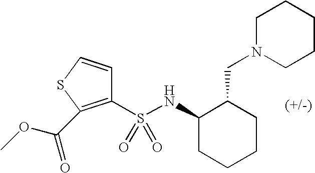 | trans-(+/−)-methyl 3-({[2- (piperidin-1- ylmethyl)cylcohexyl]amino}sulfonyl)thiophene-2- carboxylate | 400.8 | 1.88 |
| 161 | 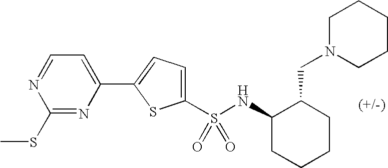 | trans-(+/−)-5-[2- (methylthio)pyrimidin-4-yl]- N-[2-(piperidin-1- ylmethyl)cyclohexyl]thio- phene-2-sulfonamide | 466.7 | 2.2 |
| 162 | 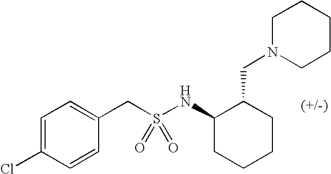 | trans-(+/−)-1-(4- chlorophenyl)-N-[2- (piperidin-1- ylmethyl)cyclohexyl]methane- sulfonamide | 384.79 | 2.12 |

### Example 163

### trans-(+/−)-N-{2-[(3-butylpiperidin-1-yl)methyl]cyclohexyl}-4-(1,3-oxazol-5-yl)benzamide

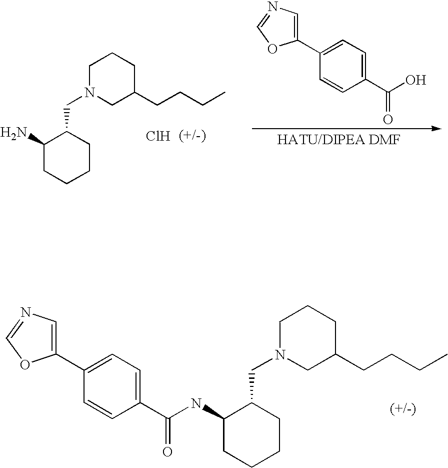

To a solution of trans-(+/−)-2-[(3-butylpiperidin-1-yl)methyl]cyclohexylamine hydrochloride (72 mg, 0.25 mmol) in dry DMF (3 mL) was added 4-(1,3-oxazol-5-yl)benzoic acid (56 mg, 0.3 mmol) followed by HATU (114 mg, 0.3 mmol) and diisopropylethylamine (0.10 mL, 0.5 mmol). The mixture was stirred at room temperature for 1 h, and the reaction was quenched with water (5 mL). The solvent was removed in vacuo. DCM (15 mL) was added and the mixture washed with saturated NaHCO₃ (10 mL) and brine (10 mL), dried over Na₂SO₄. The crude product was purified with reverse phase HPLC to yield trans-(+/−)-N-{2-[(3-butylpiperidin-1-yl)methyl]cyclohexyl}-4-(1,3-oxazol-5-yl)benzamide (52 mg, 49%) as white powders. MS (M+1): 424.3. 1H NMR (400 MHz, METHANOL-D4) δ ppm 0.68-0.90 (m, 4H), 0.98-1.17 (m, 4H), 1.22-1.46 (m, 7H), 1.50-1.62 (m, 2H), 1.64-1.81 (m, 5H), 1.81-2.01 (m, 2H), 2.02-2.27 (m, 2H), 2.34-2.53 (m, 1H), 2.63-3.08 (m, 2H), 3.48-3.69 (m, 1H), 7.64 (s, 1H), 7.79-7.85 (m, 2H), 7.86-7.92 (m, 2H), 8.29 (d, J=1.56 Hz, 1H).

### Example 164

### trans-(+/−)-N-{2-[(3-butylpiperidin-1-yl)methyl]cyclohexyl}-6-(trifluoromethyl)nicotinamide

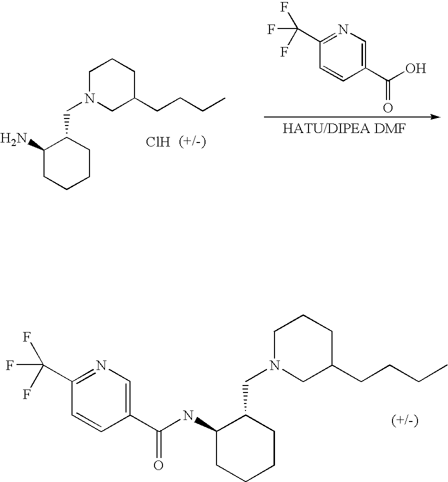

To a solution of trans-(+/−)-2-[(3-butylpiperidin-1-yl)methyl]cyclohexylamine hydrochloride (72 mg, 0.25 mmol) in dry DMF (3 mL) was added 6-(trifluoromethyl)nicotinic acid (57 mg, 0.3 mmol) followed by HATU (114 mg, 0.3 mmol) and diisopropylethylamine (0.10 mL, 0.5 mmol). The mixture was stirred at room temperature for 1 h, and the reaction was quenched with water (5 mL). The solvent was removed in vacuo. DCM (15 mL) was added and washed with saturated NaHCO₃ (10 mL) and brine (10 mL), dried over Na₂SO₄. The crude product was purified with reverse phase HPLC to yield trans-(+/−)-N-{2-[(3-butylpiperidin-1-yl)methyl]cyclohexyl}-6-(trifluoromethyl)nicotinamide (66 mg, 62%) as a white powder. MS (M+1): 426.2. 1H NMR (400 MHz, METHANOL-D4) δ ppm 0.75-0.82 (m, 2H), 0.83-0.92 (m, 2H), 0.98-1.18 (m, 5H), 1.22-1.41 (m, 6H), 1.46-1.59 (m, 2H), 1.61-1.81 (m, 5H), 1.82-1.99 (m, 1H), 2.02-2.19 (m, 2H), 2.33-2.46 (m, 1H), 2.63-3.01 (m, 2H), 3.52-3.69 (m, 1H), 7.92 (d, J=8.20 Hz, 1H), 8.34-8.44 (m, 1H), 9.08 (d, J=4.10 Hz, 1H).

### Example 165

### trans-(+/−)-N-{2-[(3-butylpiperidin-1-yl)methyl]cyclohexyl}-4-(2-methoxyethoxy)benzamide

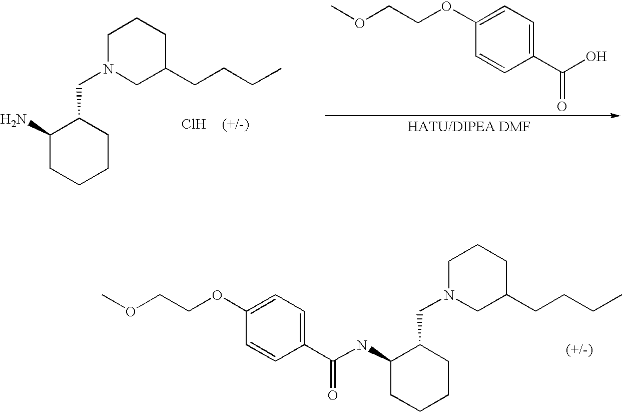

To a solution of trans-(+/−)-2-[(3-butylpiperidin-1-yl)methyl]cyclohexylamine hydrochloride (72 mg, 0.25 mmol) in dry DMF (3 mL) was added 4-(2-methoxyethoxy)benzoic acid (58 mg, 0.3 mmol) followed by HATU (114 mg, 0.3 mmol) and diisopropylethylamine (0.10 mL, 0.5 mmol). The mixture was stirred at room temperature for 1 h, and the reaction was quenched with water (5 mL). The solvent was removed in vacuo. DCM (15 mL) was added and washed with saturated NaHCO₃ (10 mL) and brine (10 mL), dried over Na₂SO₄. The crude product was purified with reverse phase HPLC to yield trans-(+/−)-N-{2-[(3-butylpiperidin-1-yl)methyl]cyclohexyl}-4-(2-methoxyethoxy)benzamide (76 mg, 71%) as a white powder. MS (M+1): 431.3. 1H NMR (400 MHz, METHANOL-D4) δ ppm 0.77-0.92 (m, 4H), 0.96-1.07 (m, 2H), 1.07-1.19 (m, 4H), 1.20-1.41 (m, 5H), 1.46-1.64 (m, 4H), 1.66-1.81 (m, 4H), 1.82-1.98 (m, 1H), 2.04-2.19 (m, 2H), 2.29-2.43 (m, 1H), 2.64-2.79 (m, 1H), 2.81-2.98 (m, 1H), 3.40 (s, 3H), 3.45-3.62 (m, 1H), 3.69-3.77 (m, 2H), 4.14 (s, 2H), 6.98 (d, J=8.59 Hz, 2H), 7.76 (d, J=7.62 Hz, 2H).

### Example 166

### trans-(+/−)-N-{2-[(3-butylpiperidin-1-yl)methyl]cyclohexyl}-3-(4-chlorophenyl)propanamide

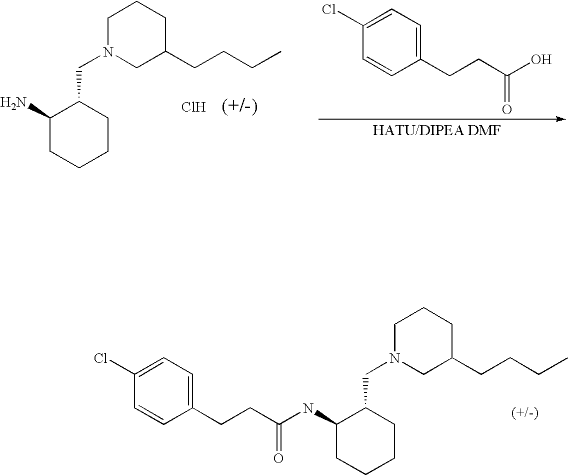

To a solution of trans-(+/−)-2-[(3-butylpiperidin-1-yl)methyl]cyclohexylamine hydrochloride (72 mg, 0.25 mmol) in dry DMF (3 mL) was added 3-(4-chlorophenyl)propanoic acid (55 mg, 0.3 mmol) followed by HATU (114 mg, 0.3 mmol) and diisopropylethylamine (0.10 mL, 0.5 mmol). The mixture was stirred at room temperature for 1 h, and the reaction was quenched with water (5 mL). The solvent was removed in vacuo. DCM (15 mL) was added and washed with saturated NaHCO₃ (10 mL) and brine (10 mL), dried over Na₂SO₄. The crude product was purified with reverse phase HPLC to yield trans-(+/−)-N-{2-[(3-butylpiperidin-1-yl)methyl]cyclohexyl}-3-(4-chlorophenyl)propanamide (65 mg, 62%) as white powders. MS (M+1): 419.3. 1H NMR (400 MHz, METHANOL-D4) δ ppm 0.83-1.02 (m, 6H), 1.05-1.20 (m, 4H), 1.21-1.41 (m, 8H), 1.48-1.62 (m, 2H), 1.61-1.77 (m, 4H), 1.79-1.88 (m, 1H), 1.91-2.10 (m, 2H), 2.39-2.49 (m, 2H), 2.65-2.79 (m, 2H), 2.80-3.00 (m, 2H), 3.30-3.38 (m, 1H), 7.11-7.31 (m, 4H).

### Example 167

### trans-(+/−)-N-{2-[(3-butylpiperidin-1-yl)methyl]cyclohexyl}-4-(1H-imidazol-1-yl)benzamide

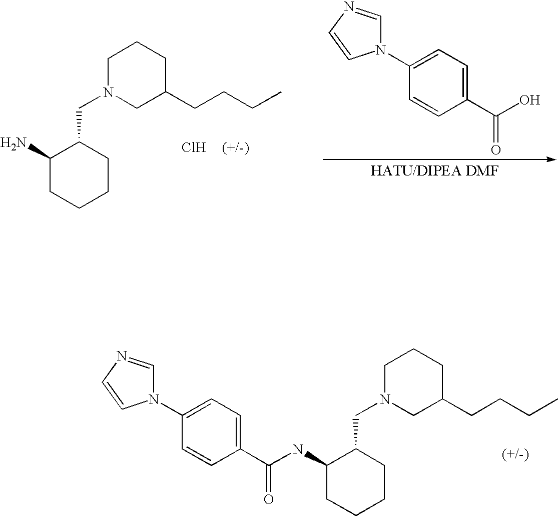

To a solution of trans-(+/−)-2-[(3-butylpiperidin-1-yl)methyl]cyclohexylamine hydrochloride (72 mg, 0.25 mmol) in dry DMF (3 mL) was added 4-(1H-imidazol-1-yl)benzoic acid (56 mg, 0.3 mmol) followed by HATU (114 mg, 0.3 mmol) and diisopropylethylamine (0.10 mL, 0.5 mmol). The mixture was stirred at room temperature for 1 h, and the reaction was quenched with water (5 mL). The solvent was removed in vacuo. DCM (15 mL) was added and washed with saturated NaHCO₃ (10 mL) and brine (10 mL), dried over Na₂SO₄. The crude product was purified with high pH HPLC to yield trans-(+/−)-N-{2-[(3-butylpiperidin-1-yl)methyl]cyclohexyl}-4-(1H-imidazol-1-yl)benzamide (52 mg, 49%) as a white powder. MS (M+1): 423.3. 1H NMR (400 MHz, METHANOL-D4) δ ppm 0.66-0.96 (m, 5H), 0.98-1.18 (m, 5H), 1.21-1.44 (m, 6H), 1.48-1.62 (m, 2H), 1.64-1.85 (m, 5H), 1.82-2.03 (m, 1H), 2.05-2.23 (m, 2H), 2.29-2.47 (m, 1H), 2.62-3.01 (m, 2H), 3.50-3.63 (m, 1H), 7.17 (s, 1H), 7.67 (s, 1H), 7.70 (dd, J=8.30, 4.39 Hz, 2H), 7.97 (d, J=8.40 Hz, 2H), 8.26 (s, 1H).

### Example 168

### trans-(+/−)-N-(2-{[3-(ethoxymethyl)piperidin-1-yl]methyl}cyclohexyl)-6-(1H-imidazol-1-yl)nicotinamide

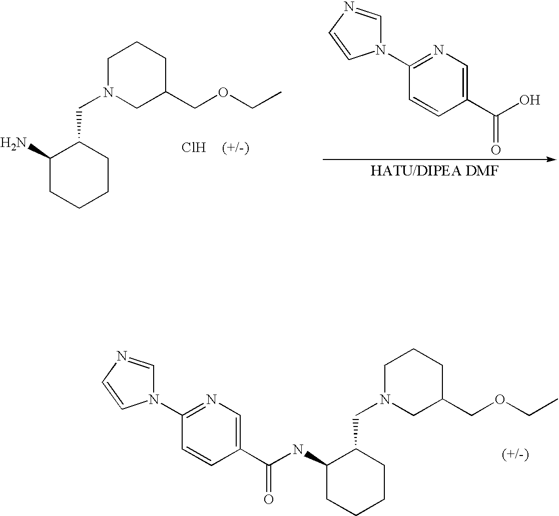

To a solution of trans-(+/−)-2-({3-[(ethoxy)methyl]piperidin-1-yl}methyl)cyclohexyl]amine hydrochloride (73 mg, 0.25 mmol) in dry DMF (3 mL) was added 6-(1H-imidazol-1-yl)nicotinic acid (57 mg, 0.3 mmol) followed by HATU (114 mg, 0.3 mmol) and diisopropylethylamine (0.10 mL, 0.5 mmol). The mixture was stirred at room temperature for 1 h, and the reaction was quenched with water (5 mL). The solvent was removed in vacuo. DCM (15 mL) was added and washed with saturated NaHCO₃ (10 mL) and brine (10 mL), dried over Na₂SO₄. The crude product was purified with high pH HPLC to yield trans-(+/−)-N-(2-{[3-(ethoxymethyl)piperidin-1-yl]methyl}cyclohexyl)-6-(1H-imidazol-1-yl)nicotinamide (67 mg, 63%) as a white powder. MS (M+1): 426.2. 1H NMR (400 MHz, METHANOL-D4) δ ppm 0.83-0.97 (m, 1H), 1.00-1.20 (m, 4H), 1.27-1.45 (m, 3H), 1.51-1.81 (m, 6H), 1.83-2.00 (m, 2H), 2.04-2.19 (m, 2H), 2.32-2.49 (m, 1H), 2.70-3.01 (m, 2H), 3.07-3.25 (m, 2H), 3.38-3.50 (m, 1H), 3.56-3.69 (m, 1H), 4.49-4.71 (m, 3H), 7.17 (s, 1H), 7.80 (d, J=8.59 Hz, 1H), 7.95 (s, 1H), 8.29-8.39 (m, 1H), 8.60 (s, 1H), 8.90 (s, 1H).

### Example 169

### trans-(+/−)-N-(2-{[3-(ethoxymethyl)piperidin-1-yl]methyl}cyclohexyl)-4-(1,3-oxazol-5-yl)benzamide

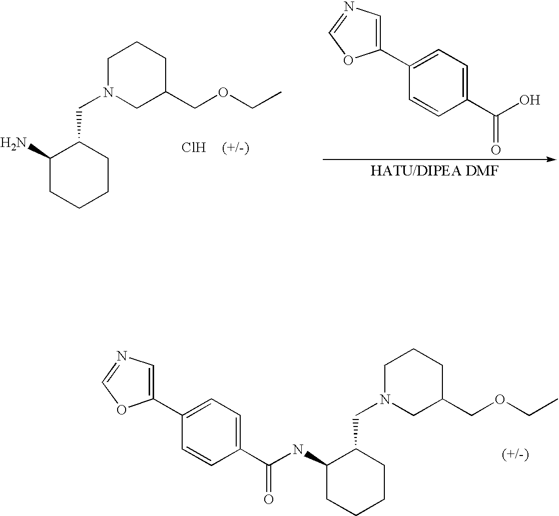

To a solution of trans-(+/−)-2-({3-[(ethoxy)methyl]piperidin-1-yl}methyl)cyclohexyl]amine hydrochloride (73 mg, 0.25 mmol) in dry DMF (3 mL) was added 4-(1,3-oxazol-5-yl)benzoic acid (56 mg, 0.3 mmol) followed by HATU (114 mg, 0.3 mmol) and diisopropylethylamine (0.10 mL, 0.5 mmol). The mixture was stirred at room temperature for 1 h, and the reaction was quenched with water (5 mL). The solvent was removed in vacuo. DCM (15 mL) was added and washed with saturated NaHCO₃ (10 mL) and brine (10 mL), dried over Na₂SO₄. The crude product was purified with high pH HPLC to yield trans-(+/−)-N-(2-{[3-(ethoxymethyl)piperidin-1-yl]methyl}cyclohexyl)-4-(1,3-oxazol-5-yl)benzamide (62 mg, 58%) as a white powder. MS (M+1): 426.2. 1H NMR (400 MHz, METHANOL-D4) δ ppm 0.92 (s, 1H), 1.00-1.19 (m, 4H), 1.24-1.44 (m, 4H), 1.51-1.81 (m, 8H), 1.84-1.97 (m, 2H), 2.06-2.20 (m, 2H), 2.36-2.48 (m, 1H), 2.69-2.88 (m, 1H), 2.88-3.04 (m, 1H), 3.07-3.24 (m, 2H), 3.37-3.48 (m, 1H), 3.53-3.64 (m, 1H), 7.64 (s, 1H), 7.80-7.85 (m, 2H), 7.87-7.94 (m, 2H), 8.29 (s, 1H).

### Example 170

### trans-(+/−)-N-(2-{[3-(ethoxymethyl)piperidin-1-yl]methyl}cyclohexyl)-4-(1H-imidazol-1-yl)benzamide

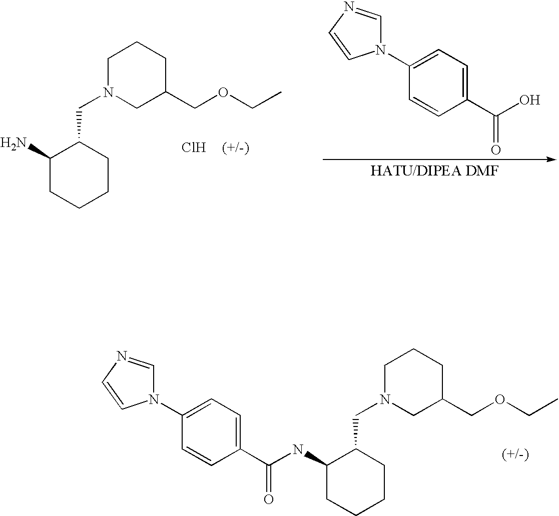

To a solution of trans-(+/−)-2-({3-[(ethoxy)methyl]piperidin-1-yl}methyl)cyclohexyl]amine hydrochloride (73 mg, 0.25 mmol) in dry DMF (3 mL) was added 4-(1H-imidazol-1-yl)benzoic acid (56 mg, 0.3 mmol) followed by HATU (114 mg, 0.3 mmol) and diisopropylethylamine (0.10 mL, 0.5 mmol). The mixture was stirred at room temperature for 1 h, and the reaction was quenched with water (5 mL). The solvent was removed in vacuo. DCM (15 mL) was added and washed with saturated NaHCO₃ (10 mL) and brine (10 mL), dried over Na₂SO₄. The crude product was purified with high pH HPLC to yield trans-(+/−)-N-(2-{[3-(ethoxymethyl)piperidin-1-yl]methyl}cyclohexyl)-4-(1H-imidazol-1-yl)benzamide (56 mg, 53%) as a white powder. MS (M+1): 425.3. 1H NMR (400 MHz, METHANOL-D4) δ ppm 0.86-0.99 (m, 1H), 1.02-1.21 (m, 5H), 1.24-1.48 (m, 4H), 1.57-1.82 (m, 8H), 1.88-2.01 (m, 2H), 2.04-2.25 (m, 2H), 2.33-2.54 (m, 1H), 2.73-3.03 (m, 1H), 3.10-3.24 (m, 2H), 3.38-3.49 (m, 1H), 3.54-3.66 (m, 1H), 7.17 (s, 1H), 7.67 (s, 1H), 7.70 (d, J=8.20 Hz, 2H), 7.91-8.00 (m, 2H), 8.25 (s, 1H).

### Example 171

### trans-(+/−)-N-2-{[3-(ethoxymethyl)piperidin-1-yl]methyl}cyclohexyl)-4-{[(methylsulfonyl)amino]methyl}benzamide

### Step A

### The Preparation of trans-(+/−)-tert-butyl(4-{[(2-{[3-(ethoxymethyl)piperidin-1-yl]methyl}cyclohexyl)amino]carbonyl}benzyl)carbamate

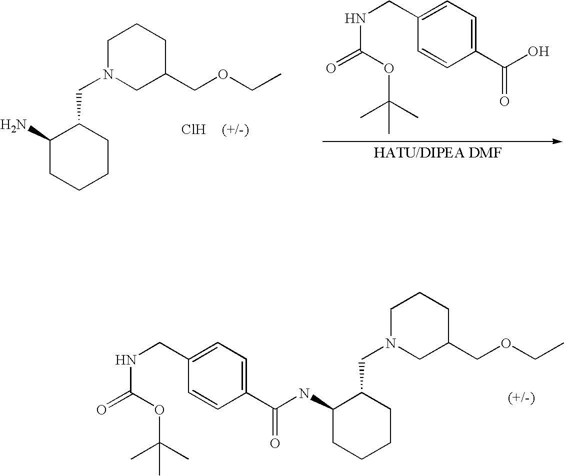

To a solution of trans-(+/−)-2-({3-[(ethoxy)methyl]piperidin-1-yl}methyl)cyclohexyl]amine hydrochloride (147 mg, 0.5 mmol) in dry DMF (5 mL) was added 4-{[(tert-butoxycarbonyl)amino]methyl}benzoic acid (126 mg, 0.5 mmol) followed by HATU (190 mg, 0.5 mmol) and diisopropylethylamine (0.10 mL, 0.5 mmol). The mixture was stirred at room temperature for 1 h, and the reaction was quenched with water (5 mL). The solvent was removed in vacuo. DCM (15 mL) was added and washed with saturated NaHCO₃ (10 mL) and brine (10 mL), dried over Na₂SO₄. The crude product trans-(+/−)-tert-butyl(4-{[(2-{[3-(ethoxymethyl)piperidin-1-yl]methyl}cyclohexyl)amino]carbonyl}benzyl)carbamate (240 mg, 98%) was used for the next step without further purification. MS (M+1): 488.36.

### Step B

### The Preparation of trans-(+/−)-4-(aminomethyl)-N-(2-{[3-(ethoxymethyl)piperidin-1-yl]methyl}cyclohexyl)benzamide HCl salt

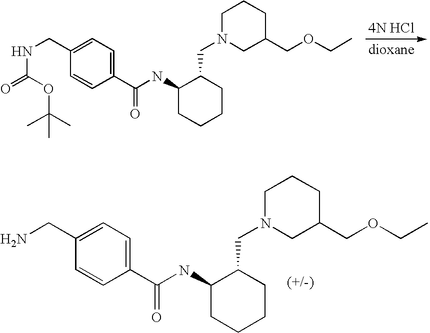

The crude product from step A (trans-(+/−)-tert-butyl(4-{[(2-{[3-(ethoxymethyl)piperidin-1-yl]methyl}cyclohexyl)amino]carbonyl}benzyl)carbamate, 122 mg, 0.25 mmol) was treated with 4N HCl in dioxane (5 mL), the reaction mixture was stirred at room temperature for 5 h. Removal of solvent afforded the desired intermediate trans-(+/−)-4-(aminomethyl)-N-(2-{[3-(ethoxymethyl)piperidin-1-yl]methyl}cyclohexyl)benzamide as its HCl salt.

### Step C

### The Preparation of trans-(+/−)-N-2-{[3-(ethoxymethyl)piperidin-1-yl]methyl}cyclohexyl)-4-{[(methylsulfonyl)amino]methyl}benzamide

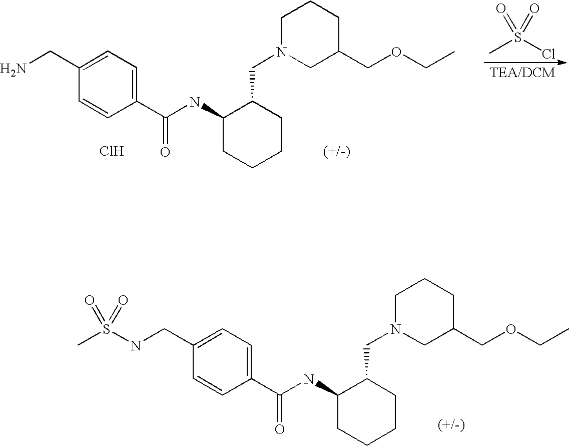

The crude product from step B (trans-(+/−)-4-(aminomethyl)-N-(2-{[3-(ethoxymethyl)piperidin-1-yl]methyl}cyclohexyl)benzamide HCl salt, ˜0.25 mmol) was taken up into dichloromethane (5 mL), triethyl amine (0.14 mL, 1.0 mmol) was added followed by methyl sulfonyl chloride (0.3 mmol). The reaction mixture was stirred at room temperature for 3 h. The reaction was quenched with water (5 mL). DCM (30 mL) was added and washed with saturated NaHCO₃ (10 mL) and brine (10 mL), dried over Na₂SO₄. The crude product was purified with high pH to yield the title compound (68 mg, 59%) as a white powder. MS (M+1): 466.3. 1H NMR (400 MHz, METHANOL-D4) δ ppm 0.84-1.03 (m, 1H), 1.05-1.19 (m, 4H), 1.26-1.45 (m, 4H), 1.51-1.71 (m, 6H), 1.83-1.98 (m, 3H), 2.06-2.22 (m, 2H), 2.38-2.52 (m, 1H), 2.67-2.80 (m, 1H), 2.87 (d, J=1.37 Hz, 3H), 2.93-3.07 (m, 1H), 3.09-3.26 (m, 2H), 3.33 (q, J=7.23 Hz, 1H), 3.39-3.49 (m, 1H), 3.52-3.65 (m, 1H), 4.29 (s, 2H), 7.47 (d, J=7.81 Hz, 2H), 7.79 (dd, J=8.10, 1.66 Hz, 2H).

### Example 172

### trans-(+/−)-N-(2-{[3-propylpiperidin-1-yl]methyl}cyclohexyl)-6-(1H-imidazol-1-yl)nicotinamide

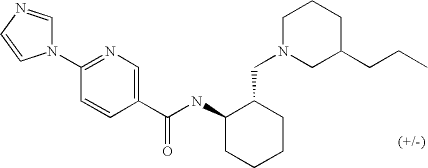

### Step A

### The Preparation of 3-propylpiperidine hydrochloride

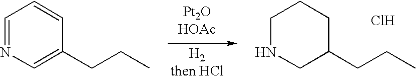

To a solution of 3-propylpyridine (5.0 g, 41.3 mmol) in HOAc (60 mL) was added Pt₂O (0.5 g) and the mixture was hydrogenated at room temperature (40 psi) for 5 h. After being filtered and concentrated, 40% aq. NaOH (50 mL) was added, extracted with EtOAc (3×50 mL), dried over Na₂SO₄, then treated with 4N HCl in dioxane, evaporated to give the HCl salt as white powders (6.56 g, 97%).

### Step B

### The Preparation of trans-(+/−)-tert-butyl{2-[(3-propylpiperidin-1-yl)methyl]cyclohexyl}carbamate

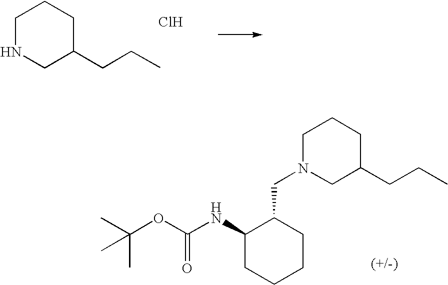

The HCl salt from step A (3-propylpiperidine hydrochloride, 328 mg, 2.0 mmol) was added to a solution of trans-(+/−)-tert-butyl[2-formylcyclohexyl]carbamate (454 mg, 2.0 mmol) in dichloromethane (16 ml). The reaction was stirred at room temperature for 30 minutes, and then sodium triacetoxyborohydride (636 mg, 3.00 mmol) was added to the reaction mixture. The reaction was stirred at room temperature for 12 hours, and then cooled to 0° C. Water (1 ml) was added dropwise. A 1N sodium hydroxide solution (20 ml) and dichloromethane (80 ml) were added to the mixture. The phases were separated and the aqueous was extracted with dichloromethane (2×30 ml). The combined organic phases were washed with brine, dried over Na₂SO₄, filtered, and concentrated in vacuo. The title compound was obtained as a crude oil (554 mg, 82%), which was used for the next step without further purification.

### Step C

### The Preparation of trans-(+/−)-tert-butyl{2-[(3-propylpiperidin-1-yl)methyl]cyclohexyl}carbamate

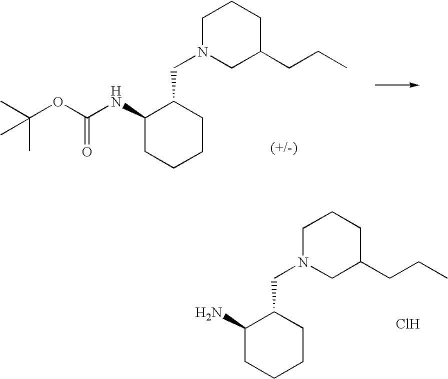

The crude product from steps B was treated with 4N HCl in dioxane (10 mL), stirred at room temperature for 3 h. After concentrated, the title compound was obtained as its HCl salt (520 mg, 95%), which was used for the next step without further purification.

### Step D

### The Preparation of trans-(+/−)-N-(2-{[3-propylpiperidin-1-yl]methyl}cyclohexyl)-6-(1H-imidazol-1-yl)nicotinamide

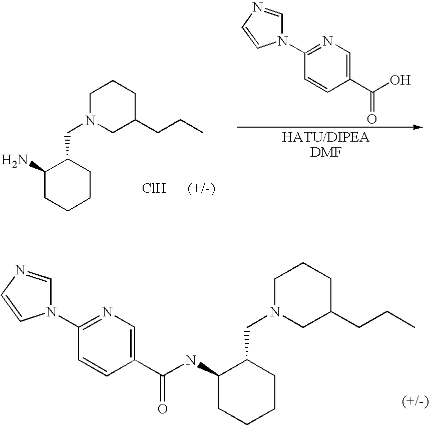

To a solution of trans-(+/−)-2-({3-propylpiperidin-1-yl}methyl)cyclohexyl]amine hydrochloride (69 mg, 0.25 mmol) in dry DMF (3 mL) was added 6-(1H-imidazol-1-yl)nicotinic acid (57 mg, 0.3 mmol) followed by HATU (114 mg, 0.3 mmol) and diisopropylethylamine (0.10 mL, 0.5 mmol). The mixture was stirred at room temperature for 1 h, and the reaction was quenched with water (5 mL). The solvent was removed in vacuo. DCM (15 mL) was added and washed with saturated NaHCO₃ (10 mL) and brine (10 mL), dried over Na₂SO₄. The crude product was purified with high pH HPLC to yield trans-(+/−)-N-(2-{[3-propylpiperidin-1-yl]methyl}cyclohexyl)-6-(1H-imidazol-1-yl)nicotinamide (65 mg, 63%) as a white powder. MS (M+1): 410.3. 1H NMR (400 MHz, METHANOL-D4) δ ppm 0.65-0.91 (m, 5H), 1.00-1.18 (m, 4H), 1.22-1.43 (m, 5H), 1.52-1.61 (m, 2H), 1.63-1.84 (m, 5H), 1.86-2.01 (m, 1H), 2.06-2.21 (m, 2H), 2.31-2.49 (m, 1H), 2.66-3.01 (m, 2H), 3.55-3.70 (m, 1H), 7.17 (s, 1H), 7.81 (dd, J=8.59, 2.15 Hz, 1H), 7.95 (s, 1H), 8.34 (dd, J=8.59, 1.56 Hz, 1H), 8.60 (s, 1H), 8.91 (s, 1H).

### Example 173

### trans-(+/−)-4-(1H-imidazol-1-yl)-N-{2-[(3-propylpiperidin-1-yl)methyl]cyclohexyl}benzamide

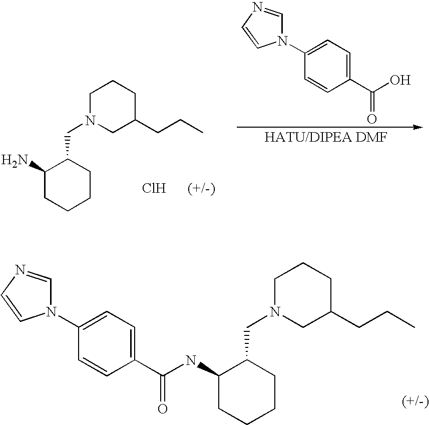

To a solution of trans-(+/−)-2-({3-propylpiperidin-1-yl}methyl)cyclohexyl]amine hydrochloride (69 mg, 0.25 mmol) in dry DMF (3 mL) was added 4-(1H-imidazol-1-yl)benzoic acid (56 mg, 0.3 mmol) followed by HATU (114 mg, 0.3 mmol) and diisopropylethylamine (0.10 mL, 0.5 mmol). The mixture was stirred at room temperature for 1 h, and the reaction was quenched with water (5 mL). The solvent was removed in vacuo. DCM (15 mL) was added and washed with saturated NaHCO₃ (10 mL) and brine (10 mL), dried over Na₂SO₄. The crude product was purified with high pH HPLC to yield trans-(+/−)-4-(1H-imidazol-1-yl)-N-{2-[(3-propylpiperidin-1-yl)methyl]cyclohexyl}benzamide (74 mg, 72%) as a white powder. MS (M+1): 409.3. 1H NMR (400 MHz, METHANOL-D4) δ ppm 0.65-0.91 (m, 5H), 0.97-1.06 (m, 1H), 1.06-1.20 (m, 3H), 1.21-1.45 (m, 5H), 1.53-1.83 (m, 7H), 1.87-2.00 (m, 1H), 2.06-2.24 (m, 2H), 2.36-2.51 (m, 1H), 2.64-3.01 (m, 2H), 3.50-3.66 (m, 1H), 7.17 (s, 1H), 7.62-7.68 (m, 1H), 7.70 (d, J=7.62 Hz, 2H), 7.97 (d, J=8.01 Hz, 2H), 8.24 (d, J=2.93 Hz, 1H).

### Example 174

### trans-(+/−)-N-(2-{[3-isobutylpiperidin-1-yl]methyl}cyclohexyl)-6-(1H-imidazol-1-yl)nicotinamide

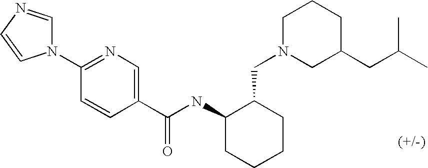

### Step A

### The Preparation of 3-isobutylpiperidine hydrochloride

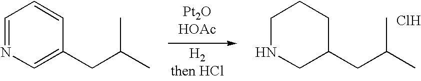

To a solution of 3-isobutylpyridine (2.5 g, 18.5 mmol) in HOAc (40 mL) was added Pt₂O (0.2 g) and the mixture was hydrogenated at room temperature (40 psi) for 5 h. After being filtered and concentrated, 40% aq. NaOH (30 mL) was added, extracted with EtOAc (3×40 mL), dried over Na₂SO₄, then treated with 4N HCl in dioxane, evaporated to give the HCl salt as white powders (2.92 g, 89%).

### Step B

### The Preparation of trans-(+/−)-tert-butyl{2-[(3-isobutylpiperidin-1-yl)methyl]cyclohexyl}carbamate

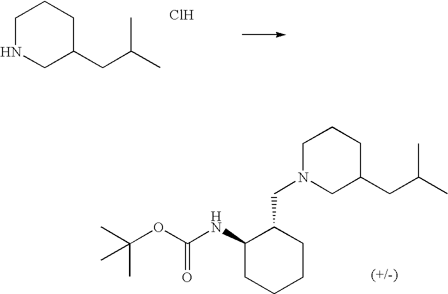

The HCl salt from step A (3-isobutylpiperidine hydrochloride, 356 mg, 2.0 mmol) was added to a solution of trans-(+/−)-tert-butyl[2-formylcyclohexyl]carbamate (454 mg, 2.0 mmol) in dichloromethane (16 ml). The reaction was stirred at room temperature for 30 minutes, and then sodium triacetoxyborohydride (636 mg, 3.00 mmol) was added to the reaction mixture. The reaction was stirred at room temperature for 12 hours, and then cooled to 0° C. Water (1 ml) was added dropwise. A 1N sodium hydroxide solution (20 ml) and dichloromethane (80 ml) were added to the mixture. The phases were separated and the aqueous was extracted with dichloromethane (2×30 ml). The combined organic phases were washed with brine, dried over Na₂SO₄, filtered, and concentrated in vacuo. The title compound was obtained as a crude oil (624 mg, 89%), which was used for the next step without further purification.

### Step C

### The preparation of trans-(+/−)-tert-butyl{2-[(3-isobutylpiperidin-1-yl)methyl]cyclohexyl}carbamate

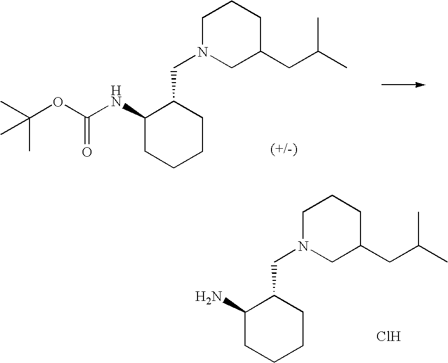

The crude product from steps B was treated with 4N HCl in dioxane (10 mL), stirred at room temperature for 3 h. After concentrated, the title compound was obtained as its HCl salt (543 mg, 94%), which was used for the next step without further purification.

### Step D

### The Preparation of trans-(+/−)-N-(2-{[3-isobutylpiperidin-1-yl]methyl}cyclohexyl)-6-(1H-imidazol-1-yl)nicotinamide

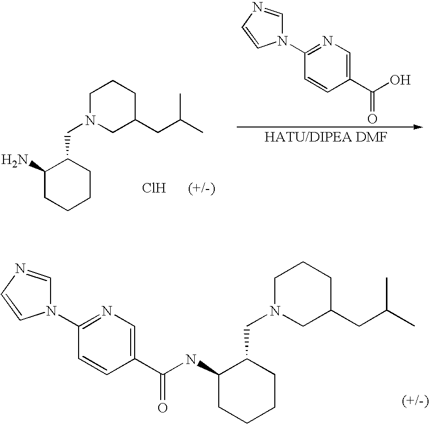

To a solution of trans-(+/−)-2-({3-isobutylpiperidin-1-yl}methyl)cyclohexyl]amine hydrochloride (73 mg, 0.25 mmol) in dry DMF (3 mL) was added 6-(1H-imidazol-1-yl)nicotinic acid (57 mg, 0.3 mmol) followed by HATU (114 mg, 0.3 mmol) and diisopropylethylamine (0.10 mL, 0.5 mmol). The mixture was stirred at room temperature for 1 h, and the reaction was quenched with water (5 mL). The solvent was removed in vacuo. DCM (15 mL) was added and washed with saturated NaHCO₃ (10 mL) and brine (10 mL), dried over Na₂SO₄. The crude product was purified with high pH HPLC to yield trans-(+/−)-N-(2-{[3-isobutylpiperidin-1-yl]methyl}cyclohexyl)-6-(1H-imidazol-1-yl)nicotinamide (62 mg, 58%) as a white powder. MS (M+1): 424.3. 1H NMR (400 MHz, METHANOL-D4) δ ppm 0.65 (dd, J=5.96, 2.44 Hz, 3H), 0.76-0.97 (m, 5H), 0.99-1.16 (m, 2H), 1.23-1.51 (m, 5H), 1.59-1.86 (m, 8H), 1.85-2.04 (m, 1H), 2.06-2.27 (m, 2H), 2.36-2.54 (m, 1H), 2.62-3.09 (m, 2H), 3.54-3.71 (m, 1H), 7.17 (s, 1H), 7.78-7.85 (m, 1H), 7.95 (s, 1H), 8.36 (dd, J=5.66, 2.93 Hz, 1H), 8.61 (s, 1H), 8.84-8.97 (m, 1H).

### Example 175

### trans-(+/−)-4-(1H-imidazol-1-yl)-N-{2-[(3-isobutylpiperidin-1-yl)methyl]cyclohexyl}benzamide

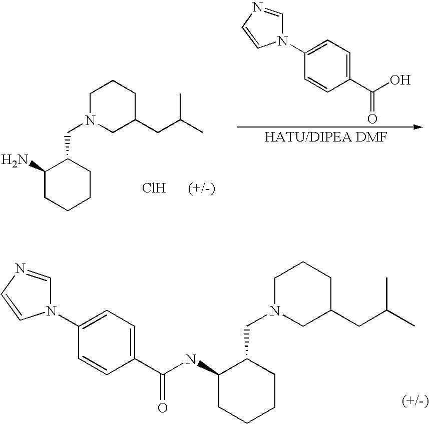

To a solution of trans-(+/−)-2-({3-isobutylpiperidin-1-yl}methyl)cyclohexyl]amine hydrochloride (73 mg, 0.25 mmol) in dry DMF (3 mL) was added 4-(1H-imidazol-1-yl)benzoic acid (56 mg, 0.3 mmol) followed by HATU (114 mg, 0.3 mmol) and diisopropylethylamine (0.10 mL, 0.5 mmol). The mixture was stirred at room temperature for 1 h, and the reaction was quenched with water (5 mL). The solvent was removed in vacuo. DCM (15 mL) was added and washed with saturated NaHCO₃ (10 mL) and brine (10 mL), dried over Na₂SO₄. The crude product was purified with high pH HPLC to yield trans-(+/−)-4-(1H-imidazol-1-yl)-N-{2-[(3-isobutylpiperidin-1-yl)methyl]cyclohexyl}benzamide (74 mg, 72%) as a white powder. MS (M+1): 423.3. 1H NMR (400 MHz, METHANOL-D4) δ ppm 0.60-0.69 (m, 3H), 0.78-0.97 (m, 5H), 0.97-1.19 (m, 2H), 1.23-1.51 (m, 5H), 1.56-1.82 (m, 8H), 1.82-2.11 (m, 2H), 2.10-2.28 (m, 1H), 2.37-2.57 (m, 1H), 2.72-3.15 (m, 2H), 3.51-3.70 (m, 1H), 7.17 (s, 1H), 7.66 (s, 1H), 7.70 (dd, J=8.50, 1.46 Hz, 2H), 7.97 (d, J=8.40 Hz, 2H), 8.25 (s, 1H).

### Example 176

### trans-(+/−)-4-Bromo-N-{2-[(3-propylpiperidin-1-yl)methyl]cyclohexyl}benzamide

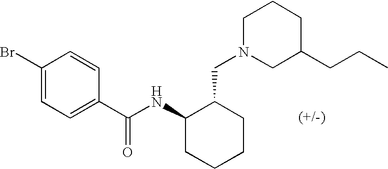

Following the HATU coupling procedure described in Example 173: The title compound was obtained as a white solid in a 50% yield (111 mg). MS (M+1): 421.3. 1H NMR (400 MHz, CHLOROFORM-D) δ ppm 0.68 (dd, J=7.13 Hz, 3H one isomer), 0.74-0.86 (m, 1H), 0.93 (t, J=7.32 Hz, 3H one isomer), 0.96-1.92 (m, 18H), 2.04 (dd, J=12.79, 3.61 Hz, 1H), 2.30-2.67 (m, 3H), 3.10 (d, J=10.35 Hz, 1H), 3.39 (t, J=10.06 Hz, 1H), 7.50-7.57 (m, 2H), 7.71 (t, J=7.71 Hz, 2H), 9.18 (d, J=17.58 Hz, 1H).

### Example 177

### trans-(+/−)-3-(4-Chlorophenyl)-N-{2-[(3-propylpiperidin-1-yl)methyl]cyclohexyl}propanamide

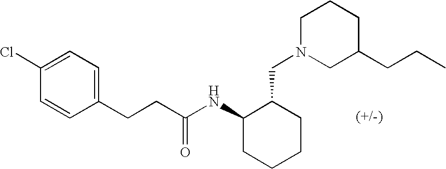

Following the procedure described in Example 173, the title compound was obtained as a white solid in a 52% yield (112 mg). MS (M+1): 405.3.1H NMR (400 MHz, CHLOROFORM-D) δ ppm 0.77-1.06 (m, 3H), 0.85 (t, J=7.23 Hz, 3H one isomer), 0.89 (t, J=7.32 Hz, 3H one isomer), 1.08-1.47 (m, 9H), 1.52-2.03 (m, 8H), 2.16-2.59 (m, 5H), 2.82-3.03 (m, 3H), 3.15-3.25 (m, 1H), 7.10-7.16 (m, 2H), 7.19-7.25 (m, 2H), 8.16 (amide NH, one isomer), 8.23 (amide NH, one isomer).

### Example 178

### trans-(+/−)-4-Bromo-N-{2-[(3-butylpiperidin-1yl)methyl]cyclohexyl}benzamide

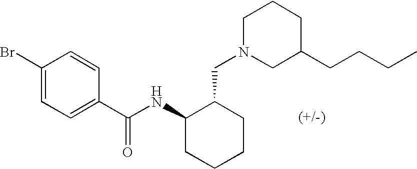

Following the procedure described in Example 165: the title compound was obtained as a white solid in a 52% yield (80 mg). MS (M+1): 435.3. 1H NMR (400 MHz, CHLOROFORM-D) δ ppm 0.72-1.46 (m, 15H), 1.48-1.87 (m, 8H), 1.99-2.25 (m, 2H), 2.37-2.86 (m, 3H), 3.20 (s, 1H), 3.51 (s, 1H), 7.48-7.59 (m, 2H), 7.77 (d, J=7.42 Hz, 2H), 9.03 (s, 1H).

### Example 179

### trans-(+/−)-N-{2-[(3-Butylpiperidin-1-yl)methyl]cyclohexyl}-4-[(diethylamino)methyl]benzamide

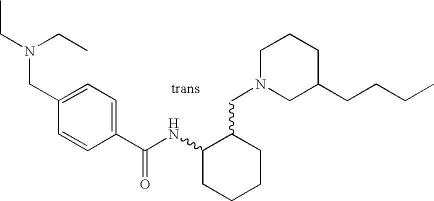

Following the procedure described in Example 165: the title compound was obtained as a yellow solid in a 12% yield (18 mg). MS (M+1): 442.3. 1H NMR (400 MHz, CHLOROFORM-D) δ ppm 0.72-0.95 (m, 5H), 0.96-1.46 (m, 17H), 1.50-1.95 (m, 7H), 2.03-2.46 (m, 3H), 2.47-2.65 (m, 5H), 2.75 (s, 1H), 3.26 (s, 1H), 3.47-3.76 (m, 3H), 7.35-7.48 (m, 2H), 7.89 (s, 2H), 8.80 (s, 1H).

### Example 180

### trans-(+/−)-3-(4-Chlorophenyl)-N-(2-{[3-(ethoxymethyl)piperidin-1-yl]methyl}cyclohexyl)propanamide

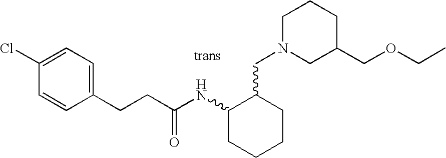

Following the procedure described in Example 2: the title compound was obtained as a white solid in a 50% yield (111 mg). MS (M+1): 421.3. 1H NMR (400 MHz, CHLOROFORM-D) δ ppm 0.85-1.05 (m, 3H), 1.16 (dd, J=7.03 Hz, 3H one isomer), 1.21 (t, J=7.03 Hz, 3H one isomer), 1.22-2.03 (m, 13H), 2.17-2.47 (m, 5H), 2.83-3.01 (m, 3H), 3.15-3.32 (m, 3H), 3.37-3.50 (m, 2H), 7.11-7.18 (m, 2H), 7.21-7.25 (m, 2H), 8.04 (br s, 1H). Anal. Calcd for C24H37ClN2O2: C, 68.47; H, 8.86; N, 6.65. Found: C, 68.03; H, 8.63; N, 6.57.

### Example 181

### N-[(1S,2R)-2-({4-[(2E)-But-2-en-1-yloxy]piperidin-1-yl}methyl)cyclohexyl]-6-(1H-pyrazol-1-yl)nicotinamide

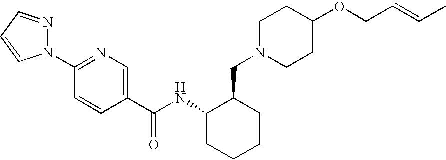

trans-(+/−)-N-[2-({4-[(2E)-But-2-en-1-yloxy]piperidin-1-yl}methyl)cyclohexyl]-6-(1H-pyrazol-1-yl)nicotinamide from Example 117 was separated on chiral AD column (10% ethanol in hexanes), and the second fraction was collected to yield the title compound as a pure enantiomer. MS (M+1): 438.3. 1H NMR (400 MHz, CHLOROFORM-D) δ ppm 1.05-1.16 (m, 2H), 1.25-1.47 (m, 2H), 1.58 (s, 3H), 1.59-1.67 (m, 3H), 1.71 (dq, J=6.27, 1.29 Hz, 2H), 1.73-1.81 (m, 3H), 2.03 (t, J=9.37 Hz, 1H), 2.10 (d, J=12.50 Hz, 1H), 2.38 (s, 1H), 2.43 (dd, J=12.89, 9.57 Hz, 1H), 2.50 (s, 1H), 2.63 (dd, J=12.69, 2.34 Hz, 1H), 2.89 (s, 1H), 3.35-3.48 (m, 2H), 3.88 (dt, J=6.01, 1.10 Hz, 2H), 5.51-5.61 (m, 1H), 5.64-5.74 (m, 1H), 6.49 (dd, J=2.64, 1.66 Hz, 1H), 7.76 (dd, J=1.66, 0.68 Hz, 1H), 8.03 (dd, J=8.50, 0.68 Hz, 1H), 8.24 (dd, J=8.59, 2.34 Hz, 1H), 8.62 (dd, J=2.64, 0.68 Hz, 1H), 8.87 (dd, J=2.25, 0.68 Hz, 1H), 9.11 (s, 1H). Anal. Calcd for C25H35N5O2.0.55H2O: C, 67.10; H, 8.13; N, 15.65. Found: C, 67.14; H, 8.19; N, 15.56. Chiralpak AD column, 4.6×250 mm column 10% isopropanol/90% hexane, 1 peak at 11.423 min, K′: 1.75>99% (215 nm), >99% (254 nm), >99% (280 nm).

### Example 182

### N-{(1S,2R)-2-[(4-Butoxypiperidin-1-yl)methyl]cyclohexyl}-6-(1H-pyrazol-1-yl)nicotinamide

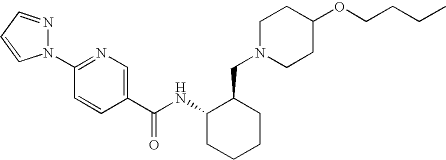

The title compound was obtained from the hydrogenation of N-[(1S,2R)-2-({4-[(2E)-But-2-en-1-yloxy]piperidin-1-yl}methyl)cyclohexyl]-6-(1H-pyrazol-1-yl)nicotinamide from Example 181. MS (M+1): 440.3. 1H NMR (400 MHz, CHLOROFORM-D) δ ppm 0.91 (t, J=7.32 Hz, 3H), 1.06-1.24 (m, 2H), 1.24-1.45 (m, 4H), 1.44-1.58 (m, 3H), 1.60-1.94 (m, 8H), 2.05-2.31 (m, 2H), 2.41-2.70 (m, 3H), 2.87-3.04 (m, 1H), 3.31-3.44 (m, 1H), 3.38 (t, J=6.54 Hz, 2H), 3.46-3.55 (m, 1H), 6.48 (dd, J=2.64, 1.66 Hz, 1H), 7.76 (d, J=0.98 Hz, 1H), 8.03 (d, J=8.40 Hz, 1H), 8.29 (d, J=7.81 Hz, 1H), 8.62 (d, J=2.73 Hz, 1H), 8.90 (s, 1H), 9.09 (s, 1H).

### Example 183 and 184

### N-(1S,2R)-2-{[(3R)-3-(2-Methoxyethoxy)piperidin-1-yl]methyl}cyclohexyl)-4-(1H-pyrazol-1-yl)benzamide and N-(1R,2S)-2-{[(3R)-3-(2-Methoxyethoxy)piperidin-1-yl]methyl}cyclohexyl)-4-(1H-pyrazol-1-yl)benzamide

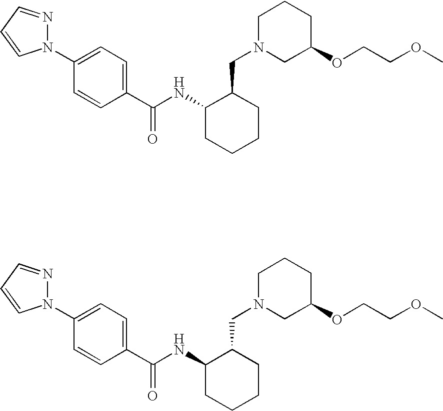

### Step A

### The Preparation of tert-butyl (3R)-3-hydroxypiperidin-1-carboxylate

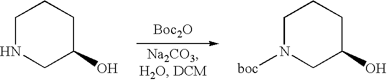

To a solution of the hydrochloric salt of (3R)-3-hydroxypiperidine (2.0 g, 14.6 mmol) in water (50 mL) and dichloromethane (40 mL) were added sodium carbonate (4.12 g, 29 mmol) and di-tert-butyl dicarbonate (3.5 g, 16 mmol). The reaction was stirred at room temperature overnight. The reaction was diluted with water (50 mL) and dichloromethane (50 mL). The phases were separated and the aqueous was extracted with dichloromethane (2×30 ml). The combined organic phases were washed with brine, dried over Na₂SO₄, filtered, and concentrated in vacuo. The product was purified by column chromatography (30% to 50% heptane in ethyl acetate). The product was obtained as colourless oil (2.32 g, 79%). 1H NMR (400 MHz, CHLOROFORM-D) δ ppm 1.42-1.54 (m, 2H), 1.46 (s, 9H), 1.69-1.80 (m, 1H), 1.86-1.93 (m, 1H), 2.20-2.72 (m, 1H), 2.99-3.16 (m, 2H), 3.56 (d, J=4.49 Hz, 1H), 3.50-3.60 (d, J=1.56 Hz, 1H), 3.73-3.84 (m, 1H).

### Step B

### The Preparation of tert-butyl (3R)-3-(2-methoxyethoxy)piperidine-1-carboxylate

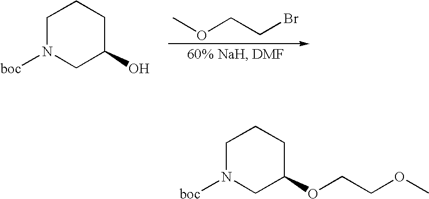

To a solution of tert-butyl (3R)-3-hydroxypiperidin-1-carboxylate (300 mg, 1.5 mmol) in dry DMF (5 mL) was added sodium hydride (60%, 115 mg, 3.0 mmol) at 0° C. under nitrogen and the suspension was stirred at room temperature for 30 min. 1-Bromo-2-methoxyethane (0.17 mL, 1.8 mmol) was added to the reaction mixture and stirred over night at room temperature. The reaction mixture was heated at 50° C. and Sodium hydride (60%, 58 mg, 1.5 mmol) was added, then 1-bromo-2-methoxyethane (0.17 mL, 1.8 mmol). The reaction mixture was stirred at 50° C. for 2 hours. Sodium hydride (60%, 58 mg, 1.5 mmol) was added, then 1-bromo-2-methoxyethane (0.17 mL, 1.8 mmol). The reaction was stirred at 50° C. for 2 hours and then cooled to room temperature. The reaction was quenched with water (1 mL) at 0° C. The solvent was removed in vacuo and the residue was dissolved in dichloromethane (30 mL) and water (25 mL). The phases were separated and the aqueous was extracted with dichloromethane (2×30 ml). The combined organic phases were washed with brine, dried over Na₂SO₄, filtered, and concentrated in vacuo. The product was purified by column chromatography (50% heptane in ethyl acetate). The product was obtained as colourless oil (328 mg, 84%). 1H NMR (400 MHz, CHLOROFORM-D) δ ppm 1.38-1.53 (m, 4H), 1.46 (s, 9H), 1.68-1.79 (m, 1H), 1.93-2.03 (m, 1H), 2.89-3.00 (m, 2H), 3.27-3.35 (m, 1H), 3.39 (s, 3H), 3.51-3.56 (m, 2H), 3.59-3.73 (m, 2H).

### Step C

### The Preparation of (3R)-3-(2-methoxyethoxy)piperidine hydrochloride

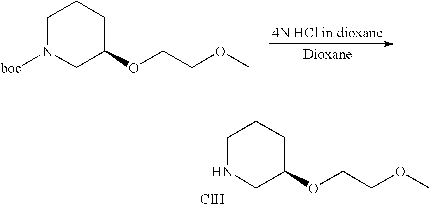

tert-butyl (3R)-3-(2-methoxyethoxy)piperidine-1-carboxylate from step A was stirred in 4N HCl in dioxane (3 mL) and dioxane (10 mL) at room temperature overnight. The solvent was removed in vacuo. The product was used directly for next step.

### Step D

### The Preparation of trans(±)-tert-butyl (2-{[(3R)-3-(2-methoxyethoxy)piperidin-1-yl]methyl}cyclohexyl)carbamate trans

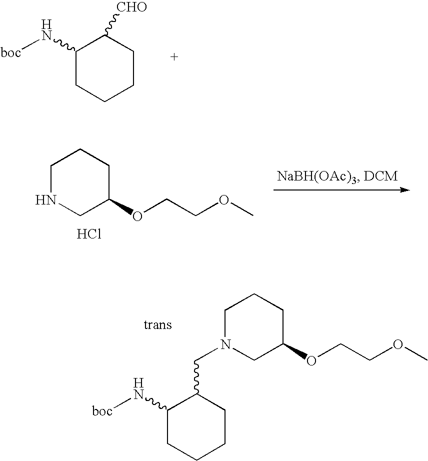

The product from step C was added to a solution of trans-(±)-tert-butyl[2-formylcyclohexyl]carbamate (290 mg, 4.40 mmol) in dichloromethane (13 ml). The reaction was stirred at room temperature for 30 minutes, and then sodium triacetoxyborohydride (530 mg, 2.54 mmol) was added portionwise to the reaction mixture. The reaction was stirred at room temperature overnight, and then cooled to 0° C. Water (5 ml) was added dropwise. A 1N sodium hydroxide solution (40 ml) and dichloromethane (50 ml) were added to the mixture. The phases were separated and the aqueous was extracted with dichloromethane (2×30 ml). The combined organic phases were washed with brine, dried over Na₂SO₄, filtered, and concentrated in vacuo. The product was used directly for the next step without further purification.

### Step E

### The Preparation of trans(±)-(2-{[(3R)-3-(2-methoxyethoxy)piperidin-1-yl]methyl}cyclohexyl)amine hydrochloride

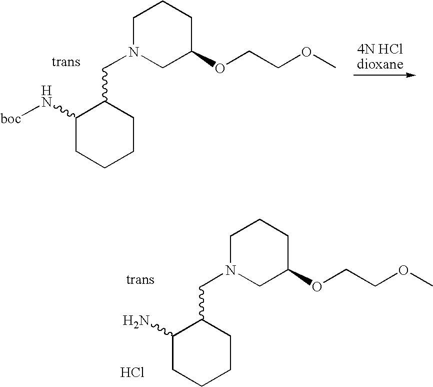

A 4N solution of hydrochloric acid in dioxane (6.0 ml, 24.0 mmol) was added to a solution of the crude product from step D trans(±)-tert-butyl (2-{[(3R)-3-(2-methoxyethoxy)piperidin-1-yl]methyl}cyclohexyl)carbamate (1.27 mmol) in dioxane (20 ml). The reaction was stirred at room temperature overnight. The solvent was removed in vacuo. MS (M+1): 271.2.

### Step F

### The Preparation of trans(±)-N-(2-{[(3R)-3-(2-methoxyethoxy)piperidin-1-yl]methyl}cyclohexyl)-4-(1H-pyrazol-1-yl)benzamide

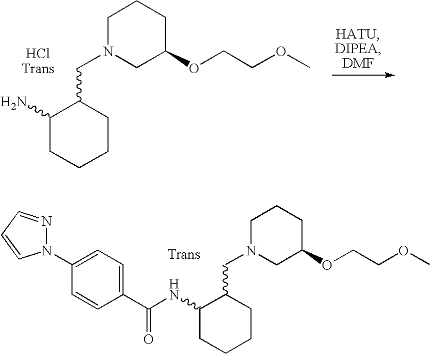

To the solution of trans(±)-(2-{[(3R)-3-(2-methoxyethoxy)piperidin-1-yl]methyl}cyclohexyl)amine hydrochloride (161 mg, 0.47 mmol) in dry DMF (10 mL) at 0° C. was added 6-(1H-imidazol-1-yl)benzoic acid (98 mg, 0.52 mmol) followed by diisopropylethylamine (0.33 mL, 1.88 mmol) and HATU (198 mg, 0.52 mmol). The mixture was stirred at room temperature overnight. The solvent was removed in vacuo. A 1N sodium hydroxide solution (20 ml) and dichloromethane (30 ml) were added to the mixture. The phases were separated and the aqueous was extracted with dichloromethane (2×30 ml). The combined organic phases were washed with brine, dried over Na₂SO₄, filtered, and concentrated in vacuo.

### Step G

### Separation of Two Diastereoisomers

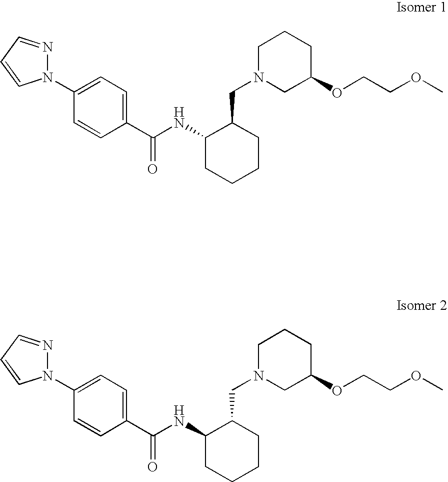

The diastereoisomer mixtures from Step F were separated with high pH reverse phase HPLC to yield both diastereoisomers.

Isomer 1 (N-(1S,2R)-2-{[(3R)-3-(2-methoxyethoxy)piperidin-1-yl]methyl}cyclohexyl)-4-(1H-pyrazol-1-yl)benzamide, white solid (41 mg, 20%)): MS (M+1): 441.3. 1H NMR (400 MHz, CHLOROFORM-D) δ ppm 1.03-1.48 (m, 5H), 1.53-1.68 (m, 3H), 1.68-1.80 (m, 3H), 1.84 (br s, 1H), 1.96 (t, J=11.03 Hz, 1H), 2.02-2.14 (m, 2H), 2.43-2.55 (m, 2H), 2.60 (d, J=10.35 Hz, 1H), 3.33 (d, J=8.59 Hz, 1H), 3.40 (s, 3H), 3.43-3.50 (m, 2H), 3.51-3.57 (m, 2H), 3.60-3.68 (m, 1H), 3.69-3.77 (m, 1H), 6.51 (dd, J=2.54, 1.76 Hz, 1H), 7.72-7.79 (m, 3H), 7.93 (d, J=8.40 Hz, 2H), 8.00 (d, J=2.54 Hz, 1H), 8.74 (s, 1H). Anal. Calcd for C25H36N4O3.0.7H2O: C, 66.26; H, 8.32; N, 12.36. Found: C, 66.96; H, 8.32; N, 12.36.

Isomer 2 (N-(1R,2S)-2-{[(3R)-3-(2-Methoxyethoxy)piperidin-1-yl]methyl}cyclohexyl)-4-(1H-pyrazol-1-yl)benzamide): White solid (37 mg, 18%), MS (M+1): 441.3. 1H NMR (400 MHz, CHLOROFORM-D) δ ppm 1.03-1.16 (m, 2H), 1.25-1.50 (m, 4H), 1.53-1.82 (m, 7H), 1.99-2.09 (m, 1H), 2.10 (d, J=12.69 Hz, 1H), 2.41 (dd, J=12.01, 9.86 Hz, 2H), 2.56-2.69 (m, 2H), 3.20 (s, 1H), 3.23 (br s, 3H), 3.35-3.51 (m, 5H), 6.50 (dd, J=2.54, 1.76 Hz, 1H), 7.74-7.77 (m, 2H), 7.77-7.80 (m, 1H), 8.00 (d, J=2.15 Hz, 1H), 8.05 (d, J=8.40 Hz, 2H), 8.96 (br s, 1H).

### Example 185 and 186

### N-[(1S,2R)-2-({(3R)-3-[(Allyloxy)methyl]piperidin-1-yl}methyl)cyclohexyl]-6-(1H-pyrazol-1-yl)nicotinamide and N-[(1R,2S)-2-({(3R)-3-[(Allyloxy)methyl]piperidin-1-yl}methyl)cyclohexyl]-6-(1H-pyrazol-1-yl)nicotinamide

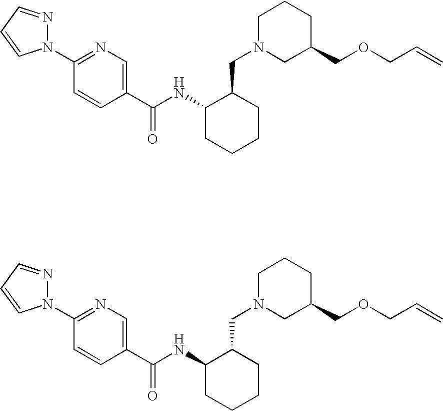

Following the HATU coupling procedure described in Example 129, Step E: the diastereo-mixture trans(±)-N-[2-({(3R)-3-[(Allyloxy)methyl]piperidin-1-yl}methyl)cyclohexyl]-6-(1H-pyrazol-1-yl)nicotinamide were prepared from trans(±)-[2-({(3R)-3-[(allyloxy)methyl]piperidin-1-yl}methyl)cyclohexyl]amine hydrochloride), then the diastereomeric mixture was separated by chiral AD column (15% isopropanol in hexanes) to yield diastereo-isomeric pure compounds.

Fraction 1: (N-[(1R,2S)-2-({(3R)-3-[(Allyloxy)methyl]piperidin-1-yl}methyl)cyclohexyl]-6-(1H-pyrazol-1-yl)nicotinamide): MS (M+1): 438.3. 1H NMR (400 MHz, CHLOROFORM-D) δ ppm 0.88-1.03 (m, 1H), 1.04-1.17 (m, 2H), 1.24-1.48 (m, 2H), 1.53-1.87 (m, 10H), 2.09 (d, J=12.69 Hz, 1H), 2.43 (dd, J=12.89, 9.77 Hz, 1H), 2.60-2.74 (m, 2H), 3.03-3.18 (m, 3H), 3.43 (tt, J=10.55, 3.12 Hz, 1H), 3.69 (d, J=5.47 Hz, 2H), 4.98-5.10 (m, 2H), 5.62-5.75 (dddd, J=17.24, 10.55, 5.57, 5.32 Hz, 1H), 6.49 (dd, J=2.54, 1.56 Hz, 1H), 7.76 (d, J=0.78 Hz, 1H), 8.01 (d, J=8.59 Hz, 1H), 8.24 (dd, J=8.50, 2.25 Hz, 1H), 8.61 (d, J=2.54 Hz, 1H), 8.86 (d, J=1.76 Hz, 1H), 9.14 (s, 1H). Anal. Calcd for C25H35N5O2: C, 68.62; H, 8.06; N, 16.00. Found: C, 68.30; H, 7.89; N, 15.93. Chiralpak AD column, 4.6×250 mm column 10% Isopropanol/90% hexane, 1 peak at 8.163 min, K′: 0.97>99% (215 nm), >99% (254 nm), >99% 280 nm)

Fraction 2: (N-[(1S,2R)-2-({(3R)-3-[(Allyloxy)methyl]piperidin-1-yl}methyl)cyclohexyl]-6-(1H-pyrazol-1-yl)nicotinamide): MS (M+1): 438.3. 1H NMR (400 MHz, CHLOROFORM-D) δ ppm 0.90 (qd, J=12.40, 3.81 Hz, 1H), 1.01-1.18 (m, 2H), 1.19-1.44 (m, 3H), 1.45-1.57 (m, 2H), 1.59-1.82 (m, 5H), 1.94 (dd, 2H), 2.07 (d, J=12.89 Hz, 1H), 2.43 (dd, J=12.50, 10.16 Hz, 1H), 2.61 (t, J=11.23 Hz, 2H), 3.23 (t, J=8.50 Hz, 1H), 3.31-3.47 (m, 3H), 3.99 (d, J=5.47 Hz, 2H), 5.18-5.35 (m, 2H), 5.94 (ddd, J=22.61, 10.60, 5.66 Hz, 1H), 6.49 (s, 1H), 7.77 (s, 1H), 8.01 (d, J=8.59 Hz, 1H), 8.25 (dd, J=8.59, 2.15 Hz, 1H), 8.62 (d, J=2.15 Hz, 1H), 8.87 (d, J=1.56 Hz, 1H), 9.21 (s, 1H). Anal. Calcd for C25H35N5O2: C, 68.82; H, 8.06; N, 16.00. Found: C, 68.30; H, 7.83; N, 15.73. Chiralpak AD column, 4.6×250 mm column 10% isopropanol, 1 peak at 12.653 min, K′: 2.05>99% (215 nm), >99% (254 nm), >99% (280 nm)

### Example 187 and 188

### N-[(1R,2S)-2-({(3R)-3-[(Allyloxy)methyl]piperidin-1-yl}methyl)cyclohexyl]-6-(1H-pyrazol-1-yl)nicotinamide and N-[(1S,2R)-2-({(3R)-3-[(Allyloxy)methyl]piperidin-1-yl}methyl)cyclohexyl]-6-(1H-imidazol-1-yl)nicotinamide

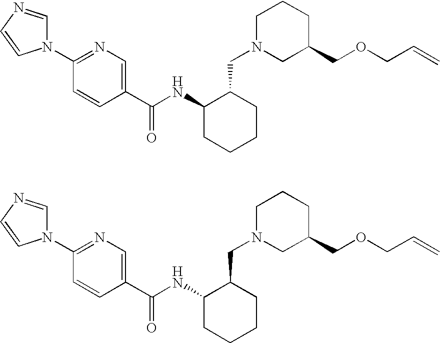

The diastereo mixture trans(±)-N-[2-({(3R)-3-[(allyloxy)methyl]piperidin-1-yl}methyl)cyclohexyl]-6-(1H-imidazol-1-yl)nicotinamide from Example 129 was separated by chiral AD column (10% ethanol in hexanes) to produce two pure diastereoisomers.

Fraction 1: (N-[(1R,2S)-2-({(3R)-3-[(Allyloxy)methyl]piperidin-1-yl}methyl)cyclohexyl]-6-(1H-pyrazol-1-yl)nicotinamide): MS (M+1): 438.3. 1H NMR (400 MHz, CHLOROFORM-D) δ ppm 0.96 (qd, J=12.11, 3.32 Hz, 1H), 1.05-1.18 (m, 2H), 1.26-1.48 (m, 2H), 1.52-1.89 (m, 10H), 2.10 (d, J=13.28 Hz, 1H), 2.44 (t, J=10.84 Hz, 1H), 2.67 (dd, J=35.74, 10.94 Hz, 2H), 3.03-3.18 (m, 3H), 3.44 (t, J=10.16 Hz, 1H), 3.71 (d, J=5.47 Hz, 2H), 5.00-5.12 (m, 2H), 5.62-5.78 (m, J=17.31, 10.67, 5.47, 5.22 Hz, 1H), 7.22 (t, 1H), 7.39 (dd, J=8.40, 0.78 Hz, 1H), 7.67 (t, J=1.37 Hz, 1H), 8.30 (dd, J=8.50, 2.25 Hz, 1H), 8.40 (s, 1H), 8.89 (d, J=1.95 Hz, 1H), 9.21 (s, 1H). Chiralpak OD column, 4.6×250 mm column 10% Ethanol/90% hexane, 1 peak at 10.672 min, K′: 1.57, >99% (215 nm), >99% (254 nm), >99% (280 nm)

Fraction 2: ((N-[(1S,2R)-2-({(3R)-3-[(Allyloxy)methyl]piperidin-1-yl}methyl)cyclohexyl]-6-(1H-pyrazol-1-yl)nicotinamide (MS (M+1): 438.3. 1H NMR (400 MHz, CHLOROFORM-D) δ ppm 0.85-0.99 (m, 1H), 1.02-1.58 (m, 6H), 1.59-1.84 (m, 6H), 1.95 (t, J=10.35 Hz, 2H), 2.09 (d, J=13.09 Hz, 1H), 2.43 (t, J=10.84 Hz, 1H), 2.60 (s, 2H), 3.24 (dd, J=9.08, 7.91 Hz, 1H), 3.31-3.48 (m, 3H), 4.00 (dt, J=5.71, 1.34 Hz, 2H), 5.20-5.34 (m, 2H), 5.89-6.00 (ddt, J=17.16, 10.42, 5.74 Hz, 1H), 7.22 (s, 1H), 7.39 (dd, J=8.50, 0.68 Hz, 1H), 7.69 (s, 1H), 8.31 (dd, J=8.40, 2.34 Hz, 1H), 8.42 (s, 1H), 8.89 (d, J=1.76 Hz, 1H), 9.29 (s, 1H). Chiralpak OD column, 4.6×250 mm column 10% Ethanol/90% hexane, 1 peak at 13.684 min, K′: 2.30, >99% (215 nm), >99% (254 nm), >99% (280 nm)

### Example 189

### (N-((1S,2R)-2-{[(3R)-3-ethoxypiperidin-1-yl]methyl}cyclohexyl)pyrazine-2-carboxamide

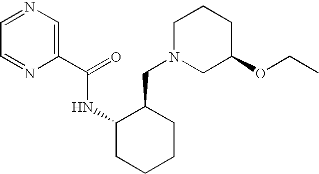

### Step A

### The Preparation of tert-butyl 3R-(ethoxy)piperidin-1-carboxylate

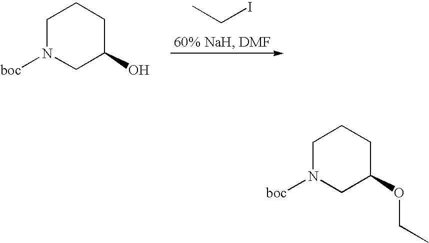

To a solution of tert-butyl 3R-(hydroxy)piperidin-1-carboxylate (145 mg, 0.72 mmol) in dry DMF (3 mL) was added NaH (60% 55 mg, 1.44 mmol) at 0° C. under nitrogen and the suspension was stirred at room temperature for 30 min. ethyl iodide (0.07 mL, 0.86 mmol) was added to the reaction mixture and stirred over night at room temperature. Quenched with water. Extracted with dichloromethane (3×20 mL), washed with brine, dried over Na₂SO₄. Removal of solvent gave 146 mg of crude product, which was used for the next step without further purification. MS (M+1): 230.1 (m−55): 174.0

### Step B

### The Preparation of 3R-(ethoxy)piperidine hydrochloride salt

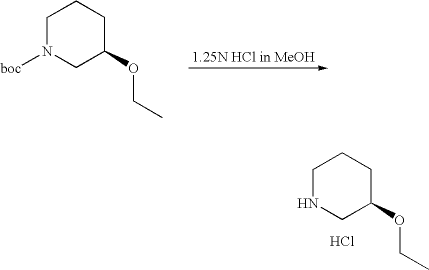

A 1.25N solution of hydrochloric acid in MeOH (8.0 mL, 10.0 mmol) was added to a solution of the crude product from step A tert-butyl 3R-(ethyloxy)piperidin-1-carboxylate (0.72 mmol). The reaction was stirred at room temperature for 3 days. The mixture was concentrated in vacuo to get 153 mg crude. The product was used directly for the next step without further purification. MS (M+1): 130.0.

### Step C

### The Preparation of tert-butyl((1R*,2S*)-2-{[(3R)-3-ethoxypiperidin-1-yl]methyl}cyclohexyl)carbamate

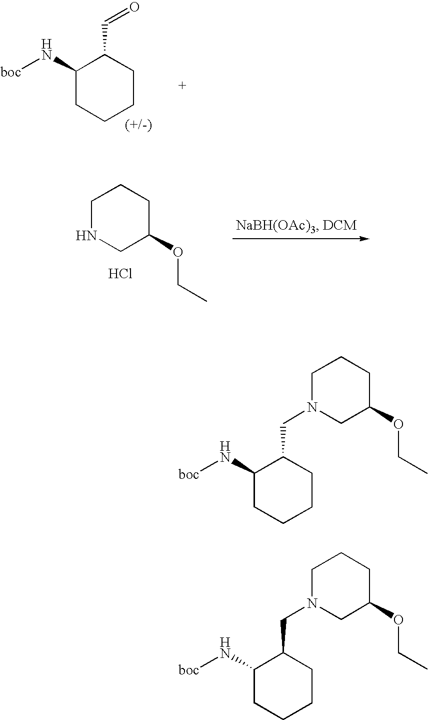

Crude product from step B (3R)-3-ethoxypiperidine hydrochloride salt (153 mg, 0.60 mmol) was added to a solution of tert-butyl trans-(+/−)-[2-formylcyclohexyl]carbamate (136 mg crude, 0.72 mmol) in dichloromethane (4 mL). The reaction was stirred at room temperature for 30 minutes, and then sodium triacetoxyborohydride (254 mg, 1.2 mmol) was added to the reaction mixture. The reaction was stirred at room temperature for 12 Hours. Water (1 mL) was added dropwise. A 2N sodium hydroxide solution (10 mL) and dichloromethane (30 mL) were added to the mixture. The phases were separated and the aqueous was extracted with dichloromethane (2×15 mL). The combined organic phases were washed with brine, dried over Na₂SO₄, filtered, and concentrated in vacuo to get 167 mg crude product. MS (m+1): 341.3. The product was used directly for the next step without further purification.

### Step D

### The Preparation of trans-2-{[(3R)-3-ethoxypiperidin-1-yl]methyl}cyclohexyl)amine hydrochloride salt

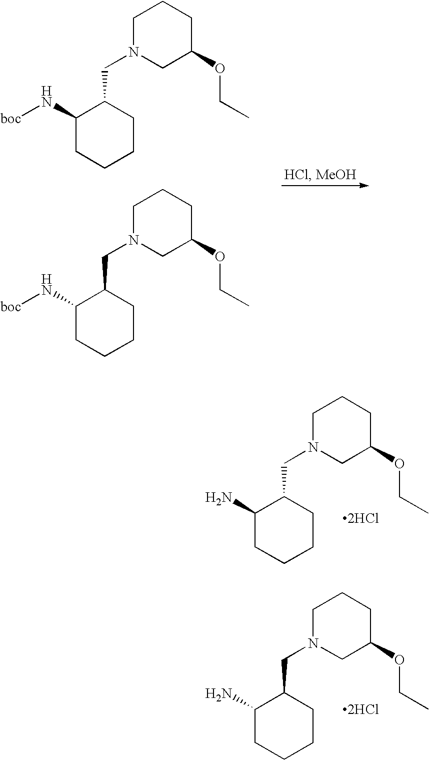

A 1.25N solution of hydrochloric acid in MeOH (8.0 mL, 10.0 mmol) was added to a solution of the crude product from step C trans-tert-butyl (2-{[(3R)-3-ethoxypiperidin-1-yl]methyl}cyclohexyl)carbamate (0.50 mmol). The reaction was stirred at room temperature for overnight. 1.25N solution of hydrochloric acid in MeOH was added until full conversion if reaction not completed. The mixture was concentrated in vacuo. The product was used directly for the next step without further purification. MS (M+1): 241.2

### Step E

### The Preparation of N-((1S,2R)-2-{[(3R)-3-ethoxypiperidin-1-yl]methyl}cyclohexyl)pyrazine-2-carboxamide

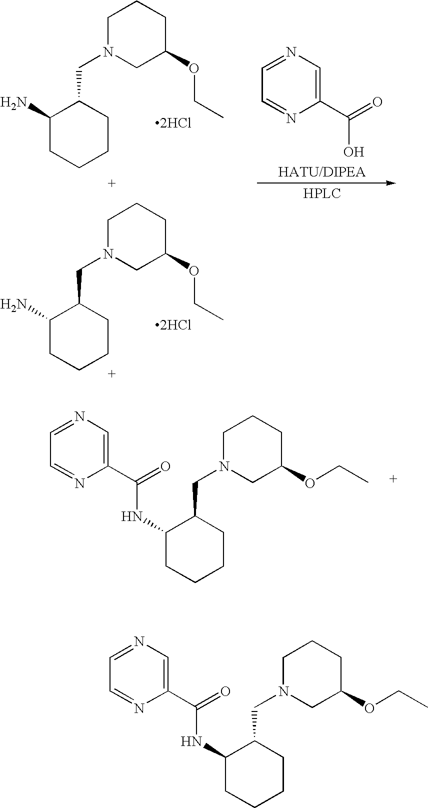

A solution of pyrazine-2-carboxylic acid (75 mg, 0.6 mmol), HATU (228 mg, 0.6 mmol) and diisopropylethylamine (0.18 mL, 1.0 mmol) in dry DMF (5 mL) was stirred at room temperature for 10 minutes. Trans-(+/−)-2-{[(3R)-3-ethoxypiperidin-1-yl]methyl}cyclohexyl)amine hydrochloride salt crude (143 mg, 0.5 mmol) was added to the solution. The mixture was stirred at room temperature for overnight, but no full conversion. Then 1.2 eq of carboxylic acid, 1.2 eq of HATU and 4 eq DIPEA were added to the mixture, which was stirred for 3 days. The solvent was removed in vacuo. DCM (15 mL) was added and washed with saturated NaHCO₃ (10 mL) and brine (10 mL), dried over Na₂SO₄. Removal of solvent gave the crude trans(±)—N-(2-{[(3R)-3-ethoxypiperidin-1-yl]methyl}cyclohexyl)pyrazine-2-carboxamide diastereomeric mixtures. The diastereoisomeric mixtures were separated with preparative high pH HPLC. The first fraction was collected to afford the title compound (N-((1S,2R)-2-{[(3R)-3-ethoxypiperidin-1-yl]methyl}cyclohexyl)pyrazine-2-carboxamide as its free base (15 mg). MS (M+1): 347.3 1H NMR (400 MHz, CDCl₃) δ ppm 0.98-1.18 (m, 3H), 1.21 (t, J=6.93 Hz, 3H), 1.24-1.51 (m, 3H), 1.51-1.82 (m, 6H), 1.87 (t, J=10.64 Hz, 1H), 1.98-2.09 (m, 1H), 2.09-2.19 (m, 1H), 2.34-2.50 (m, 2H), 2.56 (d, J=9.96 Hz, 1H), 3.20 (s, 1H), 3.39-3.69 (m, 4H), 8.51 (s, 1H), 8.73 (d, J=2.34 Hz, 1H), 9.13 (s, 1H), 9.40 (s, 1H)

### Example 190

### N-((1S,2R)-2-{[(3R)-3-ethoxypiperidin-1-yl]methyl}cyclohexyl)-6-(ethylthio)nicotinamide

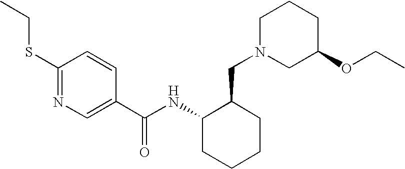

A solution of 6-(ethylthio)nicotinic acid (81 mg, 0.44 mmol), HATU (168 mg, 0.44 mmol) and diisopropylethylamine (0.12 mL, 0.88 mmol) in dry DMF (5 mL) was stirred at room temperature for 10 minutes. Trans-2-{[(3R)-3-ethoxypiperidin-1-yl]methyl}cyclohexyl)amine hydrochloride salt described in Example 189 Step D (68 mg, 0.22 mmol) was added to the solution. The mixture was stirred at room temperature for overnight, but no full conversion. Then 1.2 eq of carboxylic acid, 1.2 eq of HATU and 4 eq DIPEA were added to the mixture, which was stirred for 3 days. The solvent was removed in vacuo. DCM (15 mL) was added and washed with saturated NaHCO₃ (10 mL) and brine (10 mL), dried over Na₂SO₄. The crude product was subjected to separation on high pH preparative LC-MS. The first fraction was collected to yield the title compound N-((1S,2R)-2-{[(3R)-3-ethoxypiperidin-1-yl]methyl}cyclohexyl)-6-(ethylthio)nicotinamide (15 mg) as its free base. MS (M+1): 406.1. ¹H NMR (400 MHz, CDCl₃) δ ppm 0.99-1.42 (m, 5H), 1.18 (t, J=6.93 Hz, 3H), 1.36 (t, J=7.32 Hz, 3H), 1.48-1.82 (m, 4H), 1.88-2.16 (m, J=20.70 Hz, 4H), 2.33-2.64 (m, 3H), 3.07-3.31 (m, 4H), 3.40 (d, J=5.86 Hz, 2H), 3.47-3.66 (m, 3H), 7.17 (d, J=8.20 Hz, 1H), 7.91 (s, 1H), 8.79 (d, J=18.75 Hz, 2H).

### Example 191

### N-((1S,2R)-2-{[(3R)-3-ethoxypiperidin-1-yl]methyl}cyclohexyl)-6-pyrrolidin-1-ylnicotinamide

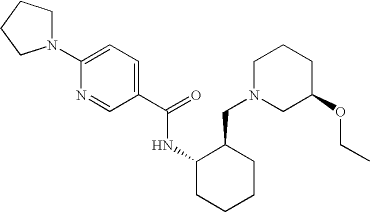

Following the same procedure as Example 189: N-((1S,2R)-2-{[(3R)-3-ethoxypiperidin-1-yl]methyl}cyclohexyl)-6-pyrrolidin-1-ylnicotinamide (16 mg, 33%) was obtained as its free base. MS (M+1): 415.3. ¹H NMR (400 MHz, CDCl₃) δ pp [symbol] 1.12 (d, J=21.09 Hz, 2H), 1.20 (t, J=6.54 Hz, 3H), 1.24-1.47 (m, 5H), 1.48-1.85 (m, 7H), 1.86-2.18 (m, 7H), 2.21-2.85 (m, 3H), 3.06-3.40 (m, J=56.44 Hz, 1H), 3.50 (s, 3H), 3.62 (d, 2H), 3.80-4.07 (m, 1H), 6.32 (d, J=8.98 Hz, 1H), 7.87 (s, 1H), 8.22 (s, 1H), 8.46-9.02 (m, 1H)

### Example 192

### N-[(1S,2R)-2-(azepan-1-ylmethyl)cyclohexyl]-4-(1H-pyrazol-1-yl)benzamide

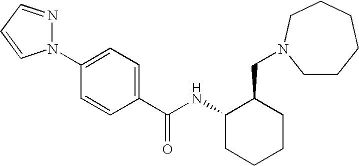

### Step A

### trans-(+/−)-tert-butyl-[2-(azepan-1-ylmethyl)cyclohexyl]carbamate

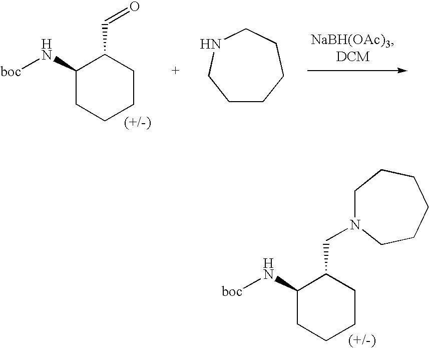

Azepane (0.27 mL, 2.40 mmol) was added to a solution of trans-(+/−)-tert-butyl[2-formylcyclohexyl]carbamate from Elise Balaux (273 mg crude, 1.2 mmol) in dichloromethane (12 mL). The reaction was stirred at room temperature for 30 minutes, and then sodium triacetoxyborohydride (254 mg, 1.2 mmol) was added to the reaction mixture. The reaction was stirred at room temperature for 12 Hours. Water (1 mL) was added dropwise. A 2N sodium hydroxide solution (15 mL) and dichloromethane (30 mL) were added to the mixture. The phases were separated and the aqueous was extracted with dichloromethane (2×20 mL). The combined organic phases were washed with brine, dried over Na₂SO₄, filtered, and concentrated in vacuo. MS (m+1): 311.3. 421 mg was obtained. The product was used directly for the next step without further purification.

### Step B

### The Preparation of trans-(+/−)-[2-(azepan-1-ylmethyl)-1-ethylpentyl]amine hydrochloride salt

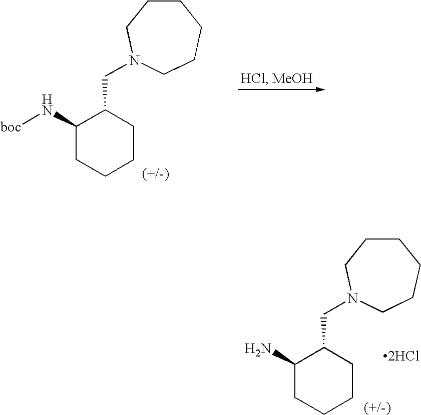

A 1.25N solution of hydrochloric acid in MeOH (6.0 mL, 7.20 mmol) was added to a solution of the crude product from step A trans-(+/−)-tert-butyl-[2-(azepan-1-ylmethyl)cyclohexyl]carbamate (1.20 mmol). The reaction was stirred at room temperature for 3 days. Reaction was not completed. 3 mL of 1.25N solution of hydrochloric acid in MeOH was added and the mixture stirred 4 hours. Still not completed, excess of 1.25N solution of hydrochloric acid in MeOH was added and stirred at room temperature for 12 Hours. The mixture was concentrated in vacuo to get 563.6 mg crude. The product was used directly for the next step without further purification. MS (M+1): 211.1.

### Step C

### The Preparation of trans-(+/−)-N-[2-(azepan-1-ylmethyl)cyclohexyl]-4-(1H-pyrazol-1-yl)benzamide

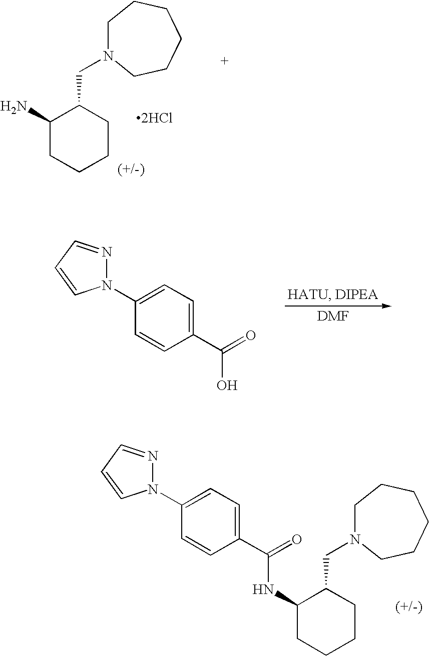

A solution of pyrazine-2-carboxylic acid (135 mg, 0.72 mmol), HATU (273 mg, 0.72 mmol) and diisopropylethylamine (0.42 mL, 2.4 mmol) in dry DMF (5 mL) was stirred at room temperature for 10 minutes. trans-(+/−)-[2-(azepan-1-ylmethyl)-1-ethylpentyl]amine hydrochloride salt crude (0.6 mmol) was added to the solution. The mixture was stirred at room temperature for overnight, but no full conversion. Then 1.2 eq of carboxylic acid, 1.2 eq of HATU and 4 eq DIPEA were added to the mixture, which was stirred for 3 days. The solvent was removed in vacuo. DCM (15 mL) was added and washed with saturated NaHCO₃ (10 mL) and brine (10 mL), dried over Na₂SO₄. The crude product was purified with reverse phase HPLC High pH to get the trans-(+/−)-N-[2-(azepan-1-ylmethyl)cyclohexyl]-4-(1H-pyrazol-1-yl)benzamide. MS (M+1): 381.2

### Step D

### The Preparation of N-[(1S,2R)-2-(azepan-1-ylmethyl)cyclohexyl]-4-(1H-pyrazol-1-yl)benzamide

The racemic mixture from step C was separated by chiral AD column with 10% EtOH/Hex. as eluent to get the yielded N-[(1S,2R)-2-(azepan-1-ylmethyl)cyclohexyl]-4-(1H-pyrazol-1-yl)benzamide (10 mg, 9% two steps) as its free base. MS (M+1): 381.3. ¹H NMR (400 MHz, CDCl₃) δ pp [symbol] 1.06 (d, J=7.42 Hz, 2H), 1.20-1.47 (m, 4H), 1.47-1.67 (m, 6H), 1.67-1.80 (m, 3H), 2.25-2.34 (m, 1H), 2.35-2.45 (m, 1H), 2.47-2.57 (m, 2H), 2.63 (d, J=11.91 Hz, 4H), 3.36-3.50 (m, 1H), 6.47-6.52 (m, 1H), 7.70-7.78 (m, 3H), 7.92 (d, J=8.20 Hz, 2H), 7.98 (d, J=2.34 Hz, 1H), 9.20 (s, 1H).

### Example 193

### N-[(1S,2R)-2-(azepan-1-ylmethyl)cyclohexyl]-6-(1H-pyrazol-1-yl)nicotinamide

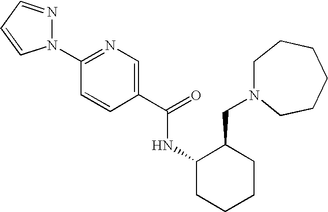

Following the same procedure as example 192, the racemic mixture of the trans-N-[2-(azepan-1-ylmethyl)cyclohexyl]-6-(1H-pyrazol-1-yl)nicotinamide was obtained and separated on AD column with 10% EtOH/Hex. as eluent. The first fraction was collected to yield N-[(1S,2R)-2-(azepan-1-ylmethyl)cyclohexyl]-6-(1H-pyrazol-1-yl)nicotinamide 15 mg (13% two steps) as its free base. MS (M+1): 382.3 ¹H NMR (400 MHz, CDCl₃) δ ppm 0.95-1.19 (m, 3H), 1.22-1.47 (m, 3H), 1.49-1.61 (m, J=6.84 Hz, 1H), 1.61 (s, 3H), 1.69-1.81 (m, 3H), 2.27-2.45 (m, 3H), 2.48-2.58 (m, 3H), 2.64 (d, J=13.28 Hz, 4H), 3.38-3.49 (m, J=10.45, 10.45 Hz, 1H), 6.48 (m, 1H), 7.76 (d, J=0.78 Hz, 1H), 8.00 (d, J=8.59 Hz, 1H), 8.20 (m, 1H), 8.60 (d, J=2.54 Hz, 1H), 8.84 (s, 1H), 9.44 (s, 1H)

### Example 194

### N-((1S,2R)-2-{[(3R)-3-(allyloxy)piperidin-1-yl]methyl}cyclohexyl)-4-(1H-pyrazol-1-yl)benzamide

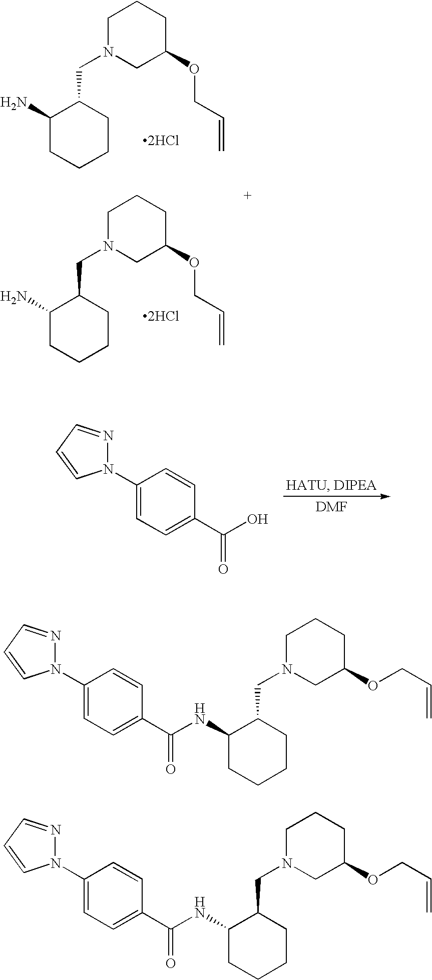

A solution of pyrazine-2-carboxylic acid (120 mg, 0.64 mmol), HATU (304 mg, 0.80 mmol) and diisopropylethylamine (0.28 mL, 1.60 mmol) in dry DMF (5 mL) was stirred at room temperature for 10 minutes. The trans-(+/−)-(2-{[(3R)-3-(allyloxy)piperidin-1-yl]methyl}cyclohexyl)amine hydrochloride salt (100 mg, 0.31 mmol) was added to the solution. The mixture was stirred at room temperature for overnight. Then 1.2 eq of pyrazine-2-carboxylic acid, 1.2 eq of HATU and 4 eq DIPEA were added to the mixture, which was stirred for 3 days. The solvent was removed in vacuo. DCM (15 mL) was added and washed with saturated NaHCO₃ (10 mL) and brine (10 mL), dried over Na₂SO₄. The crude product was purified with High pH LC-MS to separate two diastereoisomers. The first fraction was collected to yield The title compound N-((1S,2R)-2-{[(3R)-3-(allyloxy)piperidin-1-yl]methyl}cyclohexyl)-4-(1H-pyrazol-1-yl)benzamide 16 mg (25%) as its free base. MS (M+1): 423.3. ¹H NMR (400 MHz, CDCl₃) δ ppm 0.99-1.49 (m, 6H), 1.49-1.68 (m, 4H), 1.73 (q, J=9.96 Hz, 3H), 1.91-2.06 (m, 2H), 2.09 (d, J=12.89 Hz, 1H), 2.39-2.55 (m, 2H) 2.61 (d, J=10.94 Hz, 1H), 3.26 (d, J=9.37 Hz, 1H), 3.37-3.52 (m, 2H), 4.05 (ddd, J=31.10, 12.55, 5.57 Hz, 2H), 5.18 (dd, J=10.35, 0.98 Hz, 1H), 5.29 (dd, J=17.19, 1.56 Hz, 1H), 5.85-5.98 (m, 1H), 6.45-6.54 (m, 1H), 7.70-7.79 (m, 2H), 7.91 (d, J=8.59 Hz, 2H), 7.99 (d, J=2.34 Hz, 1H), 8.71 (s, 1H).

### Example 195 and 196

### N-((1R,2S)-2-{[(3R)-3-(ethoxymethyl)piperidin-1-yl]methyl}cyclohexyl)-4-(1H-pyrrol-1-yl)benzamide and N-((1S,2R)-2-{[(3R)-3-(ethoxymethyl)piperidin-1-yl]methyl}cyclohexyl)-4-(1H-pyrrol-1-yl)benzamide

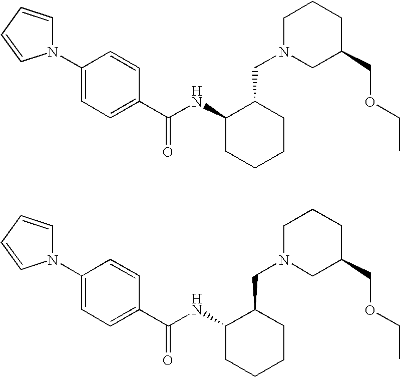

### Step A

### The Preparation of trans-N-(2-{[(3R)-3-(ethoxymethyl)piperidin-1-yl]methyl}cyclohexyl)-4-(1H-pyrrol-1-yl)benzamide

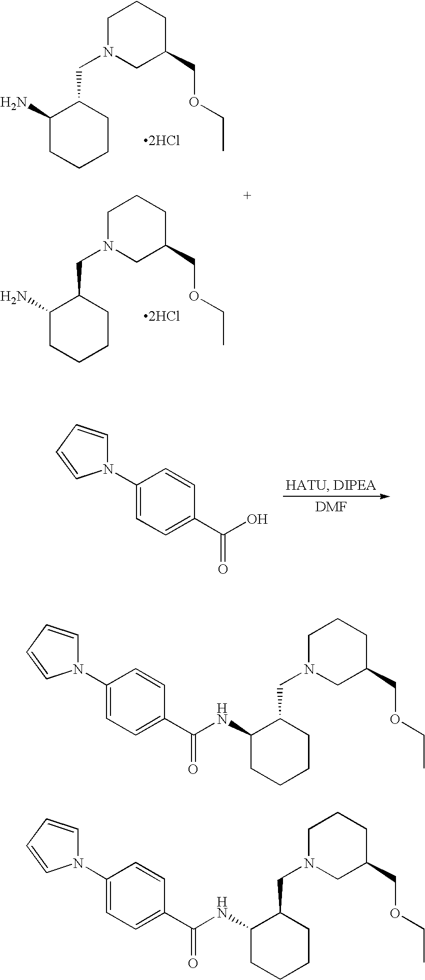

A solution 4-(1H-pyrrol-1-yl)benzoic acid (94 mg, 0.50 mmol), HATU (190 mg, 0.50 mmol) and a few drop of diisopropylethylamine in dry DMF (5 mL) was stirred at room temperature for 10 minutes. Crude trans(±)-(2-{[(3R)-3-ethoxypiperidin-1-yl]methyl}cyclohexyl)amine hydrochloride salt (162 mg, 0.50 mmol) was added to the solution. The mixture was stirred at room temperature for overnight. The solvent was removed in vacuo. Residue was dissolved in DCM (15 mL) and washed with saturated NaHCO₃ (10 mL) and brine (10 mL), dried over Na₂SO₄. The crude product was purified by prep LC-MS High pH to yield the diastereomeric mixtures trans-(±)-N-(2-{[(3R)-3-(ethoxymethyl)piperidin-1-yl]methyl}cyclohexyl)-4-(1H-pyrrol-1-yl)benzamide as its free base. MS (M+1): 424.3

### Step B

### Chiral Separation of trans-N-(2-{[(3R)-3-(ethoxymethyl)piperidin-1-yl]methyl}cyclohexyl)-4-(1H-pyrrol-1-yl)benzamide

The diastereo mixture trans-N-(2-{[(3R)-3-(ethoxymethyl)piperidin-1-yl]methyl}cyclohexyl)-4-(1H-pyrrol-1-yl)benzamide was separated on chiral AD column, eluent 10% i-PrOH/Hexane to obtain two isomers.

Isomer 1 (64 mg): N-((1R,2S)-2-{[(3R)-3-(ethoxymethyl)piperidin-1-yl]methyl}cyclohexyl)-4-(1H-pyrrol-1-yl)benzamide as its free base. MS (M+1): 424.3. ¹H NMR (400 MHz, CDCl₃) δ ppm 0.83-0.96 (m, 1H), 1.00 (t, J=6.93 Hz, 3H), 1.03-1.16 (m, 2H), 1.20-1.48 (m, 2H), 1.49-1.67 (m, 5H), 1.73 (d, J=9.57 Hz, 5H), 2.05 (d, J=12.69 Hz, 1H), 2.41 (dd, J=12.21, 10.06 Hz, 1H), 2.51-2.72 (m, 2H), 2.97-3.11 (m, 3H), 3.11-3.23 (m, 2H), 3.42 (t, J=10.45 Hz, 1H), 6.36 (t, 2H), 7.12 (t, J=2.15 Hz, 2H), 7.41 (d, J=8.40 Hz, 2H), 7.88 (d, J=8.40 Hz, 2H), 8.90 (s, 1H)

Isomer 2: N-((1S,2R)-2-{[(3R)-3-(ethoxymethyl)piperidin-1-yl]methyl}cyclohexyl)-4-(1H-pyrrol-1-yl)benzamide 68 mg was obtained as its free base. MS (M+1): 424.3. ¹H NMR (400 MHz, CDCl₃) δ ppm 0.78-0.98 (m, 1H), 1.01-1.17 (m, 2H), 1.24 (t, J=7.03 Hz, 3H), 1.27-1.44 (m, 2H), 1.42-1.53 (m, 2H), 1.53-1.70 (m, 4H), 1.70-1.81 (m, 2H), 1.84-1.98 (m, 2H), 2.05 (d, J=12.69 Hz, 1H), 2.41 (dd, J=12.69, 9.77 Hz, 1H), 2.59 (t, J=11.52 Hz, 2H), 3.21 (t, J=8.50 Hz, 1H), 3.25-3.38 (m, 2H), 3.38-3.55 (m, 3H), 6.36-6.40 (m, 2H), 7.15 (t, J=2.15 Hz, 2H), 7.42 (d, J=8.59 Hz, 2H), 7.90 (d, J=8.59 Hz, 2H), 8.98 (s, 1H)

### Examples 197 and 198

### N-((1R,2S)-2-{[(3R)-3-(ethoxymethyl)piperidin-1-yl]methyl}cyclohexyl)-6-pyrrolidin-1-ylnicotinamide and N-((1S,2R)-2-{[(3R)-3-(ethoxymethyl)piperidin-1-yl]methyl}cyclohexyl)-6-pyrrolidin-1-ylnicotinamide

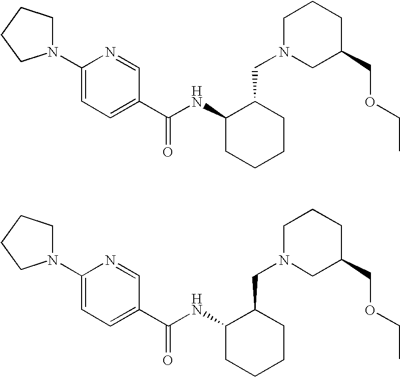

### Step A

### The Preparation of trans-N-(2-{[(3R)-3-(ethoxymethyl)piperidin-1-yl]methyl}cyclohexyl)-6-pyrrolidin-1-ylnicotinamide

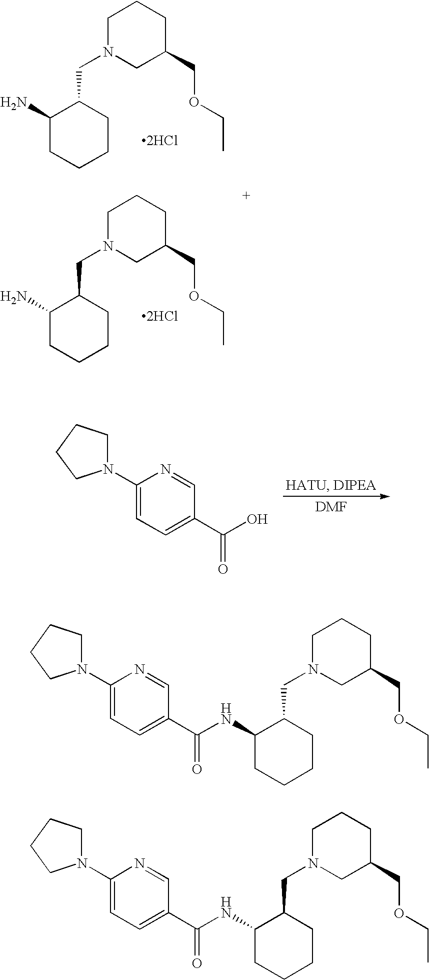

A solution 6-pyrrolidin-1-ylnicotinic acid (96 mg, 0.50 mmol), HATU (190 mg, 0.50 mmol) and a few drop of diisopropylethylamine in dry DMF (5 mL) was stirred at room temperature for 10 minutes. Crude trans(±)-(2-{[(3R)-3-ethoxypiperidin-1-yl]methyl}cyclohexyl)amine hydrochloride salt (162 mg, 0.50 mmol) was added to the solution. The mixture was stirred at room temperature for overnight. The solvent was removed in vacuo. Residue was dissolved in DCM (15 mL) and washed with saturated NaHCO₃ (10 mL) and brine (10 mL), dried over Na₂SO₄. The crude product was purified by prep LC-MS High pH to afford the diastereomeric mixture trans-N-(2-{[(3R)-3-(ethoxymethyl)piperidin-1-yl]methyl}cyclohexyl)-6-pyrrolidin-1-ylnicotinamide 123 mg (57%) as its free base. MS (M+1): 429.3

### Step B

### Chiral Separation of trans-N-(2-{[(3R)-3-(ethoxymethyl)piperidin-1-yl]methyl}cyclohexyl)-6-pyrrolidin-1-ylnicotinamide

The diastereo mixture of the trans-N-(2-{[(3R)-3-(ethoxymethyl)piperidin-1-yl]methyl}cyclohexyl)-4-(1H-pyrrol-1-yl)benzamide 123 mg (57%) was separated on chiral AD column, eluent 10% i-PrOH/Hexane to afford two isomers:

Isomer 1: N-((1R,2S)-2-{[(3R)-3-(ethoxymethyl)piperidin-1-yl]methyl}cyclohexyl)-6-pyrrolidin-1-ylnicotinamide 5 mg (8%) as its free base. MS (M+1): 429.3. ¹H NMR (400 MHz, CDCl₃) δ ppm 0.80-1.15 (m, 2H), 1.03 (t, J=7.03 Hz, 3H), 1.14-1.51 (m, 2H), 1.49-1.67 (m, 4H), 1.67-1.79 (m, 6H), 1.94-2.08 (m, 4H), 2.38 (dd, J=12.60, 9.28 Hz, 1H), 2.53 (d, J=11.33 Hz, 1H), 2.63 (d, J=6.45 Hz, 1H), 3.03 (d, J=10.35 Hz, 1H), 3.12 (d, J=5.08 Hz, 2H), 3.14-3.26 (m, 3H), 3.37-3.53 (m, 6H), 6.29 (d, J=8.79 Hz, 1H), 7.86 (dd, J=8.79, 2.15 Hz, 1H), 8.45 (s, 1H), 8.62 (d, J=1.76 Hz, 1H)

Isomer 2: N-((1S,2R)-2-{[(3R)-3-(ethoxymethyl)piperidin-1-yl]methyl}cyclohexyl)-6-pyrrolidin-1-ylnicotinamide 10 mg (16%) was obtained as its free base. MS (M+1): 429.3. ¹H NMR (400 MHz, CDCl₃) δ ppm 0.77-0.99 (m, 1H), 1.00-1.16 (m, 2H), 1.21 (t, J=6.74 Hz, 3H), 1.26-1.57 (m, 6H), 1.60-1.79 (m, 6H), 1.82-1.98 (m, 2H), 1.95-2.07 (m, 4H), 2.28-2.46 (m, 1H), 2.46-2.65 (m, 2H), 3.12-3.39 (m, 3H), 3.38-3.55 (m, 6H), 6.29 (d, J=8.79 Hz, 1H), 7.86 (d, J=7.23 Hz, 1H), 8.55 (s, 1H), 8.64 (s, 1H)

### Example 199

### N-[(1S,2R)-2-(piperidin-1-ylmethyl)cyclohexyl]-4-(1H-pyrazol-1-yl)benzamide

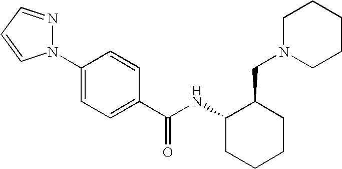

A solution of trans-(+/−)-N-[2-(piperidin-1-ylmethyl)cyclohexyl]-4-(1H-pyrazol-1-yl)benzamide (0.050 g, 0.14 mmol) in EtOH (2.5 mL) was subjected to preparative scale chiral phase HPLC (ChiralPak AD column, 21×250 mm, 20 □m, 15% EtOH/85% Hexanes with 0.1% diethylamine modifier, 18 mL/min flow rate). Fractions of the first eluting enantiomer were collected, concentrated, and lyophilized from CH₃CN/H₂O to give the title compound as a white solid (23 mg, 45%). MS (M+1): 367.3. ¹H NMR (400 MHz, METHANOL-D4) δ ppm 1.04-1.15 (m, 1H), 1.24-1.84 (m, 13H), 1.90-1.99 (m, 1H), 2.08-2.21 (m, 2H), 2.28-2.50 (m, 4H), 3.59 (td, J=10.7, 4.1 Hz, 1H), 6.56 (dd, J=2.5, 2.0 Hz, 1H), 7.73-7.78 (m, 1H), 7.85-7.90 (m, 2H), 7.92-7.99 (m, 2H), 8.33 (dd, J=2.7, 0.6 Hz, 1H).

### Example 200

### N-[(1S,2R)-2-(piperidin-1-ylmethyl)cyclohexyl]-6-(1H-pyrazol-1-yl)nicotinamide

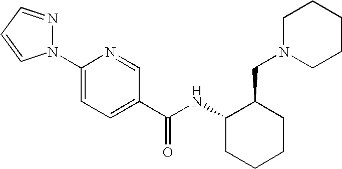 Method 1: Chiral Separation Approach

A solution of trans-(+/−)-N-[2-(piperidin-1-ylmethyl)cyclohexyl]-6-(1H-pyrazol-1-yl)nicotinamide (0.10 g, 0.27 mmol) in EtOH (2.5 mL) was subjected to preparative scale chiral phase HPLC (ChiralPak AD column, 21×250 mm, 20 □m, 15% EtOH/85% Hexanes with 0.1% diethylamine modifier, 18 mL/min flow rate). Fractions of the first eluting enantiomer were collected, concentrated, and lyophilized from CH₃CN/H₂O to give the title compound as an off-white solid (0.0372 g, 37%). MS (M+1): 368.3. ¹H NMR (400 MHz, METHANOL-D4) δ ppm 1.01-1.17 (m, 1H), 1.23-1.85 (m, 13H), 1.89-2.01 (m, 1H), 2.06-2.23 (m, 2H), 2.29-2.55 (m, 4H), 3.62 (td, J=10.7, 3.8 Hz, 1H), 6.56 (dd, J=2.6, 1.7 Hz, 1H), 7.79 (d, J=1.0 Hz, 1H), 8.02 (dd, J=8.6, 0.6 Hz, 1H), 8.32 (dd, J=8.8, 2.3 Hz, 1H), 8.65 (dd, J=2.5, 0.6 Hz, 1H), 8.87 (dd, J=2.2, 0.7 Hz, 1H). Anal. Calcd for C₂₁H₂₉N₅O.0.4H₂O: C, 67.32; H, 8.02; N, 18.69. Found: C, 67.34; H, 7.81; N, 18.52.

Method 2: Synthetic Approach from Chiral Starting Material

### Step A

### 9H-fluoren-9-ylmethyl[(1S,2S)-2-(hydroxymethyl)cyclohexyl]carbamate

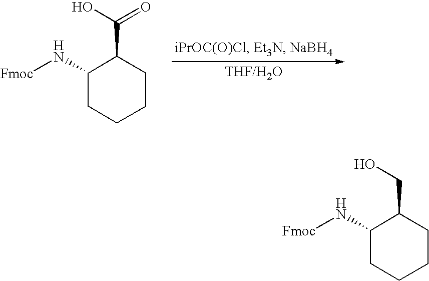

A solution of (1S,2S)-2-{[(9H-fluoren-9-ylmethoxy)carbonyl]amino}cyclohexane-carboxylic acid (0.948 g, 2.59 mmol) in THF (18 mL) was cooled to 0° C., and Et₃N (1.1 mL, 7.9 mmol) and isopropyl chloroformate (4.9 mL of 1M in toluene, 4.9 mmol) were added. The resulting solution was stirred for 10 min, and then a solution of NaBH₄ (0.353 g, 9.33 mmol) in H₂O (3.5 mL) was added. The mixture was stirred for 5 h, and additional NaBH₄ (0.050 g, 1.3 mmol) in H₂O (0.5 mL) was added. After stirring for an additional 30 min, a final portion of NaBH₄ (0.030 g, 0.79 mmol) in H₂O (0.3 mL) was added and the reaction stirred for a further 30 min. The reaction was then diluted with H₂O (50 mL) and extracted with CH₂Cl₂ (3×50 mL). The combined organic layers were dried over Na₂SO₄, filtered, and concentrated in vacuo. The residue was purified by column chromatography (95:5 CH₂Cl₂:MeOH) to provide the title compound as a white solid (0.711 g, 78%). MS (M+1): 352.2. ¹H NMR (400 MHz, CHLOROFORM-D) δ ppm 1.00-1.37 (m, 3H), 1.44-1.53 (m, 1H), 1.60-1.82 (m, 4H), 1.90-2.00 (m, 1H), 3.08-3.20 (m, 1H), 3.24-3.37 (m, 1H), 3.38-3.51 (m, 1H), 3.57-3.67 (m, 1H), 4.20 (t, J=6.4 Hz, 1H), 4.40 (dd, J=10.7, 6.4 Hz, 1H), 4.53 (dd, J=10.7, 6.6 Hz, 1H), 4.60 (d, J=9.2 Hz, 1H), 7.32 (td, J=7.4, 1.2 Hz, 2H), 7.36-7.45 (m, 2H), 7.58 (d, J=7.6 Hz, 2H), 7.71-7.80 (m, 2H).

### Step B

### tert-butyl[(1S,2S)-2-(hydroxymethyl)cyclohexyl]carbamate

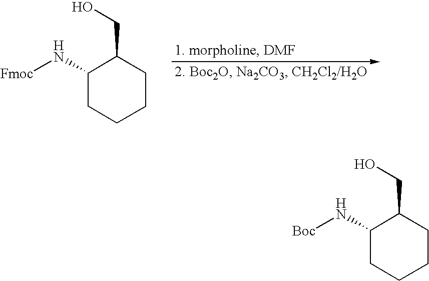

A mixture of 9H-fluoren-9-ylmethyl[(1S,2S)-2-(hydroxymethyl)cyclohexyl]carbamate (0.700 g, 1.99 mmol) and morpholine (11 mL) in DMF (11 mL) was stirred at room temperature for 30 min. The mixture was poured into H₂O (300 mL) in a separatory funnel and washed with hexanes (4×150 mL). The aqueous phase was then extracted with CH₂Cl₂ (4×150 mL). The combined CH₂Cl₂ extracts were dried over Na₂SO₄, filtered, and concentrated in vacuo. The residue was dissolved in CH₂Cl₂ (5 mL), and Na₂CO₃ (0.208 g, 1.96 mmol) dissolved in H₂O (10 mL) was added, followed by di-tert-butyl dicarbonate (0.393 g, 1.8 mmol) and additional CH₂Cl₂ (3 mL). The resulting mixture was stirred for 22 h. The layers were separated, and the aqueous phase was extracted with CH₂Cl₂ (3×15 mL). The combined organic layers were dried over Na₂SO₄, filtered, and concentrated in vacuo. The residue was purified by column chromatography (95:5 CH₂Cl₂:MeOH) to provide the title compound as a white solid (0.363 g, 79% over 2 steps). MS (M+1): 230.1. ¹H NMR (400 MHz, CHLOROFORM-D) δ ppm 0.98-1.39 (m, 4H), 1.44 (s, 9H), 1.47-1.58 (m, 1H), 1.61-1.82 (m, 3H), 1.87-2.01 (m, 1H), 3.23-3.46 (m, 2H), 3.49-3.60 (m, 1H), 3.69-3.80 (m, 1H), 4.43 (d, J=8.0 Hz, 1H).

### Step C

### tert-butyl[(1S,2S)-2-formylcyclohexyl]carbamate

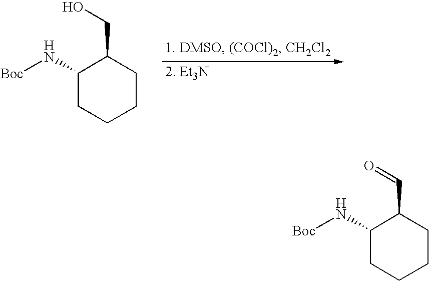

Oxalyl chloride (0.084 mL, 0.96 mmol) was added dropwise to a solution of dry DMSO (0.14 mL, 2.0 mmol) in dry CH₂Cl₂ (2 mL) cooled in a −78° C. cold bath. The resulting mixture was stirred for 10 min, and then a solution of tert-butyl[(1S,2S)-2-(hydroxymethyl)cyclohexyl]carbamate (0.148 g, 0.64 mmol) in CH₂Cl₂ (0.6 mL+2×0.3 mL) was added dropwise. After stirring an additional 10 min, Et₃N (0.36 mL, 2.6 mmol) was added dropwise. The reaction was stirred for 20 min at −78° C. and 1.5 h at 0° C. H₂O (5 mL) and CH₂Cl₂ (5 mL) were then added, the layers separated, and the aqueous phase was extracted with additional CH₂Cl₂ (3×5 mL). The combined organic layers were washed successively with a saturated solution of NH₄Cl (10 mL) and then brine (10 mL) before being dried over Na₂SO₄, filtered, and concentrated in vacuo to provide a sample of the title compound as a yellow solid (0.174 g, quantitative). The compound was used in subsequent steps without further purification. MS (M+1): 228.1.

### Step D

### [(1S,2R)-2-(piperidin-1-ylmethyl)cyclohexyl]amine hydrochloride salt

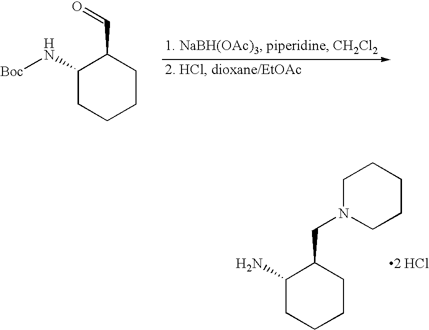

A mixture of crude tert-butyl[(1S,2S)-2-formylcyclohexyl]carbamate (0.081 g, ˜0.30 mmol) and piperidine (0.035 mL, 0.35 mmol) in dry CH₂Cl₂ (6 mL) was stirred for 30 min at 5° C. NaBH(OAc)₃ (0.127 g, 0.60 mmol) was added to the reaction and the resulting mixture was allowed to slowly warm to room temperature and stir for 14 h. The reaction was cooled to 0° C., and water (3 mL) was added, followed by 1 N NaOH (3 mL) and CH₂Cl₂ (10 mL). The layers were separated, and the aqueous phase was extracted with additional CH₂Cl₂ (2×10 mL). The combined organic layers were dried over Na₂SO₄, filtered, and concentrated in vacuo. The residue was dissolved in EtOAc (0.75 mL), and 4 N HCl in dioxane (0.75 mL, 3 mmol) was added. The mixture was stirred for 3 h and then concentrated in vacuo to provide the title compound. The compound was used in subsequent steps without further purification. MS (M+1): 197.1.

### Step E

### N-[(1S,2R)-2-(piperidin-1-ylmethyl)cyclohexyl]-6-(1H-pyrazol-1-yl)nicotin-amide

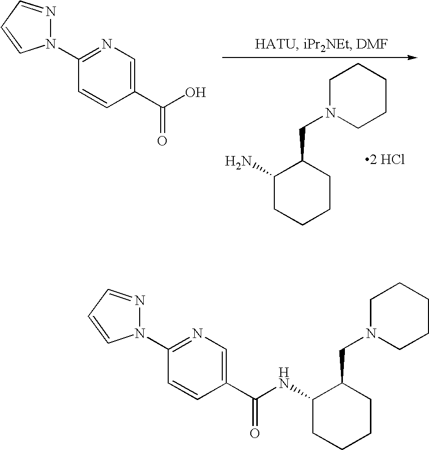

A mixture of 6-(1H-pyrazol-1-yl)nicotinic acid (0.0622 g, 0.33 mmol), HATU (0.125 g, 0.33 mmol), and diisopropylethylamine (0.073 mL, 0.42 mmol) in dry DMF (1 mL) was stirred at 0° C. for 10 min. A suspension of crude [(1S,2R)-2-(piperidin-1-ylmethyl)cyclohexyl]amine hydrochloride salt (˜0.30 mmol) and diisopropylethylamine (0.14 mL, 0.80 mmol) in DMF (0.5 mL+2×0.5 mL) was then added to the reaction, and the resulting mixture was stirred at 0° C. for 30 min and then warmed to room temperature and stirred for an additional 16 h. The reaction was concentrated in vacuo, and the residue was taken up into CH₂Cl₂ (5 mL) and a saturated solution of NaHCO₃ in water (5 mL). The mixture was passed through a Varian Chem Elut™ extraction cartridge, and the cartridge washed with additional CH₂Cl₂ (3×5 mL). The organic extract was concentrated in vacuo, and the residue was purified by preparative scale reverse phase LC/MS (gradient 55-75% CH₃CN in H₂O containing 10 mM NH₄HCO₃) to provide the title compound as a light yellow solid (0.0574 g, 52% over 3 steps) following lyophilization from CH₃CN/H₂O. MS (M+1): 368.3. ¹H NMR (400 MHz, CHLOROFORM-D) δ ppm 1.00-1.16 (m, 2H), 1.21-1.81 (m, 13H), 2.02-2.25 (m, 3H), 2.38 (dd, J=13.1, 10.0 Hz, 1H), 2.44-2.71 (m, 2H), 3.33-3.46 (m, 1H), 6.48 (dd, J=2.6, 1.7 Hz, 1H), 7.76 (dd, J=1.7, 0.7 Hz, 1H), 8.00 (dd, J=8.6, 0.8 Hz, 1H), 8.25 (dd, J=8.6, 2.3 Hz, 1H), 8.61 (dd, J=2.7, 0.8 Hz, 1H), 8.89 (dd, J=2.3, 0.8 Hz, 1H), 9.41 (s, 1H)

### Example 201

### N-((1S,2R)-2-{[(3R)-3-(allyloxy)piperidin-1-yl]methyl}cyclohexyl)-4-(1H-pyrrol-1-yl)benzamide

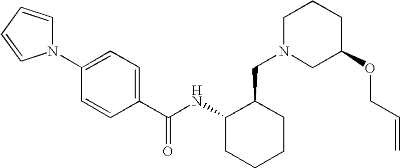

### Step A

### tert-butyl (3R)-3-hydroxypiperidine-1-carboxylate

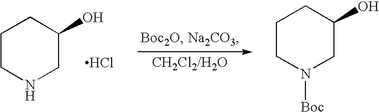

A suspension of (3R)-piperidin-3-ol hydrochloride salt (3.17 g, 0.023 mol) in CH₂Cl₂ (40 mL) was treated with Na₂CO₃ (5.13 g, 0.048 mol) dissolved in H₂O (80 mL), followed by di-tert-butyl dicarbonate (5.53 g, 0.025 mol) and additional CH₂Cl₂ (24 mL). The resulting mixture was stirred for 21 h. The layers were separated, and the aqueous phase was extracted with CH₂Cl₂ (3×50 mL). The combined organic layers were dried over Na₂SO₄, filtered, and concentrated in vacuo. The residue was purified by column chromatography (9:1 CH₂Cl₂:MeOH) to provide the title compound as a colorless oil (5.07 g, quantitative). MS (M+1): 202.0. ¹H NMR (400 MHz, CHLOROFORM-D) δ ppm 1.40-1.56 (m, 2H), 1.44 (s, 9H), 1.67-1.80 (m, 1H), 1.80-1.93 (m, 1H), 2.95-3.22 (m, 2H), 3.47 (d, J=5.1 Hz, 1H), 3.51 (br s, 1H), 3.64-3.78 (m, 2H).

### Step B

### (3R)-3-(allyloxy)piperidine hydrochloride salt

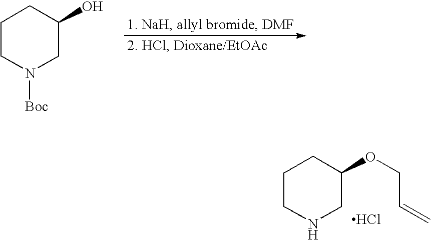

NaH (0.60 g of 60% in oil, 15 mmol) washed with hexanes (2×10 mL), and then suspended in dry DMF (12 mL) and cooled to 0° C. A solution of tert-butyl (3R)-3-hydroxypiperidine-1-carboxylate (1.51 g, 7.5 mmol) in dry DMF (6 mL+2×2 mL) was slowly added, and the resulting mixture was stirred for 30 min at 0° C. Allyl bromide (0.78 mL, 9.0 mmol) was added, and the reaction was allowed to warm to room temperature and stir for 13 h. The reaction was cooled to 0° C., H₂O (2 mL) was added, and then the reaction was concentrated in vacuo. The residue was partitioned between CH₂Cl₂ (50 mL) and H₂O (25 mL). The layers were separated, and the aqueous layer was extracted with additional CH₂Cl₂ (2×25 mL). The combined organic layers were washed with brine (2×25 mL) and then dried over Na₂SO₄, filtered, and concentrated in vacuo. The residue was dissolved in EtOAc (9 mL), and 4 N HCl in dioxane (8.9 mL, 36 mmol) was added. The mixture was stirred for 3 h and then concentrated in vacuo. The resulting solid washed with Et₂O and dried in vacuo to provide the title compound (1.19 g, 89% over 2 steps) as a hygroscopic light orange solid. The compound was used in subsequent steps without further purification. MS (M+1): 142.0.

### Step C

### ((1R,2S)-2-{[(3R)-3-(allyloxy)piperidin-1-yl]methyl}cyclohexyl)amine hydrochloride salt and ((1S,2R)-2-{[(3R)-3-(allyloxy)piperidin-1-yl]methyl}cyclohexyl)-amine hydrochloride salt

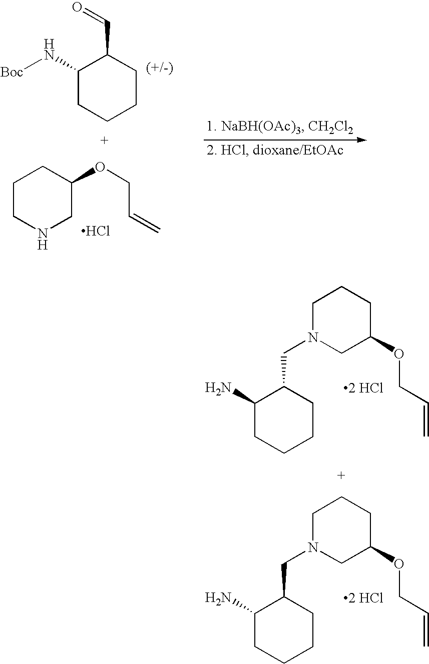

A mixture of crude tert-butyl[trans-(+/−)-2-formylcyclohexyl]carbamate (1.38 g, ˜6.1 mmol) and (3R)-3-(allyloxy)piperidine hydrochloride salt (1.19 g, 6.7 mmol) in dry CH₂Cl₂ (60 mL) was stirred for 30 min at room temperature. NaBH(OAc)₃ (2.58 g, 12 mmol) was added to the reaction and the resulting mixture was stirred for 16 h. The reaction was cooled to 0° C., and water (25 mL) was added, followed by 1 N NaOH (25 mL) and CH₂Cl₂ (60 mL). The layers were separated, and the aqueous phase was extracted with additional CH₂Cl₂ (2×60 mL). The combined organic layers were dried over Na₂SO₄, filtered, and concentrated in vacuo. The residue was dissolved in EtOAc (14 mL), and 4 N HCl in dioxane (14 mL, 56 mmol) was added. The mixture was stirred for 2 h and then concentrated in vacuo. The resulting oil was dissolved in CH₂Cl₂ and hexanes and concentrated in vacuo to give a light yellow foam. The foam was triturated with Et₂O twice and dried in vacuo to provide the title compound (1.89 g, 95% over two steps) as a yellow solid. The compound was used in subsequent steps without further purification. MS (M+1): 253.0.

### Step D

### N-((1R,2S)-2-{[(3R)-3-(allyloxy)piperidin-1-yl]methyl}cyclohexyl)-4-(1H-pyrrol-1-yl)benzamide and N-((1S,2R)-2-{[(3R)-3-(allyloxy)piperidin-1-yl]methyl}cyclohexyl)-4-(1H-pyrrol-1-yl)benzamide

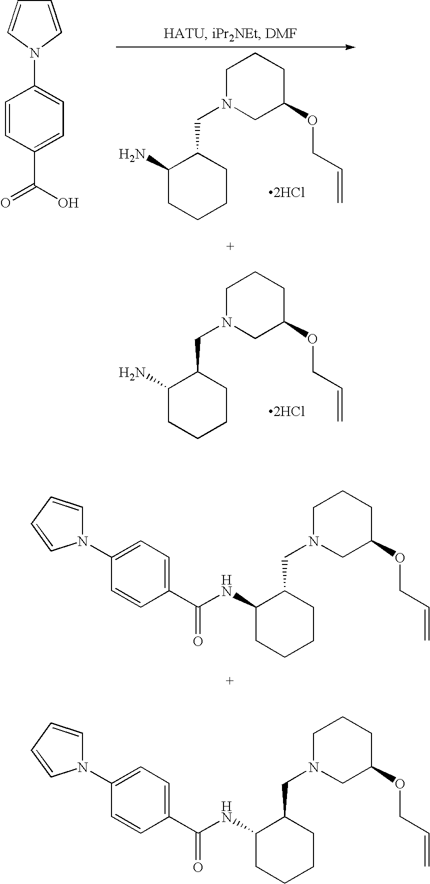

A mixture of 4-(1H-pyrrol-1-yl)benzoic acid (0.144 g, 0.77 mmol), HATU (0.293 g, 0.77 mmol), and diisopropylethylamine (0.17 mL, 0.98 mmol) in dry DMF (2 mL) was stirred at 0° C. for 10 min. A solution of a mixture of crude ((1R,2S)-2-{[(3R)-3-(allyloxy)piperidin-1-yl]methyl}cyclohexyl)amine hydrochloride salt and ((1S,2R)-2-{[(3R)-3-(allyloxy)piperidin-1-yl]methyl}cyclohexyl)-amine hydrochloride salt (0.228 g, ˜0.7 mmol) and diisopropylethylamine (0.32 mL, 1.8 mmol) in DMF (1+2×1 mL) was then added to the reaction, and the resulting mixture was stirred at 0° C. for 30 min and then warmed to room temperature and stirred for an additional 15 h. The reaction was concentrated in vacuo, and the residue was taken up into CH₂Cl₂ (8 mL) and a saturated solution of NaHCO₃ in water (8 mL). The mixture was passed through a Varian Chem Elut™ extraction cartridge, and the cartridge washed with additional CH₂Cl₂ (3×12 mL). The organic extract was concentrated in vacuo, and the residue was purified by preparative scale reverse phase LC/MS (gradient 55-75% CH₃CN in H₂O containing 10 mM NH₄HCO₃). The first stereoisomer of the product to elute, N-((1S,2R)-2-{[(3R)-3-(allyloxy)piperidin-1-yl]methyl}cyclohexyl)-4-(1H-pyrrol-1-yl)benzamide, was obtained as a white solid (0.0577 g, 20%) following lyophilization from CH₃CN/H₂O. MS (M+1): 422.2. ¹H NMR (400 MHz, CHLOROFORM-D) δ ppm 1.02-1.49 (m, 6H), 1.54-1.83 (m, 6H), 1.94-2.06 (m, 2H), 2.11 (dd, J=13.0, 1.5 Hz, 1H), 2.47 (dd, J=12.9, 9.2 Hz, 2H), 2.57-2.65 (m, 1H), 3.20-3.30 (m, 1H), 3.38-3.54 (m, 2H), 3.95-4.15 (m, 2H), 5.20 (ddd, J=10.4, 3.1, 1.4 Hz, 1H), 5.30 (ddd, J=17.2, 3.4, 1.7 Hz, 1H), 5.85-6.01 (m, 1H), 6.34-6.43 (m, 2H), 7.10-7.18 (m, 2H), 7.37-7.45 (m, 2H), 7.83-7.92 (m, 2H), 8.66 (d, J=2.9 Hz, 1H).

### Example 202

### N-((1S,2R)-2-{[(3R)-3-(allyloxy)piperidin-1-yl]-methyl}cyclohexyl)-3-cyclopentylpropanamide

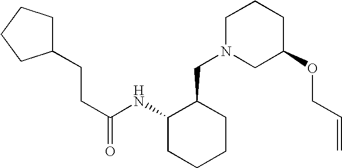

A mixture of 3-cyclopentylpropanoic acid (0.11 mL, 0.77 mmol), HATU (0.293 g, 0.77 mmol), and diisopropylethylamine (0.17 mL, 0.98 mmol) in dry DMF (2 mL) was stirred at 0° C. for 10 min. A solution of a mixture of crude ((1R,2S)-2-{[(3R)-3-(allyloxy)piperidin-1-yl]methyl}cyclohexyl)amine hydrochloride salt and ((1S,2R)-2-{[(3R)-3-(allyloxy)piperidin-1-yl]methyl}cyclohexyl)-amine hydrochloride salt (0.228 g, ˜0.7 mmol) and diisopropylethylamine (0.32 mL, 1.8 mmol) in DMF (1+2×1 mL) was then added to the reaction, and the resulting mixture was stirred at 0° C. for 30 min and then warmed to room temperature and stirred for an additional 15 h. The reaction was concentrated in vacuo, and the residue was taken up into CH₂Cl₂ (8 mL) and a saturated solution of NaHCO₃ in water (8 mL). The mixture was passed through a Varian Chem Elut™ extraction cartridge, and the cartridge washed with additional CH₂Cl₂ (3×12 mL). The organic extract was concentrated in vacuo, and the residue was purified by preparative scale reverse phase LC/MS (gradient 65-85% CH₃CN in H₂O containing 10 mM NH₄HCO₃). The first stereoisomer of the product to elute, N-((1S,2R)-2-{[(3R)-3-(allyloxy)piperidin-1-yl]-methyl}cyclohexyl)-3-cyclopentylpropanamide, was obtained as a slightly yellow oil (0.0361 g, 14%) following lyophilization from CH₃CN/H₂O. MS (M+1): 377.5. ¹H NMR (400 MHz, CHLOROFORM-D) δ ppm 0.87-1.88 (m, 23H), 1.93-2.21 (m, 5H), 2.32-2.46 (m, 2H), 2.50-2.62 (m, 1H), 3.02-3.13 (m, 1H), 3.21-3.33 (m, 1H), 3.35-3.46 (m, 1H), 3.96-4.12 (m, 2H), 5.18 (ddd, J=10.4, 2.9, 1.4 Hz, 1H), 5.29 (ddd, J=17.2, 3.4, 1.7 Hz, 1H), 5.82-6.00 (m, 1H), 7.54 (s, 1H).

### Example 203

### N-((1S,2R)-2-{[(3R)-3-(allyloxy)piperidin-1-yl]methyl}cyclohexyl)-6-(1H-pyrazol-1-yl)nicotinamide

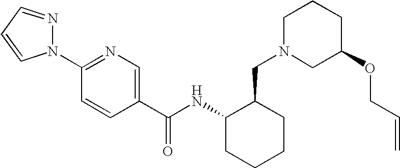

A mixture of 6-(1H-pyrazol-1-yl)nicotinic acid (0.146 g, 0.77 mmol), HATU (0.293 g, 0.77 mmol), and diisopropylethylamine (0.17 mL, 0.98 mmol) in dry DMF (2 mL) was stirred at 0° C. for 10 min. A solution of a mixture of crude ((1R,2S)-2-{[(3R)-3-(allyloxy)piperidin-1-yl]methyl}cyclohexyl)amine hydrochloride salt and ((1S,2R)-2-{[(3R)-3-(allyloxy)piperidin-1-yl]methyl}cyclohexyl)-amine hydrochloride salt (0.228 g, ˜0.7 mmol) and diisopropylethylamine (0.32 mL, 1.8 mmol) in DMF (1+2×1 mL) was then added to the reaction, and the resulting mixture was stirred at 0° C. for 20 min and then warmed to room temperature and stirred for an additional 14 h. The reaction was concentrated in vacuo, and the residue was taken up into CH₂Cl₂ (8 mL) and a saturated solution of NaHCO₃ in water (8 mL). The mixture was passed through a Varian Chem Elut™ extraction cartridge, and the cartridge washed with additional CH₂Cl₂ (3×12 mL). The organic extract was concentrated in vacuo, and the residue was purified by preparative scale reverse phase LC/MS (gradient 55-75% CH₃CN in H₂O containing 10 mM NH₄HCO₃). The first stereoisomer of the product to elute, N-((1S,2R)-2-{[(3R)-3-(allyloxy)piperidin-1-yl]methyl}cyclohexyl)-6-(1H-pyrazol-1-yl)nicotinamide, was obtained as a slightly orange solid (0.0627 g, 21%) following lyophilization from CH₃CN/H₂O. MS (M+1): 424.3. ¹H NMR (400 MHz, CHLOROFORM-D) δ ppm 1.00-1.48 (m, 6H), 1.52-1.85 (m, 6H), 1.89-2.18 (m, 3H), 2.34-2.53 (m, 2H), 2.62 (d, J=10.7 Hz, 1H), 3.19 (d, J=8.6 Hz, 1H), 3.36-3.53 (m, 2H), 3.86-4.15 (m, 2H), 5.16 (d, J=10.4 Hz, 1H), 5.27 (dd, J=17.1, 1.3 Hz, 1H), 5.80-5.98 (m, J=22.6, 10.7, 5.8 Hz, 1H), 6.42-6.54 (m, 1H), 7.76 (d, J=0.8 Hz, 1H), 7.99 (d, J=8.6 Hz, 1H), 8.21 (dd, J=8.5, 2.1 Hz, 1H), 8.60 (d, J=2.3 Hz, 1H), 8.84 (d, J=1.6 Hz, 1H), 8.90 (s, 1H). Anal. Calcd for C₂₄H₃₃N₅O₂.0.1H₂O: C, 67.77; H, 7.87; N, 16.46. Found: C, 67.84; H, 7.79; N, 16.43.

### Example 204

### N-((1S,2R)-2-{[(3S)-3-(allyloxy)piperidin-1-yl]methyl}cyclohexyl)-6-(1H-pyrazol-1-yl)nicotinamide

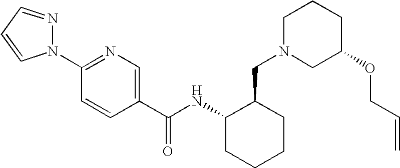

### Step A

### (3S)-3-(allyloxy)piperidine hydrochloride salt

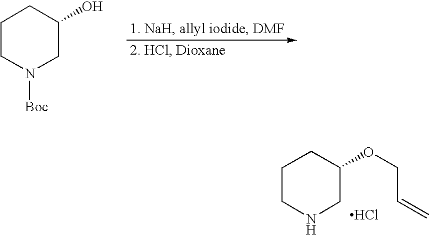

NaH (0.20 g of 60% in oil, 5.0 mmol) was added in portions to a solution of tert-butyl (3S)-3-hydroxypiperidine-1-carboxylate (0.514 g, 2.6 mmol) dissolved in dry DMF. The resulting mixture was stirred for 30 min, and then allyl iodide (0.3 mL, 2.5 mmol) was added, and the reaction was stirred for 2 h. The reaction was cooled to 0° C., H₂O was added, and then the reaction was concentrated in vacuo. The residue was partitioned between CH₂Cl₂ and H₂O. The layers were separated, and the aqueous layer was extracted with additional CH₂Cl₂. The combined organic layers were dried over Na₂SO₄, filtered, and concentrated in vacuo. The residue was dissolved in 4 N HCl in dioxane (3.8 mL, 15 mmol). The mixture was stirred for 16 h and then concentrated in vacuo. The compound was used in subsequent steps without further purification. MS (M+1): 142.1.

### Step B

### ((1S,2R)-2-{[(3S)-3-(allyloxy)piperidin-1-yl]methyl}cyclohexyl)amine hydrochloride salt

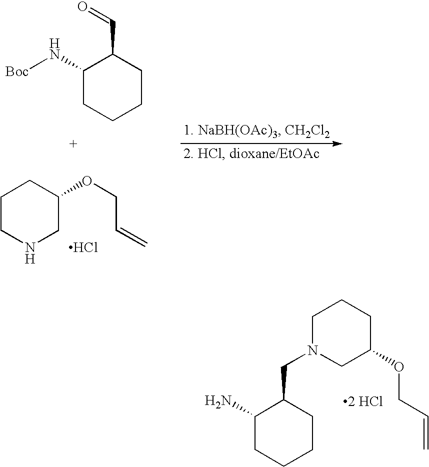

A mixture of crude tert-butyl[(1S,2S)-2-formylcyclohexyl]carbamate (0.0770 g, ˜0.3 mmol) and (3S)-3-(allyloxy)piperidine hydrochloride salt (0.0640 g, 0.36 mmol) in dry CH₂Cl₂ (6 mL) was stirred for 30 min at 5° C. NaBH(OAc)₃ (0.127 g, 0.6 mmol) was added to the reaction and the resulting mixture was allowed to warm to room temperature and stirred for 14 h. The reaction was cooled to 0° C., and water (3 mL) was added, followed by 1 N NaOH (3 mL) and CH₂Cl₂ (10 mL). The layers were separated, and the aqueous phase was extracted with additional CH₂Cl₂ (2×10 mL). The combined organic layers were dried over Na₂SO₄, filtered, and concentrated in vacuo. The residue was dissolved in EtOAc (0.75 mL), and 4 N HCl in dioxane (0.75 mL, 3 mmol) was added. The mixture was stirred for 1.5 h and then concentrated in vacuo to provide the title compound, which was used in subsequent steps without further purification. MS (M+1): 253.2.

### Step C

### N-((1S,2R)-2-{[(3S)-3-(allyloxy)piperidin-1-yl]methyl}cyclohexyl)-6-(1H-pyrazol-1-yl)nicotinamide

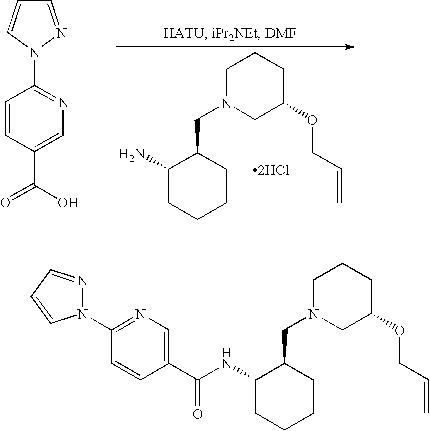

A mixture of 6-(1H-pyrazol-1-yl)nicotinic acid (0.0624 g, 0.33 mmol), HATU (0.126 g, 0.33 mmol), and diisopropylethylamine (0.073 mL, 0.42 mmol) in dry DMF (1 mL) was stirred at 0° C. for 10 min. A solution of crude ((1S,2R)-2-{[(3S)-3-(allyloxy)piperidin-1-yl]methyl}cyclohexyl)amine hydrochloride salt (˜0.3 mmol) and diisopropylethylamine (0.14 mL, 0.8 mmol) in DMF (0.5+2×0.5 mL) was then added to the reaction, and the resulting mixture was stirred at 0° C. for 30 min and then warmed to room temperature and stirred for an additional 21 h. The reaction was concentrated in vacuo, and the residue was taken up into CH₂Cl₂ (4 mL) and a saturated solution of NaHCO₃ in water (4 mL). The mixture was passed through a Varian Chem Elut™ extraction cartridge, and the cartridge washed with additional CH₂Cl₂ (3×8 mL). The organic extract was concentrated in vacuo, and the residue was purified by preparative scale reverse phase LC/MS (gradient 55-75% CH₃CN in H₂O containing 10 mM NH₄HCO₃) to provide the title compound as a white solid (0.0656 g, 52% over 3 steps) following lyophilization from CH₃CN/H₂O. MS (M+1): 424.3. ¹H NMR (400 MHz, CHLOROFORM-D) δ ppm 0.94-1.85 (m, 12H), 1.97-2.79 (m, 7H), 3.17-3.58 (m, 2H), 3.73-4.04 (m, 2H), 4.99 (d, J=10.4 Hz, 1H), 5.13 (d, J=17.4 Hz, 1H), 5.67-5.93 (m, 1H), 6.47 (dd, J=2.6, 1.7 Hz, 1H), 7.75 (d, J=1.0 Hz, 1H), 7.98 (dd, J=8.6, 0.4 Hz, 1H), 8.37 (dd, J=8.7, 1.9 Hz, 1H), 8.60 (dd, J=2.5, 0.6 Hz, 1H), 8.95 (d, J=1.0 Hz, 1H), 9.27 (s, 1H). Anal. Calcd for C₂₄H₃₃N₅O₂.0.2H₂O: C, 67.48; H, 7.88; N, 16.39. Found: C, 67.46; H, 7.65; N, 16.26.

### Example 205

### N-((1S,2R)-2-{[(3S)-3-(ethoxymethyl)piperidin-1-yl]methyl}cyclo-hexyl)-4-(2-methoxyethoxy)benzamide

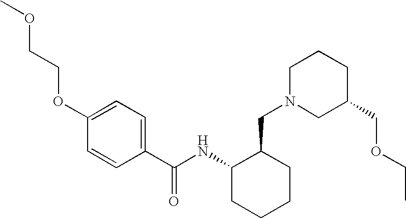

### Step A

### (3S)-3-(ethoxymethyl)piperidine hydrochloride salt

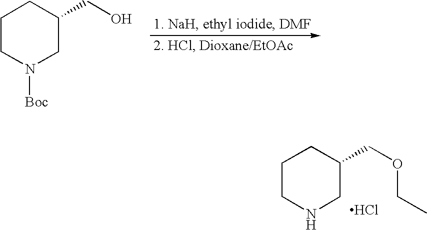

NaH (0.271 g of 60% in oil, 6.8 mmol) washed with hexanes (2×10 mL), and then suspended in dry DMF (6 mL) and cooled to 0° C. A solution of tert-butyl (3S)-3-(hydroxymethyl)piperidine-1-carboxylate (0.730 g, 3.4 mmol) in dry DMF (3 mL+2×1 mL) was slowly added, and the resulting mixture was stirred for 30 min at 0° C. Ethyl iodide (0.33 mL, 4.1 mmol) was added, and the reaction was allowed to warm to room temperature and stir for 40 h. The reaction was cooled to 0° C., H₂O (1 mL) was added, and then the reaction was concentrated in vacuo. The residue was partitioned between CH₂Cl₂ (25 mL) and H₂O (15 mL). The layers were separated, and the aqueous layer was extracted with additional CH₂Cl₂ (2×15 mL). The combined organic layers were washed with brine (2×15 mL) and then dried over Na₂SO₄, filtered, and concentrated in vacuo. The residue was dissolved in EtOAc (5 mL), and 4 N HCl in dioxane (4.3 mL, 17 mmol) was added. The mixture was stirred for 16 h and then concentrated in vacuo. The resulting solid washed with Et₂O and dried in vacuo to provide the title compound (0.725 g, quantitative over 2 steps) as a white solid. The compound was used in subsequent steps without further purification. MS (M+1): 144.1.

### Step B

### ((1S,2R)-2-{[(3S)-3-(ethoxymethyl)piperidin-1-yl]methyl}cyclohexyl)amine hydrochloride salt

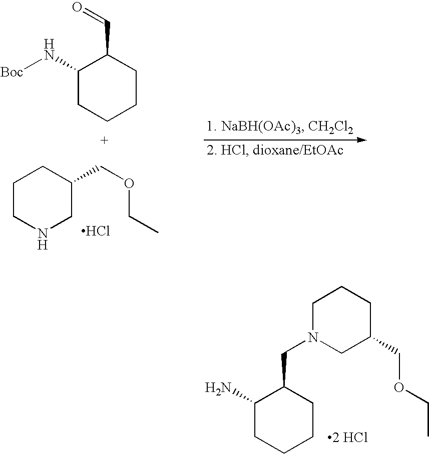

A mixture of crude tert-butyl[(1S,2S)-2-formylcyclohexyl]carbamate (0.316 g, ˜1.2 mmol) and (3S)-3-(ethoxymethyl)piperidine hydrochloride salt (0.315 g, ˜1.5 mmol) in dry CH₂Cl₂ (24 mL) was stirred for 30 min at 5° C. NaBH(OAc)₃ (0.521 g, 2.5 mmol) was added to the reaction and the resulting mixture was allowed to warm to room temperature and stirred for 15 h. The reaction was cooled to 0° C., and water (12 mL) was added, followed by 1 N NaOH (12 mL) and CH₂Cl₂ (40 mL). The layers were separated, and the aqueous phase was extracted with additional CH₂Cl₂ (2×40 mL). The combined organic layers were dried over Na₂SO₄, filtered, and concentrated in vacuo. The residue was dissolved in EtOAc (3 mL), and 4 N HCl in dioxane (3 mL, 12 mmol) was added. The mixture was stirred for 6 h and then concentrated in vacuo to provide the title compound, which was used in subsequent steps without further purification. MS (M+1): 255.2.

### Step C

### N-((1S,2R)-2-{[(3S)-3-(ethoxymethyl)piperidin-1-yl]methyl}cyclo-hexyl)-4-(2-methoxyethoxy)benzamide

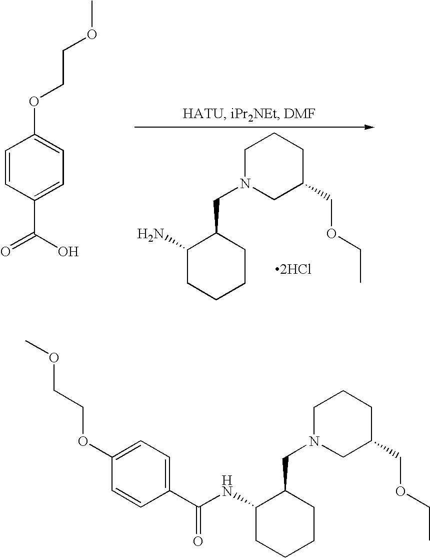

A mixture of 4-(2-methoxyethoxy)benzoic acid (0.0669 g, 0.34 mmol), crude ((1S,2R)-2-{[(3S)-3-(ethoxymethyl)piperidin-1-yl]methyl}cyclohexyl)amine hydrochloride salt (˜0.31 mmol), and diisopropylethylamine (0.14 mL, 0.80 mmol) in dry DMF (2 mL) was cooled to 0° C., and HATU (0.130 g, 0.34 mmol) in dry DMF (0.5 mL) was added. Additional diisopropylethylamine (0.073 mL, 0.42 mmol) was then added, and the resulting mixture was stirred at 0° C. for 30 min and then warmed to room temperature and stirred for an additional 15 h. The reaction was concentrated in vacuo, and the residue was taken up into CH₂Cl₂ (4 mL) and a saturated solution of NaHCO₃ in water (4 mL). The mixture was passed through a Varian Chem Elut™ extraction cartridge, and the cartridge washed with additional CH₂Cl₂ (3×8 mL). The organic extract was concentrated in vacuo, and the residue was purified by preparative scale reverse phase LC/MS (gradient 55-75% CH₃CN in H₂O containing 10 mM NH₄HCO₃) to provide the title compound as a slightly yellow oil (0.0410 g, 31% over 3 steps) following lyophilization from CH₃CN/H₂O. MS (M+1): 433.3. ¹H NMR (400 MHz, CHLOROFORM-D) δ ppm 0.84-0.98 (m, 1H), 0.98-1.12 (m, 4H), 1.14-1.45 (m, 2H), 1.47-1.82 (m, 11H), 2.02 (dd, J=12.8, 1.7 Hz, 1H), 2.39 (dd, J=12.9, 9.4 Hz, 1H), 2.49-2.67 (m, 2H), 2.96-3.28 (m, 5H), 3.34-3.43 (m, 1H), 3.44 (s, 3H), 3.71-3.78 (m, 2H), 4.09-4.17 (m, 2H), 6.87-6.94 (m, 2H), 7.73-7.80 (m, 2H), 8.68 (d, J=2.3 Hz, 1H). Anal. Calcd for C₂₅H₄₀N₂O₄.0.5H₂O: C, 68.00; H, 9.36; N, 6.34. Found: C, 67.93; H, 9.28; N, 6.64.

### Example 206

### 3-(4-chlorophenyl)-N-((1S,2R)-2-{[(3S)-3-(ethoxymethyl)piperidin-1-yl]methyl}cyclohexyl)propanamide

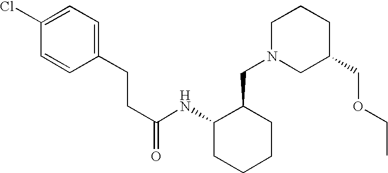

A mixture of 3-(4-chlorophenyl)propanoic acid (0.0630 g, 0.34 mmol), crude ((1S,2R)-2-{[(3S)-3-(ethoxymethyl)piperidin-1-yl]methyl}cyclohexyl)amine hydrochloride salt (˜0.31 mmol), and diisopropylethylamine (0.14 mL, 0.80 mmol) in dry DMF (2 mL) was cooled to 0° C., and HATU (0.130 g, 0.34 mmol) in dry DMF (0.5 mL) was added. Additional diisopropylethylamine (0.073 mL, 0.42 mmol) was then added, and the resulting mixture was stirred at 0° C. for 30 min and then warmed to room temperature and stirred for an additional 15 h. The reaction was concentrated in vacuo, and the residue was taken up into CH₂Cl₂ (4 mL) and a saturated solution of NaHCO₃ in water (4 mL). The mixture was passed through a Varian Chem Elut™ extraction cartridge, and the cartridge washed with additional CH₂Cl₂ (3×8 mL). The organic extract was concentrated in vacuo, and the residue was purified by preparative scale reverse phase LC/MS (gradient 65-85% CH₃CN in H₂O containing 10 mM NH₄HCO₃) to provide the title compound as a slightly yellow oil (0.0455 g, 35% over 3 steps) following lyophilization from CH₃CN/H₂O. MS (M+1): 421.3. ¹H NMR (400 MHz, CHLOROFORM-D) δ ppm 0.83-1.06 (m, 3H), 1.14 (t, J=7.0 Hz, 3H), 1.17-1.89 (m, 12H), 1.98 (dd, J=12.5, 1.8 Hz, 1H), 2.25 (dd, J=12.7, 9.2 Hz, 1H), 2.32-2.46 (m, 3H), 2.65 (d, J=8.6 Hz, 1H), 2.77-2.98 (m, 3H), 3.13-3.26 (m, 3H), 3.33-3.44 (m, 2H), 7.10-7.17 (m, 2H), 7.18-7.24 (m, 2H), 8.03 (d, J=2.9 Hz, 1H). Anal. Calcd for C₂₄H₃₇ClN₂O₂: C, 68.47; H, 8.86; N, 6.65. Found: C, 68.21; H, 8.88; N, 6.41.

### Example 207

### N-((1S,2R)-2-{[(3S)-3-(ethoxymethyl)piperidin-1-yl]methyl}cyclo-hexyl)-4-{[(methylsulfonyl)amino]methyl}benzamide

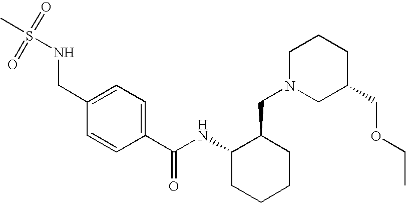

### Step A

### 4-{[(methylsulfonyl)amino]methyl}benzoic acid

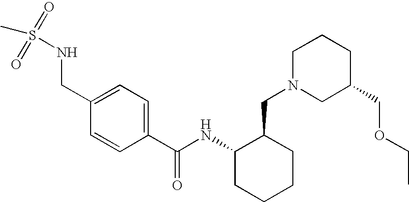

A suspension of methyl 4-(aminomethyl)benzoate hydrochloride salt (0.541 g, 2.7 mmol) in dry CH₂Cl₂ (7 mL) was cooled to 0° C., and methanesulfonyl chloride (0.48 mL, 6.2 mmol) and diisopropylethylamine (1.5 mL, 8.8 mmol) were added. The resulting mixture was allowed to warm to room temperature and stir for 15 h. The reaction was then diluted with CH₂Cl₂ (10 mL) and washed with H₂O (10 mL), a saturated aqueous solution of NaHCO₃ (10 mL), and brine (10 mL) successively. The organic layer was dried over Na₂SO₄, filtered, and concentrated in vacuo. The residue was dissolved in MeOH (14 mL), and NaOH (1.29 g, 32 mmol) dissolved in H₂O (7 mL) was added. The reaction was stirred for 16 h and was then concentrated in vacuo. The residue was dissolved in H₂O (10 mL) and acidified to pH 1 with 3 N HCl. The aqueous phase was extracted with EtOAc (3×50 mL), and the combined organic phases were dried over Na₂SO₄, filtered, and concentrated in vacuo to provide the title compound as a slightly yellow powder (0.60 g, 98% over 2 steps), which was used in subsequent steps without further purification. ¹H NMR (400 MHz, DMSO-D6) δ ppm 2.88 (s, 3H), 4.22 (d, J=6.2 Hz, 2H), 7.45 (d, J=8.6 Hz, 2H), 7.65 (t, J=6.3 Hz, 1H), 7.86-7.95 (m, 2H), 12.91 (s, 1H)

### Step B

### N-((1S,2R)-2-{[(3S)-3-(ethoxymethyl)piperidin-1-yl]methyl}cyclohexyl)-4-{[(methylsulfonyl)amino]methyl}benzamide

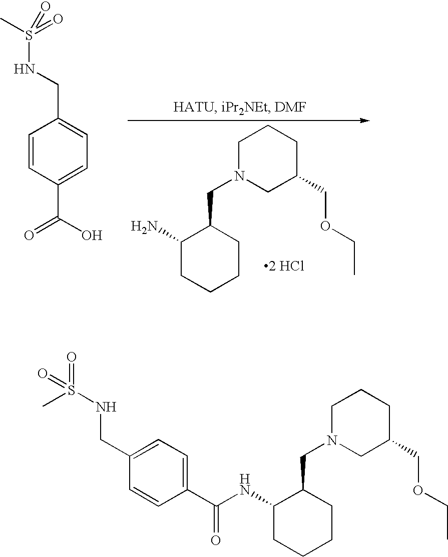

A mixture of 4-{[(methylsulfonyl)amino]methyl}benzoic acid (0.0782 g, 0.34 mmol), crude ((1S,2R)-2-{[(3S)-3-(ethoxymethyl)piperidin-1-yl]methyl}cyclohexyl)amine hydrochloride salt (˜0.31 mmol), and diisopropylethylamine (0.14 mL, 0.80 mmol) in dry DMF (2 mL) was cooled to 0° C., and HATU (0.130 g, 0.34 mmol) in dry DMF (0.5 mL) was added. Additional diisopropylethylamine (0.073 mL, 0.42 mmol) was then added, and the resulting mixture was stirred at 0° C. for 30 min and then warmed to room temperature and stirred for an additional 15 h. The reaction was concentrated in vacuo, and the residue was taken up into CH₂Cl₂ (4 mL) and a saturated solution of NaHCO₃ in water (4 mL). The mixture was passed through a Varian Chem Elut™ extraction cartridge, and the cartridge washed with additional CH₂Cl₂ (3×8 mL). The organic extract was concentrated in vacuo, and the residue was purified by preparative scale reverse phase LC/MS (gradient 45-65% CH₃CN in H₂O containing 10 mM NH₄HCO₃) to provide the title compound as a white solid (0.0453 g, 31% over 3 steps) following lyophilization from CH₃CN/H₂O. MS (M+1): 466.3. ¹H NMR (400 MHz, CHLOROFORM-D) δ ppm 0.73-0.94 (m, 1H), 0.97-1.14 (m, 5H), 1.18-1.81 (m, 13H), 2.03 (dd, J=12.9, 1.2 Hz, 1H), 2.35 (dd, J=12.9, 9.8 Hz, 1H), 2.46-2.64 (m, 2H), 2.88 (s, 3H), 2.93-3.10 (m, 3H), 3.25 (q, J=7.0 Hz, 2H), 3.39 (tt, J=10.6, 3.6 Hz, 1H), 4.21-4.42 (m, 2H), 7.38 (d, J=8.2 Hz, 2H), 7.73-7.83 (m, 2H), 8.94 (d, J=2.1 Hz, 1H).

### Example 208

### 4-[(diethylamino)methyl]-N-((1S,2R)-2-{[(3S)-3-(ethoxymethyl)-piperidin-1-yl]methyl}cyclohexyl)benzamide

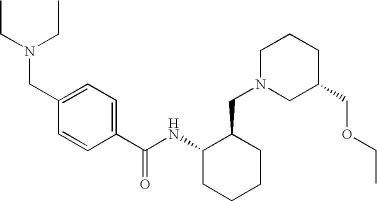

A mixture of 4-[(diethylamino)methyl]benzoic acid (0.0707 g, 0.34 mmol), crude ((1S,2R)-2-{[(3S)-3-(ethoxymethyl)piperidin-1-yl]methyl}cyclohexyl)amine hydrochloride salt (˜0.31 mmol), and diisopropylethylamine (0.14 mL, 0.80 mmol) in dry DMF (2 mL) was cooled to 0° C., and HATU (0.130 g, 0.34 mmol) in dry DMF (0.5 mL) was added. Additional diisopropylethylamine (0.073 mL, 0.42 mmol) was then added, and the resulting mixture was stirred at 0° C. for 30 min and then warmed to room temperature and stirred for an additional 15 h. The reaction was concentrated in vacuo, and the residue was taken up into CH₂Cl₂ (4 mL) and a saturated solution of NaHCO₃ in water (4 mL). The mixture was passed through a Varian Chem Elut™ extraction cartridge, and the cartridge washed with additional CH₂Cl₂ (3×8 mL). The organic extract was concentrated in vacuo, and the residue was purified by preparative scale reverse phase LC/MS (gradient 65-85% CH₃CN in H₂O containing 10 mM NH₄HCO₃) to provide the title compound as a yellow solid (0.0501 g, 36% over 3 steps) following lyophilization from CH₃CN/H₂O. MS (M+1): 444.5. ¹H NMR (400 MHz, CHLOROFORM-D) δ ppm 0.84-0.98 (m, 1H), 0.98-1.14 (m, 10H), 1.16-1.82 (m, 13H), 2.04 (dd, J=12.9, 1.4 Hz, 1H), 2.39 (dd, J=12.9, 9.4 Hz, 1H), 2.49 (q, J=7.1 Hz, 4H), 2.60 (t, J=9.8 Hz, 2H), 3.02 (d, J=10.9 Hz, 1H), 3.08 (d, J=6.4 Hz, 2H), 3.10-3.24 (m, 2H), 3.34-3.49 (m, 1H), 3.52-3.65 (m, 2H), 7.35 (d, J=8.4 Hz, 2H), 7.75 (d, J=8.4 Hz, 2H), 8.77 (s, 1H). Anal. Calcd for C₂₇H₄₅N₃O₂.0.3H₂O: C, 72.21; H, 10.23; N, 9.36. Found: C, 72.39; H, 10.21; N, 9.08.

### Example 209

### N-[(1S,2R)-2-({(3R)-3-[(allyloxy)methyl]piperidin-1-yl}methyl)cyclohexyl]-6-(1H-imidazol-1-yl)nicotinamide

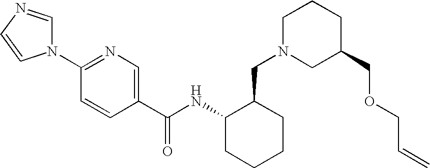

### Step A

### [(1S,2R)-2-({(3R)-3-[(allyloxy)methyl]piperidin-1-yl}methyl)cyclohexyl]amine hydrochloride salt

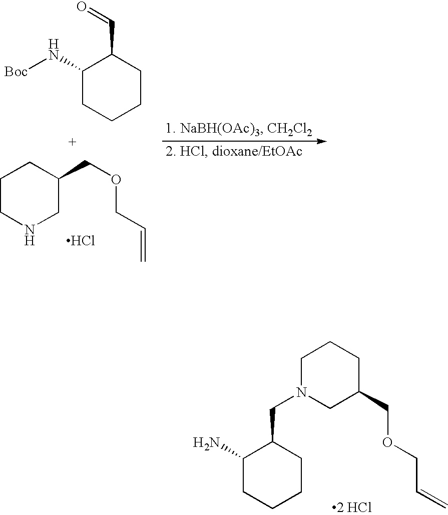

A mixture of crude tert-butyl[(1S,2S)-2-formylcyclohexyl]carbamate (1.95 g, 8.6 mmol) and (3R)-3-[(allyloxy)methyl]piperidine hydrochloride salt (2.08 g, 11 mmol) in dry CH₂Cl₂ (180 mL) was stirred for 30 min at 5° C. NaBH(OAc)₃ (3.64 g, 17 mmol) was added to the reaction and the resulting mixture was allowed to warm to room temperature and stirred for 15 h. The reaction was cooled to 0° C., and water (50 mL) was added, followed by 1 N NaOH (50 mL). The layers were separated, and the aqueous phase was extracted with additional CH₂Cl₂ (3×100 mL). The combined organic layers were dried over Na₂SO₄, filtered, and concentrated in vacuo. The intermediate, tert-butyl[(1S,2R)-2-({(3R)-3-[(allyloxy)methyl]piperidin-1-yl}methyl)cyclohexyl]carbamate, was obtained as a yellow oil (2.46 g, 78%) following purification by column chromatography (9:1 CH₂Cl₂:MeOH). MS (M+1): 367.3. The tert-butyl[(1S,2R)-2-({(3R)-3-[(allyloxy)methyl]piperidin-1-yl}methyl)cyclohexyl]-carbamate obtained above was dissolved in EtOAc (17 mL), and 4 N HCl in dioxane (17 mL, 68 mmol) was added. The mixture was stirred for 1.5 h and then concentrated in vacuo to provide the title compound (2.41 g, quantitative), which was used in subsequent steps without further purification. MS (M+1): 267.2.

### Step B

### N-[(1S,2R)-2-({(3R)-3-[(allyloxy)methyl]piperidin-1-yl}methyl)cyclohexyl]-6-(1H-imidazol-1-yl)nicotinamide

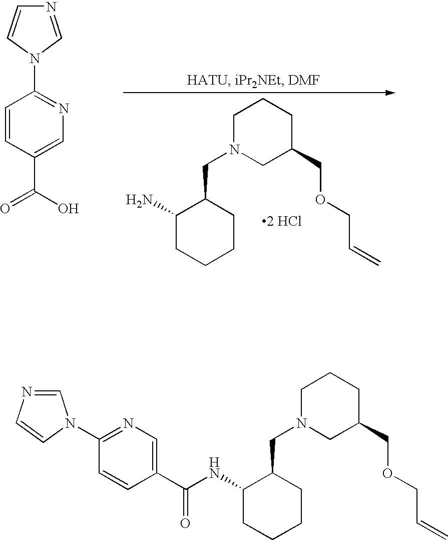

A mixture of 6-(1H-imidazol-1-yl)nicotinic acid (1.39 g, 7.4 mmol) and crude [(1S,2R)-2-({(3R)-3-[(allyloxy)methyl]piperidin-1-yl}methyl)cyclohexyl]amine hydrochloride salt (2.41 g, ˜6.7 mmol) in dry DMF (40 mL) was cooled to 0° C., and HATU (2.80 g, 7.4 mmol) and diisopropylethylamine (4.7 mL, 27 mmol) were added. The resulting mixture was allowed to slowly warm to room temperature and stirred for an additional 16 h. The reaction was concentrated in vacuo, and the residue was taken up into CH₂Cl₂ (80 mL) and a saturated solution of NaHCO₃ in water (80 mL). The layers were separated, and the aqueous phase was extracted with additional CH₂Cl₂ (3×60 mL). The combined organic phases were dried over Na₂SO₄, filtered, and concentrated in vacuo. The residue was purified by column chromatography (9:1 CH₂Cl₂:MeOH), and the product was then dissolved in CH₂Cl₂ and treated with 1 N HCl in ether (8 mL) to provide the title compound as its HCl salt (1.46 g, 43%) following lyophilization from H₂O. MS (M+1): 438.3. ¹H NMR (400 MHz, METHANOL-D4) δ ppm 1.16-2.37 (m, 14H), 2.75-2.91 (m, 1H), 2.96-3.09 (m, 1H), 3.15-3.29 (m, 2H), 3.42 (dd, J=9.6, 4.9 Hz, 1H), 3.48-3.76 (m, 3H), 3.76-3.87 (m, 1H), 3.89-4.05 (m, 2H), 5.08-5.19 (m, 1H), 5.20-5.30 (m, 1H), 5.78-5.95 (m, 1H), 7.78-7.84 (m, 1H), 8.06 (d, J=8.2 Hz, 1H), 8.39-8.48 (m, 1H), 8.62 (dd, J=8.6, 2.3 Hz, 1H), 9.10 (d, J=2.0 Hz, 1H), 9.86 (s, 1H).

### Example 210

### 4-chloro-N-((1S,2R)-2-{[(3R)-3-(ethoxymethyl)piperidin-1-yl]methyl}cyclohexyl)benzamide

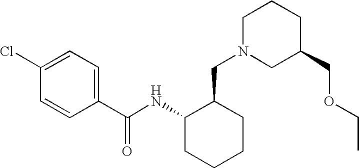

### Step A

### The Preparation of tert-butyl (3R)-3-(ethoxymethyl)piperidine-1-carboxylate

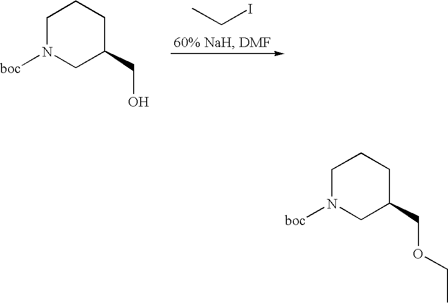

To a solution of tert-butyl (3R)-3-(hydroxymethyl)piperidine-1-carboxylate (568 mg, 2.63 mmol) in dry DMF (10 mL) was added NaH (60%, 200 mg, 5.26 mmol) at 0° C. under nitrogen and the suspension was stirred at room temperature for 30 min. ethyl iodide (0.51 mL, 6.32 mmol) was added to the reaction mixture and stirred over night at room temperature. Quenched with water. Extracted with dichloromethane (3×20 mL), washed with brine, dried over Na₂SO₄. Removal of solvent gave the crude product, which was used for the next step without further purification. MS (M+1): 244.2

### Step B

### The Preparation of (3R)-3-(ethoxymethyl)piperidine hydrochloride salt

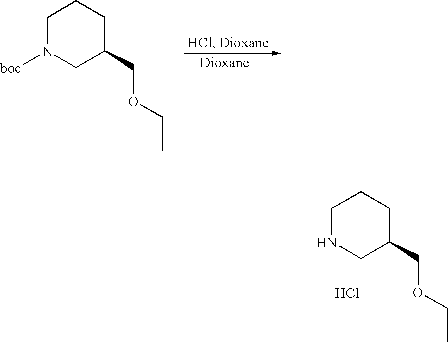

A 4N solution of hydrochloric acid in Dioxane (4.5 mL, 18.0 mmol) was added to a solution of the crude product from step A tert-butyl (3R)-3-(ethoxymethyl)piperidine-1-carboxylate (2.63 mmol) in Dioxane (5 mL). The reaction was stirred at room temperature for 5 hours. The mixture was concentrated in vacuo. The product was used directly for the next step without further purification. MS (M+1): 144.1 m: 477 mg

### Step C

### The Preparation of tert-butyl((1S,2R)-2-{[(3R)-3-(ethoxymethyl)piperidin-1-yl]methyl}cyclohexyl)carbamate

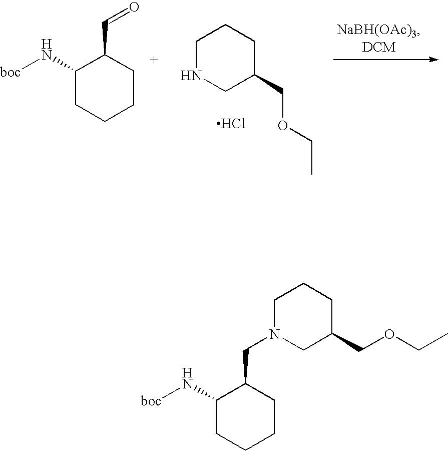

Crude product from step B (3R)-3-(ethoxymethyl)piperidine hydrochloride salt (340 mg, 1.89 mmol) was added to a solution of tert-butyl[(1S,2S)-2-formylcyclohexyl]carbamate (341 mg crude, 1.5 mmol) in dichloromethane (5 mL) at 0° C. The reaction was stirred at 0° C. for 30 min. and then sodium triacetoxyborohydride (636 mg, 3.0 mmol) was added to the reaction mixture. The reaction was stirred at 0° C. to room temperature, and stirred at room temperature for 3.5 h. Water (5 mL) was added dropwise. A 2N sodium hydroxide solution (10 mL) and dichloromethane (30 mL) were added to the mixture. The phases were separated and the aqueous was extracted with dichloromethane (2×15 mL). The combined organic phases were washed with brine, dried over Na₂SO₄, filtered, and concentrated in vacuo. MS (m+1): 355.4 The product was used directly for the next step without further purification.

### Step D

### The Preparation of ((1S,2R)-2-{[(3R)-3-ethoxypiperidin-1-yl]methyl}cyclohexyl)amine hydrochloride salt

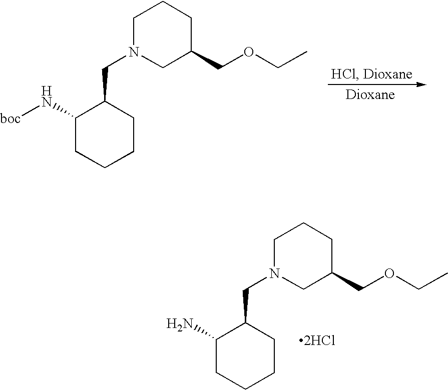

A 4N solution of hydrochloric acid in Dioxane (2.25 mL, 9.0 mmol) was added to a solution of the crude product from step C tert-butyl((1S,2R)-2-{[(3R)-3-(ethoxymethyl)piperidin-1-yl]methyl}cyclohexyl)carbamate (1.50 mmol) in Dioxane (5 mL). The reaction was stirred at room temperature for overnight. The mixture was concentrated in vacuo. The product was used directly for the next step without further purification. 572 mg MS (M+1): 255.3

### Step E

### The Preparation of 4-chloro-N-((1S,2R)-2-{[(3R)-3-(ethoxymethyl)piperidin-1-yl]methyl}cyclohexyl)benzamide

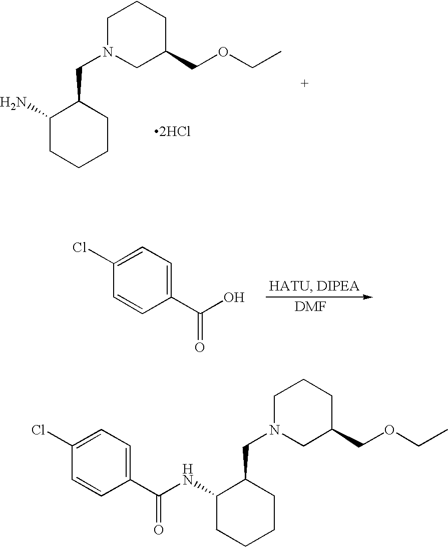

A solution of 4-chlorobenzoic acid (47 mg, 0.30 mmol), HATU (114 mg, 0.3 mmol) and diisopropylethylamine (0.07 mL, 0.40 mmol) in dry DMF (3 mL) was stirred at room temperature for 10 minutes. Crude ((1S,2R)-2-{[(3R)-3-ethoxypiperidin-1-yl]methyl}cyclohexyl)amine hydrochloride salt from step D (0.30 mmol) was added to the solution. The mixture was stirred at room temperature for overnight. The solvent was removed in vacuo. Residue was dissolved in DCM (15 mL) and washed with saturated NaHCO₃ (10 mL) and brine (10 mL), dried over Na₂SO₄. The crude product was purified with reverse phase HPLC High pH to yield 4-chloro-N-((1S,2R)-2-{[(3R)-3-(ethoxymethyl)piperidin-1-yl]methyl}cyclohexyl)benzamide 17.3 mg (15%) as its free base. MS (M+1): 393.3 ¹H NMR (400 MHz, CDCl₃) □ ppm 0.82-0.98 (m, J=9.37 Hz, 1H) 0.98-1.15 (m, J=9.18 Hz, 2H), 1.23 (t, J=7.03 Hz, 3H), 1.27-1.55 (m, 4H), 1.57-1.80 (m, 6H), 1.80-1.97 (m, 2H), 2.05 (d, J=11.91 Hz, 1H), 2.40 (s, 1H), 2.57 (s, 2H), 3.20 (t, J=8.50 Hz, 1H), 3.24-3.32 (m, J=10.16 Hz, 1H), 3.34 (dd, J=9.28, 5.18 Hz, 1H), 3.37-3.44 (m, J=11.72 Hz, 1H), 3.44-3.55 (m, 2H), 7.38 (d, J=8.40 Hz, 2H), 7.78 (d, J=7.81 Hz, 2H), 9.00 (s, 1H)

### Example 211

### N-((1S,2R)-2-{[(3R)-3-(ethoxymethyl)piperidin-1-yl]methyl}cyclohexyl)benzamide

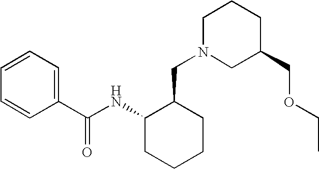

A mixture of benzoic acid (0.0148 g, 0.12 mmol) and crude ((1S,2R)-2-{[(3R)-3-(ethoxymethyl)piperidin-1-yl]methyl}cyclohexyl)amine hydrochloride salt (0.0351 g, ˜0.11 mmol) in dry DMF (1 mL) was cooled to 0° C., and HATU (0.0460 g, 0.12 mmol) and diisopropylethylamine (0.077 mL, 0.44 mmol) were added. The resulting mixture was allowed to slowly warm to room temperature and stirred for an additional 16 h. The reaction was concentrated in vacuo, and the residue was taken up into CH₂Cl₂ (2 mL) and a saturated solution of NaHCO₃ in water (2 mL). The mixture was passed through a Varian Chem Elut™ extraction cartridge, and the cartridge was washed with additional CH₂Cl₂ (3×6 mL). The organic extract was concentrated in vacuo, and the residue was purified by preparative scale reverse phase LC/MS (gradient 55-75% CH₃CN in H₂O containing 10 mM NH₄HCO₃) to provide the title compound as a yellow gum (0.0234 g, 59%) following lyophilization from CH₃CN/H₂O. MS (M+1): 359.4. ¹H NMR (400 MHz, CHLOROFORM-D) δ ppm 0.77-1.17 (m, 3H), 1.22 (t, J=7.0 Hz, 3H), 1.25-1.78 (m, 10H), 1.79-1.97 (m, J=11.3, 11.3 Hz, 2H), 2.02 (d, J=12.5 Hz, 1H), 2.39 (dd, J=12.1, 9.8 Hz, 1H), 2.48-2.66 (m, 2H), 3.18 (dd, J=9.3, 7.9 Hz, 1H), 3.25 (d, J=10.7 Hz, 1H), 3.31 (dd, J=9.4, 5.3 Hz, 1H), 3.36-3.54 (m, 3H), 7.33-7.50 (m, 3H), 7.82 (d, J=7.0 Hz, 2H), 8.89 (s, 1H). Anal. Calcd for C₂₂H₃₄N₂O₂.0.6H₂O: C, 71.55; H, 9.61; N, 7.58. Found: C, 71.74; H, 9.63; N, 7.36.

### Examples 212-231

Compounds Listed in the Following Table were Prepared as Described in Example 212

| Example | | | |
|---|---|---|---|
| No. | Structure | Name | Data |
| 212 | 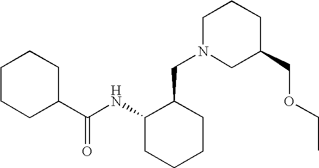 | N-((1S,2R)- 2-{[(3R)-3- (ethoxymeth- yl)piperidin-1- yl]methyl}cyclo- hexyl)cyclo- hexanecarbox- amide | ¹H NMR(400 MHz, CHLOROFORM-D) δ ppm 0.84- 1.09(m, 3H), 1.13-2.03(m, 27H), 2.32(dd, J=12.8, 9.1Hz, 1H), 2.36-2.45(m, 1H), 2.63- 2.75(m, |
| #1H), 3.07-3.26(m, 3H), 3.26-3.36(m, 1H), 3.41- 3.55(m, 2H), 7.76(s, 1H). MS: 365.3(M + 1) | | | |
| 213 | 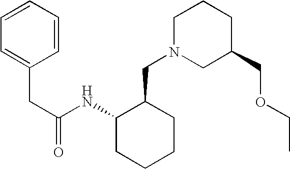 | N-((1S,2R)- 2-{[(3R)-3- (ethoxymeth- yl)piperidin-1- yl]methyl}cyclo- hexyl)-2- phenylaceta- mide | ¹H NMR(400 MHz, CHLOROFORM-D): δ ppm 0.84-1.06(m, 3H), 1.19(t, J=6.9Hz, 3H), 1.22-1.92(m, 12H), 2.00(dd, J=12.7, 2.9Hz, 1H), 2.20-2.39(m, |
| #2H), 2.61 (d, J=11.1Hz, 1H), 3.02(d, J=11.5Hz, 1H), 3.15-3.25 (m, 1H), 3.24-3.35(m, 2H), 3.39-3.53(m, 4H), 7.17-7.36 (m, 5H), 7.61(s, 1H). MS: 373.3(M + 1). | | | |
| 214 | 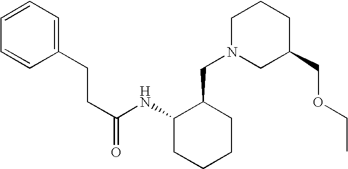 | N-((1S,2R)- 2-{[(3R)-3- (ethoxymeth- yl)piperidin-1- yl]methyl}cyclo- hexyl)-3- phenylpropan- amide | ¹H NMR(400 MHz, CHLOROFORM-D) δ ppm 0.77- 1.06(m, 3H), 1.19(t, J=7.0Hz, 3H), 1.22-2.01(m, 13H), 2.15-2.50(m, 5H), 2.82-3.03 (m, 2H), 3.07(d, J=9.6Hz, |
| #1H), 3.13-3.32(m, 3H), 3.37- 3.51(m, 2H), 7.11-7.21(m, 3H), 7.22-7.30(m, 2H), 8.00 (s, 1H). MS: (M + 1) 387.3. | | | |
| 215 | 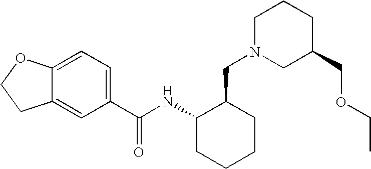 | N-((1S,2R)- 2-{[(3R)-3- (ethoxymeth- yl)piperidin-1- yl]methyl}cyclo- hexyl)-2,3- dihydro-1- benzofuran-5- carboxamide | 1H NMR(400 MHz, CHLOROFORM-D) δ ppm 0.80- 0.96(m, 1H), 0.97-1.13(m, 2H), 1.21(t, J=7.0Hz, 3H), 1.24-1.77(m, 10H), 1.81- 1.96(m, 2H), |
| #2.01(d, J=12.3Hz, 1H), 2.29-2.45(m, 1H), 2.56(d, J=11.1Hz, 2H), 3.11- 3.54(m, 8H), 4.61(t, J=8.8Hz, 2H), 6.74(d, J=8.4Hz, 1H), 7.60(d, J=8.0Hz, 1H), 7.73(s, 1H), 8.75(s, 1H), MS: M + 1) 401.4. | | | |
| 216 | 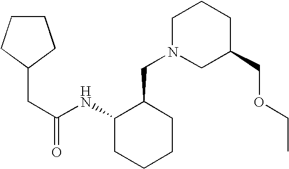 | 2-cyclopentyl- N-((1S,2R)- 2-{[(3R)-3- (ethoxymeth- yl)piperidin-1- yl]methyl}cyclo- hexyl)acet- amide | ¹H NMR(400 MHz, CHLOROFORM-D) δ ppm 0.83- 2.13(m, 29H), 2.16-2.28 (m, 1H), 2.33(dd, J=12.7, 9.2Hz, 1H), 2.37-2.47(m, 1H), 2.69(d, J=11.1Hz, 1H), |
| #3.10 (d, J=10.9Hz, 1H), 3.16-3.27 (m, 2H), 3.27-3.35(m, 1H), 3.40-3.54(m, 2h), 7.84(s, 1H). MS: (M + 1) 365.3. | | | |
| 217 | 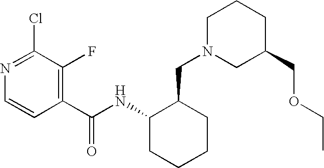 | 2-chloro-N- ((1S,2R)-2- {[(3R)-3- (ethoxymeth- yl)piperidin-1- yl]methyl}cyclo- hexyl)-3- fluoroisonico tinamide hydrochloride salt | ¹H NMR(400 MHz, METHANOL-D4) δ ppm 1.11- 1.21(m, 3H), 1.21-1.58(m, 5H), 1.71-2.17(m, 8H), 2.17- 2.34(m, |
| #1H), 2.70-2.91(m, 2H), 2.98-3.32(m, 3H), 3.38- 3.60(m, 4H), 3.65(d, J=10.9Hz, 1h), 3.70-3.81(m, 1H), 7.56-7.65(m, 1H), 8.28-8.35 (m 1H). MS: (M + 1) 412.3. | | | |
| 218 | 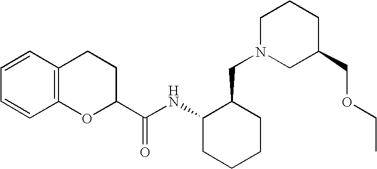 | N-((1S,2R)- 2-{[(3R)-3- (ethoxymeth- yl)piperidin-1- yl]methyl}cyclo- hexyl)chro- mane-2- carboxamide | ¹H NMR(400 MHz, METHANOL-D4) δ ppm 0.99- 1.59(m, 8H), 1.62-2.56(m, 12H), 2.68-3.15(m, 5H), 3.23-3.29(m, 1H), 3.32-3.72(m, 6H), 4.52-4.59(m, 0.5H), 4.65-4.74(m, |
| #0.5H), 6.82- 7.01(m, 2H), 7.02-7.20(m, 2H). MS: (M + 1) 415.3. | | | |
| 219 | 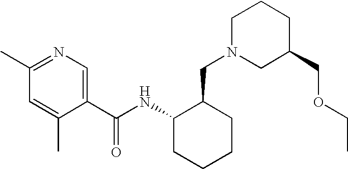 | N-((1S,2R)- 2-{[(3R)-3- (ethoxymeth- yl)piperidin-1- yl]methyl}cyclo- hexyl)-4,6- dimethylnico tinamide | ¹H NMR(400 MHz, METHANOL-D4) δ ppm 1.12- 1.22(m, 3H), 1.22-1.59(m, 5H), 1.73-2.14(m, 9H), 2.19- 2.34(m, 1H), 2.68(s, 3H), 2.77(s, 3H), 2.84-2.97(m, |
| #2H), 2.99-3.11(m, 1H), 3.24- 3.65(m, 6H), 3.70-3.82(m, 1H), 7.85(s, 1H), 8.86-8.91 (m, 1H). MS: (M + 1) 388.3. | | | |
| 220 | 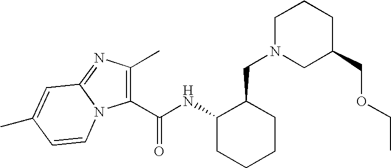 | N-((1S,2R)- 2-{[(3R)-3- (ethoxymeth- yl)piperidin-1- yl]methyl}cyclo- hexyl)-2,7- dimethylimidazo[1,2- a]pyridine-3- carboxamide | ¹H NMR(400 MHz, METHANOL-D4) δ ppm 1.11- 1.19(m, 3H), 1.22-1.70(m, 5H), 1.74-2.34(m, 9H), 2.61 (s, 3H), 2.76(s, 3H), |
| #2.77- 2.90(m, 2H), 3.02-3.12(m, 1H), 3.24-3.29(m, 1H), 3.32- 3.67(m, 6H), 3.85(td, J=10.9, 3.9Hz, 1H), 7.38-7.44(m, 1H), 7.66-7.73(m, 1H), 9.02- 9.09(m, 1H). MS: (M + 1) 427.2. | | | |
| 221 | 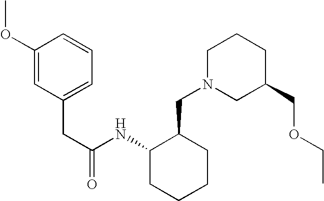 | N-((1S,2R)- 2-{[(3R)-3- (ethoxymeth- yl)piperidin-1- yl]methyl}cyclo- hexyl)-2-(3- methoxyphe- nyl)acetamide | ¹H NMR(400 MHz, METHANOL-D4) δ ppm 1.12- 1.19(m, 3H), 1.20-1.56(m, 5H), 1.63-2.24(m, 9H), 2.39(t, J=12.3Hz, 1H), 2.64-3.00 (m, 3H), |
| #3.24(dd, J=9.6, 6.8Hz, 1H), 3.33-3.55(m, 8H), 3.80(s, 3H), 6.78-6.89(m, 1H), 6.90-7.02(m, 2H), 7.26(t, J=7.8Hz, 1H). MS: (M + 1) 403.3. | | | |
| 222 | 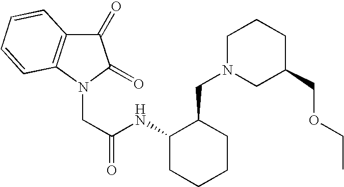 | 2-(2,3- dioxo-2,3- dihydro-1H- indol-1-yl)- N-((1S,2R)- 2-{[(3R)-3- (ethoxymeth- yl)piperidin-1- yl]methyl}cyclo- hexyl)ace- tamide | ¹H NMR(400 MHz, METHANOL-D4) δ ppm 1.13- 1.20(m, 3h), 1.20-1.60(m, 5H), 1.71-2.07(m, 9H), 2.11- 2.27(m, 1H), 2.62-3.21(m, |
| #3H), 3.34-3.68(m, 7H), 4.41- 4.54(m, 2H), 7.07-7.23(m, 2H), 7.58-7.72(m, 2H). MS: (M + 1) 442.3. | | | |
| 223 | 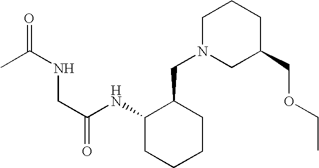 | N²-acetyl- N¹-((1S,2R)- 2-{[(3R)-3- (ethoxymeth- yl)piperidin-1- yl]methyl}cyclo- hexyl)gly- cinamide | ¹H NMR(400 MHz, METHANOL-D4) δ ppm 1.12- 1.19(m, 3H), 1.19-1.48(m, 5H), 1.68-2.11(m, 9H), 2.03 (s, 3H), 2.17-2.32(m, 1H), 2.74-2.95(m, 3H), |
| #3.16(dd, J=13.3, 2.7Hz, 1H), 3.25- 3.33(m, 1H), 3.38-3.66(m, 6H), 3.70-3.77(m, 1H), 3.78- 3.87(m, 1H). MS: (M + 1) 354.2. | | | |
| 224 | 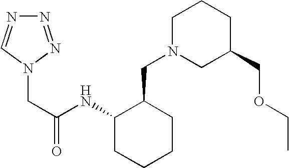 | N-((1S,2R)- 2-{[(3R)-3- (ethoxymeth- yl)piperidin-1- yl]methyl}cyclo- hexyl)-2- (1H-tetrazol-1- yl)acetamide | ¹H NMR(400 MHz, METHANOL-D4) δ ppm 1.12- 1.18(m, 3H), 1.21-1.50(m, 5H), 1.70-2.10(m, 9H), 2.15- 2.33(m, 1H), 2.76-2.90(m, 2H), 2.95(dd, J=13.3, 9.8Hz, |
| #1H), 3.16-3.24(m, 1H), 3.25- 3.33(m, 1H), 3.38-3.71(m, 5H), 5.26-5.34(m, 1H), 5.36- 5.45(m, 1H), 9.25(s, 1H). MS: (M + 1) 365.2. | | | |
| 225 | 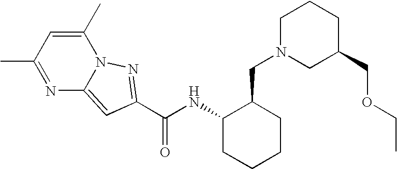 | N-((1S,2R)- 2-{[(3R)-3- (ethoxymeth- yl)piperidin-1- yl]methyl}cyclo- hexyl)-5,7- dimethylpyr- azolo[1,5- a]pyrimidine-2- carboxamide | ¹H NMR(400 MHz, METHANOL-D4) δ ppm 1.06- 1.17(m, 3H), 1.16-1.54(m, 4H), 1.57-1.71(m, 1H), 1.72- 2.30(m, 9H), 2.64(s, 3H), 2.68-2.88(m, |
| #4H), 2.97-3.27 (m, 3H), 3.32-3.57(m, 5H), 3.67(d, J=11.3Hz, 1H), 3.82 (td, J=10.8, 4.1Hz, 1H), 7.02- 7.05(m, 1H), 7.07(s, 1H). MS: (M + 1) 428.3. | | | |
| 226 | 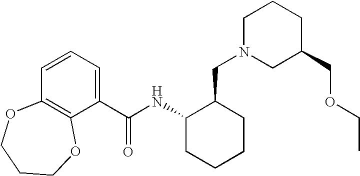 | N-((1S,2R)- 2-{[(3R)-3- (ethoxymeth- yl)piperidin-1- yl]methyl}cyclo- hexyl)- 3,4-dihydro- 2H-1,5- benzodioxe- pine-6- carboxamide | ¹H NMR(400 MHz, METHANOL-D4) δ ppm 1.11- 1.19(m, 3H), 1.19-1.65(m, 5H), 1.72-2.35(m, 11H), 2.67- 2.87(m, 2H), |
| #2.98-3.29(m, 3H), 3.33-3.59(m, 4H), 3.65 (d, J=11.3Hz, 1H), 3.76(td, J=10.6, 4.1Hz, 1H), 4.11- 4.26(m, 2H), 4.26-4.34(m, 2H), 6.97-7.04(m, 1H), 7.07- 7.13(m, 1H), 7.24-7.32(m, 1H). MS: (M + 1) 431.3. | | | |
| 227 | 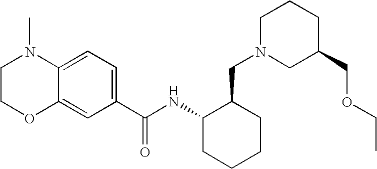 | N-((1S,2R)- 2-{[(3R)-3- (ethoxymeth- yl)piperidin-1- yl]methyl}cyclo- hexyl)-4- methyl-3,4- dihydro-2H- 1,4-benzoxazine-7- carboxamide | ¹H NMR(400 MHz, METHANOL-D4) δ ppm 1.10- 1.20(m, 3H), 1.20-1.63(m, 5H), 1.69-2.32(m, 9H), 2.66- 2.84(m, 2H), 2.91-3.03(m, 1H), 3.05(s, 3H), |
| #3.08-3.17(m, 1H), 3.24(dd, J=9.6, 7.2Hz, 1H), 3.34-3.55(m, 6H), 3.62(d, J=11.7Hz, 1H), 3.74(td, J=10.8, 4.1Hz, 1H), 4.25- 4.34(m, 2H), 6.84-6.94(m, 1H), 7.28-7.35(m, 1H), 7.41- 7.51(m, 1H). MS: (M + 1) 430.2. | | | |
| 228 | 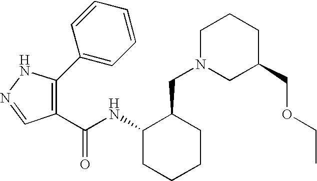 | N-((1S,2R)- 2-{[(3R)-3- (ethoxymeth- yl)piperidin-1- yl]methyl}cyclo- hexyl)-5- phenyl-1H- pyrazole-4- carboxamide | ¹H NMR(400 MHz, METHANOL-D4) δ ppm 1.11- 1.20(m, 3H), 1.21-1.47(m, 5H), 1.68-2.27(m, 9H), 2.61 (td, J=12.8, 3.3Hz, 1H), 2.74 (t, J=12.3Hz, 1H), 2.91-3.14 (m, |
| #2H), 3.26(dd, J=9.4, 7.0Hz, 1H), 3.35-3.51(m, 4H), 3.55(d, J=12.9Hz, 1H), 3.67 (td, J=10.4, 4.1Hz, 1H), 7.40- 7.51(m, 3H), 7.61-7.71(m, 2H), 8.13-8.19(m, 1H). MS: (M + 1) 425.2. | | | |
| 229 | 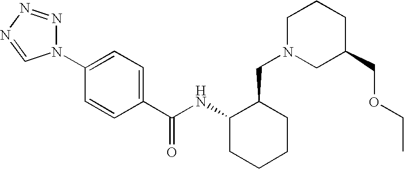 | N-((1S,2R)- 2-{[(3R)-3- (ethoxymeth- yl)piperidin-1- yl]methyl}cyclo- hexyl)-4- (1H-tetrazol-1- yl)benzamide | ¹H NMR(400 MHz, METHANOL-D4) δ ppm 1.08- 1.21(m, 3H), 1.21-1.68(m, 5H), 1.73-2.31(m, 9H), 2.71- 2.92(m, 2H), 3.00-3.28(m, 3H), 3.36-3.61(m, 4H), 3.66 (d, J=11.3Hz, |
| #1H), 3.81(td, J=10.8, 4.1Hz, 1H), 7.94- 8.07(m, 2H), 8.11-8.18(m, 2H), 9.83-9.90(m, 1H). MS: (M + 1) 427.2. | | | |
| 230 | 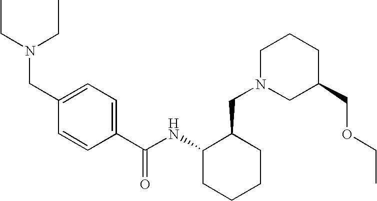 | 4- [(diethylami- no)methyl]- N-((1S,2R)- 2-{[(3R)-3- (ethoxymeth- yl)piperidin-1- yl]methyl}cyclo- hexyl)benz- amide | MS(M + 1): 444.5 ¹H NMR(400 MHz, CDCl₃) δ ppm 0.76-0.96 (m, 1H), 1.03(t, J=7.13Hz, 6H), 1.06-1.20(m, 2H), 1.23 (t, J=7.03Hz, 3H), |
| #1.27-1.51 (m, 4H), 1.53-1.69(m, 5H), 1.69-1.79(m, 2H), 1.88(t, J=11.33Hz, 2H), 2.03(d, J=12.89Hz, 1H), 2.39(dd, J=12.60, 9.67Hz, 1H), 2.50(q, J=7.10Hz, 4H), 2.55-2.65(m, 1H), 3.17-3.23(m, 1H), 3.26 (d, J=10.55Hz, 1H), 3.33(dd, J=9.28, 5.18Hz, 1H), 3.37- 3.44(m, 1H), 3.46-3.54(m, 2H), 3.59(s, 2H), 7.37(d, J=8.01Hz, 2H), 7.77(d, | | | |
| #J=8.01Hz, 2H), 8.85(s, 1H) | | | |
| 231 | 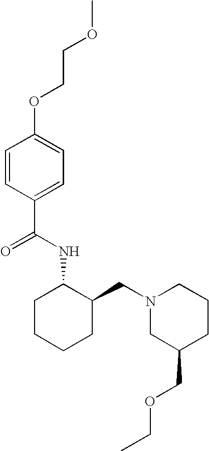 | N-((1S,2R)- 2-{[(3R)-3- (ethoxymeth- yl)piperidin-1- yl]methyl}cyclo- hexyl)-4-(2- methoxyeth- oxy)benzamide | MS(M + 1): 433.3 ¹H NMR (400 MHz, CDCl₃) δ ppm 1.17 (t, J=6.35Hz, 3H), 1.21-1.35 (m, 2H), 1.34-1.50(m, 3H), 1.58-1.83(m, 8H), 1.83-2.00 (m, 1H), 2.02-2.19(m, |
| #1H), 2.30-2.72(m, 3H), 3.10(dd, J=7.42, 3.91Hz, 1H), 3.13-3.24(m, 1H), 3.28(dd, J=9.57, 5.08Hz, 1H), 3.35-3.53 (m, 4H), 3.45(s, 3H), 3.52- 3.72(m, 1H), 3.73-3.79(m, 2H), 3.79-3.93(m, 1H), 6.97 (d, J=8.79Hz, 2H), 8.20(d, J=1.17Hz, 2H), 8.45(d, J=3.71Hz, 1H) | | | |

### Example 232

### N-((1S,2R)-2-{[(3R)-3-(ethoxymethyl)piperidin-1-yl]methyl}cyclohexyl)-4-{[(methylsulfonyl)amino]methyl}benzamide

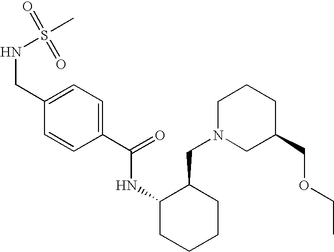

### Step A

### The Preparation of tert-butyl (4-{[((1S,2R)-2-{[(3R)-3-(ethoxymethyl)piperidin-1-yl]methyl}cyclohexyl)amino]carbonyl}benzyl)carbamate

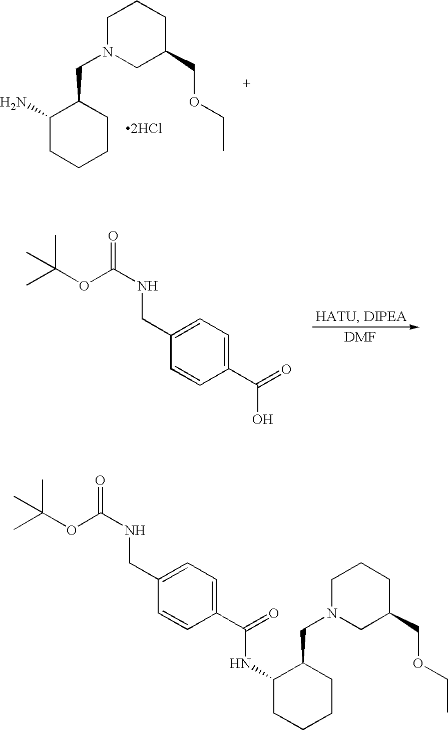

A solution 4-{[(tert-butoxycarbonyl)amino]methyl}benzoic acid (75 mg, 0.30 mmol), HATU (114 mg, 0.30 mmol) and a few drop of diisopropylethylamine in dry DMF (3 mL) was stirred at room temperature for 10 minutes. Crude ((1R,2S)-2-{[(3R)-3-ethoxypiperidin-1-yl]methyl}cyclohexyl)amine hydrochloride salt (87 mg, 0.30 mmol) was added to the solution. The mixture was stirred at room temperature for overnight. The solvent was removed in vacuo. Residue was dissolved in DCM (15 mL) and washed with saturated NaHCO₃ (10 mL) and brine (10 mL), dried over Na₂SO₄. The crude product was used for the next step without further purification. MS (M+1): 488.5

### Step B The Preparation of 4-(aminomethyl)-N-((1S,2R)-2-{[(3R)-3-(ethoxymethyl)piperidin-1-yl]methyl}cyclohexyl)benzamide hydrochloride salt

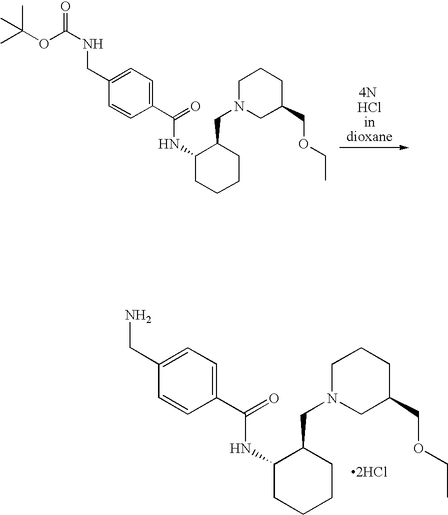

A 4N solution of hydrochloric acid in Dioxane (4.5 mL, 18.0 mmol) was added to a solution of the crude product from step A tert-butyl (4-{[((1S,2R)-2-{[(3R)-3-(ethoxymethyl)piperidin-1-yl]methyl}cyclohexyl)amino]carbonyl}benzyl)carbamate (0.30 mmol) in Dioxane (5 mL). The reaction was stirred at room temperature for 6 hours. The mixture was concentrated in vacuo. The product was used directly for the next step without further purification. MS (M+1): 388.4

### Step C

### The Preparation of N-((1S,2R)-2-{[(3R)-3-(ethoxymethyl)piperidin-1-yl]methyl}cyclohexyl)-4-{[(methylsulfonyl)amino]methyl}benzamide

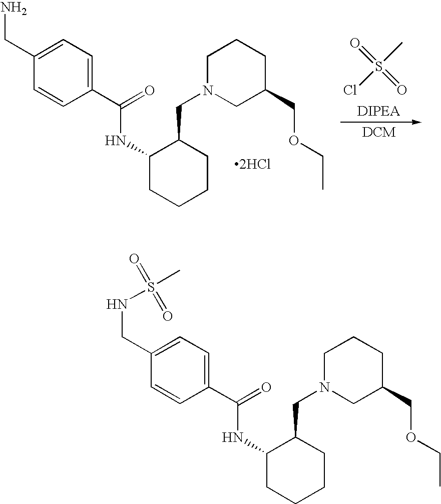

To a solution of crude 4-(aminomethyl)-N-((1S,2R)-2-{[(3R)-3-(ethoxymethyl)piperidin-1-yl]methyl}cyclohexyl)benzamide hydrochloride salt (0.3 mmol) from step B and diisopropylethylamine (few drop) was added 0.05 mL in DCM (5 mL), 0.60 mmol methanesulfonyl chloride. The mixture was stirred overnight at room temperature. Aqueous solution of NaHCO3 sat. was added (10 mL), then mixture of both layers poured into VARIAN CHEM ELUT™ cartridges. The column was rinsed with DCM (2×20 mL). Organic layer was concentrated in vacuo. The crude product was purified by prep LC-MS High pH to yield the title compound N-((1S,2R)-2-{[(3R)-3-(ethoxymethyl)piperidin-1-yl]methyl}cyclohexyl)-4-{[(methylsulfonyl)amino]methyl}benzamide (42 mg, 30%) as its free base. MS (M+1): 466.3 ¹H NMR (400 MHz, CDCl₃) δ ppm 0.79-0.96 (m, 1H), 0.98-1.21 (m, 2H), 1.24 (t, J=6.93 Hz, 3H), 1.28-1.40 (m, 2H), 1.40-1.51 (m, 2H), 1.50-1.70 (m, 3H), 1.69-1.79 (m, 3H), 1.90 (t, J=10.64 Hz, 2H), 2.04 (d, J=12.69 Hz, 1H), 2.40 (dd, J=11.52, 10.55 Hz, 1H), 2.58 (dd, J=15.23, 13.67 Hz, 2H), 2.88 (s, 3H), 3.20 (t, J=8.50 Hz, 1H), 3.25-3.32 (m, 1H), 3.34 (dd, J=9.18, 5.08 Hz, 1H), 3.37-3.46 (m, 1H), 3.45-3.55 (m, 2H), 4.37 (s, 2H), 4.62 (s, 1H), 7.39 (d, J=8.01 Hz, 2H), 7.85 (d, J=7.81 Hz, 2H), 8.99 (s, 1H)

### Example 233

### 4-[(acetylamino)methyl]-N-((1S,2R)-2-{[(3R)-3-(ethoxymethyl)piperidin-1-yl]methyl}cyclohexyl)benzamide

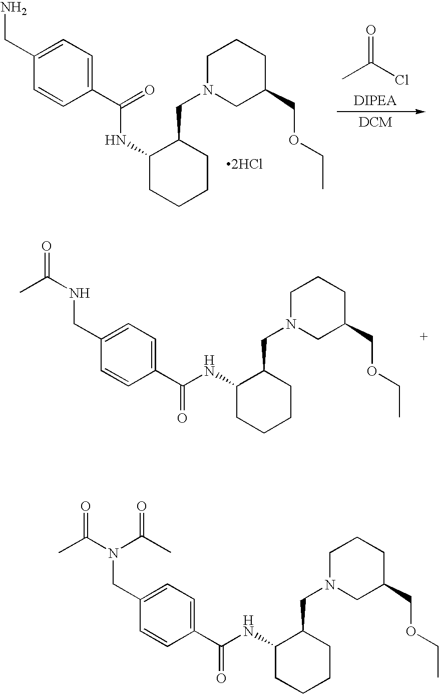

To a solution of crude 4-(aminomethyl)-N-((1S,2R)-2-{[(3R)-3-(ethoxymethyl)piperidin-1-yl]methyl}cyclohexyl)benzamide hydrochloride salt (0.3 mmol) from example 13 step B and diisopropylethylamine (few drop) was added 0.05 mL in DCM (5 mL), 0.60 mmol acetyl chloride. The mixture was stirred overnight at room temperature. Aqueous solution of NaHCO₃ sat. was added (10 mL), then mixture of both layers poured into VARIAN CHEM ELUT™ cartridges. The column was rinsed with DCM (2×20 mL). Organic layer was concentrated in vacuo. The crude product was purified by prep LC-MS High pH to obtain two fractions, and the fraction 1 is the title compound 4-[(acetylamino)methyl]-N-((1S,2R)-2-{[(3R)-3-(ethoxymethyl)piperidin-1-yl]methyl}cyclohexyl)benzamide 10 mg as its free base. MS (M+1): 430.2 1H NMR (400 MHz, CDCl₃) δ ppm 1.18 (s, 3H), 1.21-1.89 (m, 9H), 1.89-2.23 (m, 3H), 2.05 (s, 3H), 2.29-2.70 (m, 3H), 2.74-3.17 (m, J=82.22 Hz, 1H), 3.27 (s, 3H), 3.35-3.53 (m, 3H), 3.54-3.72 (m, J=5.66 Hz, 1H), 3.76-3.97 (m, 1H), 4.47 (d, J=5.66 Hz, 2H), 5.81 (s, 1H), 7.32 (d, J=8.01 Hz, 2H), 7.95 (s, 2H), 8.25 (s, 1H), 11.41 (s, 1H)

### Example 234

### 4-[(diacetylamino)methyl]-N-((1S,2R)-2-{[(3R)-3-(ethoxymethyl)piperidin-1-yl]methyl}cyclohexyl)benzamide

Fraction 2 of Example 233: 4-[(diacetylamino)methyl]-N-((1S,2R)-2-{[(3R)-3-(ethoxymethyl)piperidin-1-yl]methyl}cyclohexyl)benzamide 15 mg as its free base. MS (M+1): 472.3 ¹H NMR (400 MHz, CDCl₃) δ ppm 1.17 (t, J=6.93 Hz, 3H), 1.35 (t, 5H), 1.71-2.20 (m, 9H), 2.42 (s, 6H), 2.44-2.54 (m, 2H), 2.54-2.62 (m, 1H), 3.23-3.38 (m, 3H), 3.38-3.53 (m, 3H), 3.63 (d, J=9.57 Hz, 1H), 3.80-3.94 (m, 1H), 5.00 (s, 2H), 7.20 (d, J=8.20 Hz, 2H), 7.95 (d, J=8.20 Hz, 2H), 8.14 (t, J=9.67 Hz, 1H)

### Example 235

### N-((1S,2R)-2-{[(3R)-3-(ethoxymethyl)piperidin-1-yl]methyl}cyclohexyl)-4-{[(ethylsulfonyl)amino]methyl}benzamide

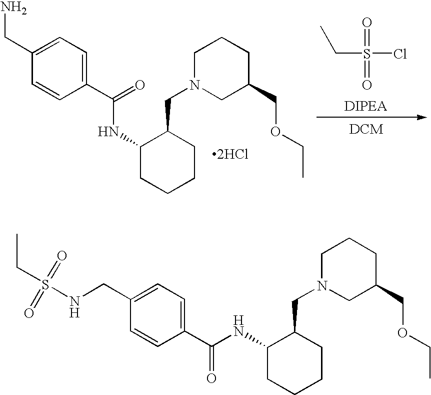

To a solution of crude 4-(aminomethyl)-N-((1S,2R)-2-{[(3R)-3-(ethoxymethyl)piperidin-1-yl]methyl}cyclohexyl)benzamide hydrochloride salt (0.3 mmol) and diisopropylethylamine (few drop) was added 0.05 mL in DCM (5 mL), ethanesulfonyl chloride (0.6 mmol). The mixture was stirred overnight at room temperature. Aqueous solution of saturated NaHCO₃ was added (10 mL), then mixture of both layers poured into VARIAN CHEM ELUT™ cartridges. The column was rinsed with DCM (2×20 mL). Organic layer was concentrated in vacuo. The crude product was purified by prep LC-MS Low pH to yield N-((1S,2R)-2-{[(3R)-3-(ethoxymethyl)piperidin-1-yl]methyl}cyclohexyl)-4-{[(ethylsulfonyl)amino]methyl}benzamide 32 mg (15%) as TFA salt. MS (M+1): 480.4. ¹H NMR (400 MHz, CDCl₃) δ ppm 1.17 (t, J=7.03 Hz, 3H), 1.23-1.50 (m, 5H), 1.34 (t, J=7.42 Hz, 3H), 1.65-1.87 (m, 3H), 1.86-2.17 (m, 4H), 2.37-2.63 (m, 4H), 2.98 (t, J=7.42 Hz, 2H), 3.22-3.33 (m, 3H), 3.39-3.52 (m, 3H), 3.63 (d, J=6.64 Hz, 1H), 3.79-3.97 (m, 1H), 4.35 (d, J=5.86 Hz, 2H), 4.57 (t, J=5.57 Hz, 1H), 7.40 (d, J=8.20 Hz, 2H), 7.98 (d, J=8.01 Hz, 2H), 8.27 (t, J=8.79 Hz, 1H), 11.23 (s, 1H)

### Example 236

### 4-{[(cyclopropylsulfonyl)amino]methyl}-N-((1S,2R)-2-{[(3R)-3-(ethoxymethyl)piperidin-1-yl]methyl}cyclohexyl)benzamide

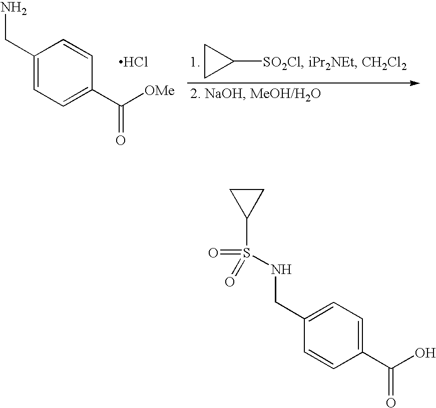

### Step A

### 4-{[(cyclopropylsulfonyl)amino]methyl}benzoic acid

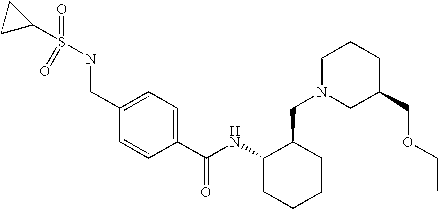

A suspension of methyl 4-(aminomethyl)benzoate hydrochloride salt (0.395 g, 2.0 mmol) in dry CH₂Cl₂ (5 mL) was cooled to 0° C., and cyclopropanesulfonyl chloride (0.46 mL, 4.5 mmol) and diisopropylethylamine (1.1 mL, 6.3 mmol) were added. The resulting mixture was allowed to warm to room temperature and stir for 89 h. The reaction was then diluted with CH₂Cl₂ (10 mL) and washed with H₂O (10 mL), a saturated aqueous solution of NaHCO₃ (10 mL), and brine (10 mL) successively. The organic layer was dried over Na₂SO₄, filtered, and concentrated in vacuo. The residue was dissolved in MeOH (10 mL), and NaOH (0.94 g, 24 mmol) dissolved in H₂O (5 mL) was added. The reaction was stirred for 20 h and was then concentrated in vacuo. The residue was dissolved in H₂O (7 mL) and acidified to pH 1 with 3 N HCl. The resulting precipitate was collected by filtration and washed with H₂O to provide the title compound as a tan solid (0.46 g, 93% over 2 steps), which was used in subsequent steps without further purification. ¹H NMR (400 MHz, METHANOL-D4) δ ppm 0.88-0.96 (m, 2H), 0.99-1.05 (m, 2H), 2.40-2.48 (m, 1H), 4.35 (s, 2H), 7.46-7.52 (m, 2H), 7.97-8.02 (m, 2H).

### Step B

### 4-{[(cyclopropylsulfonyl)amino]methyl}-N-((1S,2R)-2-{[(3R)-3-(ethoxymethyl)piperidin-1-yl]methyl}cyclohexyl)benzamide

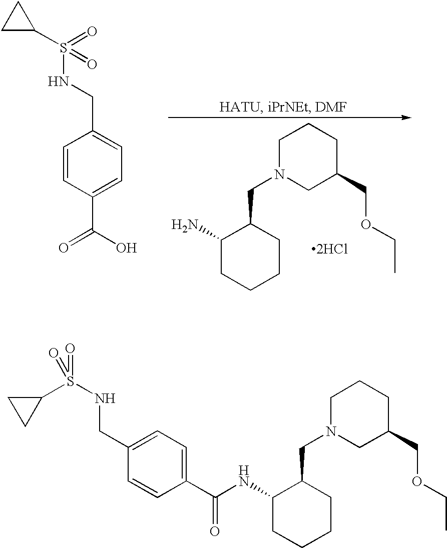

A mixture of 4-{[(cyclopropylsulfonyl)amino]methyl}benzoic acid (0.0842 g, 0.33 mmol) and crude ((1S,2R)-2-{[(3R)-3-(ethoxymethyl)piperidin-1-yl]methyl}cyclo-hexyl)amine hydrochloride salt (˜0.30 mmol) in dry DMF (3 mL) was cooled to 0° C., and HATU (0.126 g, 0.33 mmol) and diisopropylethylamine (0.21 mL, 1.2 mmol) were added. The resulting mixture was stirred at 0° C. for 30 min and then warmed to room temperature and stirred for an additional 16 h. The reaction was concentrated in vacuo, and the residue was taken up into CH₂Cl₂ (4 mL) and a saturated solution of NaHCO₃ in water (4 mL). The mixture was passed through a Varian Chem Elut™ extraction cartridge, and the cartridge washed with additional CH₂Cl₂ (3×8 mL). The organic extract was concentrated in vacuo, and the residue was purified by preparative scale reverse phase LC/MS (gradient 55-75% CH₃CN in H₂O containing 10 mM NH₄HCO₃) to provide the title compound as a white solid (0.0706 g, 48% over 3 steps) following lyophilization from CH₃CN/H₂O. MS (M+1): 492.3. ¹H NMR (400 MHz, CHLOROFORM-D) δ ppm 0.79-0.97 (m, 3H), 0.99-1.18 (m, 4H), 1.23 (t, J=7.0 Hz, 3H), 1.26-1.49 (m, 4H), 1.50-1.79 (m, 7H), 1.89 (t, J=10.7 Hz, 2H), 1.99-2.07 (m, 1H), 2.26-2.46 (m, 2H), 2.49-2.65 (m, 2H), 3.12-3.23 (m, 1H), 3.23-3.54 (m, 4H), 4.38 (d, J=2.3 Hz, 2H), 4.52-4.64 (m, 1H), 7.35-7.43 (m, 2H), 7.78-7.86 (m, 2H), 8.97 (s, 1H). Anal. Calcd for C₂₆H₄₁N₃O₄S.0.1H₂O: C, 63.28; H, 8.42; N, 8.51. Found: C, 63.25; H, 8.80; N, 8.41.

### Example 237

### N-((1S,2R)-2-{[(3R)-3-(ethoxymethyl)piperidin-1-yl]methyl}cyclohexyl)-4-({[(methylamino)carbonyl]amino}methyl)benzamide

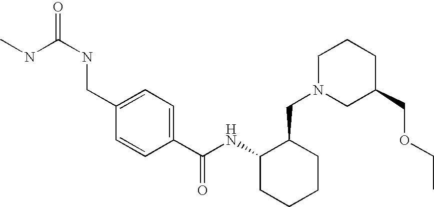

### Step A

### 4-({[(methylamino)carbonyl]amino}methyl)benzoic acid

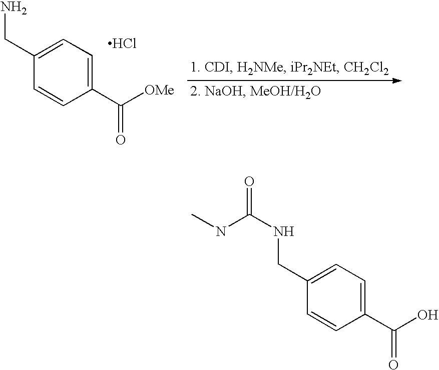

A suspension of methyl 4-(aminomethyl)benzoate hydrochloride salt (0.257 g, 1.3 mmol) in dry CH₂Cl₂ (5 mL) was treated with diisopropylethylamine (0.67 mL, 3.8 mmol) and 1,1′-carbonyldiimidazole (0.207 g, 1.3 mmol). The resulting mixture was stirred for 15 min, and then methyl amine (1.3 mL of 2M in MeOH, 2.6 mmol) was added and the reaction was stirred for an additional 132 h. Water (5 mL) was added, and the mixture was passed through a Varian Chem Elut™ extraction cartridge. The cartridge washed with additional CH₂Cl₂ (3×8 mL), and the organic extract was concentrated in vacuo. The residue was dissolved in MeOH (7 mL), and NaOH (0.61 g, 15 mmol) dissolved in H₂O (3.5 mL) was added. The reaction was stirred for 20 h and was then concentrated in vacuo. The residue was dissolved in H₂O (5 mL) and acidified to pH 1 with 3 N HCl. The resulting precipitate was collected by filtration and washed with H₂O to provide the title compound as a white solid (0.22 g, 82% over 2 steps), which was used in subsequent steps without further purification. ¹H NMR (400 MHz, METHANOL-D4) δ ppm 2.71 (s, 3H), 4.37 (s, 2H), 7.38 (d, J=4.7 Hz, 2H), 7.97 (d, J=5.5 Hz, 2H).

### Step B

### N-((1S,2R)-2-{[(3R)-3-(ethoxymethyl)piperidin-1-yl]methyl}cyclohexyl)-4-({[(methylamino)carbonyl]amino}methyl)benzamide

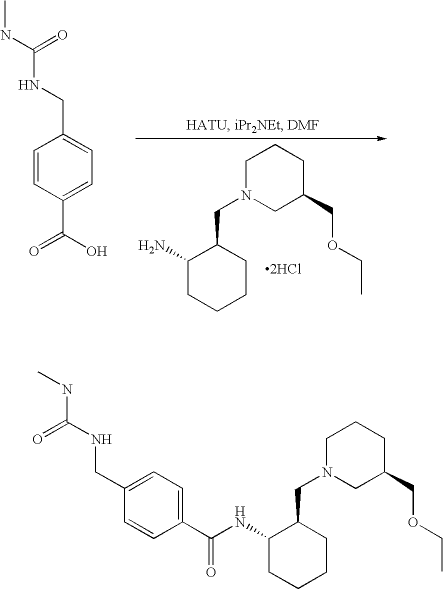

A mixture of 4-({[(methylamino)carbonyl]amino}methyl)benzoic acid (0.0687 g, 0.33 mmol) and crude ((1S,2R)-2-{[(3R)-3-(ethoxymethyl)piperidin-1-yl]methyl}cyclo-hexyl)amine hydrochloride salt (˜0.30 mmol) in dry DMF (3 mL) was cooled to 0° C., and HATU (0.126 g, 0.33 mmol) and diisopropylethylamine (0.21 mL, 1.2 mmol) were added. The resulting mixture was stirred at 0° C. for 30 min and then warmed to room temperature and stirred for an additional 16 h. The reaction was concentrated in vacuo, and the residue was taken up into CH₂Cl₂ (4 mL) and a saturated solution of NaHCO₃ in water (4 mL). The mixture was passed through a Varian Chem Elut™ extraction cartridge, and the cartridge washed with additional CH₂Cl₂ (3×8 mL). The organic extract was concentrated in vacuo, and the residue was purified by preparative scale reverse phase LC/MS (gradient 45-65% CH₃CN in H₂O containing 10 mM NH₄HCO₃) to provide the title compound as a white solid (0.0630 g, 47% over 3 steps) following lyophilization from CH₃CN/H₂O. MS (M+1): 445.2. ¹H NMR (400 MHz, CHLOROFORM-D) δ ppm 0.74-0.93 (m, 1H), 0.96-1.18 (m, 2H), 1.21 (t, J=7.0 Hz, 3H), 1.24-1.49 (m, 4H), 1.51-1.79 (m, 6H), 1.79-1.93 (m, 2H), 2.02 (d, J=12.9 Hz, 1H), 2.36 (dd, J=13.1, 9.6 Hz, 1H), 2.46-2.58 (m, 2H), 2.79 (d, J=5.1 Hz, 3H), 3.16 (dd, J=9.4, 8.2 Hz, 1H), 3.21-3.29 (m, 1H), 3.29-3.41 (m, 2H), 3.42-3.53 (m, 2H), 4.40 (d, J=5.5 Hz, 2H), 5.02 (d, J=4.3 Hz, 1H), 5.32 (t, J=5.7 Hz, 1H), 7.22 (d, J=8.6 Hz, 2H), 7.58-7.66 (m, 2H), 9.00 (d, J=2.7 Hz, 1H). Anal. Calcd for C₂₅H₄₀N₄O₃.0.3H₂O: C, 66.72; H, 9.09; N, 12.45. Found: C, 66.63; H, 8.77; N, 12.73.

### Example 238

### 4-({[(dimethylamino)carbonyl]amino}methyl)-N-((1S,2R)-2-{[(3R)-3-(ethoxymethyl)piperidin-1-yl]methyl}cyclohexyl)benzamide

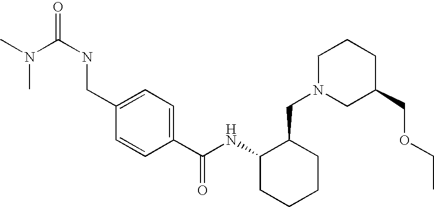

### Step A

### 4-({[(dimethylamino)carbonyl]amino}methyl)benzoic acid

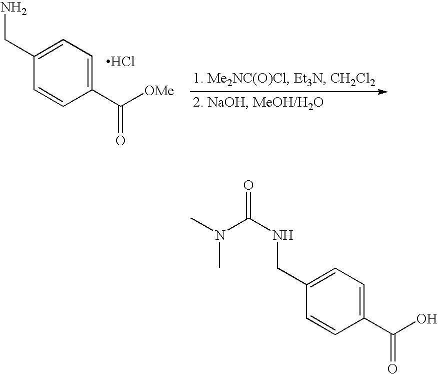

A suspension of methyl 4-(aminomethyl)benzoate hydrochloride salt (0.266 g, 1.3 mmol) in dry CH₂Cl₂ (5 mL) was treated with triethylamine (0.92 mL, 6.6 mmol) and dimethylcarbamoyl chloride (0.13 mL, 1.4 mmol). The resulting mixture was stirred for 132 h. Water (5 mL) was added, and the mixture was passed through a Varian Chem Elut™ extraction cartridge. The cartridge washed with additional CH₂Cl₂ (3×8 mL), and the organic extract was concentrated in vacuo. The residue was dissolved in MeOH (7 mL), and NaOH (0.63 g, 16 mmol) dissolved in H₂O (3.5 mL) was added. The reaction was stirred for 20 h and was then concentrated in vacuo. The residue was dissolved in H₂O (5 mL) and acidified to pH 1 with 3 N HCl. The resulting precipitate was collected by filtration and washed with H₂O to provide the title compound as a white solid (0.20 g, 70% over 2 steps), which was used in subsequent steps without further purification. ¹H NMR (400 MHz, METHANOL-D4) δ ppm 2.93 (s, 6H), 4.40 (s, 2H), 7.38 (d, J=8.2 Hz, 2H), 7.93-7.98 (m, 2H)

### Step B

### 4-({[(dimethylamino)carbonyl]amino}methyl)-N-((1S,2R)-2-{[(3R)-3-(ethoxymethyl)piperidin-1-yl]methyl}cyclohexyl)benzamide

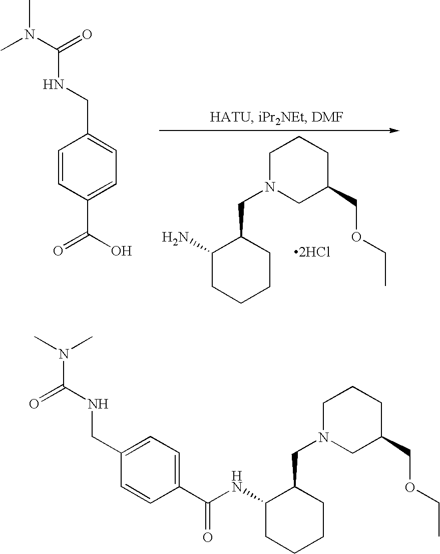

A mixture of 4-({[(dimethylamino)carbonyl]amino}methyl)benzoic acid (0.0733 g, 0.33 mmol) and crude ((1S,2R)-2-{[(3R)-3-(ethoxymethyl)piperidin-1-yl]methyl}cyclo-hexyl)amine hydrochloride salt (˜0.30 mmol) in dry DMF (3 mL) was cooled to 0° C., and HATU (0.126 g, 0.33 mmol) and diisopropylethylamine (0.21 mL, 1.2 mmol) were added. The resulting mixture was stirred at 0° C. for 30 min and then warmed to room temperature and stirred for an additional 16 h. The reaction was concentrated in vacuo, and the residue was taken up into CH₂Cl₂ (4 mL) and a saturated solution of NaHCO₃ in water (4 mL). The mixture was passed through a Varian Chem Elut™ extraction cartridge, and the cartridge washed with additional CH₂Cl₂ (3×8 mL). The organic extract was concentrated in vacuo, and the residue was purified by preparative scale reverse phase LC/MS (gradient 45-65% CH₃CN in H₂O containing 10 mM NH₄HCO₃) to provide the title compound as a white solid (0.0722 g, 52% over 3 steps) following lyophilization from CH₃CN/H₂O. MS (M+1): 459.2. ¹H NMR (400 MHz, CHLOROFORM-D) δ ppm 0.78-0.93 (m, 1H), 0.95-1.19 (m, 2H), 1.22 (t, J=7.0 Hz, 3H), 1.24-1.50 (m, 4H), 1.51-1.78 (m, 6H), 1.81-1.95 (m, 2H), 2.01 (d, J=12.9 Hz, 1H), 2.38 (dd, J=12.9, 9.4 Hz, 1H), 2.50-2.63 (m, 2H), 2.89-2.96 (m, 6H), 3.19 (dd, J=9.4, 7.8 Hz, 1H), 3.25 (dd, J=10.7, 2.9 Hz, 1H), 3.32 (dd, J=9.2, 5.3 Hz, 1H), 3.35-3.53 (m, 3H), 4.36-4.55 (m, 2H), 4.65 (t, J=5.9 Hz, 1H), 7.33 (d, J=8.2 Hz, 2H), 7.75-7.82 (m, 2H), 8.84 (d, J=2.7 Hz, 1H). Anal. Calcd for C₂₆H₄₂N₄O₃.0.4H₂O: C, 67.04; H, 9.26; N, 12.03. Found: C, 67.13; H, 9.24; N, 11.86.

### Example 239

### N-((1S,2R)-2-{[(3R)-3-(ethoxymethyl)piperidin-1-yl]methyl}cyclohexyl)-4-[(isobutyrylamino)methyl]benzamide

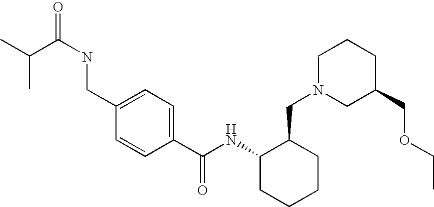

### Step A

### 4-[(isobutyrylamino)methyl]benzoic acid

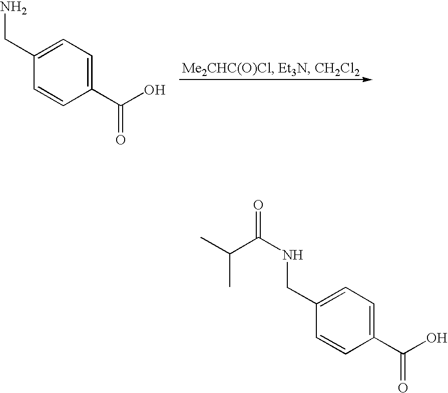

A suspension of 4-(aminomethyl)benzoic acid (0.214 g, 1.4 mmol) in dry CH₂Cl₂ (10 mL) was treated with triethylamine (0.98 mL, 7.0 mmol) and 2-methylpropanoyl chloride (0.16 mL, 1.5 mmol). The resulting mixture was stirred for 132 h. Water (5 mL) and EtOAc (10 mL) were added, and the aqueous layer was acidified to pH 1 with 1 N HCl. The layers were separated, and the aqueous phase was extracted with additional EtOAc (3×10 mL). The combined organic phases were dried over Na₂SO₄, filtered, and concentrated in vacuo to provide the title compound as a slightly yellow solid (0.318 g, quantitative), which was used in subsequent steps without further purification. ¹H NMR (400 MHz, METHANOL-D4) δ ppm 1.14 (d, J=7.0 Hz, 6H), 2.37-2.60 (m, 1H), 4.33-4.47 (m, 2H), 7.36 (d, J=8.6 Hz, 2H), 7.97 (d, J=8.6 Hz, 2H).

### Step B

### N-((1S,2R)-2-{[(3R)-3-(ethoxymethyl)piperidin-1-yl]methyl}cyclohexyl)-4-[(isobutyrylamino)methyl]benzamide

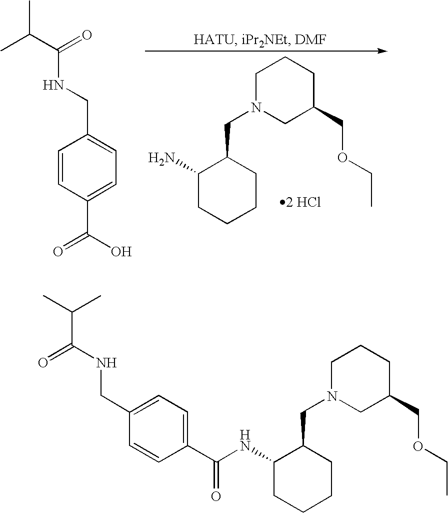

A mixture of 4-[(isobutyrylamino)methyl]benzoic acid (0.0730 g, 0.33 mmol) and crude ((1S,2R)-2-{[(3R)-3-(ethoxymethyl)piperidin-1-yl]methyl}cyclohexyl)amine hydrochloride salt (˜0.30 mmol) in dry DMF (3 mL) was cooled to 0° C., and HATU (0.126 g, 0.33 mmol) and diisopropylethylamine (0.21 mL, 1.2 mmol) were added. The resulting mixture was stirred at 0° C. for 30 min and then warmed to room temperature and stirred for an additional 16 h. The reaction was concentrated in vacuo, and the residue was taken up into CH₂Cl₂ (4 mL) and a saturated solution of NaHCO₃ in water (4 mL). The mixture was passed through a Varian Chem Elut™ extraction cartridge, and the cartridge washed with additional CH₂Cl₂ (3×8 mL). The organic extract was concentrated in vacuo, and the residue was purified by preparative scale reverse phase LC/MS (gradient 45-65% CH₃CN in H₂O containing 10 mM NH₄HCO₃) to provide the title compound as a white solid (0.0534 g, 36% over 3 steps) following lyophilization from CH₃CN/H₂O. MS (M+1): 458.3. ¹H NMR (400 MHz, METHANOL-D4) δ ppm 1.10-1.16 (m, 9H), 1.16-1.65 (m, 5H), 1.69-1.91 (m, 4H), 1.91-2.27 (m, 5H), 2.41-2.58 (m, 1H), 2.66-2.85 (m, 2H), 2.95-3.19 (m, 2H), 3.20-3.27 (m, 1H), 3.36-3.57 (m, 4H), 3.63 (d, J=12.1 Hz, 1H), 3.77 (td, J=10.8, 4.1 Hz, 1H), 4.40 (s, 2H), 7.38 (d, J=8.2 Hz, 2H), 7.78-7.86 (m, 2H). Anal. Calcd for C₂₇H₄₃N₃O₃.2.1HCl: C, 60.70; H, 8.51; N, 7.87. Found: C, 60.75; H, 8.25; N, 8.10.

### Example 240

### N-((1S,2R)-2-{[3-cyclohexylpiperidin-1-yl]methyl}cyclohexyl)-6-(1H-pyrazol-1-yl)nicotinamide

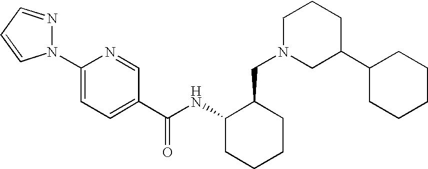

### Step A

### ((1S,2R)-2-{[3-cyclohexylpiperidin-1-yl]methyl}cyclohexyl)amine hydrochloride salt

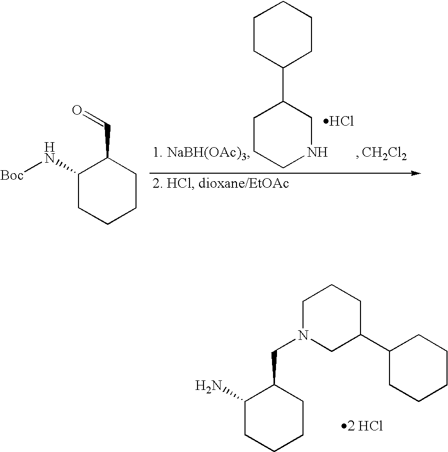

A mixture of crude tert-butyl[(1S,2S)-2-formylcyclohexyl]carbamate (0.136 g, ˜0.60 mmol) and 3-cyclohexylpiperidine hydrochloride salt (0.147 g, 0.72 mmol) in dry CH₂Cl₂ (12 mL) was stirred for 30 min at 5° C. NaBH(OAc)₃ (0.254 g, 1.2 mmol) was added to the reaction and the resulting mixture was allowed to slowly warm to room temperature and stir for 16 h. The reaction was cooled to 0° C., and water (6 mL) was added, followed by 1 N NaOH (6 mL) and CH₂Cl₂ (20 mL). The layers were separated, and the aqueous phase was extracted with additional CH₂Cl₂ (2×20 mL). The combined organic layers were dried over Na₂SO₄, filtered, and concentrated in vacuo. The residue was dissolved in EtOAc (1.5 mL), and 4 N HCl in dioxane (1.5 mL, 6 mmol) was added. The mixture was stirred for 1 h and then concentrated in vacuo to provide the title compound. The compound was used in subsequent steps without further purification. MS (M+1): 279.2.

### Step B

### N-((1S,2R)-2-{[3-cyclohexylpiperidin-1-yl]methyl}cyclohexyl)-6-(1H-pyrazol-1-yl)nicotinamide

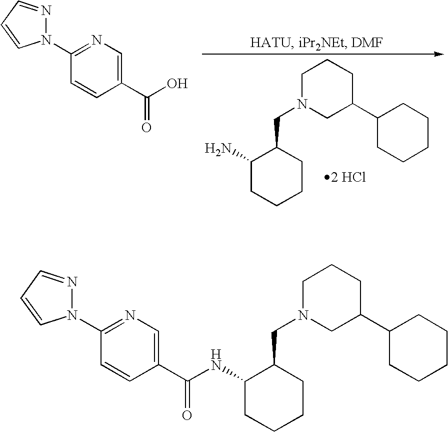

A mixture of crude ((1S,2R)-2-{[3-cyclohexylpiperidin-1-yl]methyl}cyclohexyl)amine hydrochloride salt (˜0.6 mmol) and 6-(1H-pyrazol-1-yl)nicotinic acid (0.125 g, 0.66 mmol) in dry DMF (5 mL) was cooled to 0° C. HATU (0.251 g, 0.66 mmol) and diisopropylethylamine (0.42 mL, 2.4 mmol) were then added to the reaction, and the resulting mixture was stirred at 0° C. for 30 min and then warmed to room temperature and stirred for an additional 63 h. The reaction was concentrated in vacuo, and the residue was taken up into CH₂Cl₂ (8 mL) and a saturated solution of NaHCO₃ in water (8 mL). The mixture was passed through a Varian Chem Elut™ extraction cartridge, and the cartridge washed with additional CH₂Cl₂ (2×12 mL). The organic extract was concentrated in vacuo, and the residue was purified by preparative scale reverse phase LC/MS (gradient 75-100% CH₃CN in H₂O containing 10 mM NH₄HCO₃) to provide a mixture of the title compounds as a white solid (0.0414 g, 15% over 3 steps) following lyophilization from CH₃CN/H₂O. MS (M+1): 450.2. ¹H NMR (400 MHz, CHLOROFORM-D) δ ppm 0.38-1.91 (m, 26H), 2.05 (d, J=13.3 Hz, 1H), 2.27-2.45 (m, 1H), 2.47-2.73 (m, 2H), 3.03-3.22 (m, 1H), 3.34-3.48 (m, 1H), 6.43-6.50 (m, 1H), 7.72-7.79 (m, 1H), 7.94-8.05 (m, 1H), 8.17-8.29 (m, 1H), 8.56-8.66 (m, 1H), 8.79-8.92 (m, 1H), 9.29-9.47 (m, 1H).

### Example 241

### N-((1S,2R)-2-{[3-phenylpiperidin-1-yl]methyl}cyclohexyl)-6-(1H-pyrazol-1-yl)nicotinamide

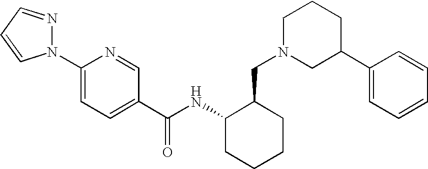

### Step A

### ((1S,2R)-2-{[3-phenylpiperidin-1-yl]methyl}cyclohexyl)amine hydrochloride salt

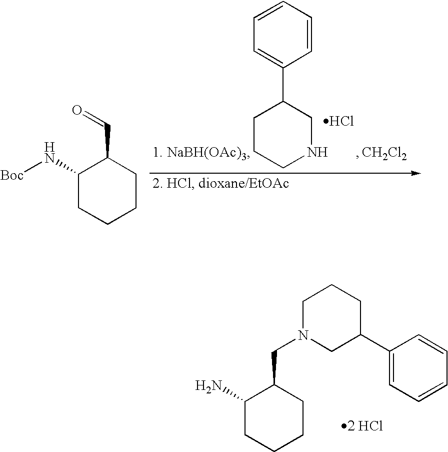

A mixture of crude tert-butyl[(1S,2S)-2-formylcyclohexyl]carbamate (0.136 g, ˜0.60 mmol) and 3-phenylpiperidine (0.116 g, 0.72 mmol) in dry CH₂Cl₂ (12 mL) was stirred for 30 min at 5° C. NaBH(OAc)₃ (0.254 g, 1.2 mmol) was added to the reaction and the resulting mixture was allowed to slowly warm to room temperature and stir for 16 h. The reaction was cooled to 0° C., and water (6 mL) was added, followed by 1 N NaOH (6 mL) and CH₂Cl₂ (20 mL). The layers were separated, and the aqueous phase was extracted with additional CH₂Cl₂ (2×20 mL). The combined organic layers were dried over Na₂SO₄, filtered, and concentrated in vacuo. The residue was dissolved in EtOAc (1.5 mL), and 4 N HCl in dioxane (1.5 mL, 6 mmol) was added. The mixture was stirred for 1 h and then concentrated in vacuo to provide the title compound. The compound was used in subsequent steps without further purification. MS (M+1): 273.2.

### Step B

### N-((1S,2R)-2-{[3-phenylpiperidin-1-yl]methyl}cyclohexyl)-6-(1H-pyrazol-1-yl)nicotinamide

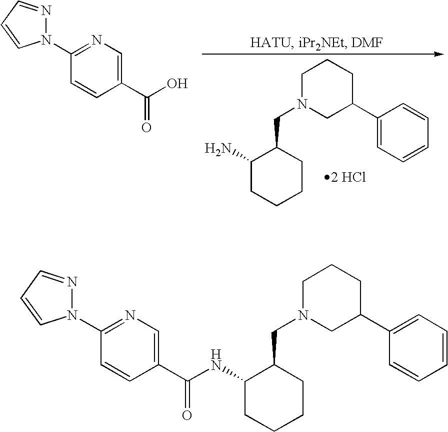

A mixture of crude ((1S,2R)-2-{[(3S)-3-phenylpiperidin-1-yl]methyl}cyclohexyl)amine hydrochloride salt and ((1S,2R)-2-{[(3R)-3-phenylpiperidin-1-yl]methyl}cyclohexyl)amine hydrochloride salt (˜0.6 mmol) and 6-(1H-pyrazol-1-yl)nicotinic acid (0.125 g, 0.66 mmol) in dry DMF (5 mL) was cooled to 0° C. HATU (0.251 g, 0.66 mmol) and diisopropylethylamine (0.42 mL, 2.4 mmol) were then added to the reaction, and the resulting mixture was stirred at 0° C. for 30 min and then warmed to room temperature and stirred for an additional 63 h. The reaction was concentrated in vacuo, and the residue was taken up into CH₂Cl₂ (8 mL) and a saturated solution of NaHCO₃ in water (8 mL). The mixture was passed through a Varian Chem Elut™ extraction cartridge, and the cartridge washed with additional CH₂Cl₂ (2×12 mL). The organic extract was concentrated in vacuo, and the residue was purified by preparative scale reverse phase LC/MS (gradient 65-85% CH₃CN in H₂O containing 10 mM NH₄HCO₃) to provide a mixture of the title compounds as a white solid (0.131 g, 49% over 3 steps) following lyophilization from CH₃CN/H₂O. MS (M+1): 444.2. ¹H NMR (400 MHz, CHLOROFORM-D) δ ppm 1.00-1.51 (m, 5H), 1.51-2.19 (m, 9H), 2.34-2.53 (m, 2H), 2.56-2.88 (m, 3H), 3.18-3.33 (m, 1H), 3.37-3.51 (m, 1H), 6.45-6.52 (m, J=2.1, 2.1 Hz, 1H), 6.86 (dd, J=7.6, 1.8 Hz, 1H), 7.05-7.16 (m, 2H), 7.20-7.38 (m, 2H), 7.74-7.80 (m, 1H), 8.01-8.09 (m, 1H), 8.22-8.34 (m, J=8.8, 8.8, 2.3 Hz, 1H), 8.63 (d, J=2.7 Hz, 1H), 8.85-8.95 (m, 1H), 9.16 (d, J=3.9 Hz, 1H). Anal. Calcd for C₂₇H₃₃N₅O: C, 73.11; H, 7.50; N, 15.79. Found: C, 72.93; H, 7.50; N, 15.89.

What is claimed is:

1. A compound of formula I, a pharmaceutically acceptable salt thereof, diastereomer, enantiomer, or mixture thereof: 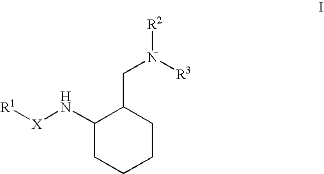
wherein
R¹ is selected from C₆₋₁₀aryl, C₂₋₉heteroaryl, C₃₋₅heterocycloalkyl, C₆₋₁₀aryl-C₁₋₃alkyl, C₂₋₉heteroaryl-C₁₋₃alkyl, C₃₋₅heterocycloalkyl-C₁₋₃alkyl, C₃₋₆cycloalkyl, C₃₋₆cycloalkyl-C₁₋₃alkyl, and C₁₋₆alkyl, wherein said C₆₋₁₀aryl, C₂₋₉heteroaryl, C₆₋₁₀aryl-C₁₋₃alkyl, C₆₋₁₀aryl-O—C₁₋₃alkyl, C₂₋₉heteroaryl-C₁₋₃alkyl, C₃₋₆cycloalkyl, C₃₋₆cycloalkyl-C₁₋₃alkyl, and C₁₋₆alkyl are optionally substituted with one or more group selected from C₆₋₁₀aryl, C₁₋₉heteroaryl, C₃₋₅heterocycloalkyl, C₆₋₁₀aryl-C₁₋₃alkyl, C₆₋₁₀aryl-O—C₁₋₃alkyl, C₂₋₉heteroaryl-C₁₋₃alkyl, C₃₋₅heterocycloalkyl-C₁₋₃alkyl, —CN, —SR, —OR, —O(CH₂)ₘ—OR, R, —C(═O)—R, —CO₂R, —SO₂R, —SO₂NR₂, halogen, —NO₂, —NR₂, —(CH₂)ₘNR₂, —(CH₂)ₘNHC(═O)—NR₂, —NHC(═O)—R, —N[C(═O)R]₂, —(CH₂)ₘNHS(═O)₂—R, —(CH₂)ₘNHC(═O)—R, —(CH₂)ₘN[C(═O)—R]₂ and —C(═O)—NR₂;
R² and R³ are independently selected from C₁₋₆alkyl, C₂₋₆alkenyl, and C₁₋₆alkoxy wherein said C₁₋₆alkyl, C₂₋₆alkenyl, and C₁₋₆alkoxy are optionally substituted by one or more groups selected from amino, halogen, C₁₋₆alkoxy and —CN; or R² and R³ together with the nitrogen connected thereto form a heterocycloalkyl, wherein said heterocycloalkyl is optionally substituted with one or more group selected from C₆₋₁₀aryl, C₂₋₉heteroaryl, C₃₋₆cycloalkyl, C₃₋₅heterocycloalkyl, C₆₋₁₀aryl-C₁₋₃alkyl, C₂₋₉heteroaryl-C₁₋₃alkyl, C₃₋₅heterocycloalkyl-C₁₋₃alkyl, —CN, —SR, —OR, —(CH₂)ₘOR, R, —CO₂R; —SO₂R; —SO₂NR₂, halogen, —NO₂, —NR₂, —(CH₂)ₘNR₂, and —C(═O)—NR₂;
each R is independently hydrogen, C₁₋₆alkyl, C₂₋₆alkenyl or halogenated C₁₋₆alkyl; and
X is selected from —C(═O)—, —C(═O)—NH—, —C(═O)—O— and —S(═O)₂—,
with a proviso that
when X is —C(═O)— and R² and R³ together with the nitrogen connected thereto form said piperidinyl; R¹ is not 4-amino-5-chloro-2-alkoxylphenyl, 4-amino-5-chloro-2-cycloalkoxyphenyl, 4-amino-5-chloro-2-cycloalkyl-alkoxy-phenyl, 4-butoxyphenyl, 3-butoxyphenyl, 4-pentyloxyphenyl, 4-isobutoxyphenyl, 4-benzoyloxyphenyl and 7-(2,3-dihydro)benzofuranyl.

2. A compound as claimed in claim 1, wherein
said R² and R³ together with the nitrogen connected thereto form a heterocycloalkyl, wherein said heterocycloalkyl is optionally substituted with one or more group selected from C₆₋₁₀aryl, C₂₋₉heteroaryl, C₃₋₆cycloalkyl, C₃₋₅heterocycloalkyl, C₆₋₁₀aryl-C₁₋₃alkyl, C₂₋₉heteroaryl-C₁₋₃alkyl, C₃₋₅heterocycloalkyl-C₁₋₃alkyl —CN, —SR, —OR, —(CH₂)ₘOR, R, —CO₂R; —SO₂R; —SO₂NR₂, halogen, —NO₂, —NR₂, —(CH₂)ₘNR₂, and —C(═O)—NR₂.

3. A compound as claimed in claim 1, wherein
said R² and R³ together with the nitrogen connected thereto form a group selected from piperidinyl, 1,4-dixo-8-azaspiro[4,5]dec-8-yl, piperazinyl, methyl(2-phenylethyl)amino, methyl(pyridin-3-ylmethyl)amino, (4-ethylbenzyl)(methyl)amino, methyl(1-methylpyrrolidin-3-yl)amino, methyl(3-methylbutyl)amino, methyl(propyl)amino, methyl(butyl)amino, butyl(ethyl)amino, diethylamino, benzyl(methyl)amino, morpholin-4-yl, pyrrolidin-1-yl, and azepan-1-yl, wherein said piperidinyl, 1,4-dixo-8-azaspiro[4,5]dec-8-yl, piperazinyl, methyl(2-phenylethyl)amino, methyl(pyridin-3-ylmethyl)amino, (4-ethylbenzyl)(methyl)amino, methyl(1-methylpyrrolidin-3-yl)amino, methyl(3-methylbutyl)amino, methyl(propyl)amino, methyl(butyl)amino, butyl(ethyl)amino, diethylamino, benzyl(methyl)amino, morpholin-4-yl, pyrrolidin-1-yl, and azepan-1-yl are optionally substituted with one or more group selected from C₆₋₁₀aryl, C₂₋₉heteroaryl, C₃₋₆cycloalkyl, C₃₋₅heterocycloalkyl, C₆₋₁₀aryl-C₁₋₃alkyl, C₂₋₉heteroaryl-C₁₋₃alkyl, C₃₋₅heterocycloalkyl-C₁₋₃alkyl —CN, —SR, —OR, —(CH₂)ₘOR, R, —CO₂R; —SO₂R; —SO₂NR₂, halogen, —NO₂, —NR₂, —(CH₂)ₘNR₂, and —C(═O)—NR₂.

4. A compound as claimed in claim 1, wherein
said R¹ is selected from 2-cyclopentylethyl, cyclopropylmethyl, methyl, cyclohexyl, cyclopentylmethyl, chromanyl, ethyl, pentyl, 2-phenylethyl, phenyl, benzyl, pyridinyl, pyridinylethyl, 1-benzofuranyl, benzothienyl, furyl, imidazolyl, pyrazolo[1,5-a]pyrimidinyl, pyrazinyl, 1,3-benzothiazolyl, indolyl, indazolyl, thienyl, 1,3-benzodioxinyl, tetrahydro-2H-pyran-4-ylmethyl, 1-H-1,2,3-benzotriazol-1-yl, 2-(thien-2-yl)ethyl, (1-benzofuran-4-yl)methyl, 1,3-oxazolyl, 1H-pyrazol-1-yl, 2,3-dihydro-1-benzofuran-5-yl, 1,3-benzodioxol-5-yl, 2-oxo-2,3-dihydro-2H-benzimidazolyl, isoxazolyl, imidazo[1,2,a]pyridinyl, 2-3-dioxo-2,3-dihydro-1H-indol-1-yl, 3,4-dihydro-2H-1,4-benzoxazinyl; pyrazolyl, 1H-tetrazol-1-yl-methyl, and 3,4-dihydro-2H-1,5-benzodioxepinyl, optionally substituted by 1H-pyrazol-1-yl, fluoro, chloro, trifluoromethyl, methoxy, difluoromethoxy, trifluoromethoxy, 2-methoxyethoxy, 2-ethoxyethoxy, t-butyl, cyano, bromo, 1,3-oxazol-5-yl, 1H-imidazol-1-yl, (4-oxopiperidin-1-yl)carbonyl, pyridin-3-ylmethyl, [(butylamino)carbonyl]amino, 1,1-dioxidothiomorpholin-4-yl, aminosulfonyl, morpholin-4-yl, diethylaminomethyl, acetyl, (3-oxo-2,3-dihydro-4H-1,4-benzoxazin-4-yl)methyl, 1-oxo-indan-4-yl, dimethylaminomethyl, methyl, pyrrolidin-1-yl, ethylthio, acetylamino, dimethylamino, 1H-pyrrol-1-yl, ethyl, ethoxy, fluorophenoxy, propyl, phenyl, methoxycarbonyl, diacetylamino, (methylsulfonylamino)methyl, (cyclopropylsulfonylamino)methyl, 1H-tetrazol-1-yl, pyrazolyl, methylaminocarbonylamino, dimethylaminocarbonylamino, and (methylthio)pyrimidin-4-yl.

5. A compound as claimed in claim 1, wherein
said R² and R³ together with the nitrogen connected thereto form a group selected from piperidinyl, 1,4-dixo-8-azaspiro[4,5]dec-8-yl, piperazinyl, methyl(2-phenylethyl)amino, methyl(pyridin-3-ylmethyl)amino, (4-ethylbenzyl)(methyl)amino, methyl(1-methylpyrrolidin-3-yl)amino, methyl(3-methylbutyl)amino, methyl(propyl)amino, methyl(butyl)amino, butyl(ethyl)amino, diethylamino, benzyl(methyl)amino, morpholin-4-yl, pyrrolidin-1-yl, and azepan-1-yl, wherein said piperidinyl, 1,4-dixo-8-azaspiro[4,5]dec-8-yl, piperazinyl, methyl(2-phenylethyl)amino, methyl(pyridin-3-ylmethyl)amino, (4-ethylbenzyl)(methyl)amino, methyl(1-methylpyrrolidin-3-yl)amino, methyl(3-methylbutyl)amino, methyl(propyl)amino, methyl(butyl)amino, butyl(ethyl)amino, diethylamino, benzyl(methyl)amino, morpholin-4-yl, pyrrolidin-1-yl, and azepan-1-yl are optionally substituted with one or more group selected from phenyl, benzyl, methyl, fluoro, trifluoromethyl, methoxy, allyloxy, (2E)-but-2-en-1-yloxy, (allyloxy)methyl, methoxymethyl, ethoxymethyl, propyl, butyl, pentyl, hexyl, cyclopentyl, pyridin-4-ylmethyl, ethoxy, butoxy, 2-methoxyethoxy, cyclohexyl, and thienylmethyl.

6. A compound as claimed in claim 1, wherein said R² and R³ together with the nitrogen connected thereto form a group selected from piperidinyl, wherein said piperidinyl is optionally substituted with one or more group selected from phenyl, benzyl, methyl, fluoro, trifluoromethyl, methoxy, allyloxy, (2E)-but-2-en-1-yloxy, (allyloxy)methyl, methoxymethyl, ethoxymethyl, propyl, butyl, pentyl, hexyl, cyclopentyl, pyridin-4-ylmethyl, ethoxy, butoxy, 2-methoxyethoxy, cyclohexyl, and thienylmethyl.

7. A compound selected from
trans-(+/−)-4-fluoro-N-[2-(piperidin-1-ylmethyl)cyclohexyl]benzamide;
trans-(+/−)-N-[2-(piperidin-1-ylmethyl)cyclohexyl]-6-(1H-pyrazol-1-yl)nicotinamide;
trans-(+/−)-N-[2-(piperidin-1-ylmethyl)cyclohexyl]-6-(trifluoromethyl)nicotinamide;
trans-(+/−)-N-[2-(piperidin-1-ylmethyl)cyclohexyl]-4-(1H-pyrazol-1-yl)benzamide;
trans-(+/−)-5-chloro-N-[2-(piperidin-1-ylmethyl)cyclohexyl]-1-benzofuran-2-carboxamide;
trans-(+/−)-2-(4-methoxyphenyl)-N-[2-(piperidin-1-ylmethyl)cyclohexyl]acetamide;
trans-(+/−)-4-(difluoromethoxy)-N-[2-(piperidin-1-ylmethyl)cyclohexyl]benzamide;
trans-(+/−)-4-(2-methoxyethoxy)-N-[2-(piperidin-1-ylmethyl)cyclohexyl]benzamide;
trans-(+)-4-(2-methoxyethoxy)-N-[2-(piperidin-1-ylmethyl)cyclohexyl]benzamide;
trans-(−)4-(2-methoxyethoxy)-N-[2-(piperidin-1-ylmethyl)cyclohexyl]benzamide;
trans-(+/−)-3-cyclopentyl-N-[2-(piperidin-1-ylmethyl)cyclohexyl]propanamide;
trans-(+/−)-3-(4-chlorophenyl)-N-[2-(piperidin-1-ylmethyl)cyclohexyl]propanamide;
trans-(+/−)-3-(2-methoxyphenyl)-N-[2-(piperidin-1-ylmethyl)cyclohexyl]propanamide;
trans-(+/−)-4-tert-butyl-N-[2-(piperidin-1-ylmethyl)cyclohexyl]benzamide;
trans-(+/−)-4-methoxy-N-[2-(piperidin-1-ylmethyl)cyclohexyl]benzamide;
trans-(+/−)-4-cyano-N-[2-(piperidin-1-ylmethyl)cyclohexyl]benzamide;
trans-(+/−)-4-bromo-N-[2-(piperidin-1-ylmethyl)cyclohexyl]benzamide;
trans-(+/−)-4-chloro-N-[2-(piperidin-1-ylmethyl)cyclohexyl]benzamide;
trans-(+/−)-6-(1H-imidazol-1-yl)-N-[2-(piperidin-1 ylmethyl)cyclohexyl]nicotinamide;
trans-(+/−)-4-(1,3-oxazol-5-yl)-N-[-2-(piperidin-1-ylmethyl)cyclohexyl]benzamide;
trans-(+/−)-6-methoxy-N-[2-(piperidin-1-ylmethyl)cyclohexyl]nicotinamide;
trans-(+/−)-4-(1H-imidazol-1-yl)-N-[2-(piperidin-1-ylmethyl)cyclohexyl]benzamide;
trans-(+/−)-4-[(4-oxopiperidin-1-yl)carbonyl]-N-[2-(piperidin-1-ylmethyl)cyclohexyl]benzamide;
trans-(+/−)-N-[2-(piperidin-1-ylmethyl)cyclohexyl]-2-pyridin-3-ylacetamide;
trans-(+/−)-2-{[(butylamino)carbonyl]amino}-N-[2-(piperidin-1-ylmethyl)cyclohexyl]benzamide;
trans-(+/−)-4-(1,1-dioxidothiomorpholin-4-yl)-N-[2-(piperidin-1-ylmethyl)cyclohexyl]benzamide;
trans-(+/−)-4-(aminosulfonyl)-N-[2-(piperidin-1-ylmethyl)cyclohexyl]benzamide;
trans-(+/−)-2-morpholin-4-yl-N-[2-(piperidin-1-ylmethyl)cyclohexyl]isonicotinamide;
trans-(+/−)-4-[(diethylamino)methyl]-N-[2-(piperidin-1-ylmethyl)cyclohexyl]benzamide;
trans-(+/−)-N-[2-(piperidin-1-ylmethyl)cyclohexyl]-1-benzothiophene-3-carboxamide;
trans-(+/−)-4-acetyl-N-[2-(piperidin-1-ylmethyl)cyclohexyl]benzamide;
trans-(+/−)-4-[(3-oxo-2,3-dihydro-4H-1,4-benzoxazin-4-yl)methyl]-N-[2-(piperidin-1-ylmethyl)cyclohexyl]benzamide;
trans-(+/−)-1-oxo-N-[2-(piperidin-1-ylmethyl)cyclohexyl]indane-4-carboxamide;
trans-(+/−)-5-[(dimethylamino)methyl]-N-[2-(piperidin-1-ylmethyl)cyclohexyl]-2-furamide;
trans-(+/−)-1-methyl-N-[2-(piperidin-1-ylmethyl)cyclohexyl]-1H-imidazole-4-carboxamide;
trans-(+/−)-2-(4-chlorophenyl)-N-[2-(piperidin-1-ylmethyl)cyclohexyl]acetamide;
trans-(+/−)-N-[2-(piperidin-1-ylmethyl)cyclohexyl]-6-pyrrolidin-1-ylnicotinamide;
trans-(+/−)-5-methyl-N-[2-(piperidin-1-ylmethyl)cyclohexyl]-7-(trifluoromethyl)pyrazolo[1,5-a]pyrimidine-2-carboxamide;
trans-(+/−)-N-[2-(piperidin-1-ylmethyl)cyclohexyl]pyrazine-2-carboxamide;
trans-(+/−)-4-(ethylthio)-N-[2-(piperidin-1-ylmethyl)cyclohexyl]benzamide;
trans-(+/−)-N-[2-(piperidin-1-ylmethyl)cyclohexyl]-1,3-benzothiazole-6-carboxamide;
trans-(+/−)-4-(acetylamino)-N-[2-(piperidin-1-ylmethyl)cyclohexyl]benzamide;
trans-(+/−)-5-methoxy-N-[2-(piperidin-1-ylmethyl)cyclohexyl]-1H-indole-2-carboxamide;
trans-(+/−)-N-[2-(piperidin-1-ylmethyl)cyclohexyl]thiophene-3-carboxamide;
trans-(+/−)-2-phenyl-N-[2-(piperidin-1-ylmethyl)cyclohexyl]acetamide;
trans-(+/−)-N-[2-(piperidin-1-ylmethyl)cyclohexyl]-4-(trifluoromethoxy)benzamide;
trans-(+/−)-3-(2-chlorophenyl)-N-[2-(piperidin-1-ylmethyl)cyclohexyl]propanamide;
trans-(+/−)-N-[2-(piperidin-1-ylmethyl)cyclohexyl]pyrazolo[1,5-a]pyrimidine-3-carboxamide;
trans-(+/−)-N-[2-(piperidin-1-ylmethyl)cyclohexyl]-4-cyano benzamide;
trans-(+/−)-3-(3-chlorophenyl)-N-[2-(piperidin-1-ylmethyl)cyclohexyl]propanamide;
trans-(+/−)-6-fluoro-N-[2-(piperidin-1-ylmethyl)cyclohexyl]-4H-1,3-benzodioxine-8-carboxamide;
trans-(+/−)-N-[2-(piperidin-1-ylmethyl)cyclohexyl]-2-(tetrahydro-2H-pyran-4-yl)acetamide;
trans-(+/−)-4-chloro-2,5-difluoro-N-[2-(piperidin-1-ylmethyl)cyclohexyl]benzamide;
trans-(+/−)-N-[2-(piperidin-1-ylmethyl)cyclohexyl]-1H-indole-6-carboxamide;
trans-(+/−)-3-(1H-1,2,3-benzotriazol-1-yl)-N-[2-(piperidin-1-ylmethyl)cyclohexyl]propanamide;
trans-(+/−)-N-[2-(piperidin-1-ylmethyl)cyclohexyl]-3-(2-thienyl)propanamide;
trans-(+/−)-2-(1-benzofuran-4-yl)-N-[2-(piperidin-1-ylmethyl)cyclohexyl]acetamide;
trans-(+/−)-4-(dimethylamino)-N-[2-(piperidin-1-ylmethyl)cyclohexyl]benzamide;
trans-(+/−)-N-[2-(piperidin-1-ylmethyl)cyclohexyl]-3-pyridin-3-ylpropanamide;
trans-(+/−)-4,6-dimethyl-N-[2-(piperidin-1-ylmethyl)cyclohexyl]nicotinamide;
trans-(+/−)-3-(5-methyl-2-furyl)-N-[2-(piperidin-1-ylmethyl)cyclohexyl]-1H-pyrazole-5-carboxamide;
trans-(+/−)-2-cyclopropyl-N-[2-(piperidin-1-ylmethyl)cyclohexyl]acetamide;
trans-(+/−)-5-methoxy-N-[2-(piperidin-1-ylmethyl)cyclohexyl]-1-benzofuran-2-carboxamide;
trans-(+/−)-N-[2-(piperidin-1-ylmethyl)cyclohexyl]-1H-indazole-3-carboxamide;
trans-(+/−)-6-(ethylthio)-N-[2-(piperidin-1-ylmethyl)cyclohexyl]nicotinamide;
trans-(+/−)-N-[2-(piperidin-1-ylmethyl)cyclohexyl]-4-(1H-pyrrol-1-yl)benzamide;
trans-(+/−)-N-[2-(piperidin-1-ylmethyl)cyclohexyl]-1H-indole-4-carboxamide;
trans-(+/−)-2-chloro-N-[2-(piperidin-1-ylmethyl)cyclohexyl]benzamide;
trans-(+/−)-3-cyano-N-[2-(piperidin-1-ylmethyl)cyclohexyl]benzamide;
trans-(+/−)-2-methyl-N-[2-(piperidin-1-ylmethyl)cyclohexyl]-5-(trifluoromethyl)-1,3-oxazole-4-carboxamide;
trans-(+/−)-3-chloro-4-methyl-N-[2-(piperidin-1-ylmethyl)cyclohexyl]thiophene-2-carboxamide;
trans-(+/−)-3-(5-methyl-1H-pyrazol-1-yl)-N-[2-(piperidin-1-ylmethyl)cyclohexyl]propanamide;
trans-(+/−)-3-methoxy-N-[2-(piperidin-1-ylmethyl)cyclohexyl]benzamide;
trans-(+/−)-2-(2,3-dihydro-1-benzofuran-5-yl)-N-[2-(piperidin-1-ylmethyl)cyclohexyl]acetamide;
trans-(+/−)-N-[2-(piperidin-1-ylmethyl)cyclohexyl]-1,3-benzodioxole-5-carboxamide;
trans-(+/−)-5-methyl-N-[2-(piperidin-1-ylmethyl)cyclohexyl]thiophene-2-carboxamide;
trans-(+/−)-1-ethyl-5-methyl-N-[2-(piperidin-1-ylmethyl)cyclohexyl]-1H-pyrazole-4-carboxamide;
trans-(+/−)-5-ethoxy-N-[2-(piperidin-1-ylmethyl)cyclohexyl]-2-furamide;
trans-(+/−)-3-(4-fluorophenoxy)-N-[2-(piperidin-1-ylmethyl)cyclohexyl]propanamide;
trans-(+/−)-3-fluoro-4-methoxy-N-[2-(piperidin-1-ylmethyl)cyclohexyl]benzamide;
trans-(+/−)-N-[2-(piperidin-1-ylmethyl)cyclohexyl]-4-propylbenzamide;
trans-(+/−)-N-[2-(piperidin-1-ylmethyl)cyclohexyl]hexanamide;
trans-(+/−)-4-butoxy-N-[2-(piperidin-1-ylmethyl)cyclohexyl]benzamide;
trans-(+/−)-4-chloro-2-fluoro-N-[2-(piperidin-1-ylmethyl)cyclohexyl]benzamide;
trans-(+/−)-2-oxo-N-[2-(piperidin-1-ylmethyl)cyclohexyl]-2,3-dihydro-1H-benzimidazole-5-carboxamide;
trans-(+/−)-2-(4-ethoxyphenyl)-N-[2-(piperidin-1-ylmethyl)cyclohexyl]acetamide;
trans-(+/−)-3-phenyl-N-[2-(piperidin-1-ylmethyl)cyclohexyl]isoxazole-5-carboxamide;
trans-(+/−)-2-methoxy-5-methyl-N-[2-(piperidin-1-ylmethyl)cyclohexyl]benzamide;
trans-(+/−)-4-methoxy-N-{2-[(4-phenylpiperidin-1-yl)methyl]cyclohexyl}benzamide;
trans-(+/−)-N-[2-(1,4-dioxa-8-azaspiro[4,5]dec-8-ylmethyl)cyclohexyl]-4-methoxybenzamide;
trans-(+/−)-N-{2-[(3,5-dimethylpiperidin-1-yl)methyl]cyclohexyl}-4-methoxybenzamide;
trans-(+/−)-N-{2-[(4-fluoropiperidin-1-yl)methyl]cyclohexyl}-4-methoxybenzamide;
trans-(+/−)-4-methoxy-N-(2-{[4-(trifluoromethyl)piperidin-1-yl]methyl}cyclohexyl)benzamide;
trans-(+/−)-4-methoxy-N-{2-[(4-methoxypiperidin-1-yl)methyl]cyclohexyl}benzamide;
trans-(+/−)-4-methoxy-N-(2-{[3-(trifluoromethyl)piperidin-1-yl]methyl}cyclohexyl)benzamide;
trans-(+/−)-4-methoxy-N-{2-[(3-phenylpiperidin-1-yl)methyl]cyclohexyl}benzamide;
trans-(+/−)-N-[2-({3-[(allyloxy)methyl]piperidin-1-yl}methyl)cyclohexyl]-4-methoxybenzamide;
trans-(+/−)-N-[2-({3-[(allyloxy)methyl]piperidin-1-yl}methyl)cyclohexyl]-6-(1H-pyrazol-1-yl)nicotinamide;
trans-(+/−)-N-(2-{[3-(methoxymethyl)piperidin-1-yl]methyl}cyclohexyl)-6-(1H-pyrazol-1-yl)nicotinamide;
trans-(+/−)-N-(2-{[3-(ethoxymethyl)piperidin-1-yl]methyl}cyclohexyl)-6-(1H-pyrazol-1-yl)nicotinamide;
trans-(+/−)-N-{2-[(3-pentylpiperidin-1-yl)methyl]cyclohexyl}-6-(1H-pyrazol-1-yl)nicotinamide;
trans-(+/−)-N-{2-[(3-pentylpiperidin-1-yl)methyl]cyclohexyl}-4-(1H-pyrazol-1-yl)benzamide;
trans-(+/−)-6-(1H-imidazol-1-yl)-N-{2-[(3-pentylpiperidin-1-yl)methyl]cyclohexyl}nicotinamide;
trans-(+/−)-N-{2-[(3-pentylpiperidin-1-yl)methyl]cyclohexyl}-6-pyrrolidin-1-ylnicotinamide;
trans(±)-6-(1H-imidazol-1-yl)-N-(2-{[(3R)-3-pentylpiperidin-1-yl]methyl}cyclohexyl)nicotinamide;
trans(±)-6-(1H-imidazol-1-yl)-N-(2-{[(3S)-3-pentylpiperidin-1-yl]methyl}cyclohexyl)nicotinamide;
trans-(+/−)-N-{(2-[(3-hexylpiperidin-1-yl)methyl]cyclohexyl}-6-(1H-pyrazol-1-yl)nicotinamide;
trans-(+/−)-N-{2-[(3-hexylpiperidin-1-yl)methyl]cyclohexyl}-6-(1H-imidazol-1-yl)nicotinamide;
trans-(+/−)-N-{2-[(3-hexylpiperidin-1-yl)methyl]cyclohexyl}-4-(1H-pyrazol-1-yl)benzamide;
trans-(+/−)-N-{2-[(3-hexylpiperidin-1-yl)methyl]cyclohexyl}-4-pyrrolidin-1-ylbenzamide;
trans-(+/−)-N-{(2-[(3-butylpiperidin-1-yl)methyl]cyclohexyl}-6-(1H-pyrazol-1-yl)nicotinamide;
trans-(+/−)-N-{2-[(3-butylpiperidin-1-yl)methyl]cyclohexyl}-4-pyrrolidin-1-ylbenzamide;
trans-(+/−)-N-{2-[(3-butylpiperidin-1-yl)methyl]cyclohexyl}-6-(1H-imidazol-1-yl)nicotinamide;
trans-(+/−)-N-{2-[(3-butylpiperidin-1-yl)methyl]cyclohexyl}-4-(1H-pyrazol-1-yl)benzamide;
cis-(+/−)-N-{2-[(3-butylpiperidin-1-yl)methyl]cyclohexyl}-6-(1H-imidazol-1-yl)nicotinamide;
trans-(+/−)-N-(2-{[4-(Allyloxy)piperidin-1-yl]methyl}cyclohexyl)-6-(1H-pyrazol-1-yl)nicotinamide;
trans-(+/−)-N-[2-({4-[(2E)-But-2-en-1-yloxy]piperidin-1-yl}methyl)cyclohexyl]-6-(1H-pyrazol-1-yl)nicotinamide;
trans-(+/−)-N-[2-({3-[(Allyloxy)methyl]piperidin-1-yl}methyl)cyclohexyl]-6-pyrrolidin-1-ylnicotinamide;
trans-(+/−)-N-[2-({3-[(Allyloxy)methyl]piperidin-1-yl}methyl)cyclohexyl]-4-(1H-pyrazol-1-yl)benzamide;
trans-(+/−)-N-[2-({3-[(Allyloxy)methyl]piperidin-1-yl}methyl)cyclohexyl]-6-(1H-imidazol-1-yl)nicotinamide;
trans-(±)-N-2-({3-[(Allyloxy)methyl]piperidin-1-yl}methyl)cyclohexyl]-4-bromobenzamide;
Trans-(±)-(N-2-({3-[(Allyloxy)methyl]piperidin-1-yl}methyl)cyclohexyl]-3-(4-chlorophenyl)propanamide
Trans-(±)-N-[2-({3-[(Allyloxy)methyl]piperidin-1-yl}methyl)cyclohexyl]-3-(2-methoxyphenyl)propanamide
Trans-(±)-N-[2-({3-[(Allyloxy)methyl]piperidin-1-yl}methyl)cyclohexyl]-4-cyanobenzamide
Trans-(±)-N-[(2-({3-[(Allyloxy)methyl]piperidin-1-yl}methyl)cyclohexyl]-4-fluorobenzamide
Trans-(±)-N-[(2-({3-[(Allyloxy)methyl]piperidin-1-yl}methyl)cyclohexyl]-4-chlorobenzamide
Trans-(±)-N-[2-({3-[(Allyloxy)methyl]piperidin-1-yl}methyl)cyclohexyl]-4-[(diethylamino)methyl]benzamide
Trans-(±)-N-[2-({3-[(Allyloxy)methyl]piperidin-1-yl}methyl)cyclohexyl]-4-[(4-methylpiperazin-1-yl)methyl]benzamide;
trans(±)-[2-({(3R)-3-[(Allyloxy)methyl]piperidin-1-yl}methyl)cyclohexyl]-6-(1H-imidazol-1-yl)nicotinamide;
trans(±)-[2-({(3S)-3-[(Allyloxy)methyl]piperidin-1-yl}methyl)cyclohexyl]-6-(1H-imidazol-1-yl)nicotinamide;
trans-(+/−)-N-{2-[(4-benzylpiperidin-1-yl)methyl]cyclohexyl}-6-(1H-pyrazol-1-yl)nicotinamide;
trans-(+/−)-N-{2-[(4-cyclopentylpiperazin-1-yl)methyl]cyclohexyl}-6-(1H-pyrazol-1-yl)nicotinamide;
trans-(+/−)-N-(2-{[methyl(2-phenylethyl)amino]methyl}cyclohexyl)-6-(1H-pyrazol-1-yl)nicotinamide;
trans-(+/−)-6-(1H-pyrazol-1-yl)-N-(2-{[4-(pyridin-4-ylmethyl)piperazin-1-yl]methyl}cyclohexyl)nicotinamide;
trans-(+/−)-N-(2-{[methyl(pyridin-3-ylmethyl-)amino]methyl}cyclohexyl)-6-(1H-pyrazol-1-yl)nicotinamide;
trans-(+/−)-N-(2-{[(4-ethylbenzyl)(methyl)amino]methyl}cyclohexyl)-6-(1H-pyrazol-1-yl)nicotinamide;
trans-(+/−)-N-(2-{[methyl(1-methylpyrrolidin-3-yl)amino]methyl}cyclohexyl)-6-(1H-pyrazol-1-yl)nicotinamide;
trans-(+/−)-N-(2-{[methyl(3-methylbutyl)amino]methyl}cyclohexyl)-6-(1H-pyrazol-1-yl)nicotinamide;
trans-(+/−)-N-(2-{[methyl(propyl)amino]methyl}cyclohexyl)-6-(1H-pyrazol-1-yl)nicotinamide;
trans-(+/−)-N-(2-{[benzyl(methyl)amino]methyl}cyclohexyl)-6-(1H-pyrazol-1-yl)nicotinamide;
trans-(+/−)-N-{2-[(4-propylpiperidin-1-yl)methyl]cyclohexyl}-6-(1H-pyrazol-1-yl)nicotinamide;
trans-(+/−)-N-(2-{[2-(methoxymethyl)piperidin-1-yl]methyl}cyclohexyl)-6-(1H-pyrazol-1-yl)nicotinamide;
trans-(+/−)-N-(2-{[butyl(methyl)amino]methyl}cyclohexyl)-6-(1H-pyrazol-1-yl)nicotinamide;
trans-(+/−)-N-(2-{[butyl(ethyl)amino]methyl}cyclohexyl)-6-(1H-pyrazol-1-yl)nicotinamide;
trans-(+/−)-6-(1H-pyrazol-1-yl)-N-(2-{[2-(3-thienylmethyl)piperidin-1-yl]methyl}cyclohexyl)nicotinamide;
trans-(+/−)-N-{2-[(4,4-difluoropiperidin-1-yl)methyl]cyclohexyl}-4-methoxybenzamide;
trans-(+/−)-4-methoxy-N-{2-[(4-methylpiperidin-1-yl)methyl]cyclohexyl}benzamide;
trans-(+/−)-4-(2-methoxyethoxy)-N-{2-[(4-methylpiperidin-1-yl)methyl]cyclohexyl}benzamide;
trans-(+/−)-4-methoxy-N-[2-(morpholin-4-ylmethyl)cyclohexyl]benzamide;
cis-(+/−)-4-(2-ethoxyethoxy)-N-[2-(piperidin-1-ylmethyl)cyclohexyl]benzamide;
cis-(+/−)-4-(2-ethoxyethoxy)-N-[2-(pyrrolidin-1-ylmethyl)cyclohexyl]benzamide;
cis-(+/−)-N-{2-[(diethylamino)methyl]cyclohexyl}-4-(2-ethoxyethoxy)benzamide;
trans-(+/−)-4-(2-ethoxyethoxy)-N-[2-(piperidin-1-ylmethyl)cyclohexyl]benzamide;
trans-(+/−)-N-[2-(azepan-1-ylmethyl)cyclohexyl]-4-(2-ethoxyethoxy)benzamide;
trans-(+/−)-N-{2-[(diethylamino)methyl]cyclohexyl}-4-(2-ethoxyethoxy)benzamide;
trans-(+/−)-N-(4-chlorophenyl)-N′-[2-(piperidin-1-ylmethyl)cyclohexyl]urea;
trans-(+/−)-N-(4-cyanophenyl)-N′-[2-(piperidin-1-ylmethyl)cyclohexyl]urea;
trans-(+/−)-N-(4-methoxyphenyl)-N′-[2-(piperidin-1-ylmethyl)cyclohexyl]urea;
trans-(+/−)-2-methoxy-4-methyl-N-[2-(piperidin-1-ylmethyl)cyclohexyl]benzenesulfonamide;
trans-(+/−)-methyl 3-({[2-(piperidin-1-ylmethyl)cyclohexyl]amino}sulfonyl)thiophene-2-carboxylate;
trans-(+/−)-5-[2-(methylthio)pyrimidin-4-yl]-N-[2-(piperidin-1-ylmethyl)cyclohexyl]thiophene-2-sulfonamide;
trans-(+/−)-1-(4-chlorophenyl)-N-[2-(piperidin-1-ylmethyl)cyclohexyl]methanesulfonamide;
trans-(+/−)-N-{2-[(3-butylpiperidin-1-yl)methyl]cyclohexyl}-4-(1,3-oxazol-5-yl)benzamide;
trans-(+/−)-N-{2-[(3-butylpiperidin-1-yl)methyl]cyclohexyl}-6-(trifluoromethyl)nicotinamide;
trans-(+/−)-N-{2-[(3-butylpiperidin-1-yl)methyl]cyclohexyl}-4-(2-methoxyethoxy)benzamide;
trans-(+/−)-N-{2-[(3-butylpiperidin-1-yl)methyl]cyclohexyl}-3-(4-chlorophenyl)propanamide;
trans-(+/−)-N-{2-[(3-butylpiperidin-1-yl)methyl]cyclohexyl}-4-(1H-imidazol-1-yl)benzamide;
trans-(+/−)-N-(2-{[3-(ethoxymethyl)piperidin-1-yl]methyl}cyclohexyl)-6-(1H-imidazol-1-yl)nicotinamide;
trans-(+/−)-N-(2-{[3-(ethoxymethyl)piperidin-1-yl]methyl}cyclohexyl)-4-(1,3-oxazol-5-yl)benzamide;
trans-(+/−)-N-(2-{[3-(ethoxymethyl)piperidin-1-yl]methyl}cyclohexyl)-4-(1H-imidazol-1-yl)benzamide;
trans-(+/−)-N-2-{[3-(ethoxymethyl)piperidin-1-yl]methyl}cyclohexyl)-4-{[(methylsulfonyl)amino]methyl}benzamide;
trans-(+/−)-N-(2-{[3-propylpiperidin-1-yl]methyl}cyclohexyl)-6-(1H-imidazol-1-yl)nicotinamide;
trans-(+/−)-4-(1H-imidazol-1-yl)-N-{2-[(3-propylpiperidin-1-yl)methyl]cyclohexyl}benzamide;
trans-(+/−)-N-(2-{[3-isobutylpiperidin-1-yl]methyl}cyclohexyl)-6-(1H-imidazol-1-yl)nicotinamide;
trans-(+/−)-4-(1H-imidazol-1-yl)-N-{2-[(3-isobutylpiperidin-1-yl)methyl]cyclohexyl}benzamide;
trans-(+/−)-4-Bromo-N-{2-[(3-propylpiperidin-1-yl)methyl]cyclohexyl}benzamide;
trans-(+/−)-3-(4-Chlorophenyl)-N-{2-[(3-propylpiperidin-1-yl)methyl]cyclohexyl}propanamide;
trans-(+/−)-4-Bromo-N-{2-[(3-butylpiperidin-1-yl)methyl]cyclohexyl}benzamide;
trans-(+/−)-N-{2-[(3-Butylpiperidin-1-yl)methyl]cyclohexyl}-4-[(diethylamino)methyl]benzamide;
trans-(+/−)-3-(4-Chlorophenyl)-N-(2-{[3-(ethoxymethyl)piperidin-1-yl]methyl}cyclohexyl)propanamide;
N-[(1S,2R)-2-({4-[(2E)-But-2-en-1-yloxy]piperidin-1-yl}methyl)cyclohexyl]-6-(1H-pyrazol-1-yl)nicotinamide;
N-{(1S,2R)-2-[(4-Butoxypiperidin-1-yl)methyl]cyclohexyl}-6-(1H-pyrazol-1-yl)nicotinamide;
N-(1S,2R)-2-{[(3R)-3-(2-Methoxyethoxy)piperidin-1-yl]methyl}cyclohexyl)-4-(1H-pyrazol-1-yl)benzamide;
N-(1R,2S)-2-{[(3R)-3-(2-Methoxyethoxy)piperidin-1-yl]methyl}cyclohexyl)-4-(1H-pyrazol-1-yl)benzamide;
N-[(1S,2R)-2-({(3R)-3-[(Allyloxy)methyl]piperidin-1-yl}methyl)cyclohexyl]-6-(1H-pyrazol-1-yl)nicotinamide;
N-[(1R,2S)-2-({(3R)-3-[(Allyloxy)methyl]piperidin-1-yl}methyl)cyclohexyl]-6-(1H-pyrazol-1-yl)nicotinamide;
N-[(1R,2S)-2-({(3R)-3-[(Allyloxy)methyl]piperidin-1-yl}methyl)cyclohexyl]-6-(1H-pyrazol-1-yl)nicotinamide
N-[(1S,2R)-2-({(3R)-3-[(Allyloxy)methyl]piperidin-1-yl}methyl)cyclohexyl]-6-(1H-imidazol-1-yl)nicotinamide;
(N-((1S,2R)-2-{[(3R)-3-ethoxypiperidin-1-yl]methyl}cyclohexyl)pyrazine-2-carboxamide;
N-((1S,2R)-2-{[(3R)-3-ethoxypiperidin-1-yl]methyl}cyclohexyl)-6-(ethylthio)nicotinamide;
N-((1S,2R)-2-{[(3R)-3-ethoxypiperidin-1-yl]methyl}cyclohexyl)-6-pyrrolidin-1-ylnicotinamide;
N-[(1S,2R)-2-(azepan-1-ylmethyl)cyclohexyl]-4-(1H-pyrazol-1-yl)benzamide;
N-[(1S,2R)-2-(azepan-1-ylmethyl)cyclohexyl]-6-(1H-pyrazol-1-yl)nicotinamide;
N-((1S,2R)-2-{[(3R)-3-(allyloxy)piperidin-1-yl]methyl}cyclohexyl)-4-(1H-pyrazol-1-yl)benzamide;
N-((1R,2S)-2-{[(3R)-3-(ethoxymethyl)piperidin-1-yl]methyl}cyclohexyl)-4-(1H-pyrrol-1-yl)benzamide;
N-((1S,2R)-2-{[(3R)-3-(ethoxymethyl)piperidin-1-yl]methyl}cyclohexyl)-4-(1H-pyrrol-1-yl)benzamide;
N-((1R,2S)-2-{[(3R)-3-(ethoxymethyl)piperidin-1-yl]methyl}cyclohexyl)-6-pyrrolidin-1-ylnicotinamide;
N-((1S,2R)-2-{[(3R)-3-(ethoxymethyl)piperidin-1-yl]methyl}cyclohexyl)-6-pyrrolidin-1-ylnicotinamide;
N-[(1S,2R)-2-(piperidin-1-ylmethyl)cyclohexyl]-4-(1H-pyrazol-1-yl)benzamide;
N-[(1S,2R)-2-(piperidin-1-ylmethyl)cyclohexyl]-6-(1H-pyrazol-1-yl)nicotinamide;
N-((1S,2R)-2-{[(3R)-3-(allyloxy)piperidin-1-yl]methyl}cyclohexyl)-4-(1H-pyrrol-1-yl)benzamide;
N-((1S,2R)-2-{[(3R)-3-(allyloxy)piperidin-1-yl]-methyl}cyclohexyl)-3-cyclopentylpropanamide;
N-((1S,2R)-2-{[(3R)-3-(allyloxy)piperidin-1-yl]methyl}cyclohexyl)-6-(1H-pyrazol-1-yl)nicotinamide;
N-((1S,2R)-2-{[(3S)-3-(allyloxy)piperidin-1-yl]methyl}cyclohexyl)-6-(1H-pyrazol-1-yl)nicotinamide;
N-((1S,2R)-2-{[(3S)-3-(ethoxymethyl)piperidin-1-yl]methyl}cyclo-hexyl)-4-(2-methoxyethoxy)benzamide;
3-(4-chlorophenyl)-N-((1S,2R)-2-{[(3S)-3-(ethoxymethyl)piperidin-1-yl]methyl}cyclohexyl)propanamide;
N-((1S,2R)-2-{[(3S)-3-(ethoxymethyl)piperidin-1-yl]methyl}cyclo-hexyl)-4-{[(methylsulfonyl)amino]methyl}benzamide;
4-[(diethylamino)methyl]-N-((1S,2R)-2-{[(3S)-3-(ethoxymethyl)-piperidin-1-yl]methyl}cyclohexyl)benzamide;
N-[(1S,2R)-2-({(3R)-3-[(allyloxy)methyl]piperidin-1-yl}methyl)cyclohexyl]-6-(1H-imidazol-1-yl)nicotinamide;
4-chloro-N-((1S,2R)-2-{[(3R)-3-(ethoxymethyl)piperidin-1-yl]methyl}cyclohexyl)benzamide;
N-((1S,2R)-2-{[(3R)-3-(ethoxymethyl)piperidin-1-yl]methyl}cyclohexyl)benzamide;
N-((1S,2R)-2-{[(3R)-3-(ethoxymethyl)piperidin-1-yl]methyl}cyclohexyl)cyclohexanecarboxamide;
N-((1S,2R)-2-{[(3R)-3-(ethoxymethyl)piperidin-1-yl]methyl}cyclohexyl)-2-phenylacetamide;
N-((1S,2R)-2-{[(3R)-3-(ethoxymethyl)piperidin-1-yl]methyl}cyclohexyl)-3-phenylpropanamide;
N-((1S,2R)-2-{[(3R)-3-(ethoxymethyl)piperidin-1-yl]methyl}cyclohexyl)-2,3-dihydro-1-benzofuran-5-carboxamide;
2-cyclopentyl-N-((1S,2R)-2-{[(3R)-3-(ethoxymethyl)piperidin-1-yl]methyl}cyclohexyl)acetamide;
2-chloro-N-((1S,2R)-2-{[(3R)-3-(ethoxymethyl)piperidin-1-yl]methyl}cyclohexyl)-3-fluoroisonicotinamide;
N-((1S,2R)-2-{[(3R)-3-(ethoxymethyl)piperidin-1-yl]methyl}cyclohexyl)chromane-2-carboxamide;
N-((1S,2R)-2-{[(3R)-3-(ethoxymethyl)piperidin-1-yl]methyl}cyclohexyl)-4,6-dimethylnicotinamide;
N-((1S,2R)-2-{[(3R)-3-(ethoxymethyl)piperidin-1-yl]methyl}cyclohexyl)-2,7-dimethylimidazo[1,2-a]pyridine-3-carboxamide hydrochloride salt;
N-((1S,2R)-2-{[(3R)-3-(ethoxymethyl)piperidin-1-yl]methyl}cyclohexyl)-2-(3-methoxyphenyl)acetamide;
2-(2,3-dioxo-2,3-dihydro-1H-indol-1-yl)-N-((1S,2R)-2-{[(3R)-3-(ethoxymethyl)piperidin-1-yl]methyl}cyclohexyl)acetamide hydrochloride salt;
N2-acetyl-N1-((1S,2R)-2-{[(3R)-3-(ethoxymethyl)piperidin-1-yl]methyl}cyclohexyl)glycinamide;
N-((1S,2R)-2-{[(3R)-3-(ethoxymethyl)piperidin-1-yl]methyl}cyclohexyl)-2-(1H-tetrazol-1-yl)acetamide;
N-((1S,2R)-2-{[(3R)-3-(ethoxymethyl)piperidin-1-yl]methyl}cyclohexyl)-5,7-dimethylpyrazolo[1,5-a]pyrimidine-2-carboxamide;
N-((1S,2R)-2-{[(3R)-3-(ethoxymethyl)piperidin-1-yl]methyl}cyclohexyl)-3,4-dihydro-2H-1,5-benzodioxepine-6-carboxamide;
N-((1S,2R)-2-{[(3R)-3-(ethoxymethyl)piperidin-1-yl]methyl}cyclohexyl)-4-methyl-3,4-dihydro-2H-1,4-benzoxazine-7-carboxamide;
N-((1S,2R)-2-{[(3R)-3-(ethoxymethyl)piperidin-1-yl]methyl}cyclohexyl)-5-phenyl-1H-pyrazole-4-carboxamide;
N-((1S,2R)-2-{[(3R)-3-(ethoxymethyl)piperidin-1-yl]methyl}cyclohexyl)-4-(1H-tetrazol-1-yl)benzamide;
4-[(diethylamino)methyl]-N-((1S,2R)-2-{[(3R)-3-(ethoxymethyl)piperidin-1-yl]methyl}cyclohexyl)benzamide;
N-((1S,2R)-2-{[(3R)-3-(ethoxymethyl)piperidin-1-yl]methyl}cyclohexyl)-4-(2-methoxyethoxy)benzamide;
N-((1S,2R)-2-{[(3R)-3-(ethoxymethyl)piperidin-1-yl]methyl}cyclohexyl)-4-{[(methylsulfonyl)amino]methyl}benzamide;
4-[(acetylamino)methyl]-N-((1S,2R)-2-{[(3R)-3-(ethoxymethyl)piperidin-1-yl]methyl}cyclohexyl)benzamide;
4-[(diacetylamino)methyl]-N-((1S,2R)-2-{[(3R)-3-(ethoxymethyl)piperidin-1-yl]methyl}cyclohexyl)benzamide;
N-((1S,2R)-2-{[(3R)-3-(ethoxymethyl)piperidin-1-yl]methyl}cyclohexyl)-4-{[(ethylsulfonyl)amino]methyl}benzamide;
4-{[(cyclopropylsulfonyl)amino]methyl}-N-((1S,2R)-2-{[(3R)-3-(ethoxymethyl)piperidin-1-yl]methyl}cyclohexyl)benzamide;
N-((1S,2R)-2-{[(3R)-3-(ethoxymethyl)piperidin-1-yl]methyl}cyclohexyl)-4-({[(methylamino)carbonyl]amino}methyl)benzamide;
4-({[(dimethylamino)carbonyl]amino}methyl)-N-((1S,2R)-2-{[(3R)-3-(ethoxymethyl)piperidin-1-yl]methyl}cyclohexyl)benzamide;
N-((1S,2R)-2-{[(3R)-3-(ethoxymethyl)piperidin-1-yl]methyl}cyclohexyl)-4-[(isobutyrylamino)methyl]benzamide;
N-((1S,2R)-2-{[3-cyclohexylpiperidin-1-yl]methyl}cyclohexyl)-6-(1H-pyrazol-1-yl)nicotinamide;
N-((1S,2R)-2-{[3-phenylpiperidin-1-yl]methyl}cyclohexyl)-6-(1H-pyrazol-1-yl)nicotinamide;
and pharmaceutically acceptable salts thereof.

8. A compound of formula V, a pharmaceutically acceptable salt thereof, diastereomer, enantiomer, or mixture thereof: 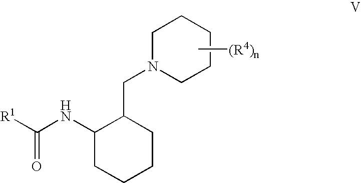
wherein
R¹ is selected from C₆₋₁₀aryl, C₂₋₉heteroaryl, C₃₋₅heterocycloalkyl, C₆₋₁₀aryl-C₁₋₃alkyl, C₂₋₉heteroaryl-C₁₋₃alkyl, C₃₋₅heterocycloalkyl-C₁₋₃alkyl, C₃₋₆cycloalkyl, C₃₋₆cycloalkyl-C₁₋₃alkyl, and C₁₋₆alkyl, wherein said C₆₋₁₀aryl, C₂₋₉heteroaryl, C₆₋₁₀aryl-C₁₋₃alkyl, C₆₋₁₀aryl-O—C₁₋₃alkyl, C₂₋₉heteroaryl-C₁₋₃alkyl, C₃₋₆cycloalkyl, C₃₋₆cycloalkyl-C₁₋₃alkyl, and C₁₋₆alkyl are optionally substituted with one or more group selected from C₆₋₁₀aryl, C₁₋₉heteroaryl, C₃₋₅heterocycloalkyl, C₆₋₁₀aryl-C₁₋₃alkyl, C₆₋₁₀aryl-O—C₁₋₃alkyl, C₂₋₉heteroaryl-C₁₋₃alkyl, C₃₋₅heterocycloalkyl-C₁₋₃alkyl, —CN, —SR, —OR, —O(CH₂)ₘ—OR, R, —C(═O)—R, —CO₂R, —SO₂R, —SO₂NR₂, halogen, —NO₂, —NR₂, —(CH₂)ₘNR₂, —(CH₂)ₘNHC(═O)—NR₂, —NHC(═O)—R, —N[C(═O)R]₂, —(CH₂)ₘNHC(═O)—R, —(CH₂)ₘN[C(═O)—R]₂—(CH₂)ₘNHS(═O)₂—R, and —C(═O)—NR₂; and
R⁴ is selected from C₆₋₁₀aryl, C₂₋₉heteroaryl, C₃₋₆cycloalkyl, C₃₋₅heterocycloalkyl, C₆₋₁₀aryl-C₁₋₃alkyl, C₂₋₉heteroaryl-C₁₋₃alkyl, C₃₋₅heterocycloalkyl-C₁₋₃alkyl, —CN, —SR, —OR, —(CH₂)ₘOR, —O(CH₂)ₘOR, —O(CH₂)ₘNR₂, —(CH₂)ₘ—O—(CH₂)ₘOR, —(CH₂)ₘ—O—(CH₂)ₙNR₂, R, —CO₂R; —SO₂R; —SO₂NR₂, halogen, —NO₂, —NR₂, —(CH₂)ₘNR₂, and —C(═O)—NR₂;
each R is independently hydrogen, C₁₋₆alkyl, C₂₋₆alkenyl or halogenated C₁₋₆alkyl;
with a proviso that
R¹ is not 4-amino-5-chloro-2-alkoxylphenyl, 4-amino-5-chloro-2-cycloalkoxyphenyl, 4-amino-5-chloro-2-cycloalkyl-alkoxy-phenyl, 4-butoxyphenyl, 3-butoxyphenyl, 4-pentyloxyphenyl, 4-isobutoxyphenyl, 4-benzoyloxyphenyl and 7-(2,3-dihydro)benzofuranyl.

9. A compound as claimed in claim 8, wherein
R¹ is selected from C₆₋₁₀aryl, C₂₋₉heteroaryl, C₃₋₅heterocycloalkyl, C₆₋₁₀aryl-C₁₋₃alkyl, C₂₋₉heteroaryl-C₁₋₃alkyl, C₃₋₅heterocycloalkyl-C₁₋₃alkyl, C₃₋₆cycloalkyl, C₃₋₆cycloalkyl-C₁₋₃alkyl, and C₃₋₆alkyl, wherein said C₆₋₁₀aryl, C₂₋₉heteroaryl, C₆₋₁₀aryl-C₁₋₃alkyl, C₆₋₁₀aryl-O—C₁₋₃alkyl, C₂₋₉heteroaryl-C₁₋₃alkyl, C₃₋₆cycloalkyl, C₃₋₆cycloalkyl-C₁₋₃alkyl, and C₃₋₆alkyl are optionally substituted by one or more groups selected from 1H-pyrazol-1-yl, fluoro, chloro, trifluoromethyl, methoxy, difluoromethoxy, trifluoromethoxy, 2-methoxyethoxy, 2-ethoxyethoxy, t-butyl, cyano, bromo, 1,3-oxazol-5-yl, 1H-imidazol-1-yl, (4-oxopiperidin-1-yl)carbonyl, pyridin-3-ylmethyl, [(butylamino)carbonyl]amino, 1,1-dioxidothiomorpholin-4-yl, aminosulfonyl, morpholin-4-yl, diethylaminomethyl, acetyl, (3-oxo-2,3-dihydro-4H-1,4-benzoxazin-4-yl)methyl, 1-oxo-indan-4-yl, dimethylaminomethyl, methyl, pyrrolidin-1-yl, ethylthio, acetylamino, dimethylamino, 1H-pyrrol-1-yl, ethyl, ethoxy, fluorophenoxy, propyl, phenyl, methoxycarbonyl, diacetylamino, (methylsulfonylamino)methyl, (cyclopropylsulfonylamino)methyl, 1H-tetrazol-1-yl, pyrazolyl, methylaminocarbonylamino, dimethylaminocarbonylamino, and (methylthio)pyrimidin-4-yl.

10. A compound as claimed in claim 8, wherein said R¹ is selected from 2-cyclopentylethyl, cyclopropylmethyl, ethyl, methyl, cyclohexyl, cyclopentylmethyl, chromanyl, pentyl, 2-phenylethyl, phenyl, benzyl, pyridinyl, pyridinylethyl, 1-benzofuranyl, benzothienyl, furyl, imidazolyl, pyrazolo[1,5-a]pyrimidinyl, pyrazinyl, 1,3-benzothiazolyl, indolyl, indazolyl, thienyl, 1,3-benzodioxinyl, tetrahydro-2H-pyran-4-ylmethyl, 1-H-1,2,3-benzotriazol-1-yl, 2-(thien-2-yl)ethyl, (1-benzofuran-4-yl)methyl, 1,3-oxazolyl, 1H-pyrazol-1-yl, 2,3-dihydro-1-benzofuran-5-yl, 1,3-benzodioxol-5-yl, 2-oxo-2,3-dihydro-2H-benzimidazolyl, isoxazolyl, imidazo[1,2,a]pyridinyl, 2-3-dioxo-2,3-dihydro-1H-indol-1-yl, 3,4-dihydro-2H-1,4-benzoxazinyl; pyrazolyl, 1H-tetrazol-1-yl-methyl, and 3,4-dihydro-2H-1,5-benzodioxepinyl, which are optionally substituted by one or more groups selected from C₆₋₁₀aryl, C₂₋₉heteroaryl, C₃₋₅heterocycloalkyl, C₆₋₁₀aryl-C₁₋₃alkyl, C₆₋₁₀aryl-O—C₁₋₃alkyl, C₂₋₉heteroaryl-C₁₋₃alkyl, C₃₋₅heterocycloalkyl-C₁₋₃alkyl, —CN, —SR, —OR, —O(CH₂)ₘ—OR, R, —C(═O)—R, —CO₂R, —SO₂R, —SO₂NR₂, halogen, —NO₂, —NR₂, —(CH₂)ₘNR₂, —(CH₂)ₘNHC(═O)—NR₂, —NHC(═O)—R, —N[C(═O)R]₂, —(CH₂)ₘNHC(═O)—R—(CH₂)ₘN[C(═O)—R]₂—(CH₂)ₘNHS(═O)₂—R, and —C(═O)—NR₂.

11. A compound as claimed in claim 8, wherein said R¹ is selected from 2-cyclopentylethyl, cyclopropylmethyl, ethyl, methyl, cyclohexyl, cyclopentylmethyl, chromanyl, pentyl, 2-phenylethyl, phenyl, benzyl, pyridinyl, pyridinylethyl, 1-benzofuranyl, benzothienyl, furyl, imidazolyl, pyrazolo[1,5-a]pyrimidinyl, pyrazinyl, 1,3-benzothiazolyl, indolyl, indazolyl, thienyl, 1,3-benzodioxinyl, tetrahydro-2H-pyran-4-ylmethyl, 1-H-1,2,3-benzotriazol-1-yl, 2-(thien-2-yl)ethyl, (1-benzofuran-4-yl)methyl, 1,3-oxazolyl, 1H-pyrazol-1-yl, 2,3-dihydro-1-benzofuran-5-yl, 1,3-benzodioxol-5-yl, 2-oxo-2,3-dihydro-2H-benzimidazolyl, isoxazolyl, imidazo[1,2,a]pyridinyl, 2-3-dioxo-2,3-dihydro-1H-indol-1-yl, 3,4-dihydro-2H-1,4-benzoxazinyl; pyrazolyl, 1H-tetrazol-1-yl-methyl, and 3,4-dihydro-2H-1,5-benzodioxepinyl, which are optionally substituted by are optionally substituted by one or more groups selected from 1H-pyrazol-1-yl, fluoro, chloro, trifluoromethyl, methoxy, difluoromethoxy, trifluoromethoxy, 2-methoxyethoxy, 2-ethoxyethoxy, t-butyl, cyano, bromo, 1,3-oxazol-5-yl, 1H-imidazol-1-yl, (4-oxopiperidin-1-yl)carbonyl, pyridin-3-ylmethyl, [(butylamino)carbonyl]amino, 1,1-dioxidothiomorpholin-4-yl, aminosulfonyl, morpholin-4-yl, diethylaminomethyl, acetyl, (3-oxo-2,3-dihydro-4H-1,4-benzoxazin-4-yl)methyl, 1-oxo-indan-4-yl, dimethylaminomethyl, methyl, pyrrolidin-1-yl, ethylthio, acetylamino, dimethylamino, 1H-pyrrol-1-yl, ethyl, ethoxy, fluorophenoxy, propyl, phenyl, methoxycarbonyl, diacetylamino, (methylsulfonylamino)methyl, (cyclopropylsulfonylamino)methyl, 1H-tetrazol-1-yl, pyrazolyl, methylaminocarbonylamino, dimethylaminocarbonylamino, and (methylthio)pyrimidin-4-yl.

12. A compound as claimed in claim 8, wherein
R⁴ is selected from phenyl, benzyl, methyl, fluoro, trifluoromethyl, methoxy, allyloxy, (2E)-but-2-en-1-yloxy, (allyloxy)methyl, methoxymethyl, ethoxymethyl, propyl, butyl, pentyl, hexyl, cyclopentyl, pyridin-4-ylmethyl, ethoxy, butoxy, 2-methoxyethoxy, cyclohexyl, and thienylmethyl.

13. A pharmaceutical composition comprising a compound according to claim 1 and a pharmaceutically acceptable carrier.

14. A pharmaceutical composition comprising a compound according to claim 7 and a pharmaceutically acceptable carrier.

15. A pharmaceutical composition comprising a compound according to claim 8 and a pharmaceutically acceptable carrier.

16. A method for the therapy of pain in a warm-blooded animal, comprising the step of administering to said animal in need of such therapy a therapeutically effective amount of a compound according to claim 1.

17. A method for the therapy of pain in a warm-blooded animal, comprising the step of administering to said animal in need of such therapy a therapeutically effective amount of a compound according to claim 7.

18. A method for the therapy of pain in a warm-blooded animal, comprising the step of administering to said animal in need of such therapy a therapeutically effective amount of a compound according to claim 8.

19. A method for the therapy of Alzheimer's disease in a warm-blooded animal, comprising the step of administering to said animal in need of such therapy a therapeutically effective amount of a compound according to claim 1.

20. A method for the therapy of Alzheimer's disease in a warm-blooded animal, comprising the step of administering to said animal in need of such therapy a therapeutically effective amount of a compound according to claim 7.

21. A method for the therapy of Alzheimer's disease in a warm-blooded animal, comprising the step of administering to said animal in need of such therapy a therapeutically effective amount of a compound according to claim 8.

22. A method for the therapy of schizophrenia in a warm-blooded animal, comprising the step of administering to said animal in need of such therapy a therapeutically effective amount of a compound according to claim 1.

23. A method for the therapy of schizophrenia in a warm-blooded animal, comprising the step of administering to said animal in need of such therapy a therapeutically effective amount of a compound according to claim 7.

24. A method for the therapy of schizophrenia in a warm-blooded animal, comprising the step of administering to said animal in need of such therapy a therapeutically effective amount of a compound according to claim 8.

25. A process for preparing a compound of Formula II, comprising: 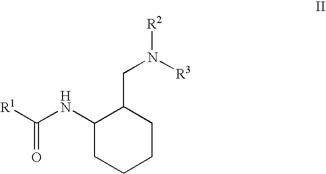
reacting a compound of Formula III with a compound of R¹—COCl or R¹—COOH, 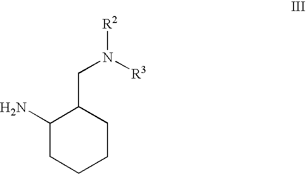
wherein
R¹ is selected from C₆₋₁₀aryl, C₂₋₉heteroaryl, C₃₋₅heterocycloalkyl, C₆₋₁₀aryl-C₁₋₃alkyl, C₂₋₉heteroaryl-C₁₋₃alkyl, C₃₋₅heterocycloalkyl-C₁₋₃alkyl, C₃₋₆cycloalkyl, C₃₋₆cycloalkyl-C₁₋₃alkyl, and C₁₋₆alkyl, wherein said C₆₋₁₀aryl, C₂₋₉heteroaryl, C₆₋₁₀aryl-C₁₋₃alkyl, C₆₋₁₀aryl-O—C₁₋₃alkyl, C₂₋₉heteroaryl-C₁₋₃alkyl, C₃₋₆cycloalkyl, C₃₋₆cycloalkyl-C₁₋₃alkyl, and C₁₋₆alkyl are optionally substituted with one or more group selected from C₆₋₁₀aryl, C₂₋₉heteroaryl, C₃₋₅heterocycloalkyl, C₆₋₁₀aryl-C₁₋₃alkyl, C₆₋₁₀aryl-O—C₁₋₃alkyl, C₂₋₉heteroaryl-C₁₋₃alkyl, C₃₋₅heterocycloalkyl-C₁₋₃alkyl, —CN, —SR, —OR, —O(CH₂)ₘ—OR, R, —C(═O)—R, —CO₂R, —SO₂R, —SO₂NR₂, halogen, —NO₂, —NR₂, —(CH₂)ₘNR₂, —(CH₂)ₘNHC(═O)—NR₂—NHC(═O)—R, —N[C(═O)R]₂, —(CH₂)ₘNHC(═O)—R, —(CH₂)ₘN[C(═O)—R]₂—(CH₂)ₘNHS(═O)₂—R, and —C(═O)—NR₂;
R² and R³ are independently selected from C₁₋₆alkyl, C₂₋₆alkenyl, and C₁₋₆alkoxy wherein said C₁₋₆alkyl, C₂₋₆alkenyl, and C₁₋₆alkoxy are optionally substituted by one or more groups selected from amino, halogen, C₁₋₆alkoxy and —CN; or R² and R³ together with the nitrogen connected thereto form a heterocycloalkyl, wherein said heterocycloalkyl is optionally substituted with one or more group selected from C₆₋₁₀aryl, C₂₋₉heteroaryl, C₃₋₆cycloalkyl, C₃₋₅heterocycloalkyl, C₆₋₁₀aryl-C₁₋₃alkyl, C₂₋₉heteroaryl-C₁₋₃alkyl, C₃₋₅heterocycloalkyl-C₁₋₃alkyl, —CN, —SR, —OR, —(CH₂)ₘOR, R, —CO₂R; —SO₂R; —SO₂NR₂, halogen, —NO₂, —NR₂, —(CH₂)ₘNR₂, and —C(═O)—NR₂;
each R is independently hydrogen, C₁₋₆alkyl, C₂₋₆alkenyl or halogenated C₁₋₆alkyl; and
with a proviso that
when R² and R³ together with the nitrogen connected thereto form said piperidinyl; R¹ is not 4-amino-5-chloro-2-alkoxylphenyl, 4-amino-5-chloro-2-cycloalkoxyphenyl, 4-amino-5-chloro-2-cycloalkyl-alkoxy-phenyl, 4-butoxyphenyl, 3-butoxyphenyl, 4-pentyloxyphenyl, 4-isobutoxyphenyl, 4-benzoyloxyphenyl and 7-(2,3-dihydro)benzofuranyl.

26. A process for preparing a compound of Formula IV, comprising: 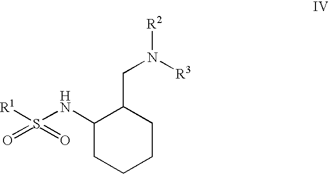
reacting a compound of Formula III with a compound of R¹SO₂Cl, 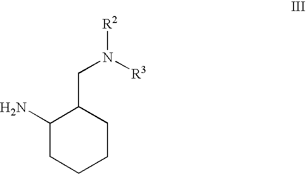
wherein
R¹ is selected from C₆₋₁₀aryl, C₂₋₉heteroaryl, C₃₋₅heterocycloalkyl, C₆₋₁₀aryl-C₁₋₃alkyl, C₂₋₉heteroaryl-C₁₋₃alkyl, C₃₋₅heterocycloalkyl-C₁₋₃alkyl, C₃₋₆cycloalkyl, C₃₋₆cycloalkyl-C₁₋₃alkyl, and C₁₋₆alkyl, wherein said C₆₋₁₀aryl, C₂₋₉heteroaryl, C₆₋₁₀aryl-C₁₋₃alkyl, C₆₋₁₀aryl-O—C₁₋₃alkyl, C₂₋₉heteroaryl-C₁₋₃alkyl, C₃₋₆cycloalkyl, C₃₋₆cycloalkyl-C₁₋₃alkyl, and C₁₋₆alkyl are optionally substituted with one or more group selected from C₆₋₁₀aryl, C₂₋₉heteroaryl, C₃₋₅heterocycloalkyl, C₆₋₁₀aryl-C₁₋₃alkyl, C₆₋₁₀aryl-O—C₁₋₃alkyl, C₂₋₉heteroaryl-C₁₋₃alkyl, C₃₋₅heterocycloalkyl-C₁₋₃alkyl, —CN, —SR, —OR, —O(CH₂)ₘ—OR, R, —C(═O)—R, —CO₂R, —SO₂R, —SO₂NR₂, halogen, —NO₂, —NR₂, —(CH₂)ₘNR₂, —(CH₂)ₘNHC(═O)—NR₂—NHC(═O)—R, —N[C(═O)R]₂, —(CH₂)ₘNHC(═O)—R, —(CH₂)ₘN[C(═O)—R]₂—(CH₂)ₘNHS(═O)₂—R, and —C(═O)—NR₂;
R² and R³ are independently selected from C₁₋₆alkyl, C₂₋₆alkenyl, and C₁₋₆alkoxy wherein said C₁₋₆alkyl, C₂₋₆alkenyl, and C₁₋₆alkoxy are optionally substituted by one or more groups selected from amino, halogen, C₁₋₆alkoxy and —CN; or R² and R³ together with the nitrogen connected thereto form a heterocycloalkyl, wherein said heterocycloalkyl is optionally substituted with one or more group selected from C₆₋₁₀aryl, C₂₋₉heteroaryl, C₃₋₆cycloalkyl, C₃₋₅heterocycloalkyl, C₆₋₁₀aryl-C₁₋₃alkyl, C₂₋₉heteroaryl-C₁₋₃alkyl, C₃₋₅heterocycloalkyl-C₁₋₃alkyl, —CN, —SR, —OR, —(CH₂)ₘOR, R, —CO₂R; —SO₂R; —SO₂NR₂, halogen, —NO₂, —NR₂, —(CH₂)ₘNR₂, and —C(═O)—NR₂; and
each R is independently hydrogen, C₁₋₆alkyl, C₂₋₆alkenyl or halogenated C₁₋₆alkyl.

27. A process for preparing a compound of Formula VI, comprising 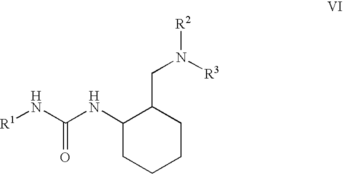
reacting a compound of Formula III with R¹NCO, 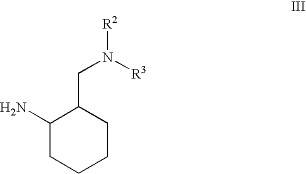
wherein
R¹ is selected from C₆₋₁₀aryl, C₂₋₉heteroaryl, C₃₋₅heterocycloalkyl, C₆₋₁₀aryl-C₁₋₃alkyl, C₂₋₉heteroaryl-C₁₋₃alkyl, C₃₋₅heterocycloalkyl-C₁₋₃alkyl, C₃₋₆cycloalkyl, C₃₋₆cycloalkyl-C₁₋₃alkyl, and C₁₋₆alkyl, wherein said C₆₋₁₀aryl, C₂₋₉heteroaryl, C₆₋₁₀aryl-C₁₋₃alkyl, C₆₋₁₀aryl-O—C₁₋₃alkyl, C₂₋₉heteroaryl-C₁₋₃alkyl, C₃₋₆cycloalkyl, C₃₋₆cycloalkyl-C₁₋₃alkyl, and C₁₋₆alkyl are optionally substituted with one or more group selected from C₆₋₁₀aryl, C₂₋₉heteroaryl, C₃₋₅heterocycloalkyl, C₆₋₁₀aryl-C₁₋₃alkyl, C₆₋₁₀aryl-O—C₁₋₃alkyl, C₂₋₉heteroaryl-C₁₋₃alkyl, C₃₋₅heterocycloalkyl-C₁₋₃alkyl, —CN, —SR, —OR, —O(CH₂)ₘ—OR, R, —C(═O)—R, —CO₂R, —SO₂R, —SO₂NR₂, halogen, —NO₂, —NR₂, —(CH₂)ₘNR₂, —(CH₂)ₘNHC(═O)—NR₂—NHC(═O)—R, —N[C(═O)R]₂, —(CH₂)ₘNHC(═O)—R, —(CH₂)ₘN[C(═O)—R]₂—(CH₂)ₘNHS(═O)₂—R, and —C(═O)—NR₂;
R² and R³ are independently selected from C₁₋₆alkyl, C₂₋₆alkenyl, and C₁₋₆alkoxy wherein said C₁₋₆alkyl, C₂₋₆alkenyl, and C₁₋₆alkoxy are optionally substituted by one or more groups selected from amino, halogen, C₁₋₆alkoxy and —CN; or R² and R³ together with the nitrogen connected thereto form a heterocycloalkyl, wherein said heterocycloalkyl is optionally substituted with one or more group selected from C₆₋₁₀aryl, C₂₋₉heteroaryl, C₃₋₆cycloalkyl, C₃₋₅heterocycloalkyl, C₆₋₁₀aryl-C₁₋₃alkyl, C₂₋₉heteroaryl-C₁₋₃alkyl, C₃₋₅heterocycloalkyl-C₁₋₃alkyl, —CN, —SR, —OR, —(CH₂)ₘOR, R, —CO₂R; —SO₂R; —SO₂NR₂, halogen, —NO₂, —NR₂, —(CH₂)ₘNR₂, and —C(═O)—NR₂; and
each R is independently hydrogen, C₁₋₆alkyl, C₂₋₆alkenyl or halogenated C₁₋₆alkyl.

28. A method for the therapy of anxiety in a warm-blooded animal, comprising the step of administering to said animal in need of such therapy a therapeutically effective amount of a compound according to claim 1.

29. A method for the therapy of anxiety in a warm-blooded animal, comprising the step of administering to said animal in need of such therapy a therapeutically effective amount of a compound according to claim 7.

30. A method for the therapy of anxiety in a warm-blooded animal, comprising the step of administering to said animal in need of such therapy a therapeutically effective amount of a compound according to claim 8.

31. A method for the therapy of depression in a warm-blooded animal, comprising the step of administering to said animal in need of such therapy a therapeutically effective amount of a compound according to claim 1.

32. A method for the therapy of depression in a warm-blooded animal, comprising the step of administering to said animal in need of such therapy a therapeutically effective amount of a compound according to claim 7.

33. A method for the therapy of depression in a warm-blooded animal, comprising the step of administering to said animal in need of such therapy a therapeutically effective amount of a compound according to claim 8.
